(19) United States
(12) Patent Application Publication
(10) Pub. No.: US 2005/0090529 A1
(43) Pub. Date: Apr. 28, 2005

(54) 3,5 Disubstituted indazole compounds with nitrogen-bearing 5-membered heterocycles, pharmaceutical compositions, and methods for mediating or inhibiting cell proliferation

(75) Inventors: Indrawan James McAlpine, San Diego, CA (US); Judith Gail Deal, Wildomar, CA (US); Mary Catherine Johnson, San Diego, CA (US); Susan Elizabeth Kephart, San Diego, CA (US); Julie Yongsun Park, Cambridge, MA (US); William Henry Romines III, San Diego, CA (US); Jayashree G. Tikhe, San Diego, CA (US)

Correspondence Address: AGOURON PHARMACEUTICALS, INC., 10350 NORTH TORREY PINES ROAD, LA JOLLA, CA, 92037, US

(73) Assignee: PFIZER INC

(21) Appl. No.: 10/910,217

(22) Filed: Aug. 2, 2004

Related U.S. Application Data
(60) Provisional application No. 60/491,821, filed on Jul. 31, 2003.

(51) Int. Cl. A61K 31/4439; C07D 43/14

(52) U.S. Cl. 514/338; 546/268.1

(57) ABSTRACT

3,5 disubstituted indazole compounds with substituted nitrogen bearing 5-membered heterocycles in the 3-position that modulate and/or inhibit cell proliferation, such as the activity of protein kinases are described. These compounds and pharmaceutical compositions containing them are capable of mediating CDK dependent diseases to modulate and/or inhibit unwanted cell proliferation. The invention is also directed to the therapeutic or prophylactic use of pharmaceutical compositions containing such compounds, and to methods of treating cancer as well as other disease states associated with unwanted angiogenesis and/or cellular proliferation, such as diabetic retinopathy, neovascular glaucoma, rheumatoid arthritis, and psoriasis, by administering effective amounts of such compounds.

This application claims the benefit of U.S. Provisional Application No. 60/491,821 filed on Jul. 31, 2003, the contents of which is hereby incorporated by reference in its entirety.

### FIELD OF THE INVENTION

This invention is directed to 3,5 disubstituted indazoles with substituted nitrogen bearing 5-membered heterocycles in the 3-position which mediate and/or inhibit cell proliferation through the activity of protein kinases, particularly through mediation of cyclin dependent kinases such as CDK1, CDK2, CDK4, and CDK6. The invention is further related to pharmaceutical compositions containing such compounds and compositions, and to methods of treating cancer as well as other disease states associated with unwanted angiogenesis and/or cellular proliferation, by administering effective amounts of such compounds.

### BACKGROUND OF THE INVENTION

Cell proliferation occurs in response to various stimuli and may stem from de-regulation of the cell division cycle (or cell cycle), the process by which cells multiply and divide. Hyperproliferative disease states, including cancer, are characterized by cells rampantly winding through the cell cycle with uncontrolled vigor due to, for example, damage to the genes that directly or indirectly regulate progression through the cycle. Thus, agents that modulate the cell cycle, and thus hyperproliferation, could be used to treat various disease states associated with uncontrolled or unwanted cell proliferation.

Mechanisms of cell proliferation are under active investigation at cellular and molecular levels. At the cellular level, de-regulation of signaling pathways, loss of cell cycle controls, unbridled angiogenesis or stimulation of inflammatory pathways are under scrutiny, while at the molecular level, these processes are modulated by various proteins, among which protein kinases are prominent suspects. Overall abatement of proliferation may also result from programmed cell death, or apoptosis, which is also regulated via multiple pathways, some involving proteolytic enzyme proteins. Among the candidate regulatory proteins, protein kinases are a family of enzymes that catalyze phosphorylation of the hydroxyl group of specific tyrosine, serine or threonine residues in proteins. Typically, such phosphorylation dramatically perturbs the function of the protein, and thus protein kinases are pivotal in the regulation of a wide variety of cellular processes. For example, without wishing to be bound to a particular theory, it is believed that as inhibitors of protein kinases, such as, for example, cyclin dependent kinases (“CDK”), the inventive agents can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like. (See Schang, et al, J. Virol. 74, 2107-2120 (2000)). Additionally, CDK5 has been implicated in the phosphorylation of tau protein, suggesting potential methods of treating or preventing Alzheimer's disease (Hosoi, et al, J. Biochem. (Tokyo), 117, 741-749 (1995)). CDKs are serine-threonine protein kinases that play critical roles in regulating the transitions between different phases of the cell-cycle, such as the progression from a quiescent stage in G₁ (the gap between mitosis and the onset of DNA replication for a new round of cell division) to S (the period of active DNA synthesis), or the progression from G₂ to M phase, in which active mitosis and cell-division occurs. CDK complexes are formed through association of a regulatory cyclin subunit (e.g., cyclin A, B1, B2, D1, D2, D3, and E) and a catalytic kinase subunit (e.g., CDK1, CDK2, CDK4, CDK5, and CDK6). As the name implies, the CDKs display an absolute dependence on the cyclin subunit in order to phosphorylate their target substrates, and different kinase/cyclin pairs function to regulate progression through specific phases of the cell-cycle.

A number of indazole derivatives have thus far been identified to have therapeutic potential: GB 2345486 discloses indazole derivatives as tyrosine kinase inhibitors, EP0518805 identifies indazoles substituted with piperidines having sigma receptor activity; WO 89/43969 discloses indazoles of cyclic ureas useful as HIV protease inhibitors; U.S. Pat. No. 4,415,569 identifies pyrazoloindazole derivatives having bronchodilating action; U.S. Pat. No. 5,208,248 discloses indazoles for the treatment of migraines. Other therapeutic applications for indazole derivatives are discussed in WO 96/20192, EP 04994774, JP 60/004184, EP0023633, U.S. Pat. No. 4,051,145, JP59/228248, GB 1/376600, U.S. Pat. No. 4,978,603, EP0904769 and in the literature by De Lucca et al, Journal of Medicinal Chemistry, 42, 135-52 (1999). General synthetic schemes for the preparation of indazole derivatives are disclosed Wentrup et al, Journal of Organic Chemistry, 43, 2037-41(1978); Fugimura et al, Chemical Abbstracts, 1070, 749 (1987). More particularly, 3, 5 substituted indazoles have been identified as protein kinase inhibitors: WIPO International Publication No. 01/85726 discloses indazole compounds substituted with 1,1-dioxoisothiazolidine as CDK inhibitors; WO 02/10137 discloses 3,5 substituted indazoles as inhibitors of Jun N-terminal kinase inhibitors; and U.S. Pat. No. 6,555,539 and WO 03/004488discloses 3,5 substituted indazoles with a benzimidazole in the 3-position.

There is still a need, however, for potent inhibitors of CDK which also have, in particular, high solubility for formulation purposes. The inventive CDK inhibitors are generally potent and more soluble than the compounds described in previous publications.

### SUMMARY OF THE INVENTION

An object of the invention is to provide potent and highly soluble inhibitors of CDK. Accordingly, one object of the invention is to attain compounds and drug compositions that inhibit CDK activity, or cyclin complexes thereof. A further object is to provide an effective method of treating cancer indications through CDK inhibition. Another object is to achieve pharmaceutical compositions containing compounds effective to block the transition of cancer cells into their proliferative phase. These and other objects and advantages of the invention, which will become apparent in light of the detailed description below, are achieved through use of cell-cycle control agents of the invention described below.

According to these objectives, there is provided in accordance with the present invention a compound, or pharmaceutically salt or solvate of the compound, of Formula I: 1. A compound or pharmaceutically acceptable salt or solvate of the Formula I: 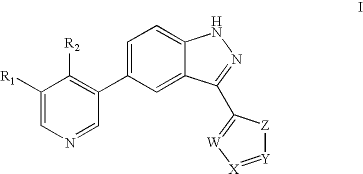
wherein W is —C— or —N—;
X and Y are independently —N—, —C—R³, —C—R⁴;
Z is —C—, —NH—, —O—, or —S—;
wherein R¹ and R² are selected from the group consisting of H, C₁-C₆alkyl, NR⁹R¹⁰, (C₁-C₆alkyl)NR⁹R¹⁰, R⁹, (C₁-C₆alkyl)OR⁹, and R¹ and R² may together optionally cyclize to form a C₃-C₁₀cycloalkyl or a 4-10 membered heterocyclic;
wherein R³ and R⁴ are independently H, halo, cyano, nitro, trifluoromethoxy, trfluoromethyl, azido, hydroxy, or a group, optionally substituted with at least one R⁹, selected from C₁-C₆ alkoxy, C₁-C₁₀ alkyl, C₂-C₆ alkenyl, C₂-C₆ alkynyl, —C(O)R⁵, C(O)OR⁵OC(O)R⁵, NR⁵C(O)R⁶, C(O)NR⁷R⁸, (CR⁵R⁶)ₜNR⁷R⁸, NR⁵OR⁶, —SO₂NR⁷R⁸, S(O)ⱼ(C₁-C₆ alkyl) wherein j is an integer from 0 to 2, (CR⁵R⁶)ₜ(C₆-C₁₀ aryl), (CR⁵R⁶)ₜ(C₃-C₁₀ cycloalkyl), (CR⁵R⁶)ₜ(4-10 membered heterocyclic), (CR⁵R⁶)_{q}C(O)(CR⁷R⁸)ₜ(C₆-C₁₀ aryl), (CR⁵R⁶)_{q}C(O)(CR⁷R⁸)ₜ(C₃-C₁₀cycloalkyl), (CR⁵R⁶)_{q}C(O)(CR⁷R⁸)ₜ(4-10 membered heterocyclic), (CR⁵R⁶)ₜO(CR⁷R⁸)_{q}(C₆-C₁₀ aryl), (CR⁵R⁶)ₜO(CR⁷R⁸)_{q}(C₃-C₁₀ cycloalkyl), (CR⁵R⁶)ₜO(CR⁷R⁸)_{q}(4-10 membered heterocyclic), (CR⁵R⁶)_{q}SO₂(CR⁷R⁸)ₜ(C₆-C₁₀ aryl), (CR⁵R⁶)_{q}SO₂(CR⁷R⁸)ₜ(C₃-C₁₀cycloalkyl) and (CR⁵R⁶)_{q}SO₂(CR⁷R⁸)ₜ(4-10 membered heterocyclic), wherein q and t are each independently an integer from 0 to 5, wherein when R³ and R⁴ are each attached to different carbons, may together optionally cyclize to form a fused 6-membered cycloalkyl ring; wherein R¹, R², R³ and R⁴ are not H at the same time;
wherein each R⁵, R⁶, R⁷ and R⁸ is independently H or a group, optionally substituted with at least one R⁹, selected from C₁-C₆ alkyl, C₁-C₈alkenyl, OR¹⁰, (C₁-C₆alkyl)-OR¹⁰, NR¹⁰R¹¹, (C₁-C₆alkyl)-NR¹⁰R¹¹, C(O)NR¹⁰R¹¹, (C₁-C₆alkyl)C(O)NR¹⁰R¹¹, (C₁-C₆alkyl)-SR¹⁰, aryl, 3-10 membered cycloalkyl, 4-10 membered heterocyclic, (C₁-C₆alkyl)-aryl, (C₁-C₆alkyl)-cycloalkyl, and (C₁-C₆alkyl)-4-10 membered heterocyclic, or wherein when R⁷ and R⁸ are both attached to the same N, may together cyclize to form a 4-10 membered heterocyclic;
wherein R⁹ may be halo, CF₃, CN, C₁-C₆alkyl, OR¹², (C₁-C₆alkyl)-OR¹², COR¹², COOR¹², CONR¹²R¹³, NR¹²R¹³, SR¹², SO₂R¹², NHC(O)CF₃, aryl, haloaryl, O-aryl, (C₁-C₆alkyl)-aryl, or (C₁-C₆alkyl)-4-10 membered heterocyclic;
wherein each R¹⁰, R¹¹, R¹² and R¹³ is independently H, C₁-C₆alkyl, C₁-C₆alkoxyl, C₁-C₆alkenyl, aryl, (C₁-C₆alkyl)-aryl, or when attached to the same N may optionally cyclize to form a 4-10 membered heterocyclic; and
wherein 1 or 2 ring carbon atoms of any of the foregoing cycloalkyl or heterocyclic moieties are optionally substituted with an oxo (═O) moiety.

In one embodiment of the compound of Formula I, W is N, Z is NH, and X and Y are CH, C—R³ or C—R⁴. In a further embodiment, R¹ is ethylaminomethyl and R² is methyl. In a still further embodiment, X is C—R³ and Y is C—R⁴, where R³ and R⁴ are C₁-C₆alkyl.

In an alternative embodiment of the compound of Formula I, W is N, Z is NH, X is CH, Y is C—R³ where R³ is CONR⁷R⁸, R¹ is ethylaminomethyl and R² is ethyl.

The invention further comprises a compound, or pharmaceutically acceptable salt or solvate selected from the group consisting of

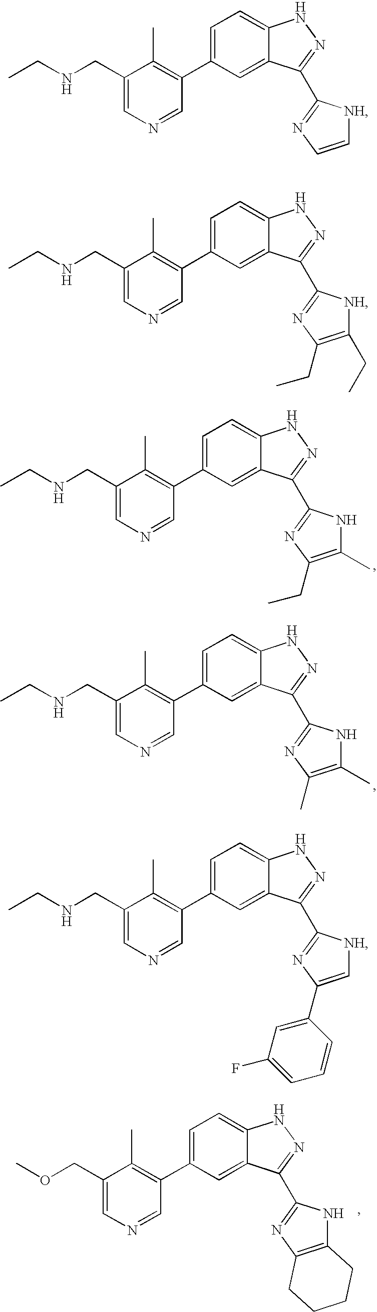

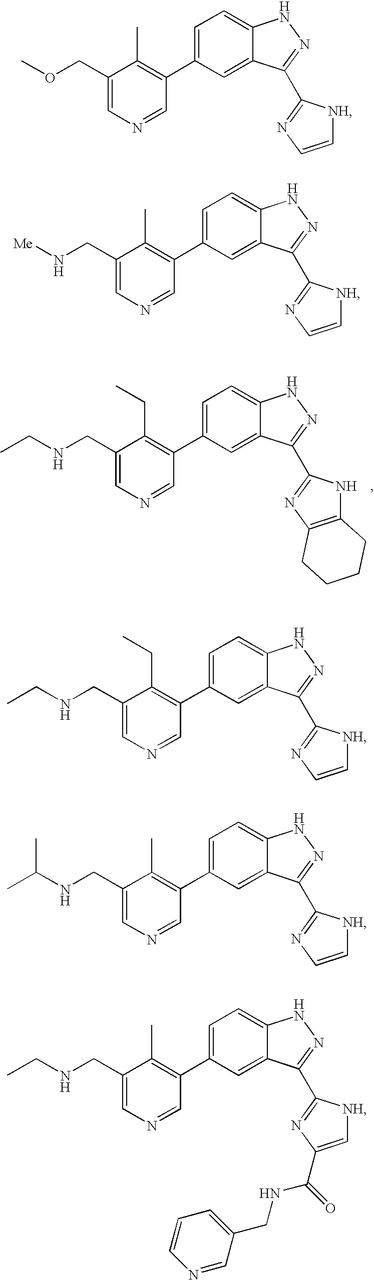

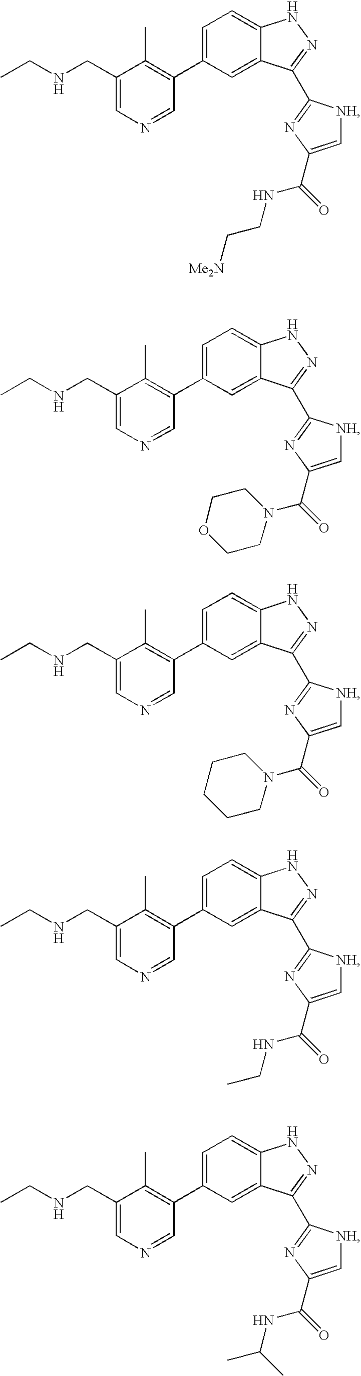

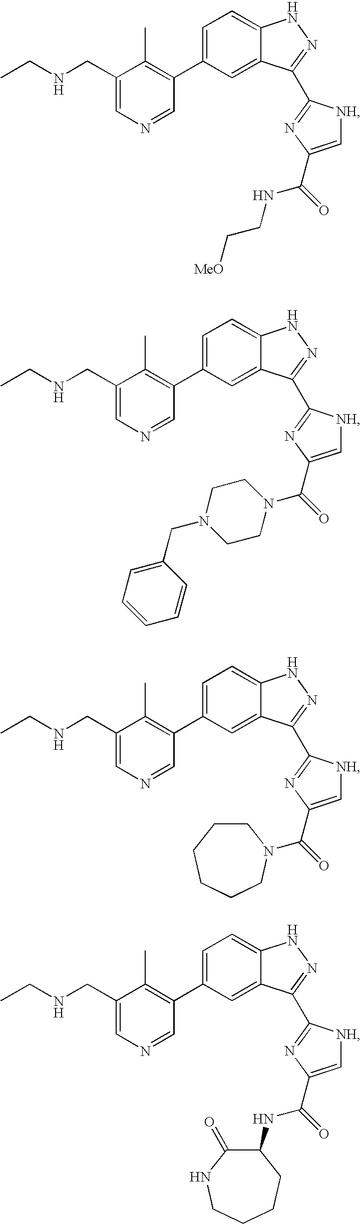

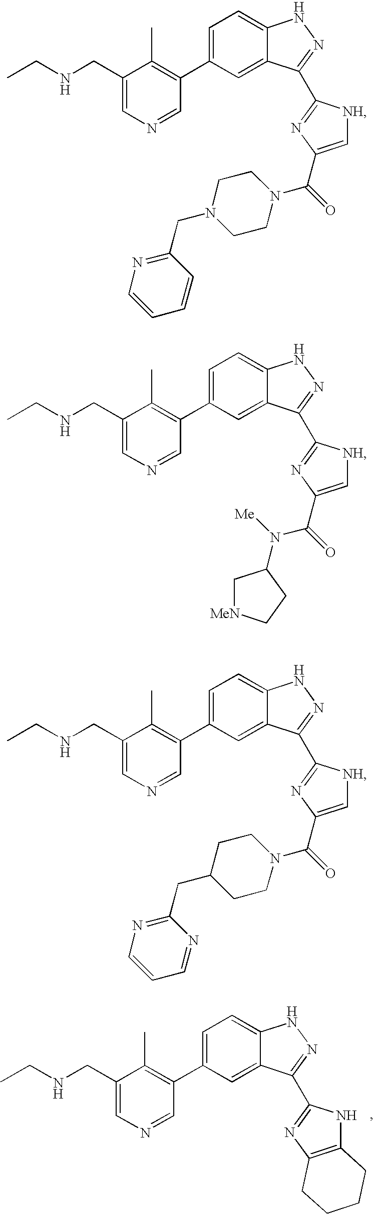

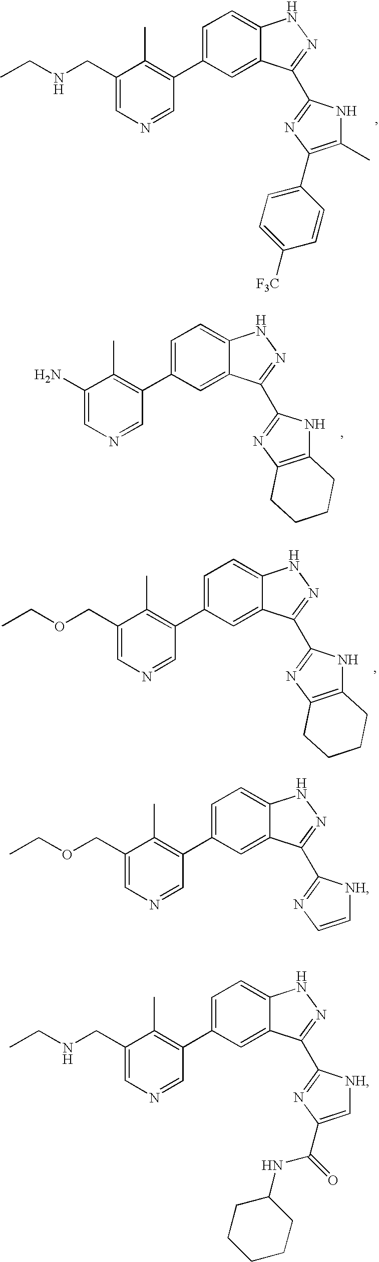

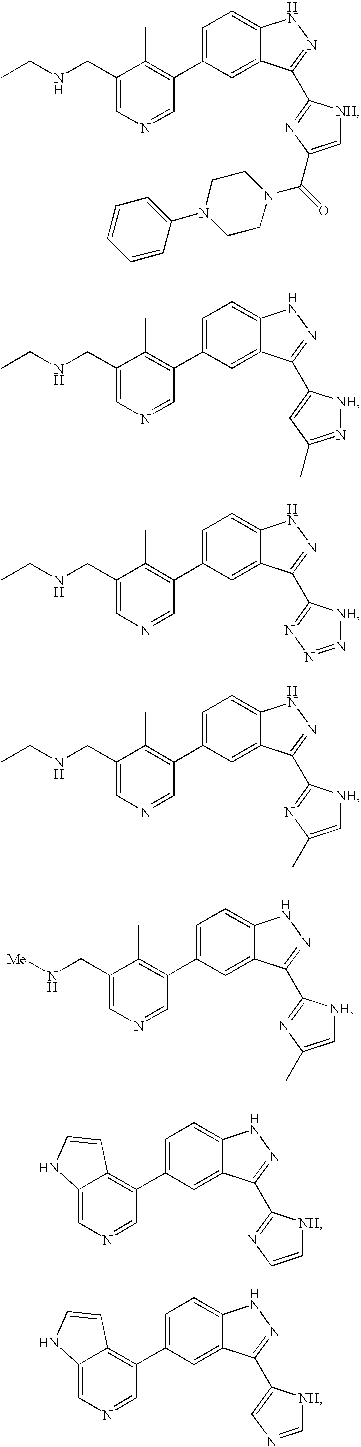

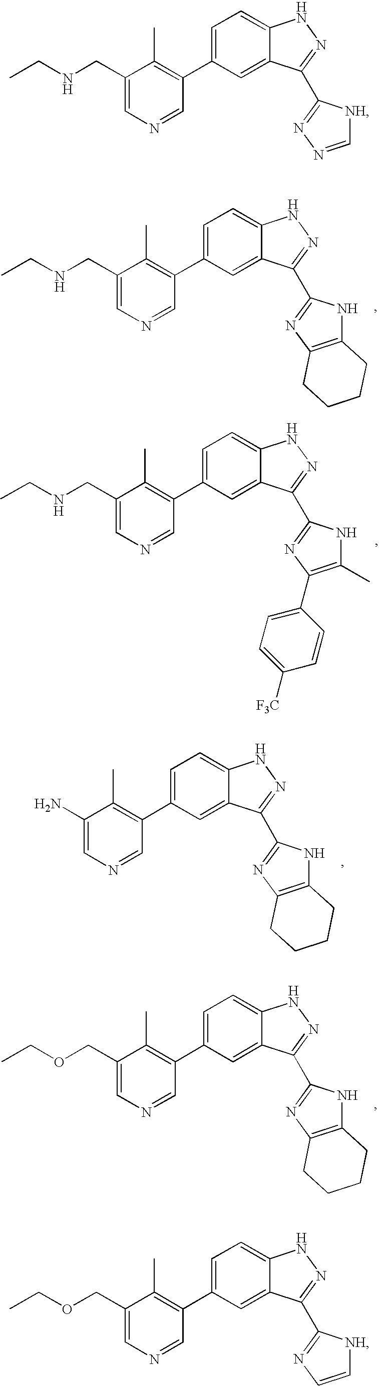

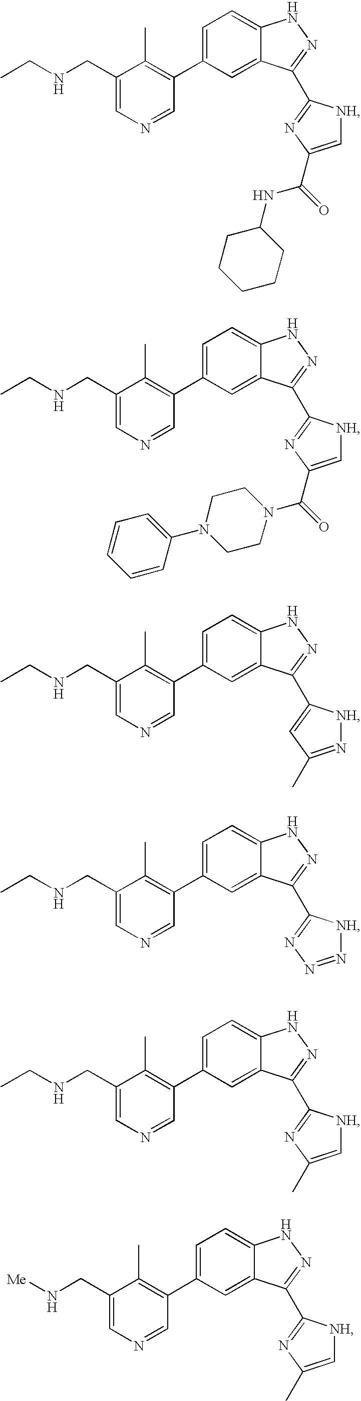

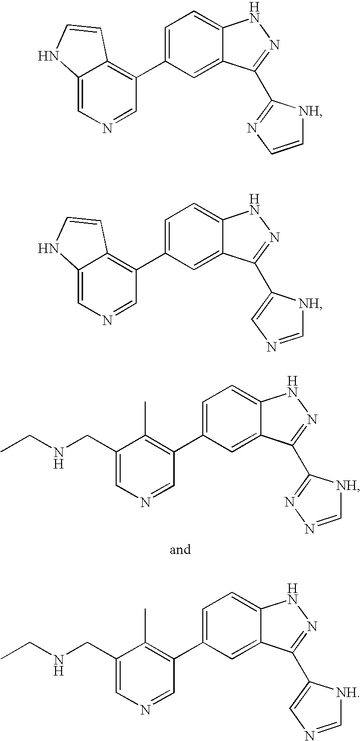

The invention also provides a method of modulating and/or inhibiting kinase activity of by administering a compound of the Formula I or a pharmaceutically acceptable salt or solvate of a compound of the Formula I, to a patient in need thereof.

There is also provided in accordance with the invention, a pharmaceutical composition containing a compound of the Formula I or a pharmaceutically acceptable salt or solvate of a compound of the Formula I, and the therapeutic use of the composition in treating diseases mediated by kinase activity, such as cancer, as well as other disease states associated with unwanted angiogenesis and/or cellular proliferation, such as diabetic retinopathy, neovascular glaucoma, rheumatoid arthritis, and psoriasis.

The inventive agents and compositions containing such agents may be useful in treating various disorders or disease states associated with uncontrolled or unwanted cellular proliferation, such as cancer, autoimmune disorders, viral diseases, fungal diseases, neurodegenerative disorders, and cardiovascular diseases. Thus, the invention is also directed to methods of treating such diseases by administering an effective amount of the inventive agent.

Other aspects, advantages, and features of the invention will become apparent from the detailed description below.

The compounds and compositions of the present invention, are useful as anti-proliferative agents and as inhibitors of mammalian kinase complexes, insect kinase or fungal kinase complexes. For example, CDK complexes can be inhibited. Such compounds and compositions are also useful for controlling proliferation, differentiation, and/or apoptosis.

The term “halo”, as used herein, unless otherwise indicated, means fluoro, chloro, bromo or iodo. Preferred halo groups are fluoro, chloro and bromo.

The term “alkyl”, as used herein, unless otherwise indicated, includes saturated monovalent hydrocarbon radicals having straight or branched moieties. A “C₁-C₆ alkyl” indicates a straight or branched alkyl moiety having 1 to 6 carbon atoms, and so forth.

The term “alkenyl” refers to a straight- or branched-chain alkenyl group having 2 to 12 carbon atoms in the chain. Illustrative alkenyl groups include prop-2-enyl, but-2-enyl, but-3-enyl, 2-methylprop-2-2nyl, hex-2-2nyl, ethenyl, pentenyl, and the like.

The term “alkynyl” refers to a straight- or branched-chain alkynyl group having from 2 to 12 carbon atoms in the chain. Illustrative alkynyl groups include prop-2-ynyl, but-2-ynyl, but-3-ynyl, 2-methylbut-2-ynyl, hex-2-ynyl, ethynyl, propynyl, pentynyl and the like.

The term “cycloalkyl” refers to a saturated or partially saturated, monocyclic or fused or spiro polycyclic, carbocycle having from three to twelve ring atoms per ring. Illustrative examples of cycloalkyl groups include the following moieties:

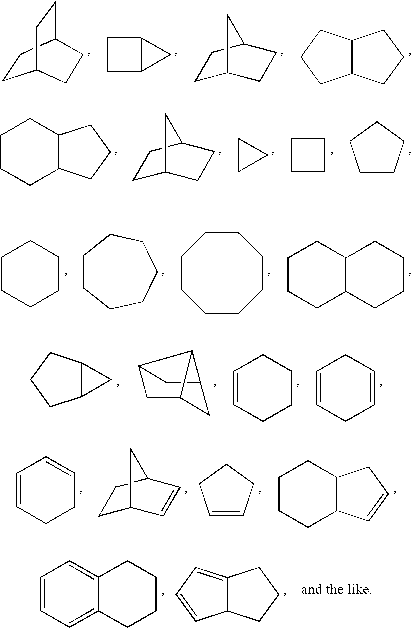

The term “aryl”, as used herein, unless otherwise indicated, includes an organic radical derived from an aromatic hydrocarbon by removal of one hydrogen, such as phenyl or naphthyl.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azebdinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). The 4-10 membered heterocyclic may be optionally substituted on any ring carbon, sulfur, or nitrogen atom(s) by one to two oxo, per ring. An example of a heterocyclic group wherein 2 ring carbon atoms are substituted with oxo moieties is 1,1-dioxo-thiomorpholinyl. Other Illustrative examples of 4-10 membered heterocyclic are derived from, but not limited to, the following:

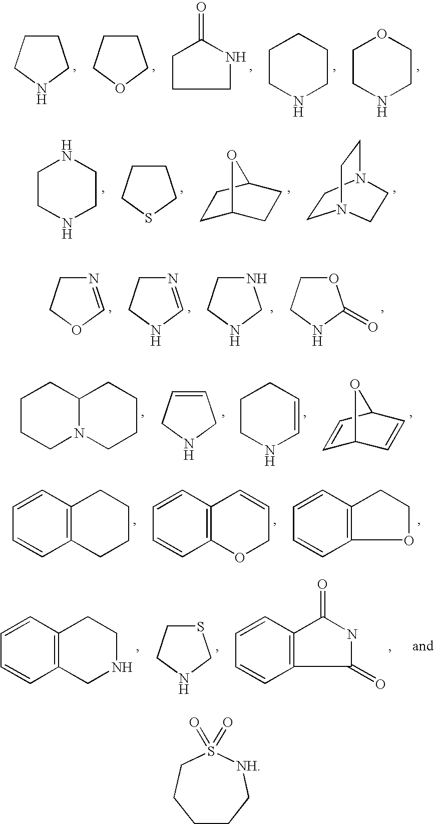

Unless otherwise indicated, the term “oxo” refers to ═O.

The term “amide” refers to the radical —C(O)N(R′)(R″) where R′ and R″ are each independently selected from hydrogen, alkyl, alkenyl, alkynyl, —OH, alkoxy, cycloalkyl, heterocycloalkyl, heteroaryl, aryl as defined above; or R′ and R″ cyclize together with the nitrogen to form a heterocycloalkyl or heteroaryl as defined above.

The term “substituted” means that the specified group or moiety bears one or more substituents. The term “unsubstituted” means that the specified group bears no substituents.

Within the invention it is understood that a compound of Formula I may exhibit the phenomenon of tautomerism and that the formula drawings within this specification represent only one of the possible tautomeric forms. It is to be understood that the invention encompasses any tautomeric form which modulates and/or inhibits kinase activity and is not to be limited merely to any one tautomeric form utilized within the formula drawings.

Some of the inventive compounds may exist as single stereoisomers (i.e., essentially free of other stereoisomers), racemates, and/or mixtures of enantiomers and/or diastereomers. All such single stereoisomers, racemates and mixtures thereof are intended to be within the scope of the present invention. Preferably, the inventive compounds that are optically active are used in optically pure form.

As generally understood by those skilled in the art, an optically pure compound having one chiral center (i.e., one asymmetric carbon atom) is one that consists essentially of one of the two possible enantiomers (i.e., is enantiomerically pure), and an optically pure compound having more than one chiral center is one that is both diastereomerically pure and enantiomerically pure. Preferably, the compounds of the present invention are used in a form that is at least 90% optically pure, that is, a form that contains at least 90% of a single isomer (80% enantiomeric excess (“e.e.”) or diastereomeric excess (“d.e.”)), more preferably at least 95% (90% e.e. or d.e.), even more preferably at least 97.5% (95% e.e. or d.e.), and most preferably at least 99% (98% e.e. or d.e.).

Additionally, Formulas I and II are intended to cover solvated as well as unsolvated forms of the identified structures. For example, Formulas I and II include compounds of the indicated structure in both hydrated and non-hydrated forms. Other examples of solvates include the structures in combination with isopropanol, ethanol, methanol, DMSO, ethyl acetate, acetic acid, or ethanolamine.

“A pharmaceutically acceptable salt” is intended to mean a salt that retains the biological effectiveness of the free acids and bases of the specified compound and that is not biologically or otherwise undesirable. A compound of the invention may possess a sufficiently acidic, a sufficiently basic, or both functional groups, and accordingly react with any of a number of inorganic or organic bases, and inorganic and organic acids, to form a pharmaceutically acceptable salt. Exemplary pharmaceutically acceptable salts include those salts prepared by reaction of the compounds of the present invention with a mineral or organic acid or an inorganic base, such as salts including sulfates, pyrosulfates, bisulfates, sulfites, bisulfites, phosphates, monohydrogenphosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, xylenesulfonates, phenylacetates, phenylpropionates, phenylbutyrates, citrates, lactates, γ-hydroxybutyrates, glycollates, tartrates, methane-sulfonates, propanesulfonates, naphthalene-1-sulfonates, naphthalene-2-sulfonates, and mandelates.

If the inventive compound is a base, the desired pharmaceutically acceptable salt may be prepared by any suitable method available in the art, for example, treatment of the free base with an inorganic acid, such as hydrochloric acid, hydrobromic acid, sulfuric acid, nitric acid, phosphoric acid and the like, or with an organic acid, such as acetic acid, maleic acid, succinic acid, mandelic acid, fumaric acid, malonic acid, pyrovic acid, oxalic acid, glycolic acid, salicylic acid, a pyranosidyl acid, such as glucuronic acid or galacturonic acid, an alpha-hydroxy acid, such as citric acid or tartaric acid, an amino acid, such as aspartic acid or glutamic acid, an aromatic acid, such as benzoic acid or cinnamic acid, a sulfonic acid, such as p-toluenesulfonic acid or ethanesulfonic acid, or the like.

If the inventive compound is an acid, the desired pharmaceutically acceptable salt may be prepared by any suitable method, for example, treatment of the free acid with an inorganic or organic base, such as an amine (primary, secondary or tertiary), an alkali metal hydroxide or alkaline earth metal hydroxide, or the like. Illustrative examples of suitable salts include organic salts derived from amino acids, such as glycine and arginine, ammonia, primary, secondary, and tertiary amines, and cyclic amines, such as piperidine, morpholine and piperazine, and inorganic salts derived from sodium, calcium, potassium, magnesium, manganese, iron, copper, zinc, aluminum and lithium.

In the case of agents that are solids, it is understood by those skilled in the art that the inventive compounds and salts may exist in different crystal or polymorphic forms, all of which are intended to be within the scope of the present invention and specified formulas.

Cell-cycle control agents in accordance with the invention are useful as pharmaceuticals for treating proliferative disorders in mammals, especially humans, marked by unwanted proliferation of endogenous tissue. Compounds of the Formula I may be used for treating subjects having a disorder associated with excessive cell proliferation, e.g., cancers, psoriasis, immunological disorders involving undesired proliferation of leukocytes, and restenosis and other smooth-muscle disorders. Furthermore, such compounds may be used to prevent de-differentiation of post-mitotic tissue and/or cells.

Diseases or disorders associated with uncontrolled or abnormal cellular proliferation include, but are not limited to, the following:
a variety of cancers, including, but not limited to, carcinoma, hematopoietic tumors of lymphoid lineage, hematopoietic tumors of myeloid lineage, tumors of mesenchymal origin, tumors of the central and peripheral nervous system and other tumors including melanoma, seminoma and Kaposi's sarcoma and the like.
a disease process which features abnormal cellular proliferation, e.g., benign prostatic hyperplasia, familial adenomatosis polyposis, neuro-fibromatosis, atherosclerosis, pulmonary fibrosis, arthritis, psoriasis, glomerulonephritis, restenosis following angioplasty or vascular surgery, hypertrophic scar formation, inflammatory bowel disease, transplantation rejection, endotoxic shock, and fungal infections.
defective apoptosis-associated conditions, such as cancers (including but not limited to those types mentioned hereinabove), viral infections (including but not limited to herpesvirus, poxvirus, Epstein-Barr virus, Sindbis virus and adenovirus), prevention of AIDS development in HIV-infected individuals, autoimmune diseases (including but not limited to systemic lupus erythematosus, rheumatoid arthritis, psoriasis, autoimmune mediated glomerulonephritis, inflammatory bowel disease and autoimmune diabetes mellitus), neurodegenerative disorders (including but not limited to Alzheimer's disease, amyotrophic lateral sclerosis, retinitis pigmentosa, Parkinson's disease, AIDS-related dementia, spinal muscular atrophy and cerebellar degeneration), myelodysplastic syndromes, aplastic anemia, ischemic injury associated with myocardial infarctions, stroke and reperfusion injury, arrhythmia, atherosclerosis, toxin-induced or alcohol related liver diseases, hematological diseases (including but not limited to chronic anemia and aplastic anemia), degenerative diseases of the musculoskeletal system (including but not limited to osteroporosis and arthritis), aspirin-sensitive rhinosinusitis, cystic fibrosis, multiple sclerosis, kidney diseases and cancer pain.

The active agents of the invention may also be useful in the inhibition of the development of invasive cancer, tumor angiogenesis and metastasis.

Moreover, the active agents of the invention, for example, as inhibitors of the CDKs, can modulate the level of cellular RNA and DNA synthesis and therefore are expected to be useful in the treatment of viral infections such as HIV, human papilloma virus, herpesvirus, Epstein-Barr virus, adenovirus, Sindbis virus, poxvirus and the like.

Compounds and compositions of the invention inhibit the kinase activity of, for example, CDK/cyclin complexes, such as those active in the G₀ or G₁ stage of the cell cycle, e.g., CDK2, CDK4, and/or CDK6 complexes.

The specific dosage amount of a cell-cycle control agent being administered to obtain therapeutic or inhibitory effects may be determined in a manner known in the art according to the particular circumstances surrounding the case, including, e.g., the specific agent being administered, the route of administration, the condition being treated, and the subject or host being treated. An exemplary total daily dose of a cell-cycle control agent, which may be administered in single or multiple doses, contains a dosage level of from about 0.01 mg/kg body weight to about 50 mg/kg body weight.

The cell-cycle control agents of the invention may be administered by any of a variety of suitable routes, such as orally, rectally, transdermally, subcutaneously, intravenously, intramuscularly, or intranasally. The cell-cycle control agents are preferably formulated into compositions suitable for the desired routes before being administered.

A pharmaceutical composition or preparation according to the invention comprises an effective amount of a cell-cycle control agent, optionally one or more other active agents, and a pharmaceutically acceptable carrier, such as a diluent or excipient for the agent; when the carrier serves as a diluent, it may be a solid, semi-solid, or liquid material acting as a vehicle, excipient, or medium for the active ingredient(s). Compositions according to the invention may be made by admixing the active ingredient(s) with a carrier, or diluting it with a carrier, or enclosing or encapsulating it within a carrier, which may be in the form of a capsule, sachet, paper container, or the like. Exemplary ingredients, in addition to one or more cell-cycle control agents and any other active ingredients, include Avicel (microcrystalline cellulose), starch, lactose, calcium sulfate dihydrate, terra alba, sucrose, talc, gelatin, agar, pectin, acacia, magnesium stearate, stearic acid, peanut oil, olive oil, glyceryl monostearate, Tween 80 (polysorbate 80), 1,3-butanediol, cocoa butter, beeswax, polyethylene glycol, propylene glycol, sorbitan monostearate, polysorbate 60, 2-octyldodecanol, benzyl alcohol, glycine, sorbic acid, potassium sorbate, disodium hydrogen phosphate, sodium chloride, and water.

The compositions may be prepared in any of a variety of forms suitable for the desired mode of administration. For example, pharmaceutical compositions may be prepared in the form of tablets, pills, powders, lozenges, sachets, cachets, elixirs, suspensions, emulsions, solutions, syrups, aerosols (as solids or in liquid media), ointments (e.g., containing up to 10% by weight of a cell-cycle control agent), soft-gel and hard-gel capsules, suppositories, sterile injectable solutions, sterile packaged powders, and the like.

Similarly, the carrier or diluent may include time-delay or time-release material known in the art, such as glyceryl monostearate or glyceryl distearate alone or with a wax, ethylcellulose, hydroxypropylmethylcellulose, methylmethacrylate and the like.

A variety of pharmaceutical forms can be employed. Thus, if a solid carrier is used, the preparation can be tableted, placed in a hard gelatin capsule in powder or pellet form or in the form of a troche or lozenge. The amount of solid carrier may vary, but generally will be from about 25 mg to about 1 g. If a liquid carrier is used, the preparation can be in the form of syrup, emulsion, soft gelatin capsule, sterile injectable solution or suspension in an ampoule or vial or non-aqueous liquid suspension.

To obtain a stable water-soluble dose form, a pharmaceutically acceptable salt of an inventive agent is dissolved in an aqueous solution of an organic or inorganic acid, such as 0.3M solution of succinic acid or citric acid. If a soluble salt form is not available, the agent may be dissolved in a suitable cosolvent or combinations of cosolvents. Examples of suitable cosolvents include, but are not limited to, alcohol, propylene glycol, polyethylene glycol 300, polysorbate 80, gylcerin and the like in concentrations ranging from 0-60% of the total volume. A compound of Formula I may be dissolved in DMSO and diluted with water. The composition may also be in the form of a solution of a salt form of the active ingredient in an appropriate aqueous vehicle such as water or isotonic saline or dextrose solution.

The compositions of the invention may be manufactured in manners generally known for preparing pharmaceutical compositions, e.g., using conventional techniques such as mixing, dissolving, granulating, dragee-making, levigating, emulsifying, encapsulating, entrapping or lyophilizing. Pharmaceutical compositions may be formulated in a conventional manner using one or more physiologically acceptable carriers, which may be selected from excipients and auxiliaries that facilitate processing of the active compounds into preparations which can be used pharmaceutically.

Proper formulation is dependent upon the route of administration chosen. For injection, the agents of the invention may be formulated into aqueous solutions, preferably in physiologically compatible buffers such as Hanks's solution, Ringer's solution, or physiological saline buffer. For transmucosal administration, penetrants appropriate to the barrier to be permeated are used in the formulation. Such penetrants are generally known in the art.

For oral administration, the compounds can be formulated readily by combining the active compounds with pharmaceutically acceptable carriers known in the art. Such carriers enable the compounds of the invention to be formulated as tablets, pills, dragees, capsules, liquids, gels, syrups, slurries, suspensions and the like, for oral ingestion by a patient to be treated. Pharmaceutical preparations for oral use can be obtained using a solid excipient in admixture with the active ingredient (agent), optionally grinding the resulting mixture, and processing the mixture of granules after adding suitable auxiliaries, if desired, to obtain tablets or dragee cores. Suitable excipients include: fillers such as sugars, including lactose, sucrose, mannitol, or sorbitol; and cellulose preparations, for example, maize starch, wheat starch, rice starch, potato starch, gelatin, gum, hydroxypropylmethyl-cellulose, sodium carboxymethylcellulose, methyl cellulose, or polyvinylpyrrolidone (PVP). If desired, disintegrating agents may be added, such as crosslinked polyvinyl pyrrolidone, agar, or alginic acid or a salt thereof such as sodium alginate.

Dragee cores are provided with suitable coatings. For this purpose, concentrated sugar solutions may be used, which may optionally contain gum arabic, polyvinyl pyrrolidone, Carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active agents.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with fillers such as lactose, binders such as starches, and/or lubricants such as talc or magnesium stearate, and, optionally, stabilizers. In soft capsules, the active agents may be dissolved or suspended in suitable liquids, such as fatty oils, liquid paraffin, or liquid polyethylene glycols. In addition, stabilizers may be added. All formulations for oral administration should be in dosages suitable for such administration. For buccal administration, the compositions may take the form of tablets or lozenges formulated in conventional manner.

For administration intranasally or by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of gelatin for use in an inhaler or insufflator and the like may be formulated containing a powder mix of the compound and a suitable powder base such as lactose or starch.

The compounds may be formulated for parenteral administration by injection, e.g., by bolus injection or continuous infusion. Formulations for injection may be presented in unit-dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

Pharmaceutical formulations for parenteral administration include aqueous solutions of the active compounds in water-soluble form. Additionally, suspensions of the active agents may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

For administration to the eye, the active agent is delivered in a pharmaceutically acceptable ophthalmic vehicle such that the compound is maintained in contact with the ocular surface for a sufficient time period to allow the compound to penetrate the corneal and internal regions of the eye, including, for example, the anterior chamber, posterior chamber, vitreous body, aqueous humor, vitreous humor, cornea, iris/ciliary, lens, choroid/retina and sclera. The pharmaceutically acceptable ophthalmic vehicle may be an ointment, vegetable oil, or an encapsulating material. A compound of the invention may also be injected directly into the vitreous and aqueous humor.

Alternatively, the active ingredient may be in powder form for constitution with a suitable vehicle, e.g., sterile pyrogen-free water, before use. The compounds may also be formulated in rectal compositions such as suppositories or retention enemas, e.g., containing conventional suppository bases such as cocoa butter or other glycerides.

The compounds may also be formulated as a depot preparation. Such long-acting formulations may be administered by implantation (for example, subcutaneously or intramuscularly) or by intramuscular injection. Thus, for example, the compounds may be formulated with suitable polymeric or hydrophobic materials (for example, as an emulsion in an acceptable oil) or ion-exchange resins, or as sparingly soluble derivatives, for example, as a sparingly soluble salt.

A pharmaceutical carrier for hydrophobic compounds is a cosolvent system comprising benzyl alcohol, a nonpolar surfactant, a water-miscible organic polymer, and an aqueous phase. The cosolvent system may be a VPD co-solvent system. VPD is a solution of 3% w/v benzyl alcohol, 8% w/v of the nonpolar surfactant polysorbate 80, and 65% w/v polyethylene glycol 300, made up to volume in absolute ethanol. The VPD co-solvent system (VPD:5W) contains VPD diluted 1:1 with a 5% dextrose in water solution. This co-solvent system dissolves hydrophobic compounds well, and itself produces low toxicity upon systemic administration. Naturally, the proportions of a co-solvent system may be varied considerably without destroying its solubility and toxicity characteristics. Furthermore, the identity of the co-solvent components may be varied: for example, other low-toxicity nonpolar surfactants may be used instead of polysorbate 80; the fraction size of polyethylene glycol may be varied; other biocompatible polymers may replace polyethylene glycol, e.g. polyvinyl pyrrolidone; and other sugars or polysaccharides may be substituted for dextrose.

Alternatively, other delivery systems for hydrophobic pharmaceutical compounds may be employed. Liposomes and emulsions are known examples of delivery vehicles or carriers for hydrophobic drugs. Certain organic solvents such as dimethylsulfoxide also may be employed, although usually at the cost of greater toxicity. Additionally, the compounds may be delivered using a sustained-release system, such as semipermeable matrices of solid hydrophobic polymers containing the therapeutic agent. Various sustained-release materials have been established and are known by those skilled in the art. Sustained-release capsules may, depending on their chemical nature, release the compounds for a few weeks up to over 100 days. Depending on the chemical nature and the biological stability of the therapeutic reagent, additional strategies for protein stabilization may be employed.

The pharmaceutical compositions also may comprise suitable solid- or gel-phase carriers or excipients. Examples of such carriers or excipients include calcium carbonate, calcium phosphate, sugars, starches, cellulose derivatives, gelatin, and polymers such as polyethylene glycols.

Some of the compounds of the invention may be provided as salts with pharmaceutically compatible counter ions. Pharmaceutically compatible salts may be formed with many acids, including hydrochloric, sulfuric, acetic, lactic, tartaric, malic, succinic, etc. Salts tend to be more soluble in aqueous or other protonic solvents than are the corresponding free-base forms.

A pharmaceutical composition according to the invention comprises a cell-cycle control agent and, optionally, one or more other active ingredients, such as a known antiproliferative agent that is compatible with the cell-cycle control agent and suitable for the indication being treated.

The compounds are useful as anti-angiogenesis agents and as agents for modulating and/or inhibiting the activity of protein kinases, thus providing treatments for cancer or other diseases associated with cellular proliferation mediated by protein kinases.

Therapeutically effective amounts of the agents of the invention may be used to treat diseases mediated by modulation or regulation of protein kinases. An “effective amount” is intended to mean that amount of an agent that, when administered to a mammal in need of such treatment, is sufficient to effect treatment for a disease mediated by the activity of one or more kinases. Thus, e.g., a therapeutically effective amount of a compound of the Formula I, salt, active metabolite or prodrug thereof is a quantity sufficient to modulate, regulate, or inhibit the activity of one or more kinases such that a disease condition which is mediated by that activity is reduced or alleviated.

“Treating” is intended to mean at least the mitigation of a disease condition in a mammal, such as a human, that is affected, at least in part, by the activity of one or more kinases, and includes: preventing the disease condition from occurring in a mammal, particularly when the mammal is found to be predisposed to having the disease condition but has not yet been diagnosed as having it; modulating and/or inhibiting the disease condition; and/or alleviating the disease condition.

In a specific embodiment of any of the inventive methods described herein, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In further specific embodiments of any of the inventive methods described herein, the method further comprises administering to the mammal an amount of one or more substances selected from anti-tumor agents, anti-angiogenesis agents, signal transduction inhibitors, and antiproliferative agents, which amounts are together effective in treating said abnormal cell growth. The compounds of the present invention may be combined with other anti-tumor agents, the methods of which are disclosed in WO038716, WO038717, WO038715, WO038730, WO038718, WO038665, WO037107, WO038786, WO038719, the contents of which are herein incorporated by reference in their entireties. Examples of anti-tumor agents include mitotic inhibitors, for example vinca alkaloid derivatives such as vinblastine vinorelbine, vindescine and vincristine; colchines allochochine, halichondnne, N-benzoyltrmethyl-methyl ether colchicinic acid, dolastatin 10, maystansine, rhizoxine, taxanes such as taxol (paclitaxel), docetaxel (Taxotere), 2′-N-[3-(dimethylamino)propyl]glutaramate (taxol derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, trethylenemelamine, uracil nitrogen mustard, bis(3-mesyloxypropyl)amine hydrochloride, mitomycin, nitrosoureas agents such as cyclohexyl-chloroethylnitrosourea, methylcyclohexyl-chloroethylnitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxuridine and fludarabine. DNA/RNA antimetabolites, for example, L-alanosine, 5-azacytidine, acivicin, aminopterin and derivatives thereof such as N-[2-chloro-5-[[(2, 4-diamino-5-methyl-6-quinazolinyl)methyl]amino]benzoyl]-L-aspartic acid, N-[4-[[(2, 4-diamino-5-ethyl-6-quinazolinyl)methyl]amino]benzoyl]-L-aspartic acid, N -[2-chloro-4-[[(2,4-diaminopteridinyl)methyl]amino]benzoyl]-L-aspartic acid, soluble Baker's antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred anti-metabolites disclosed in European Patent Application No. 239362 such as N-(5-LN-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as Nolvadex™ (tamoxifen) or, for example anti-androgens such as Casodexm (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents include MMP-2 (matrix-metalloprotienase 2) inhibitors, MMP-9 (matrix-metalloprotienase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26,1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (ie. MMP-1, MMP-3, MMP-4, MMP-5, MMP-6, MMP-7, MMP-8, MMP-10, MMP-11, MMP-12, and MMP-13).

Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example SU-5416 and SU-6668 (Sugen Inc. of South San Francisco, Calif., USA), can also be combined or co-administered with a compound of formula 1. VEGF inhibitors are described in, for example in WO 99/24440 (published May 20,1999), PCT International Application PCT/IB99/00797 (filed May 3,1999), in WO 95/21613 (published Aug. 17, 1995), WO 99/61422 (published Dec. 2, 1999), U.S. Pat. No. 5,834,504 (issued Nov. 10,1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11,1998), WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); anti-VEGF monoclonal antibody of Genentech, Inc. of South San Francisco, Calif.; and angiozyme, a synthetic ribozyme from Ribozyme (Boulder, Colo.) and Chiron (Emeryville, Calif.). ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with a compound of formula 1. Such erbB2 inhibitors include those described in WO 98/02434 (published Jan. 22,1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used include inhibitors of the enzyme farnesyl protein transferase and inhibitors of the receptor tyrosine kinase PDGFr, including the compounds disclosed and claimed in the following U.S. patent applications: Ser. No. 09/221946 (filed Dec. 28, 1998); Ser. No. 09/454058 (filed Dec. 2, 1999); Ser. No. 09/501163 (filed Feb. 9, 2000); Ser. No. 09/539930 (filed Mar. 31, 2000); Ser. No. 09/202796 (filed May 22, 1997); Ser. No. 09/384339 (filed Aug. 26, 1999); and Ser. No. 09/383755 (filed Aug. 26,1999); and the compounds disclosed and claimed in the following U.S. provisional patent applications: 60/168207 (filed Nov. 30, 1999); 60/170119 (filed Dec. 10, 1999); 60/177718 (filed Jan. 21, 2000); 60/168217 (filed Nov. 30, 1999), and 60/200834 (filed May 1, 2000). Each of the foregoing patent applications and provisional patent applications is herein incorporated by reference in their entirety.

The compound of formula 1 may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

### DETAILED DESCRIPTION AND PREFERRED EMBODIMENTS OF THE INVENTION

The inventive agents may be prepared using the reaction routes and synthesis schemes as described below, employing the techniques available in the art using starting materials that are readily available.

The preparation of specific preferred compounds of the invention is described in detail in the following examples. The artisan will recognize that the chemical reactions described may be readily adapted to prepare a number of other kinase inhibitors of the invention. For example, the synthesis of non-exemplified compounds according to the invention may be successfully performed by modifications apparent to those skilled in the art, e.g., by appropriately protecting interfering groups, by changing to other suitable reagents known in the art, or by making routine modifications of reaction conditions. Alternatively, other reactions disclosed herein or known in the art will be recognized as having applicability for preparing other compounds of the invention.

In the examples described below, unless otherwise indicated all temperatures are set forth in degrees Celsius and all parts and percentages are by weight. Reagents were purchased from commercial suppliers such as Aldrich Chemical Company or Lancaster Synthesis Ltd. and were used without further purification unless otherwise indicated. Tetrahydrofuran (THF) distilled from calcium hydride and N, N-dimethylformamide (DMF) were purchased from Aldrich in Sure seal bottles and used as received. All solvents were purified using standard methods readily known to those skilled in the art, unless otherwise indicated.

The reactions set forth below were done generally under a positive pressure of argon or with a drying tube, at ambient temperature (unless otherwise stated), in anhydrous solvents, and the reaction flasks were fitted with rubber septa for the introduction of substrates and reagents via syringe. Glassware was oven dried and/or heat dried. Analytical thin layer chromatography (TLC) was performed on glass-backed silica gel 60 F 254 plates Analtech (0.25 mm) and eluted with the appropriate solvent ratios (v/v), and are denoted where appropriate. The reactions were assayed by TLC and terminated as judged by the consumption of starting material.

Visualization of the TLC plates was done with a panisaldehyde spray reagent or phosphomolybdic acid reagent (Aldrich Chemical 20 wt % in ethanol) and activated with heat. Work-ups were typically done by doubling the reaction volume with the reaction solvent or extraction solvent and then washing with the indicated aqueous solutions using 25% by volume of the extraction volume unless otherwise indicated. Product solutions were dried over anhydrous Na₂SO₄ or MgSO₄ prior to filtration and evaporation of the solvents under reduced pressure on a rotary evaporator and noted as solvents removed in vacuo. Flash column chromatography (Still et al., J. Org. Chem., 43, 2923 (1978)) was done using Baker grade flash silica gel (47-61 μm) and a silica gel: crude material ratio of about 20:1 to 50:1 unless otherwise stated. Hydrogenation was done at the pressure indicated in the examples or at ambient pressure.

¹H-NMR spectra were recorded on a Bruker instrument operating at 300 MHz or 500 MHz and ¹³C-NMR spectra were recorded operating at 75 MHz. NMR spectra were obtained as CDCl₃ solutions (reported in ppm), using chloroform as the reference standard (7.25 ppm and 77.00 ppm) or CD₃OD (3.4 ppm and 4.8 ppm and 49.3 ppm), or internal tetramethylsilane (0.00 ppm) when appropriate. Other NMR solvents were used as needed. When peak multiplicities are reported, the following abbreviations are used: s (singlet), d (doublet), t (triplet), m (multiplet), br (broadened), dd (doublet of doublets), dt (doublet of triplets). Coupling constants, when given, are reported in Hertz (Hz).

Infrared (IR) spectra were recorded on a Perkin-Elmer FT-IR Spectrometer as neat oils, or as KBr pellets, and when given are reported in wave numbers (cm⁻¹). The mass spectra were obtained using LSIMS or electrospray. All melting points (mp) are uncorrected.

The starting materials used in the examples are commercially available and/or can be prepared by techniques known in the art.

### EXAMPLE 1

### Method A: 5-(5-ethylaminomethyl-4-methylpyridin-3-yl)-3-(1H-imidazol-2-yl)-1H-indazole

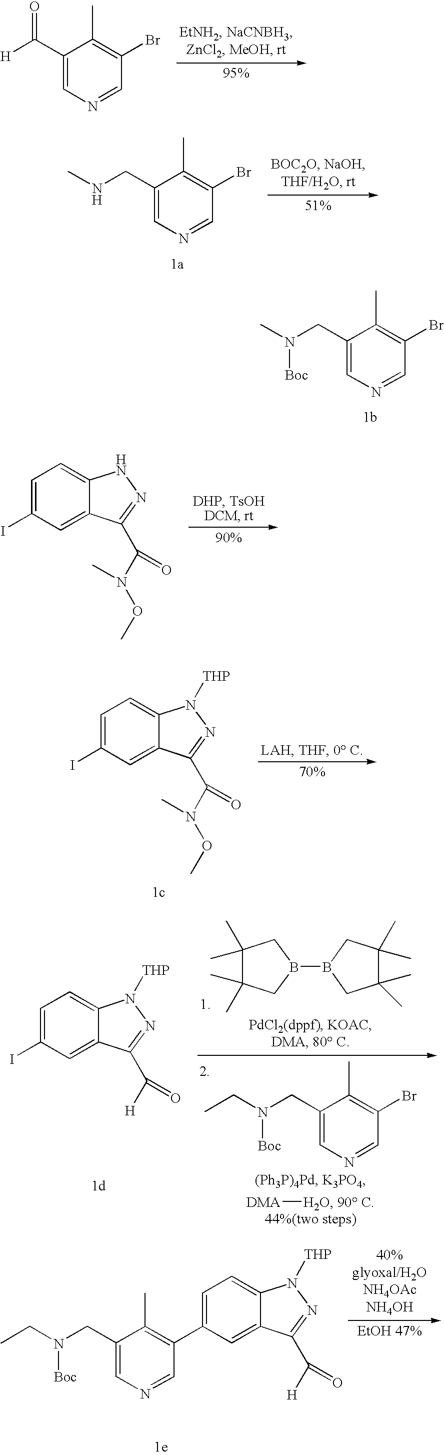

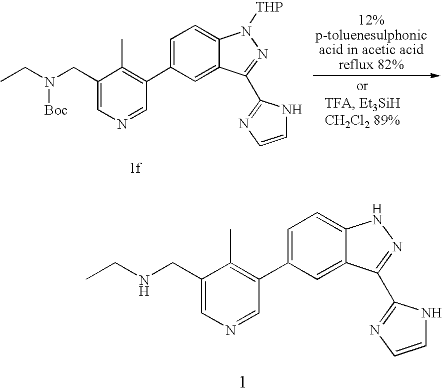

### (5-Bromo-4-methyl-pyridin-3-ylmethyl)-ethyl-amine (1a)

5-Bromo-4-methyl-pyridine-3-carbaldehyde (6.74 g, 33.7 mmol) [for the preparation of this compound see: Reich, S. R.; Bleckman, T. M.; Kephart, S. E.; Romines, W. H.; Wallace, M. B., U.S. Pat. No. 6,555,539 B2, Apr. 29, 2003.] was dissolved in methanol (290 mL) under a nitrogen atmosphere. A solution of ethylamine in methanol (2.0 M, 90 ml, 180 mmol) was added dropwise over 30 minutes. Stirring was continued at room temperature for 30 minutes further.

In a separate flask, sodium cyanoborohydride (2.33 9, 37.1 mmol) was dissolved in methanol (150 mL). Anhydrous zinc chloride (2.53 9, 18.5 mmol) was added and stirring continued at room temperature for 20 minutes. This solution (zinc/cyanoborohydride) was then slowly added to the above aldehyde/ethylamine solution. The reaction solution was acidified to pH 4 with 2.0 M HCl in methanol (120 mL), and then stirred at room temperature for 18 hours.

The solvents were removed by rotary evaporation and the residue partitioned between ethyl acetate and 10% aqueous sodium carbonate. The organic extracts were dried over magnesium sulfate and concentrated, affording crude amine la (7.36 9, 95%) as an orange oil, which was used in the next step without further purification: ¹H NMR (CDCl₃) δ 8.53 (s, 1H), 8.31 (s, 1H), 3.77 (s, 2H), 2.67 (q, J =7.0 Hz, 2 H), 2.42 (s, 3H), 1.11 (t, J=7.0 Hz, 3H).

### (5-Bromo-4-methyl-pyridin-3-ylmethyl)-ethyl-carbamic acid tert-butyl ester (1b)

Di-tert-butyl dicarbonate (10.43 g, 47.8 mmol) was added to a solution of crude amine 1a (7.36 9, 32.1 mmol) in THF (400 mL), followed by aqueous sodium hydroxide solution (1.0 M, 101 mL). The biphasic solution was stirred vigorously for 20 hours at room temperature. The solution was partitioned between water and ethyl acetate; the organic extracts were dried over magnesium sulfate, filtered, and concentrated. The crude yellow oil thus obtained was purified by silica gel chromatography (eluting with a gradient of 10% to 30% ethyl acetate in hexanes), yielded bromopyridine 1b (5.37 9, 51%) as a yellow oil: ¹H NMR (CDCl₃) δ 8.58 (s, 1H), 8.22 (s, 1H), 4.47 (s, 2H), 3.17 (br s, 2H), 2.37 (s, 3H), 1.45 (s, 9H), 1.03 (t, J=7.2 Hz, 3H).

### 5-Iodo-1-(tetrahydro-pyran-2-yl)-1H-indazole-3-carboxylic acid methoxy-methyl-amide (1c)

5-Iodo-1H-indazole-3-carboxylic acid methoxy-methyl-amide [for the preparation of this compound see: Reich, S. R.; Bleckman, T. M.; Kephart, S. E.; Romines, W. H.; Wallace, M. B., U.S. Pat. No. 6,555,539 B2, Apr. 29, 2003.] was alkylated with dihydropyran according to the method of Sun, et. al. [Sun, J.-H.; Teleha, C. A.; Yan, J.-S.; Rogers, J. D.; and Nugiel, D. A., J. Org. Chem. 1997, 62, 5627], affording amide 1c (typically >90%) as an off-white powder:

¹H NMR (DMSO-d₆) δ 8.37 (s, 1H), 7.74 (dd, J=1.5, 8.8 Hz, 1H), 7.68 (d, J=8.8 Hz, 1H), 5.97 (dd, J=2.3, 9.0 Hz, 1H), 3.88 (m, 2H), 3.79 (s, 3H), 3.42 (s, 3H), 2.35 (m, 1H), 2.03 (m, 2H), 1.75 (m, 1H), 1.58 (m, 2H).

### 5-Iodo-1-(tetrahydro-pyran-2-yl)-1H-indazole-3-carbaldehyde (1d)

Lithium aluminum hydride (1.2 equiv.) is added portionwise to a cooled (<5° C.) solution of amide 1c (1.0 equiv.) in THF. Stirring is continued at <5° C. until the reaction is complete, typically 30 minutes. The reaction was quenched by the slow addition of ethyl acetate at <5° C., and the whole mixture poured into 0.4 N NaHSO₄. The organic layer was washed with brine, dried over magnesium sulfate, concentrated, and purified by silica gel chromatography to give aldehyde 1d (typically ˜70%) as an off-white powder: ¹H NMR (CDCl₃) δ 10.15 (s, 1H), 8.47 (s, 1H), 7.82 (dd, J=1.5, 8.7 Hz, 1H), 7.78 (d, J=8.5 Hz, 1H), 6.04 (dd, J=2.3, 9.28 Hz, 1H), 3.85 (m, 2H), 2.35 (m, 1H), 2.05 (m, 2H), 1.76 (m, 1H), 1.60 (m, 2H).

### Ethyl-{5-[3-formyl-1-(tetrahydro-pyran-2-yl)-1H-indazol-5-yl]-4-methyl-pyridin-3-ylmethyl}-carbamic acid tert-butyl ester (1e)

Iodoindazole 1d (3.56 g, 10.0 mmol), bis(pinacolato)diboron (2.79 g, 11 mmol), potassium acetate (2.74 g, 30 mmol) and [1,1′-bis(diphenylphosphino)-ferrocene]dichloropalladium(II)complex with dichloromethane (245 mg, 0.3 mmol) were dissolved in N,N-dimethylacetamide (60 mL). The solution was degassed by evacuating (until the solvent begins to bubble) and purging with Argon (3 cycles), then heated in an 80° C. oilbath for 2 hours. After cooling slightly (to ˜50° C.), a solution of bromopyridine 1 b (3.62 g, 11 mmol) in N,N-dimethylacetamide (40 mL) was added, followed by deionized water (10 mL) and potassium phosphate (3.18 g, 15 mmol). The solution was degassed, tetrakis(triphenylphosphine) palladium (0) (347 mg, 0.3 mmol) added, and degassed again. The mixture was stirred in a 90° C. oilbath for 4.5 hours. After cooling to room temperature, the mixture was diluted with ethyl acetate (300 mL), washed with deionized water (150 mL), and saturated sodium chloride (100 mL). The organic layer was dried over magnesium sulfate, filtered, and concentrated to a crude red-black oil (9.43g). Purification by silica gel chromatography (eluting with 50-100% ethyl acetate in hexanes) afforded coupled product 1e (2.9462 g) as an orange oil. ¹H NMR of this product showed it was contaminated with ˜1 equivalent of pinacol. Trituration from hexanes afforded pure 1e (2.0853 g, 44%) as a fine yellow powder: ¹H NMR (CDCl₃) δ 10.25 (s, 1H), 8.39 (s, 1H), 8.34 (s, 1H), 8.22 (s, 7.74 (d, J=8.7 Hz, 1H), 7.38 (dd, J=1.5, 8.5 Hz, 1H), 5.88 (dd, J=2.8, 9.2 Hz, 1H), 4.53 (s, 2H), 4.03 (m, 1H), 3.81 (m, 1H), 3.24 (br s, 2H), 2.60 (m, 1H), 2.18 (s, 3H), 2.15 (m, 2H), 1.7 (m, 1H), 1.65 (m, 2H), 1.47 (s, 9H), 1.09 (t, J=7.0 Hz, 3H).

### Method A: Ethyl-{5-[3-(1H-imidazol-2-yl)-l-(tetrahydro-pyran-2-yl)-1H-indazol-5-yl]-4-methyl-pyridin-3-ylmethyl}-carbamic acid tert-butyl ester (1f)

Aldehyde 1e (200 mg, 0.42 mmole) was dissolved in warm (50° C.) ethanol (5 mL). The solution was cooled to room temperature. Ammonium acetate (130 mg, 1.69 mmole) was added followed by 30% ammonium hydroxide solution (2 mL) and 40% (by wt) glyoxal solution (135 uL, 0.93 mmole). The reaction mixture was stirred at room temperature for 18 hours then poured into 10% sodium bicarbonate solution (50 mL). The mixture was extracted with ethyl acetate (3×50 mL). The organic layer was dried over magnesium sulfate, filtered and concentrated to a crude oil. Purification by silica gel chromatography (eluting with 45-100% ethyl acetate in hexanes) afforded if (74 mg, 43%) as a clear oil: ¹H NMR (CDCl₃) δ 10.02 (s, 1H), 8.44 (s, 2H), 8.34 (s, 1H), 7.65 (d, J=8.4 Hz, 1H), 7.35 (d, J=8.7 Hz, 1H), 7.26 (s, 2H), 5.78 (dd, J=2.6, 9.8 Hz, 1H), 4.54 (s, 2H), 4.09 (m, 1H), 3.81 (m, 1H), 3.24 (br s, 2H), 2.62 (m, 1H), 2.20 (s, 3H), 2.15 (m, 2H), 1.78 (m, 4H), 1.48 (s, 9H), 1.11 (t, J=7.0 Hz, 3H).

### 5-(5-ethylaminomethyl-4-methylpyridin-3-yl)-3-(1H-imidazol-2-yl)-1H-indazole (1)

Imidazole 1f (164 mg, 0.32 mmole) was dissolved in ethanol (10 mL). A solution of 12% p-toluenesulphonic acid in acetic acid was added (8.5 mL, 5.57 mmole) and the reaction mixture heated to reflux for 16 hours. The solution was allowed to cool and the poured into 2N sodium hydroxide solution (100 mL). The mixture was extracted with 20% isopropanol in chloroform (3×80 mL). The organic layer was dried over magnesium sulfate, filtered and concentrated to a crude paste. Purification by silica gel chromatography {eluting with 15% (5% concentrate ammonium hydroxide in ethanol) in chloroform) afforded 1 (87 mg, 82%).

Alternatively, imidazole 1f (598 mg, 1.16 mmole) was dissolved in methylene chloride (12 mL), trifluoroacetic acid (2.75 mL, 35.7 mmole) and triethylsilane (270 uL, 1.68 mmole). The reaction mixture stirred at room temperature for 14 hours and then was poured 2N sodium hydroxide solution (250 mL). The mixture was extracted with 20% isopropanol in chloroform (3×100 mL). The organic layer was dried over magnesium sulfate, filtered and concentrated to a crude paste. Purification by silica gel chromatography {eluting with 15% (5% concentrate ammonium hydroxide in ethanol) in chloroform) afforded 1 (341 mg, 89%) as a light brown powder: ¹H NMR (CD₃OD) δ 8.43 (s, 1H), 8.36 (s, 1H, ), 8.25 (s, 1H), 7.65 (d, J=8.7 Hz, 1H), 7.39 (dd, J=1.5, 8.7 Hz, 1H), 7.18 (s, 2H), 3.88 (s, 2H), 2.75 (q, J=7.2 Hz, 2H), 2.34 (s, 3H), 1.19 (t, J=7.3 Hz, 3H).

Anal. Calcd. for C₁₉H₂₀N₈.0.2 H₂O.0.1 CH₃OH: C, 67.63; H, 6.18; N, 24.78. Found C, 67.52; H, 6.17; N, 24.67

### EXAMPLE 1

### Method B: 5-(5-ethylaminomethyl-4-methylpyridin-3-yl)-3-(1H-imidazol-2-yl)-1H-indazole-2-yl)-1H-indazole

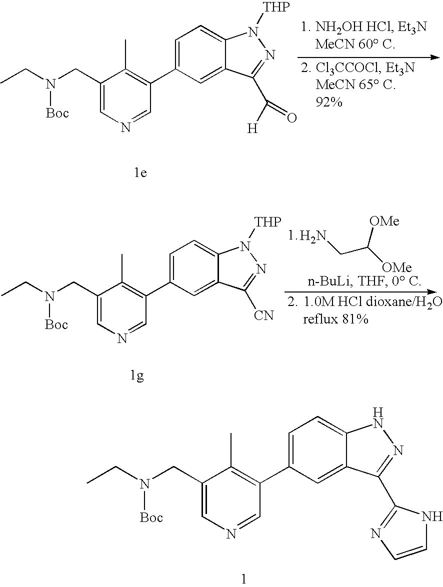

### Method B: {5-[3-Cyano-1-(tetrahydro-pyran-2-yl)-1H-indazol-5-yl]-4-methyl-pyridin-3-ylmethyl}-ethyl-carbamic acid tert-butyl ester (1g)

A suspension of aldehyde 1e (1.0 g, 2.09 mmole) in acetonitrile (20 mL) was created. To this was added triethylamine (320 uL, 2.30 mmole) followed by hydroxylamine hydrochloride (154 mg, 2.22 mmole). The reaction mixture was heated at 60° C. for 90 minutes. After allowing the reaction to cool, more triethylamine was added (960 uL, 7.0 mmole) followed by trichloroacetyl chloride (550 uL, 4.9 mmole). After stirring at room temperature for 30 minutes, the reaction mixture was heated to 65° C. for 16 hours. The mixture was allowed to cool and then poured into brine (200 mL). The solution was extracted with ethyl acetate (3×150 mL). The organic layer was dried over magnesium sulfate, filtered and concentrated to a crude oil. Purification by silica gel chromatography (eluting with 40-80% ethyl acetate in hexanes) afforded 19 (910 mg, 92%) as a white foam: ¹H NMR (CDCl₃) δ 8.38 (s, 1H), 8.37 (s, 1H), 7.81 (d, J=8.7 Hz, 1H), 7.73 (s, 1H), 7.40 (dd, J=1.5, 8.8 Hz, 1H), 5.86 (dd, J=2.9, 8.5 Hz, 1H), 4.53 (s, 2H), 3.96 (m, 1H), 3.78 (m, 1H), 3.25 (br s, 2H), 2.52 (m, 1H), 2.18 (s, 3H), 2.13 (m, 2H), 1.75 (m, 3H), 1.47 (s, 9H), 1.09 (t, J=7.2 Hz, 3H).

### 5-(5-ethylaminomethyl-4-methylpyridin-3-yl)-3-(1H-imidazol-2-yl)-1H-indazole (1)

A 2.5 M solution of n-butyllithium in hexanes (6.4 ml, 16 mmole) was added under argon to a solution of aminoacetaldehyde dimethyl acetal (1.8 ml, 1.74 g, 16.5 mmole) in THF (11.8 ml) at −75°. After stirring for a further 30 minutes at −70°, a portion (14 ml, 111.2 mmole, 2.5 eq.) of this lithium 2,2-dimethoxyethylamide solution was added to a solution of t.-butyl N-[(5-[3-cyano-1-tetrahydropyran-2-yl)-1H-indazol-5-yl]-4-methylpyridin-3-yl)methyl]-N-ethylcarbamate 1g (2.09 g, 4.4 mmole) in THF (40 ml). The resultant solution was stirred, under argon, at 0° for 2 hours, then quenched by addition of 50% aqueous CH₃OH (4 ml). The volatiles were removed by concentration, in vacuo, and the residue obtained was dissolved in 4.0 M HCl in 1,4-dioxane (20 ml). This solution was diluted with water (20 ml), then heated at reflux overnight. After cooling to room temperature, the crude reaction mixture was diluted with 2 N NaOH (60 ml) and extracted with 20% iPrOH in CHCl₃ (3×50 ml). The combined organic extracts were dried over Na₂SO₄ and concentrated, in vacuo, to give a brown foam which was purified by silica gel chromatography. Elution with CHCl₃:CH₃OH:NH₃ (94:5:1) and evaporation of the appropriate fractions gave 1.19 g (81%) ofa beige solid. ¹H NMR (CD₃OD) δ 8.44 (s,1H), 8.37 (s, 1H), 8.25 (d, J=1.5 Hz,1H), 7.66 (d, J=8.7 Hz, 1H), 7.40 (dd, J=1.5, 8.7 Hz, 1H), 7.18 (s, 2H), 3.89 (s, 2H), 2.77 (q, J=7.3 Hz, 2H), 2.35 (s, 3H), 1.30 (t, J=7.3 Hz, 3H).

Anal. Calcd. for C₁₉H₂₀N₆.0.25 CHCl₃.0.4 CH₃OH: C, 62.98; H, 5.87; N, 22.41. Found: C, 62.97; H, 5.80; N, 22.21.

### EXAMPLE 2

### 2-[5-(5-Ethylaminomethyl-4-methyl-pyridin-3-yl)-1H-indazol-3-yl]-5-phenyl-3H-imidazole-4-carboxylic acid ethylamide

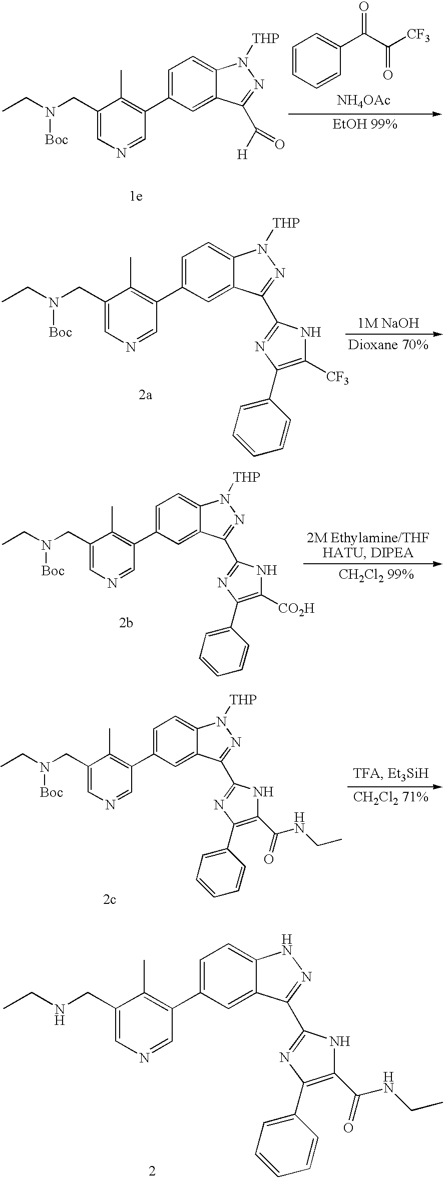

### Ethyl-{4-methyl-5-[3-(4-phenyl-5-trifluoromethyl-1H-imidazol-2-yl)-1-(tetrahydro-pyran-2-yl)-1H-indazol-5-yl]-pyridin-3-ylmethyl}-carbamic acid tert-butyl ester (2a)

The aldehyde 1e (4.11g, 8.59 mmol) was dissolved in 50 ml of ethanol at 55° C. To this was added the 1,1,1-triflouro-3-phenyl-2,3-dione hydrate (1.84g, 9.10 mmol) followed by ammonium acetate (3.10 g, 40.26 mmol). The reaction mixture was heated to reflux for 14 h. After cooling the reaction, solvents were removed in vacuo. The crude mixture was dissolved in EtOAc (75 ml) and washed with brine (3×50 ml). The organic layer was dried over magnesium sulfate, filtered and concentrated to a crude oil. Purification by silica gel chromatography (eluting with 50-100% ethyl acetate in hexanes) afforded 2a (5.62 g, 99%) as an orange solid. 1H NMR (300 MHz, MeOH) δ ppm 8.39 (s, 1 H) 8.33 (s, 1 H) 8.28 (s, 1 H) 7.85 (d, J=8.67 Hz, 1 H) 7.53-7.61 (m, 2 H) 7.40-7.53 (m, 4 H) 5.95 (d, J=9.23 Hz, 1 H) 4.59 (s, 2 H) 3.99-4.10 (m, J=9.09, 6.83, 6.69 Hz, 1 H) 3.79-3.90 (m, 1 H) 2.56-2.70 (m, 1 H) 2.28 (s, 3 H) 2.08-2.22 (m, 2 H) 1.78-1.91 (m, 1 H) 1.70 (bs, 2 H) 1.46 (bs, 8 H), 1.13 (t, J=6.97 Hz, 3 H).

### 2-[5-(5-[(tert-Butoxycarbonyl-ethyl-amino)-methyl]4-methyl-pyddin-3-yl}-1-(tetrahydro-pyran-2-yl)-1H-indazol-3-yl]-5-phenyl-3H-imidazole-4-carboxylic acid (2b)

The imidazole 2a (5.62 g, 8.51 mmol) was dissolved in dioxane (85 ml). To this was added 1 N NaOH solution (60 ml). The reaction mixture was heated to reflux for 2h. After cooling, the mixture was diluted with methylene chloride (100 ml) and acidified to pH 4 using 2N HCl. The mixture was extracted with methylene chloride (3×75 ml). The organic layer was dried over magnesium sulfate, filtered and concentrated to a crude paste. Purification by silica gel chromatography {eluting with 0-20% (5% concentrate ammonium hydroxide in ethanol) in chloroform) afforded 2b (4.66 g, 70%) as an orange powder: 1H NMR (300 MHz, MeOH) δ ppm 8.45 (s, 1 H) 8.40 (s, 1 H) 8.27 (s, 1H) 7.79-7.86 (m, 3 H) 7.45 (s,1H) 7.41 (d, J=7.54 Hz, 3 H) 5.95 (d, J=9.23 Hz, 1 H) 4.59 (s, 2 H) 4.05 (d, J=11.30 Hz, 1 H) 3.86 (t, J=10.55 Hz, 1 H) 3.34 (s,2 H) 2.56-2.67 (m, J=11.49 Hz, 1 H) 2.27 (s, 3 H) 2.15 (t, J=14.88 Hz, 2 H) 1.79-1.90 (m, J=12.43 Hz, 1 H) 1.70 (bs, 2 H) 1.46 (bs, 8 H) 1.13 (t, J=6.97 Hz, 3 H). Ethyl-{5-[3-(5-ethylcarbamoyl-4-phenyl-1H-imidazol-2-yl)-1-(tetrahydro-pyran-2-yl)-1H-indazol-5-yl]-4-methyl-pyridin-3-ylmethyl}-carbamic acid tert-butyl ester (2c)

The acid 2b (289 mg, 0.454 mmol) was dissolved in methylene chloride (5 ml). To this was added 2.0M Ethylamine in THF solution (454 ul, 0.455 mmol), DIPEA (158 ul, 0.908 mmol) and HATU (348 mg, 0.916 mmol). The reaction mixture stirred for 16 h at room temperature. The mixture was then diluted with EtOAc (25 ml), washed with brine (3×15 ml) and extracted with EtOAc (3×25 ml). The organic layer was dried over magnesium sulfate, filtered and concentrated to a crude oil. Purification by silica gel chromatography (eluting with 50-100% ethyl acetate in hexanes) afforded 2c (336 mg, quantitative yield) as a clear glass: 1H NMR (300 MHz, CHLOROFORM-D) δ ppm 8.41 (s,1 H) 8.30 (s, 1 H) 8.19 (s,1 H) 7.81 (d, J=6.78 Hz, 1 H) 7.62 (d, J=8.67 Hz, 1 H) 7.27-7.41 (m, 3 H) 5.74 (d, J=8.29 Hz, 1 H), 3.97-4.12 (m, 1 H) 3.67-3.85 (m, 1 H) 3.26-3.37 (m, 1 H) 3.21 (s, 1 H) 3 H) 2.43-2.58 (m, 1 H) 2.11 (t, J=12.81 Hz, 3 H) 1.65-1.80 (m, 2 H) 1.43 J=7.06 Hz, 4 H).

### 2-[5-(5-Ethylaminomethyl-4-methyl-pyridin-3-yl)-1H-indazol-3-yl]-5-phenyl-3H-imidazole-4-carboxylic acid ethylamide (2)

The amide 2c (336 mg, 0.507 mmol) was dissolved in methylene chloride (4 mL). To this solution was added TFA (2.0 mL, 25.96 mmol) and triethylsilane (200 uL, 1.242 mmol). The reaction mixture stirred for 16 h at room temperature. The solvent was removed in vacuo. The crude mixture was redissolved in EtOAc (100 ml) and washed with 2N NaOH solution (3×50 ml). The aqueous was back extracted with EtOAc (2×50 ml). The combined organics were dried over magnesium sulfate, filtered and concentrated to a crude oil. Purification by silica gel chromatography {eluting with 15% (5% concentrate ammonium hydroxide in ethanol) in chloroform} afforded 2 (172 mg, 71%) as a yellow glass: 1H NMR (300 MHz, MeOH) δ ppm 8.92 (s, 1 H) 8.24 (s, 1 H) 7.88 (s, 1 H) 7.81 (d, J=7.35 Hz, 2 H) 7.63 (d, J=8.48 Hz, 1 H) 7.35-7.49 (m, 3 H) 7.23 (d, J=8.48 Hz, 1 H) 3.61 (s, 2 H) 2.98 (d, J=7.16 Hz, 2 H) 2.70 (q, J=6.97 Hz, 2 H) 1.92 (s, 3 H) 1.17 (t, J=7.16 Hz, 3 H) 0.76 (t, J=6.97 z, 3 H).

Anal. Calcd. for C₂₈H₂₉N₇O.0.2 CHCl₃.0.1 CH₃OH: C, 67.09; H, 5.89; N, 19.35. Found: C, 67.15; H, 5.84; N, 19.33.

### EXAMPLE 3

### 2-15-(5-Ethylaminomethyl-4-methyl-pyridin-3-yl)-1H-indazol-3-yl]-5-phenyl-3H-imidazole-4-carboxylic acid (2-methoxy-ethyl)-amide

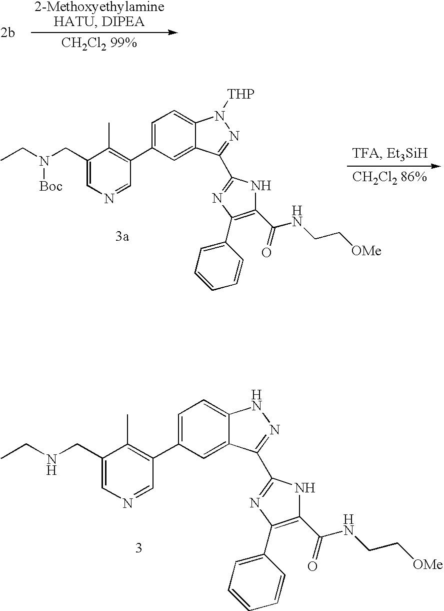

### Ethyl-{5-[3-[5-(2-methoxy-ethylcarbamoyl)-4-phenyl-1H-imidazol-2-yl]-1-(tetrahydro-pyran-2-yl)-1H-indazol-5-yl]-4-methyl-pyridin-3-ylmethyl}-carbamic acid tert-butyl ester (3a)

Example 3a was prepared similarly to Example 2c using acid 2b (278 mg, 0.437 mmol), 2-methoxyethylamine (80 ul, 0.922 mmol), DIPEA (155 ul, 0.892 mmol) and HATU (345 mg, 0.908 mmol). Analogous chromatography conditions afforded 3a (300 mg, 99%) as a glass: 1H NMR (300 MHz, MeOH) δ ppm 8.75 (s, 1 H) 8.11 (s, 1 H) 7.72-7.89 (m, 4 H) 7.27-7.52 (m, 4 H) 5.87-6.06 (m, 1 H) 4.87 (s, 2 H) 4.44 (s, 1 H) 4.00-4.17 (m, 2 H) 3.78-3.94 (m, 1 H) 3.13-3.46 (m, J=1.51 Hz, 5 H) 3.03 (s, 2 H) 2.76-2.86 (m, 2 H) 2.59-2.70 (m, 1 H) 2.17 (s, 2 H) 1.95-2.07 (m, 2 H) 1.83-1.96 (m, 1 H) 1.63-1.77 (m, 1 H) 1.49 (s, 9 H) 1.23 (t, J=7.16 Hz, 2 H) 1.11 (t, J=6.97 Hz, 3 H).

### 2-[5-(5-Ethylaminomethyl-4-methyl-pyridin-3-yl)-1H-indazol-3-yl]-5-phenyl-3H-imidazol-4-carboxylic acid (2-methoxy-ethyl)-amide (3)

Example 3 was prepared similarly to Example 2. Analogous chromatography conditions afforded 3 (190 mg, 86%) as a clear glass: 1H NMR (300 MHz, MeOH) δ ppm 8.72 (s, 1 H) 8.35 (s, 1 H) 8.11 (s, 1 H) 7.82 (d, J=7.16 Hz, 2 H) 7.66 (d, J=8.48 Hz, 1 H) 7.36-7.50 (m, 3 H) 7.31 (d, J=8.48 Hz, 1 H) 3.81 (s, 2 H) 3.25 (s, 3 H) 3.07 (s, 3 H) 2.79 (q, J=7.16 Hz, 2 H) 2.11 (s, 3 H) 1.21 (t, J=6.97 Hz, 3 H). Anal. Calcd. for C₂₉H₃₁N₇O₂.0.35 CHCl₃.0.2 CH₃OH: C, 63.33; H, 5.89; N, 17.38. Found: C, 63.36; H, 5.88; N, 17.32.

### EXAMPLE 4

### {2-[5-(5-Ethylaminomethyl-4-methyl-pyridin-3-yl)-1H-indazol-3-yl]-5-phenyl-3H-imidazol-4-yl}pyrrolidin-1-yl-methanone

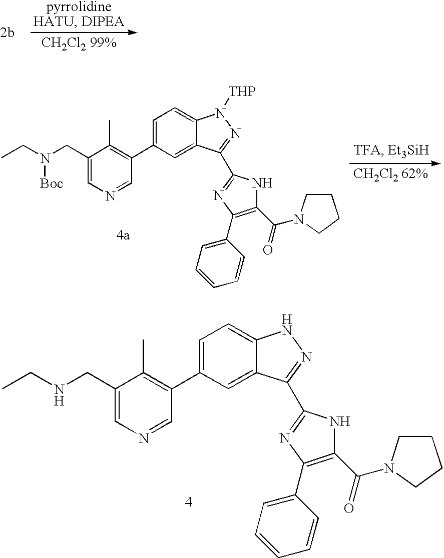

### Ethyl-{4-methyl-5-[3-[4-phenyl-5-(pyrrolidine-1-Carbonyl)-1H-imidazol-2-yl]-1-tetrayhydro-pyran-2-yl)-1H-indazol-5-yl]-pyridin-3-ylmethyl}-carbamic acid tert-butyl ester (4a)

Example 4a was prepared similarly to Example 2c using acid 2b (300 mg, 0.472 mmol), pyrrolidine (80 ul, 0.962 mmol), DIPEA (165 ul, 0.95 mmol) and HATU (360 mg, 0.947 mmol). Analogous chromatography conditions afforded 4a (249 mg, 77%) as a glass: 1H NMR (300 MHz, MeOH) δ ppm 8.38 (d, J=7.16 Hz, 2 H) 8.28 (s1 H) 7.84 (d, J=8.85 Hz, 1 H) 7.65 (d, J7.16 Hz, 2 H) 7.29-7.51 (m, 4 H) 5.95 (dd, 1 H) 4.59 (s, 2 H) 3.97-4.11 (m, 1 H) 3.78-3.91 (m, 1 H) 3.65-3.77 (m, 1 H) 3.58 (t, J=6.88 Hz, 2 H) 3.36-3.49 (m, 2 H) 3.21 (q, J=7.47 Hz, 1 H) 2.52-2.72 (m, 1 H) 2.28 (s, 3 H) 2.07-2.21 (m, 1 H) 1.82-1.94 (m 2 H) 1.74-1.82 (m, 2 H) 1.60-1.72 (m, 1 H) 1.47 (s, 9 H) 1.28-1.40 (m, 4 H) 1.13 (t, J=6.97 Hz, 3 H).

### {2-[5-(5-Ethylaminomethyl-4-methyl-pyridin-3-yl)-1H-indazol-3-yl]-5-phenyl-3H-imidazol-4-yl}-pyrrolidin-1-yl-methanone (4)

Example 4 was prepared similarly to Example 2. Analogous chromatography conditions afforded 4 (113 mg, 62%) as a clear glass: 1H NMR (300 MHz, MeOH) δ ppm 8.44 (s,1 H) 8.35 (d, J=8.10 Hz, 2 H) 7.58-7.75 (m, 3 H) 7.28-7.50 (m, 4 H) 3.90 (s, 2 H) 3.51-3.68 (m, J=6.78, 6.78 Hz, 2 H) 3.36-3.48 (m, 2 H) 2.69-2.89 (m, 2 H) 2.35 (s, 3 H) 1.67-2.00 (m, 4 H) 1.20 (t, J=7.16 Hz, 3 H).

Anal. Calcd. for C₃₀H₃₁N₇O.0.1 CHCl₃.0.3 CH₃OH: C, 69.26; H, 6.18; N, 18.60. Found: C, 69.22; H, 6.17; N, 18.70.

### EXAMPLE 5

### 2-[5-(5-Ethylaminomethyl-4-methyl-pyridin-3-yl)-1H-indazol-3-yl]-5-phenyl-3H-imidazole-4-carboxylic acid benzylamide

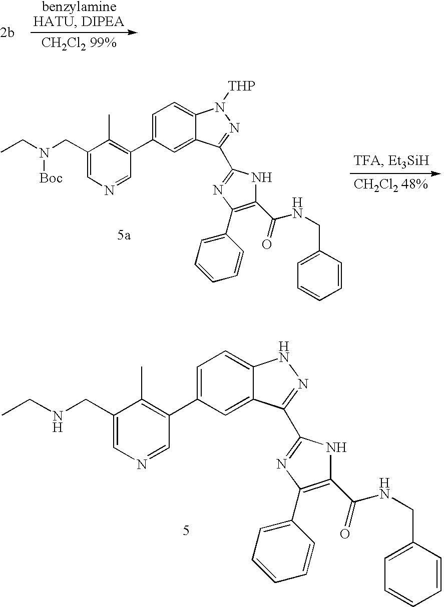

### {5-[3-(5-Benzylcarbamoyl-4-phenyl-1H-imidazol-2-yl)-1-(tetrahydro-pyran-2-yl)-1H-indazol-5-yl]-4-methyl-pyridin-3-ylmethyl}-ethyl-carbamic acid tert-butyl ester (5a)

Example 5a was prepared similarly to Example 2c using acid 2b (300 mg, 0.472 mmol), benzylamine (105 ul, 0.963 mmol), DIPEA (165 ul, 0.95 mmol) and HATU (360 mg, 0.947 mmol). Analogous chromatography conditions afforded 4a (345 mg, 99%) as a glass: 1H NMR (300 MHz, MeOH) δ ppm 7.77-7.90 (m, 1 H) 7.66-7.78 (m, 2 H) 7.33-7.49 (m, 2 H) 7.16-7.32 (m, 6 H) 7.12 (d, J=7.91 Hz, 1 H) 6.60-6.91 (m, 3 H) 5.91 (d, J=9.04 Hz, 1 H) 4.31 (s, 4 H) 3.92-4.09 (m, 3 H) 3.78-3.92 (m, 1 H) 3.16 (s,1 H) 2.14 (d, J=10.36 Hz, 2 H) 1.88-2.01 (m, 5 H) 1.61-1.76 (m, 3 H) 1.32-1.61 (m, 9 H) 0.98-1.12 (m, 3 H).

### 2-[5-(5-Ethylaminomethyl-4-methyl-pyridin-3-yl)-1H-indazol-3-yl]-5-phenyl-3H-imidazole-4-carboxylic acid benzylamide (5)

Example 5 was prepared similarly to Example 2. Analogous chromatography conditions afforded 5 (117 mg, 46%) as a clear glass: 1H NMR (300 MHz, MeOH) δ ppm 9.04 (s, 1 H) 8.11 (s,1 H) 7.79 (d, J=7.16 Hz, 2 H) 7.65 (s, 1 H) 7.55 (d, J=8.48 Hz, I H) 7.35-7.51 (m, 4 H) 7.11 (d, J=8.29 Hz, 1 H) 6.83 (d, J=5.65 Hz, 3 H) 6.71 (s, 2 H) 3.95 (s, 2 H) 3.38 (s, 2 H) 2.57 (q, J=6.59 Hz, 2 H) 1.69 (s, 3 H) 1.11 (t, J=6.97 Hz, 3 H).

Anal. Calcd. for C₃₃H₃₁N₇O.0.5 H₂O: C, 71.98; H, 5.86; N, 17.81. Found: C, 71.91; H, 5.80; N, 17.98.

### EXAMPLE 6

### 2-[5-(5-Ethylaminomethyl-4-methyl-pyridin-3-yl)-1H-indazol-3-yl]-5-phenyl-3H-imidazole-4-carboxylic acid methylamide

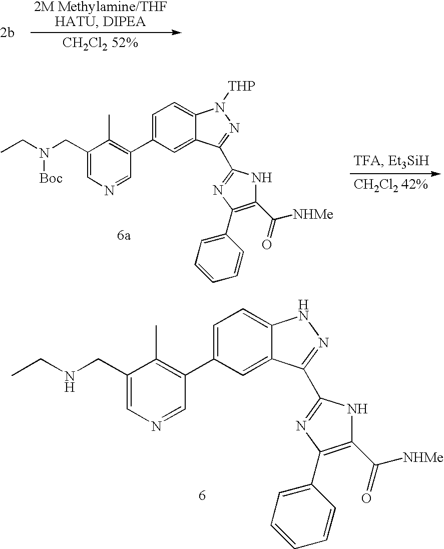

### Ethyl-(4-methyl-5-[3-(5-methylcarbamoyl-4-phenyl-1H-imidazol-2-yl)-1-(tetrahydro-pyran-2-yl)-1H-indazol-5-yl]-pyridin-3-ylmethyl)carbamic acid tert-butyl ester (6a)

Example 6a was prepared similarly to Example 2c using acid 2b (256 mg, 0.403 mmol), 2.0 M methylamine in THF solution (405 ul, 0.810 mmol), DIPEA (140 ul, 0.805 mmol) and HATU (306 mg, 0.805 mmol). Analogous chromatography conditions afforded 6a (137 mg, 52%) as a glass: 1H NMR (300 MHz, MeOH) δ ppm 8.72 (s, 1 H) 8.07-8.23 (m, J=14.32 Hz, 2 H) 7.71-7.85 (m, 3 H) 7.28-7.48 (m, 5 H) 5.90-6.00 (m, 1 H) 4.44 (s, 2 H) 4.00-4.11 (m, 1 H) 3.80-3.92 (m, 1 H) 3.25-3.32 (m, 2 H) 2.76-2.85 (m, 5 H) 2.59-2.73 (m, 2 H) 2.08-2.23 (m, 3 H) 1.82-2.02 (m,1 H) 1.70 (s, 2 H) 1.43 (t, 9 H) 1.10 (t, J=6.97 Hz, 3 H).

### 2-[5-(5-Ethylaminomethyl-4-methyl-pyridin-3-yl)-1H-indazol-3-yl]-5-phenyl-3H-imidazole-4-carboxylic acid methylamide (6)

Example 6 was prepared similarly to Example 2. HPLC chromatography conditions (0.1% AcOH/CH₃CN and 0.1% AcOH/H₂O) afforded 6(41 mg, 42%) as a clear glass: 1H NMR (300 MHz, MeOH) δ ppm 8.63 (s,1 H) 8.47 (s,1 H) 8.32 (s,1 H) 7.80 (d, J=6.78 Hz, 2 H) 7.68 (d, J=8.67 Hz, 1 H) 7.31-7.51 (m, 4 H) 4.14 (s, 2 H) 3.08 (q, 2 H) 2.73 (s, 3 H) 2.25 (s, 2 H) 1.89-2.00 (m, 3 H) 1.32 (t, J=7.25 Hz, 3 H).

Anal. Calcd. for C₂₇H₂₇N₇O.0.25 H₂O.0.5 AcOH: C, 64.58; H, 5.61; N, 18.66. Found: C, 64.61; H, 5.66; N, 18.66.

### EXAMPLE 7

### 2-[5-(5-Ethylaminomethyl-4-methyl-pyridin-3-yl)-1H-indazol-3-yl]-5-phenyl-3H-imidazole-4-carboxylic acid isobutyl-amide

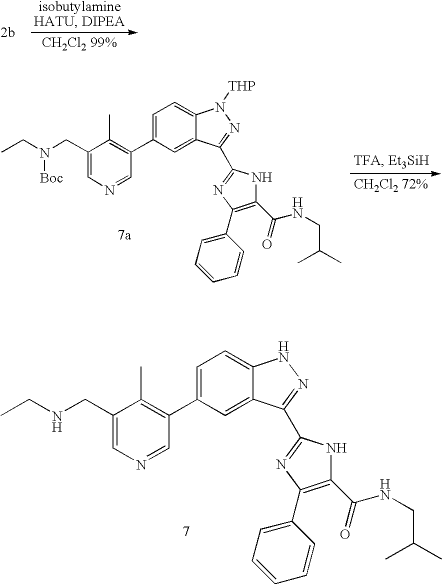

### Ethyl-{5-[3-(5-isobutylcarbamoyl-4-phenyl-1H-imidazol-2-yl)-1-(tetrahydro-pyran-2-yl)-1H-indazol-5-yl]-4-methyl-pyridin-3-ylmethyl}-carbamic acid tert-butyl ester (7a)

Example 7a was prepared similarly to Example 2c using acid 2b (300 mg, 0.472 mmol), isobutylamine (95 ul, 0.963 mmol), DIPEA (165 ul, 0.95 mmol) and HATU (355 mg, 0.934 mmol). Analogous chromatography conditions afforded 7a (328 mg, 99%) as a glass: 1H NMR (300 MHz, MeOH) δ ppm 8.84 (s, 1 H) 8.01-8.13 (m, 2 H) 7.70-7.86 (m, 3 H) 7.25-7.50 (m, 4 H) 5.96 (d, J=9.42 Hz, 1 H) 4.40 (s, 2 H) 4.03-4.12 (m, 1 H) 3.8-3.94 (m, 1 H) 2.75-2.85 (m, 5 H) 2.62-2.73 (m, 1 H) 2.11-2.25 (m, 2 H) 1.95-2.03 (m, 3 H) 1.84-1.95 (m, 1 H) 1.66-1.77 (m, 2 H) 1.48 (s, 9 H) 1.23 (t, J=7.16 Hz, 3 H) 0.68 (s, 6 H).

### 2-[5-(5-Ethylaminomethyl-4-methyl-pyridin-3-yl)-1H-indazol-3-yl]-5-phenyl-3H-imidazol-4-carboxylic acid isobutyl-amide (7)

Example 7 was prepared similarly to Example 2. Analogous chromatography conditions afforded 7 (171 mg, 72%) as a clear glass: 1H NMR (400 MHz, MeOD) δ ppm 8.96 (s, 1 H) 8.25 (s, 1 H) 7.89 (s, 1 H) 7.80 (d, J=7.07 Hz, 2 H) 7.63 (d, J=8.34 Hz, 1 H) 7.35-7.51 (m, 4 H) 7.23 (d, J=8.34 Hz, 1 H) 3.65 (s, 2 H) 2.67-2.81 (m, J=6.32 Hz, 4 H) 1.92 (s, 3 H) 1.34-1.46 (m, 1 H) 1.19 (t, 3 H) 0.61 (d, J=6.06 Hz, 6 H).

Anal. Calcd. for C₃₀H₃₃N₇O.0.3 CHCl₃: C, 66.96; H, 6.18; N, 18.04. Found: C, 67.10; H, 6.12; N, 17.92.

### EXAMPLE 8

### 2-[5-(5-Ethylaminomethyl-4-methyl-pyridin-3-yl)-1H-indazol-3-yl1-5-phenyl-3H-imidazole-4-carboxylic acid cyclopentylamide

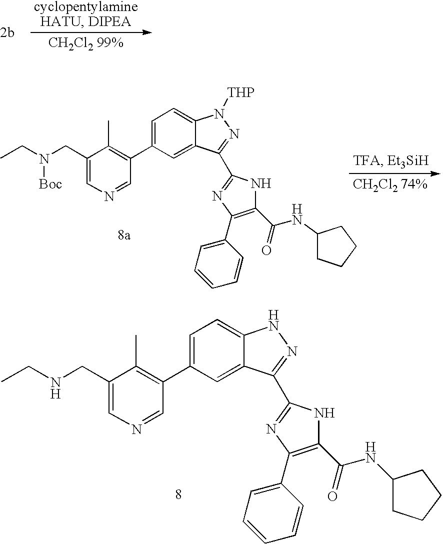

### {5-[3-(5-Cyclopentylcarbamoyl-4-phenyl-1H-imidazol-2-yl)-1-(tetrahydro-pyran-2-yl)-1H-indazol-5-yl]-4-methyl-pyridin-3-ylmethyl}-ethyl-carbamic acid tert-butyl ester (8a)

Example 8a was prepared similarly to Example 2c using acid 2b (300 mg, 0.472 mmol), cyclopentylamine (95 ul, 0.965 mmol), DIPEA (165 ul, 0.95 mmol) and HATU (355 mg, 0.934 mmol). Analogous chromatography conditions afforded 8a (334 mg, 99%) as a glass: 1H NMR (300 MHz, MeOH) δ ppm 8.95 (s, 1 H) 8.06 (s, 2 H) 7.74-7.88 (m, 3 H) 7.39-7.53 (m, 4 H) 7.25-7.35 (m, 1 H) 5.98 (d, J=8.29 Hz, 1 H) 4.46 (s, 2 H) 4.05-4.25 (m, 4 H) 3.85-3.97 (m, 1 H) 3.32-3.42 (m, 2 H) 2.84 (s, 3 H) 2.65-2.77 (m, 1 H) 2.13-2.26 (m, 2 H) 1.87-1.96 (m, 2 H) 1.66-1.83 (m, 4 H) 1.37-1.61 (m, 13 H) 1.09-1.20 (m, 3 H).

### 2-[5-(5-Ethylaminomethyl-4-methyl-pyridin-3-yl)-1H-indazol-3-yl]-5-phenyl-3H-imidazole-4-carboxylic acid cyclopentylamide (8)

Example 8 was prepared similarly to Example 2. Analogous chromatography conditions afforded 8 (180 mg, 74%) as a clear glass: 1H NMR (400 MHz, MeOD) δ ppm 8.74 (s, 1 H) 8.36 (s, 1 H) 8.07 (s, 1 H) 7.78 (d, J=7.07 Hz, 2 H) 7.64 (d, J=8.34 Hz, 1 H) 7.39-7.54 (m, 5 H) 7.28 (d, J=8.59 Hz, 1 H) 4.14-4.23 (m, 1 H) 3.83 (s, 2 H) 2.81 (q, J=6.99 Hz, 2 H) 2.06 (s, 3 H) 1.78 (s, 2 H) 1.45 (s, 4 H) 1.11-1.31 (m, 5 H).

Anal. Calcd. for C₃₁H₃₃N₇O0.1 MeOH.0.3 CHCl₃: C, 66.30; H, 5.97; N, 17.18. Found: C, 66.44; H, 6.00; N, 16.99.

### EXAMPLE 9

### {2-[5-(5-Ethylaminomethyl-4-methyl-pyridin-3-yl)-1H-indazol-3-yl]-5-phenyl-3H-imidazol-4-yl}-piperidin-1-yl-methanone

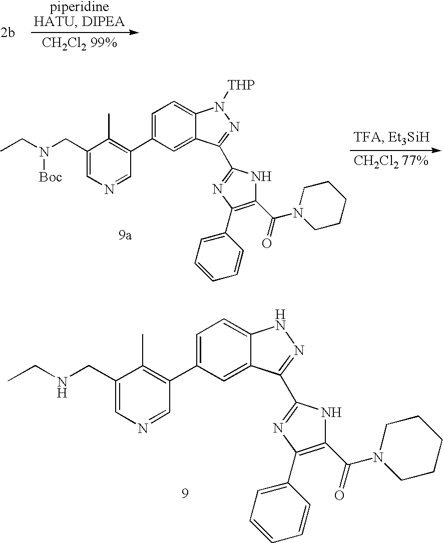

### Ethyl-{4-methyl-5-[3-[4-phenyl-5-(piperidine-1-carbonyl)-1H-imidazol-2-yl]-1-(tetrahydro-pyran-2-yl)-1H-indazol-5-yl]-pyridin-3-ylmethyl}-carbamic acid tert-butyl ester (9a)

Example 9a was prepared similarly to Example 2c using acid 2b (300 mg, 0.472 mmol), piperidine (95 ul, 0.961 mmol), DIPEA (165 ul, 0.95 mmol) and HATU (355 mg, 0.934 mmol). Analogous chromatography conditions afforded 9a (335 mg, 99%) as a glass: 1H NMR (300 MHz, MeOH) δ ppm 8.37 (d, J=4.33 Hz, 2 H) 8.27 (s, 1 H) 7.83 (d, J=8.67 Hz, 1 H) 7.63 (d, J=7.35 Hz, 2 H) 7.30-7.46 (m, 4 H) 5.93 (d, J=9.42 Hz, 1 H) 4.58 (s, 2 H) 3.97-4.08 (m, 1 H) 3.78-3.91 (m, 1 H) 3.62-3.73 (m, J=6.41, 6.41, 6.41 Hz, 2 H) 3.33 (s, 1 H) 3.20 (q, J=7.35 Hz, 1 H) 2.92 (d, J=7.35 Hz, 2 H) 2.80 (s, 2 H) 2.56-2.69 (m,1 H) 2.27 (s, 3 H) 2.05-2.20 (m, 1 H) 1.69 (s, 1 H) 1.57 (s, 2 H) 1.46 (s, 6 H) 1.29-1.37 (m, J=6.41 Hz, 9 H) 1.12 (t, J=6.97 Hz, 3 H).

### {2-[5-(5-Ethylaminomethyl-4-methyl-pyridin-3-yl)-1H-indazol-3-yl]-5-phenyl-3H-imidazol-4-yl}piperidin-1-yl-methanone (9)

Example 9 was prepared similarly to Example 2. Analogous chromatography conditions afforded 9 (188 mg, 77%) as a clear glass: 1H NMR (300 MHz, MeOH) δ ppm 8.51 (s, 1 H) 8.44 (s, 1 H) 8.35 (s, 1 H) 7.57-7.77 (m, 3 H) 7.33-7.50 (m, 4 H) 4.14 (s, 2 H) 3.65-3.75 (m, 2 H) 3.30-3.41 (m, 2 H) 2.94-3.08 (m, 2 H) 2.38(s,3 H) 1.58(s,4 H) 1.22-1.46 (m, 5 H).

Anal. Calcd. for C₃₁H₃₃N₇O.0.8 EtOH.0.7 CHCl₃: C, 62.49; H, 6.06; N, 15.32. Found: C, 62.32; H, 5.93; N, 15.10.

### EXAMPLE 10

### {2-[5-(5-Ethylaminomethyl-4-methyl-pyridin-3-yl)-1H-indazol-3-yl]-5-phenyl-3H-imidazol-4-yl]morpholin-4-yl-methanone

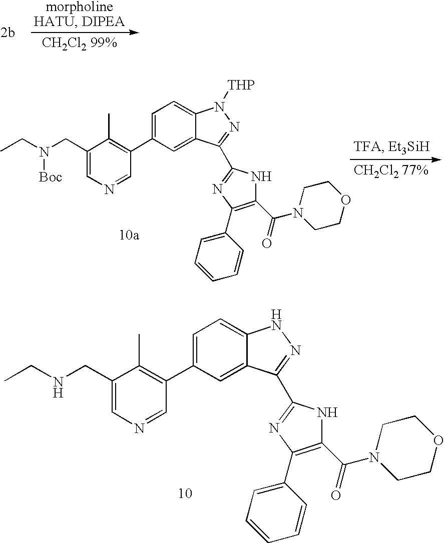

### Ethyl-{4-methyl-5-[3-[5-(morpholine-4-carbonyl)-4-phenyl-1H-imidazol-2-yl]-1(tetrahydro-pyran-2-yl)-1H-indazol-5-yl]-pyridin-3-ylmethyl}-carbamic acid tert-butyl ester (10a)

Example 10a was prepared similarly to Example 2c using acid 2b (300 mg, 0.472 mmol), morpholine (85 ul, 0.961 mmol), DIPEA (165 ul, 0.95 mmol) and HATU (355 mg, 0.934 mmol). Analogous chromatography conditions afforded 10a (340 mg, 99%) as a glass: 1H NMR (300 MHz, MeOH) δ ppm 8.33-8.39 (m, 2 H) 8.27 (s, 1 H) 7.83 (d, J=8.67 Hz, 1 H) 7.62 (d, J=7.35 Hz, 2 H) 7.35-7.50 (m, 4 H) 5.93 (d, J=9.42 Hz, 1 H) 4.58 (s, 2 H) 3.99-4.11 (m, 1 H) 3.79-3.90 (m, 1 H) 3.62-3.76 (m, 4 H) 3.45 (s, 1 H) 3.31-3.38 (m, 2 H) 3.20 (q, J=7.41 Hz, 2 H) 2.75-2.84 (m, 4 H) 2.54-2.68 (m, 1 H) 2.26 (s, 3 H) 2.12 (s, 1 H) 1.64-1.74 (m, 1 H) 1.46 (s, 9 H) 1.12 (t, J=6.97 Hz, 3 H).

### {2-[5-(5-Ethylaminomethyl-4-methyl-pyridin-3-yl)-1H-indazol-3-yl]-5-phenyl-3H-imidazol-4-yl}-morpholin-4-yl-methanone (10)

Example 10 was prepared similarly to Example 2. Analogous chromatography conditions afforded 10 (230 mg, 94%) as a clear glass: 1H NMR (300 MHz, MeOH) δ ppm 8.53 (s, 1 H) 8.46 (s, 1 H) 8.34 (s, 1 H) 7.58-7.77 (m, 3 H) 7.36-7.52 (m, 4 H) 4.23 (s, 2 H) 3.61-3.79 (m, J=13.19, 6.59 Hz, 5 H) 3.37 (s, 2 H) 3.16-3.23 (m, J=7.35 Hz, 1 H) 3.03-3.14 (m, 2 H) 2.39 (s, 3 H) 1.27-1.42 (m, 3 H).

Anal. Calcd. for C₃₁H₃₃N₇O₂.1.7 EtOH.0.9 CHCl₃: C, 58.24; H, 6.00; N, 13.86. Found: C, 58.09; H, 5.83; N, 13.77.

### EXAMPLE 11

### 2-[5-(5-Ethylaminomethyl-4-methyl-pyridin-3-yl)-1H-indazol-3-yl]-5-phenyl-3H-imidazole-4-carboxylic acid (3-methoxy-propyl)-amide

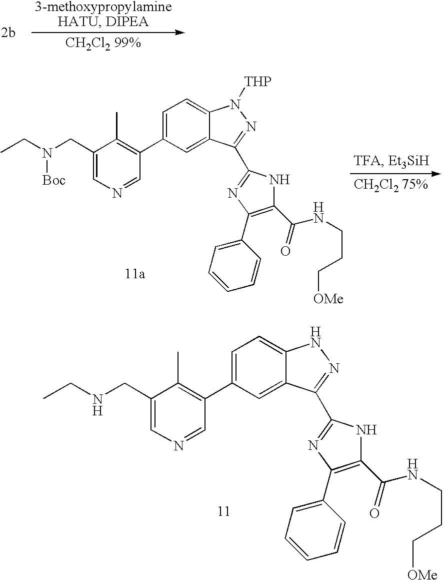

### Ethyl-{5-[3-[5-(3-methoxy-propylcarbamoyl)-4-phenyl-1H-imidazol-2-yl]-1-(tetrahydro-pyran-2-yl)-1H-indazol-5-yl]-4-methyl-pyridin-3-ylmethyl)carbamic acid tert-butyl ester (11a)

Example 11 a was prepared similarly to Example 2c using acid 2b (300 mg, 0.472 mmol), 3-methoxypropylamine (100 ul, 0.982 mmol), DIPEA (165 ul, 0.95 mmol) and HATU (355 mg, 0.934 mmol). Analogous chromatography conditions afforded 1 la (341 mg, 99%) as a glass: 1H NMR (300 MHz, MeOH) δ ppm 8.82 (s, 1 H) 8.05 (s, 1 H) 7.72-7.85 (m, 3 H) 7.37-7.49 (m, 3 H) 7.27-7.33 (m, 1 H) 5.96 (d, J=8.29 Hz, 1 H) 4.40 (s, 2 H) 4.02-4.10 (m, 1 H) 3.83-3.91 (m, 1 H) 3.66-3.77 (m, 1 H) 3.08-3.26 (m, 6 H) 2.95-3.04 (m, 3 H) 2.63-2.72 (m, 1 H) 2.11-2.24 (m, J=0.75 Hz, 3 H) 1.82-1.94 (m, J=11.49 Hz, 1 H) 1.67-1.75 (m, 1 H) 1.45-1.58 (m, 9 H) 1.29-1.38 (m, J=5.84, 5.84 Hz, 4 H) 1.19-1.26 (m, J=7.16, 7.16 Hz, 2 H) 1.10 (t, J=6.88 Hz, 3 H).

### 2-[5-(5-Ethylaminomethyl-4-methyl-pyridin-3-yl)-1H-indazol-3-yl]-5-phenyl-3H-imidazole-4-carboxylic acid (3-methoxy-propyl)-amide (11)

Example 11 was prepared similarly to Example 2. Analogous chromatography conditions afforded 11 (186 mg, 75%) as a clear glass: 1H NMR (300 MHz, MeOH) δ ppm 8.74 (s, 1 H) 8.39 (s, 1 H) 8.15 (s, 1 H) 7.76-7.85 (m, 2 H) 7.67 (d, J=8.67 Hz, 1 H) 7.38-7.51 (m, 3 H) 7.31 (dd, J=8.57, 1.22 Hz, 1 H) 3.94 (s, 2 H) 3.14-3.28 (m, 4 H) 3.06 (s, 3 H) 2.88-2.98 (m, 2 H) 2.13 (s, 3 H) 1.52-1.64 (m, 2 H) 1.26 (t, J=7.16 Hz, 3 H).

Anal. Calcd. for C₃₀H₃₃N₇O₂.0.9 EtOH.0.3 TFA.0.35 CHCl₃: C, 61.36; H, 6.14; N, 15.29. Found: C, 61.44; H, 6.12; N, 15.25.

### EXAMPLE 12

### 2-[5-(5-Ethylaminomethyl-4-methyl-pyridin-3-yl)-1H-indazol-3-yl]-5-phenyl-3H-imidazole-4-carboxylic acid cyclohexylamide

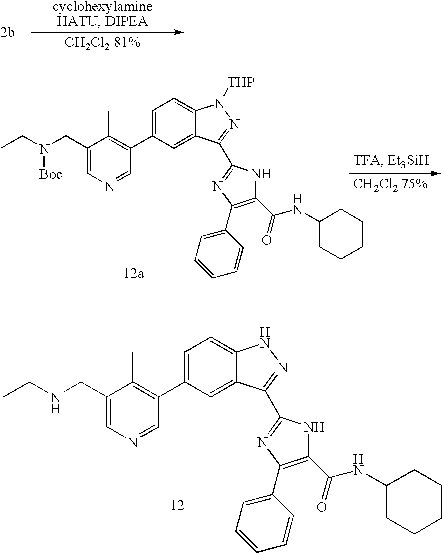

### {5-[3-(5-Cyclohexylcarbamoyl-4-phenyl-1H-imidazol-2-yl)-1-(tetrahydro-pyran-2-yl)-1H-indazol-5-yl]-4-methyl-pyridin-3-ylmethyl}-ethyl-carbamic acid tert-butyl ester (12a)

Example 12a was prepared similarly to Example 2c using acid 2b (300 mg, 0.472 mmol), cyclohexylamine (110 ul, 0.963 mmol), DIPEA (165 ul, 0.95 mmol) and HATU (355 mg, 0.934 mmol). Analogous chromatography conditions afforded 12a (275 mg, 81%) as a glass: 1H NMR (300 MHz, MeOH) δ ppm 8.85 (s, 1 H) 8.06 (s, 2 H) 7.75 (d, J=0.75 Hz, 3 H) 7.22-7.50 (m, 4 H) 5.96 (d, J=8.48 Hz, 1 H) 4.36-4.57 (m, J=8.67 Hz, 2 H) 4.01-4.12 (m, 1 H) 3.79-3.95 (m, 1 H) 3.61-3.74 (m, 1 H) 2.77-2.85 (m,1 H) 2.58-2.74 (m, 1 H) 2.07-2.31 (m, J=3.01 Hz, 4 H) 1.79-1.95 (m, 2 H) 1.64-1.78 (m, 6 H) 1.47 (s, 13 H) 1.16 -1.27 (m, 4 H) 1.03-1.16 (m, 3 H) 0.90 (s, 2 H).

### 2-[5-(5-Ethylaminomethyl-4-methyl-pyridin-3-yl)-1H-indazol-3-yl]-5-phenyl-3H-imidazole-4-carboxylic acid cyclohexylamide (12)

Example 12 was prepared similarly to Example 2. Analogous chromatography conditions afforded 12 (163 mg, 65%) as a clear glass: 1H NMR (300 MHz, MeOH) δ ppm 9.04 (s, 1 H) 8.23 (s, 1 H) 7.70-7.86 (m, 3 H) 7.57 (d, J=8.48 Hz, 1 H) 7.32-7.50 (m, 4 H) 7.13 (d, J=8.48 Hz, 1 H) 3.52-3.67 (m, 3 H) 2.58-2.78 (m, J=6.84, 6.84, 6.84 Hz, 2 H) 1.76 (s, 3 H) 1.35-1.60 (m, 5 H) 1.04-1.25 (m, 6 H) 0.62-0.83 (m, 3 H).

Anal. Calcd. for C₃₃H₃₇N₇O.0.2 H₂O.0.1 TFA: C, 70.49; H, 6.52; N, 17.87. Found: C, 70.41; H, 6.45; N, 18.01.

### EXAMPLE 13

### 2-[5-(5-Ethylaminomethyl-4-methyl-pyridin-3-yl)-1H-indazol-3-yl]-5-phenyl-3H-imidazole-4-carboxylic acid cyclohexyl-methyl-amide

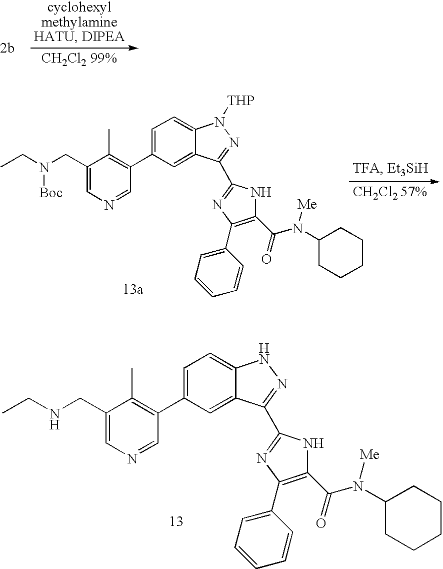

### {5-[3-[5-(Cyclohexyl-methyl-carbamoyl)-4-phenyl-1H-imidazol-2-yl]-1-(tetrahydro-pyran-2-yl)-1H-indazol-5-yl]-4-methyl-pyridin-3-ylmethyl}-ethyl-carbamic acid tert-butyl ester (13a)

Example 13a was prepared similarly to Example 2c using acid 2b (300 mg, 0.472 mmol), cyclohexylmethylamine (125 ul, 0.960 mmol), DIPEA (165 ul, 0.95 mmol) and HATU (355 mg, 0.934 mmol). Analogous chromatography conditions afforded 13a (350 mg, 99%) as a glass: 1H NMR (300 MHz, MeOH) δ ppm 8.32-8.37 (m, 2 H) 8.24-8.28 (m, 1 H) 7.82 (d, J=8.67 Hz, 1 H) 7.60 (d, J=7.35 Hz, 2 H) 7.27-7.44 (m, 4 H) 5.92 (d, J=8.67 Hz, 1 H) 4.57 (s, 2 H) 3.98-4.06 (m, 1 H) 3.76-3.88 (m, 1 H) 2.91-2.97 (m, 3 H) 2.77-2.82 (m, 3 H) 2.62-2.68 (m, 2 H) 2.25 (s, 3 H) 2.02-2.17 (m, 2 H) 1.77-1.88 (m, J=7.72 Hz, 3 H) 1.61-1.71 (m, 3 H) 1.36-1.53 (m, 15 H) 1.11 (t, J=7.06 Hz, 3 H).

### 2-[5-(5-Ethylaminomethyl-4-methyl-pyridin-3-yl)-1H-indazol-3-yl]-5-phenyl-3H-imidazole-4-carboxylic acid cyclohexyl-methyl-amide (13)

Example 13 was prepared similarly to Example 2. Analogous chromatography conditions afforded 13 (146 mg, 57%) as a clear glass: 1H NMR (300 MHz, MeOH) δ ppm 8.45 (s, 1 H) 8.29-8.38 (m, J=11.49 Hz, 2 H) 7.65 (dd, 3 H) 7.31-7.50 (m, 4 H) 3.90 (s, 2 H) 2.96 (s, 3 H) 2.69-2.82 (m, 3 H) 2.34 (s, 3 H) 1.66-1.90 (m, J=37.11 Hz, 2 H) 1.40-1.60 (m, 6 H) 1.21 (t, J=7.16 Hz, 3 H) 0.94-1.05 (m, 2 H).

Anal. Calcd. for C₃₃H₃₇N₇O.0.2 H₂O.0.2 TFA: C, 70.99; H, 6.69; N, 17.35. Found: C, 70.97; H, 6.70; N, 17.40.

### EXAMPLE 14

### {2-[5-(5-Ethylaminomethyl-4-methyl-pyridin-3-yl)-1H-indazol-3-yl]-5-phenyl-3H-imidazol-4-yl}-(2-methoxymethyl-pyrrolidin-1-yl)-methanone

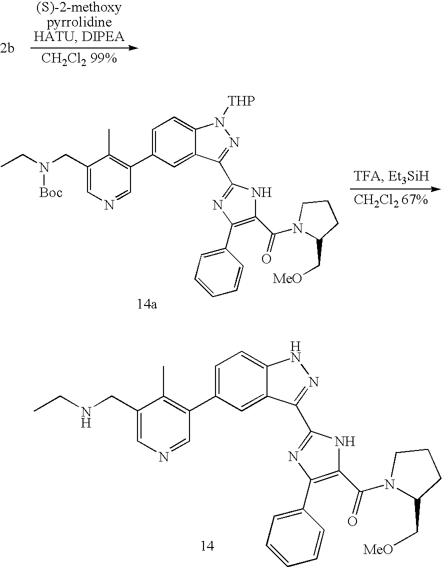

### Ethyl-{5-[3-[5-(2-methoxymethyl-pyrrolidine-1-carbonyl)-4-phenyl-1H-imidazol-2-yl]-1-(tetrahydro-pyran-2-yl)-1H-indazol-5-yl]-4-methyl-pyridin-3-ylmethyl}-carbamic acid tert-butyl ester (14a)

Example 14a was prepared similarly to Example 2c using acid 2b (300 mg, 0.472 mmol), (S)-2-methoxymethylpyrrolidine (120 ul, 0.974 mmol), DIPEA (165 ul, 0.95 mmol) and HATU (355 mg, 0.934 mmol). Analogous chromatography conditions afforded 14a (349 mg, 99%) as a glass: 1H NMR (300 MHz, MeOH) δ ppm 8.39 (s, 2 H) 8.28 (s, 1 H) 7.84 (d, J=8.67 Hz, 1 H) 7.67 (d, J=6.78 Hz, 2 H) 7.32-7.50 (m, 4 H) 5.94 (d, J=9.42 Hz, 1 H) 4.59 (s, 2 H) 4.02-4.11 (m, 1 H) 3.81-3.93 (m, 1 H) 3.65-3.77 (m, 1 H) 3.58-3.64 (m, J=3.01 Hz, 1 H) 3.43-3.52 (m, 1 H) 3.37-3.45 (m, 1 H) 3.15-3.26 (m, 2 H) 2.83-3.00 (m, 5 H) 2.56-2.69 (m, J=9.98 Hz, 1 H) 2.27 (s, 3 H) 2.08-2.19 (m, 1 H) 1.81-1.94 (m, 1 H) 1.65-1.77 (m, 2 H) 1.47 (s, 4 H) 1.29-1.39 (m, 9 H) 1.13 (t, J=6.97 Hz, 3 H).

### {2-[5-(5-Ethylaminomethyl-4-methyl-pyridin-3-yl)-1H-indazol-3-yl]-5-phenyl-3H-imidazol-4-yl}-(2-methoxymethyl-pyrrolidin-1-yl)-methanone (14)

Example 14 was prepared similarly to Example 2. Analogous chromatography conditions afforded 14 (173 mg, 67%) as a clear glass: 1H NMR (300 MHz, MeOH) δ ppm 8.47 (s, 1 H) 8.39 (s, 1 H) 8.35 (s, 1 H) 7.67 (d, J=6.97 Hz, 3 H) 7.32-7.49 (m, 4 H) 4.30-4.45 (m, 1 H) 3.99 (s, 2 H) 3.58-3.69 (m, 2 H) 3.35-3.47 (m, 3 H) 3.12-3.24 (m, J=7.35 Hz, 1 H) 2.96 (s, 1 H) 2.81-2.92 (m, J=6.97 Hz, 2 H) 2.35 (s, 3 H) 1.60-2.07 (m, 2 H) 1.30-1.40 (m, J=5.93, 5.93 Hz, 2 H) 1.23 (t, J=7.16 Hz, 3 H).

Anal. Calcd. for C₃₃H₃₅N₇O₂.0.3 H₂O.0.3 MeOH.0.5 TFA: C, 64.33; H, 6.05; N, 15.77. Found: C, 64.31; H, 6.08; N, 15.87.

### EXAMPLE 15

### {2-[5-(5-Ethylaminomethyl-4-methyl-pyridin-3-yl)-1H-indazol-3-yl]-5-phenyl-3H-imidazol-4-yl}-(2-methoxymethyl-pyrrolidin-1-yl)-methanone

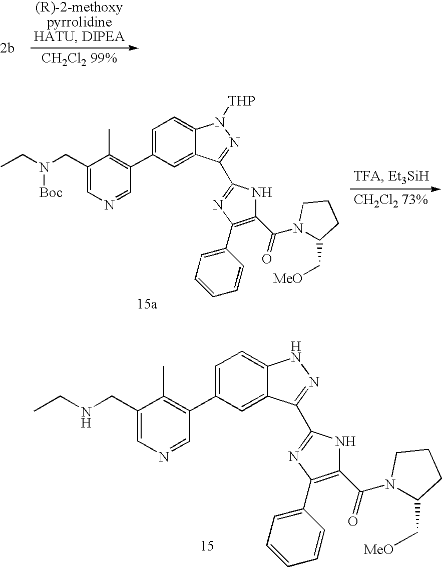

### Ethyl-{5-[3-[5-(2-methoxymethyl-pyrrolidine-1-carbonyl)-4-phenyl-1H-imidazol-2-yl]-1-(tetrahydro-pyran-2-yl)-1H-indazol-5-yl]-4-methyl-pyridin-3-ylmethyl}-carbamic acid tert-butyl ester (15a)

Example 15a was prepared similarly to Example 2c using acid 2b (300 mg, 0.472 mmol), (R)-2-methoxymethylpyrrolidine (120 ul, 0.974 mmol), DIPEA (165 ul, 0.95 mmol) and HATU (355 mg, 0.934 mmol). Analogous chromatography conditions afforded 15a (352 mg, 99%) as a glass: 1H NMR (300 MHz, MeOH) δ ppm 8.39 (s, 2 H) 8.28 (s, 1 H) 7.84 (d, J=8.67 Hz, 1 H) 7.67 (d, J=6.59 Hz, 2 H) 7.31-7.51 (m, 4 H) 5.93 (d, J=9.23 Hz, 1 H) 4.59 (s, 2 H) 3.99-4.11 (m, 1 H) 3.78-3.92 (m, J=10.55 Hz, 1 H) 3.58-3.75 (m, 3 H) 3.37-3.50 (m, 2 H) 3.13-3.23 (m, 2 H) 2.85-3.00 (m, 3 H) 2.54-2.69 (m, J=5.09 Hz, 1 H) 2.27 (s, 3 H) 2.07-2.18 (m, 1 H) 1.81-1.94 (m, 2 H) 1.63-1.75 (m, 2 H) 1.41-1.55 (m, 4 H) 1.28-1.37 (m, 9 H) 1.14 (t, J=6.78 Hz, 3 H).

### {2-[5-(5-Ethylaminomethyl-4-methyl-pyridin-3-yl)-1H-indazol-3-yl]-5-phenyl-3H-imidazol-4-yl}-(2-methoxymethyl-pyrrolidin-1-yl)-methanone (15)

Example 15 was prepared similarly to Example 2. Analogous chromatography conditions afforded 15 (188 mg, 73%) as a clear glass: 1H NMR (300 MHz, MeOH) δ ppm 8.46 (s, 1 H) 8.36 (d, J=7.91 Hz, 2 H) 7.63-7.73 (m, 3 H) 7.31-7.49 (m, 4 H) 4.33-4.44 (m, 1 H) 3.95 (s, 2 H) 3.57-3.70 (m, 2 H) 3.39 (s, 3 H) 3.13-3.24 (m, 1 H) 2.96 (s, 1 H) 2.83 (q, J=7.16 Hz, 2 H) 2.32-2.40 (m, J=3.58 Hz, 3 H) 1.61-2.03 (m, 3 H) 1.30-1.36 (m, 1 H) 1.22 (t, J=7.16 Hz, 3 H).

Anal. Calcd. for C₃₃H₃₅N₇O₂.0.4 MeOH.0.2 TFA: C, 67.31; H, 6.34; N, 16.75. Found: C, 67.28; H, 6.29; N, 16.74.

### EXAMPLE 16

### 2-[5-(5-Ethylaminomethyl-4-methyl-pyridin-3-yl)-1H-indazol-3-yl]-5-phenyl-3H-imidazole-4-carboxylic acid (2-dimethylamino-ethyl)-amide

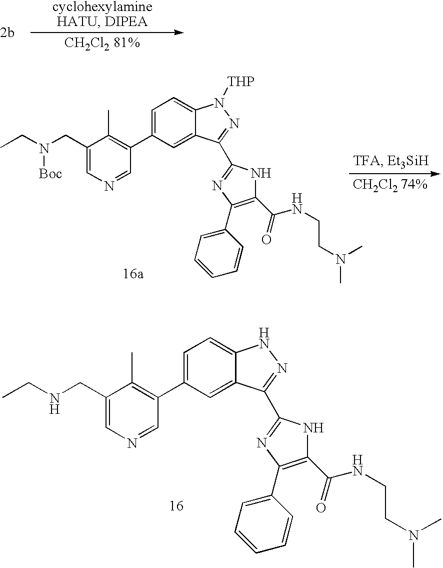

### {5-[3-[5-(2-Dimethylamino-ethylcarbamoyl)-4-phenyl-1H-imidazol-2-yl]-1-(tetrahydro-pyran-2-yl)-1H-indazol-5-yl]-4-methyl-pyridin-3-ylmethyl}-ethyl-carbamic acid tert-butyl ester (16a)

Example 16a was prepared similarly to Example 2c using acid 2b (300 mg, 0.472 mmol), dimethylaminoethylamine (110 ul, 1.01 mmol), DIPEA (165 ul, 0.95 mmol) and HATU (355 mg, 0.934 mmol). Analogous chromatography conditions afforded 16a (226 mg, 67%) as a crude oil (LC/MS M+H=707 MW). The crude oil was carried on to the deprotection step.

### 2-[5-(5-Ethylaminomethyl-4-methyl-pyridin-3-yl)-1H-indazol-3-yl]-5-phenyl-3H-imidazole-4-carboxylic acid (2-dimethylamino-ethyl)-amide (16)

Example 16 was prepared similarly to Example 2. HPLC chromatography conditions (0.1% AcOH/CH₃CN and 0.1% AcOH/H₂O) afforded 16 (124 mg, 74%) as a clear glass: 1H NMR (300 MHz, MeOH) δ ppm 8.62 (s, 1 H) 8.52 (s, 1 H) 8.35 (s, 1 H) 7.79-7.86 (m, 2 H) 7.71 (d, J=8.67 Hz, 1 H) 7.31-7.52 (m, 4 H) 4.20 (s, 2 H) 3.47-3.57 (m, 2 H) 3.07-3.16 (m, 2 H) 2.87-2.97 (m, 2 H) 2.58-2.66 (m, 6 H) 2.26 (s, 3 H) 1.34 (t, J=7.06 Hz, 3 H).

Anal. Calcd. for C₃₀H₃₄N₈O.0.25 AcOH.0.8 H₂O.1.0 TFA: C, 58.60; H, 5.69; N, 16.82. Found: C, 58.60; H, 5.62; N, 16.80.

### EXAMPLE 17

### 2-[5-(5-Ethylaminomethyl-4-methyl-pyridin-3-yl)-1H-indazol-3-yl]-5-phenyl-3H-imidazole-4-carboxylic acid (3-hydroxy-propyl)-amide

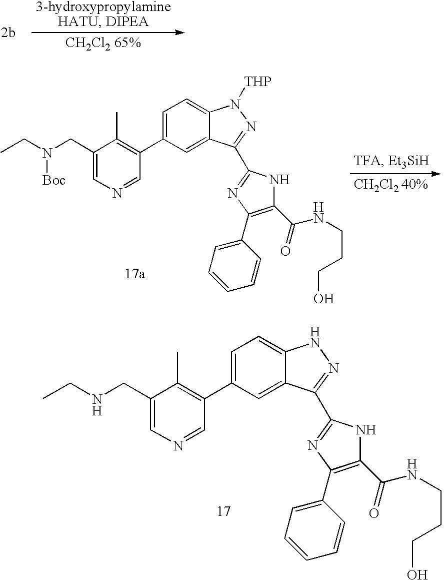

### Ethyl-{5-[3-[5-(3-hydroxy-propylcarbamoyl)-4-phenyl-1H-imidazol-2-yl]-1-(tetrahydro-pyran-2-yl)-1H-indazol-5-yl]-4-methyl-pyridin-3-ylmethyl}-carbamic acid tert-butyl ester (17a)

Example 17a was prepared similarly to Example 2c using acid 2b (300 mg, 0.472 mmol), 3-hydroxypropylamine (75 ul, 0.982 mmol), DIPEA (165 ul, 0.95 mmol) and HATU (355 mg, 0.934 mmol). Analogous chromatography conditions afforded 17a (215 mg, 65%) as a crude oil (LC/MS M+H=694 MW). The crude oil was carried on to the deprotection step.

### 2-[5-(5-Ethylaminomethyl-4-methyl-pyridin-3-yl)-1H-indazol-3-yl]-5-phenyl-3H-imidazole-4-carboxylic acid (3-hydroxy-propyl)-amide (17)

Example 17 was prepared similarly to Example 2. HPLC chromatography conditions (0.1% AcOH/CH₃CN and 0.1% AcOH/H₂O) afforded 17 (89 mg, 40%) as a clear glass: 1H NMR (300 MHz, MeOH) δ ppm 8.67 (s, 1 H) 8.49 (s, 1 H) 8.32 (s, 1 H) 7.80 (d, J=6.97 Hz, 2 H) 7.69 (d, J=8.67 Hz, 1 H) 7.31-7.54 (m, 4 H) 4.25 (s, 2 H) 3.48 (t, J=6.12 Hz, 2 H) 3.25-3.32 (m, 2 H) 3.13-3.23 (m, 2 H) 2.23 (s, 3 H) 1.54-1.67 (m, 2 H) 1.36 (t, J=7.25 Hz, 3 H).

Anal. Calcd. for C₂₉H₃₁N₇O₂.0.25 H₂O.1.1 TFA: C, 58.59; H, 5.14; N, 15.33. Found: C, 58.58; H, 5.11; N, 15.35.

### EXAMPLE 18

### 2-[5-(5-Ethylaminomethyl-4-methyl-pydridin-3-yl)-1H-indazol-3-yl]-5-phenyl-3H-imidazole-4-carboxylic acid (4-hydroxy-butyl)-amide

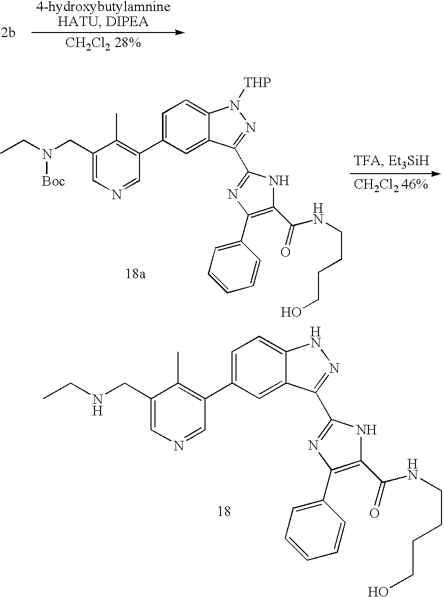

### Ethyl-{5-[3-[5-(4-hydroxy-butylcarbamoyl)-4-phenyl-1H-imidazol-2-yl]-1-(tetrahydro-pyran-2-yl)-1H-indazol-5-yl]-4-methyl-pyridin-3-ylmethyl}-carbamic acid tert-butyl ester (18)

Example 18a was prepared similarly to Example 2c using acid 2b (300 mg, 0.472 mmol), 4-hydroxybutylamine (90 ul, 0.978 mmol), DIPEA (165 ul, 0.95 mmol) and HATU (355 mg, 0.934 mmol). Analogous chromatography conditions afforded 18a (94 mg, 28%) as a crude oil (LC/MS M+H=708 MW). The crude oil was carried on to the deprotection step.

### 2-[5-(5-Ethylaminomethyl-4-methyl-pyridin-3-yl)-1H-indazol-3-yl]-5-phenyl-3H-imidazole-4-carboxylic acid (4-hydroxy-butyl)-amide (18)

Example 18 was prepared similarly to Example 2. HPLC chromatography conditions (0.1% AcOH/CH₃CN and 0.1% AcOH/H₂O) afforded 18 (95 mg, 46%) as a clear glass: 1H NMR (300 MHz, MeOH) δ ppm 8.70 (s, 1 H) 8.48 (s, 1 H) 8.29 (s, 1 H) 7.80 (d, J=6.78 Hz, 2 H) 7.69 (d, J=8.85 Hz, 1 H) 7.39-7.52 (m, 4 H) 7.34 (d, J=8.67 Hz, 1 H) 4.21 (s, 2 H) 3.40 (s, 2 H) 3.10-3.24 (m, 4 H) 2.20 (s, 3 H) 1.27-1.50 (m, 7 H).

Anal. Calcd. for C₃₀H₃₃N₇O₂.0.5 H₂O.1.1 TFA: C, 58.77; H, 5.38; N, 14.90. Found: C, 58.70; H, 5.42; N, 14.96.

### EXAMPLE 19

### Ethyl-{4-methyl-5-[3-(5-methyl-4-propyl-1H-imidazol-2-yl)-1H-indazol-5-yl]-pyridin-3-ylmethyl}-amine

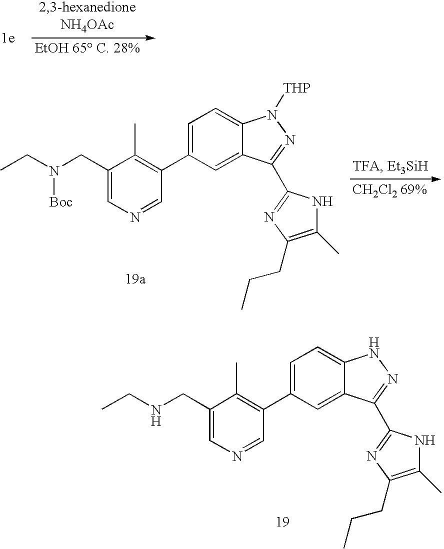

### Ethyl-{4-methyl-5-[3-(5-methyl-4-propyl-1H-imidazol-2-yl)-1-(tetrahydro-pyran-2-yl)-1H-indazol-5-yl]-pyridin-3-ylmethyl}-carbamic acid tert-butyl ester (19a)

A suspension of aldehyde 1e (301 mg, 0.628 mmol) in EtOH (6 ml) was heated to 60° C. to form a homogenous solution. To this reaction mixture was added 2,3-hexanedione (81 mg, 0.711 mmol) and ammonium acetate (248 mg, 3.22 mmol). The mixture was heated to reflux for 16 hr. The mixture was then poured into EtOAc (50 ml) and washed with brine (3×50 ml). The aqueous layer was back extracted with EtOAc (2×50 ml). The combined organic layers were dried over magnesium sulfate, filtered and concentrated to a crude oil. Purification by silica gel chromatography (eluting with 50-100% ethyl acetate in hexanes) afforded 19a (102 mg, 28%) as a clear glass: 1H NMR (300 MHz, CHLOROFORM-D) δ ppm 9.52 (s, 1 H) 8.44 (d, J=3.58 Hz, 2 H) 8.35 (s, 1 H) 7.61 (d, J=8.48 Hz, 1 H) 7.32 (dd, J=8.67, 1.51 Hz, 1 H) 5.75 (dd, J=9.70, 2.54 Hz, 1 H) 4.54 (s, 2 H) 4.07-4.17 (m, 1 H) 3.80 (s, 1 H) 3.25 (s, 2 H) 2.47-2.68 (m, 5 H) 2.23-2.29 (m, 3 H) 2.19 (s, 3 H) 2.07-2.14 (m, 1 H) 1.57-1.89 (m, 4 H) 1.49 (s, 9 H) 1.11 (t, J=6.88 Hz, 3 H) 0.89-1.01 (m, 3 H).

### Ethyl-{4-methyl-5-[3-(5-methyl-4-propyl-1H-imidazol-2-yl)-1H-indazol-5-yl]-pyridin-2-ylmethyl}-amine (19)

Example 19 was prepared similarly to Example 2. Analogous chromatography conditions afforded 19 (100 mg, 69%) as a clear glass: 1H NMR (300 MHz, MeOH) δ ppm 8.44 (s, 1 H) 8.36 (s, 1 H) 8.23 (s, 1 H) 7.63 (d, J=8.48 Hz, 1 H) 7.37 (d, J=8.48 Hz, 1 H) 3.88 (s, 2 H) 2.76 (q, J=6.97 Hz, 2 H) 2.54-2.64 (m, J=7.25, 7.25 Hz, 2 H) 2.33 (s, 3 H) 2.22 (s, 3 H) 1.59-1.73 (m, 2 H) 1.19 (t, J=7.06 Hz, 3 H) 0.95 (t, J=7.25 Hz, 3 H).

Anal. Calcd. for C₂₃H₂₈N₆.0.1 iPOH.0.12 CH₂Cl₂: C, 69.50; H, 7.23; N, 20.77. Found: C, 69.81; H, 7.21; N, 20.43.

### EXAMPLE 20

### Ethyl-{5-[3-(4-isobutyl-5-methyl-1H-imidazol-2-yl)-1H-indazol-5-yl]-4-methyl-pyridin-3-ylmethyl}-amine

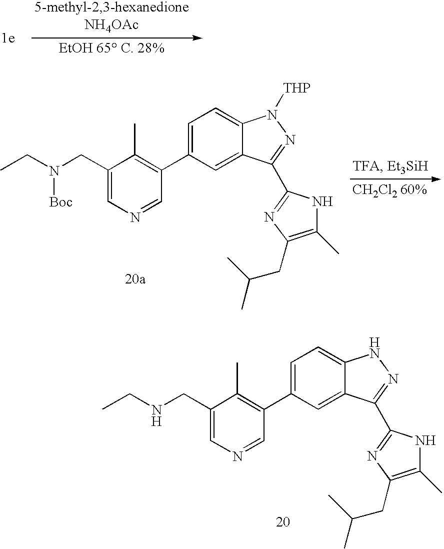

### Ethyl-{5-[3-(4-isobutyl-5-methyl-1H-imidazol-2-yl)-1-(tetrahydro-pyran-2-yl)-1H-indazol-5-yl]-4-methyl-pyridin-3-ylmethyl}-carbamic acid tert-butyl ester (20a)

Example 20a was prepared similarly to Example 19a using aldehyde 1e (300 mg, 0.626 mmol), ammonium acetate (242 mg, 3.14 mmol) and 5-methyl-2,3-hexanedione (90 mg, 0.703 mmol). Analogous chromatography conditions afforded 20a (182 mg, 50%) as a glass: 1H NMR (300 MHz, CHLOROFORM-D) δ ppm 8.43 (d, J=10.17 Hz, 2 H) 8.31 (s, 1 H) 7.62 (d, 1 H) 7.31 (dd, J=8.67, 1.51 Hz, 1 H) 5.73 (dd, J=9.61, 2.45 Hz, 1 H) 4.51 (s, 2 H) 4.02-4.15 (m, 1 H) 3.72-3.86 (m, J=8.10 Hz, 1 H) 3.24 (s, 2 H) 2.53-2.68 (m, 1 H) 2.45 (d, J=7.16 Hz, 2 H) 2.24 (s, 3 H) 2.18 (s, 3 H) 2.07-2.13 (m, 1 H) 1.89-2.00 (m, J=6.78 Hz, 1 H) 1.65-1.82 (m, 4 H) 1.47 (s, 9 H) 1.09 (t, J=6.97 Hz, 3 H) 0.86-0.98 (m, 6 H).

### Ethyl-{5-[3-(4-isobutyl-5-methyl-1H-imidazol-2-yl)-1H-indazol-5-yl]-4-methyl-pyridin-3-ylmethyl}-amine (20)

Example 20 was prepared similarly to Example 2. Analogous chromatography conditions afforded 20 (75 mg, 60%) as a clear glass: 1 H NMR (300 MHz, MeOH) δ ppm 8.43 (s, 1 H) 8.36 (s, 1 H) 8.23 (s, 1 H) 7.62 (d, J=8.29 Hz, 1 H) 7.37 (d, J=8.48 Hz, 1 H) 3.88 (s, 2 H) 2.76 (q, J=6.91 Hz, 2 H) 2.46 (d, J=6.97 Hz, 2 H) 2.33 (s, 3 H) 2.21 (s, 3 H) 1.88-2.00 (m, J=6.78 Hz, 1 H) 1.19 (t, J=7.06 Hz, 3 H) 0.86-1.02 (m, J=6.59 Hz, 6 H).

Anal. Calcd. for C₂₄H₃₀N₆.0.1 H₂O.0.12 CH₂Cl₂: C, 69.88; H, 7.40; N; 20.27. Found: C, 69.87; H, 7.37; N, 20.28.

### EXAMPLE 21

### Ethyl-{4-methyl-5-[3-(4,5,6,7-tetrahydro-1H-benzoimidazol-2-yl)-1H-indazol-5-yl]-pyridin-3-ylmethyl}-amine

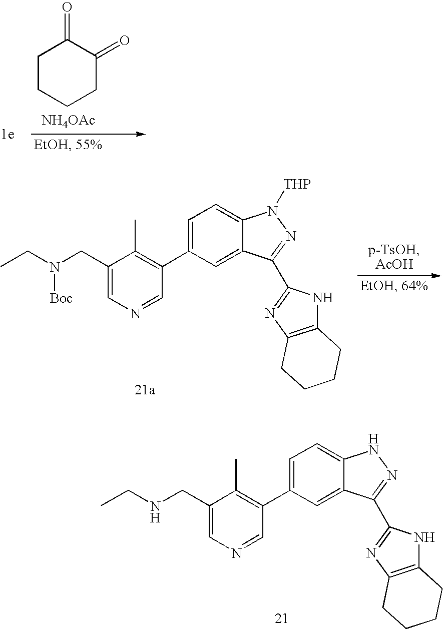

### Ethyl-{4-methyl-5-[3-(4,5,6,7-tetrahydro-1H-benzoimidazol-2-yl)-1-(tetrahydro-pyran-2-yl)-1H-indazol-5-yl]-pyridin-3-ylmethyl)-carbamic acid tert-butyl ester (21a)

Aldehyde 1e (611.6 mg, 1.28 mmol), 1,2-cyclohexanedione (150 mg, 1.34 mmol) and ammonium acetate (591 mg, 7.67 mmol) were dissolved in 12.8 mL ethanol and the mixture heated to reflux for 2 hours, 20 minutes. After cooling to room temperature, the solvents were removed in vacuo. The residue was dissolved in 100 mL ethyl acetate and washed with a mixture of 50 mL deionized water plus 50 mL brine. The organic layer was dried over magnesium sulfate, filtered and concentrated to a crude orange foam. Purification by silica gel chromatography (eluting with 100% ethyl acetate) afforded 21a (398.2 mg, 55%) as a yellow foam. ¹H NMR (DMSO-d₆) δ 12.25 (br s, 1H), 8.34 (s, 1H), 8.28 (s, 1H), 8.26 (s, 1H), 7.84 (d, J=8.6 Hz, 1H), 7.41 (dd, J=1.5, 8.7 Hz, 1H), 5.93 (dd, J=2.1, 10.0 Hz, 1H), 4.51 (s, 2H), 3.92 (m, 1H), 3.80 (m, 1H), 3.20 (m, 2H), 2.51 (m, 3H), 2.15 (s, 3H), 2.05 (m, 1H), 1.99 (m, 1H), 1.75 (br s, 5H), 1.61 (br s, 2H), 1.40 (br s, 9H), 1.03 (t, J=7.2 Hz, 3H).

### Ethyl-{4-methyl-5-[3-(4,5,6,7-tetrahydro-1H-benzoimidazol-2-yl)-1H-indazol-5-yl]-pyridin-3-ylmethyl}-amine (21)

Compound 21a (245.2 mg, 0.43 mmol) was dissolved in 6.25 mL ethanol, and 6.23 mL of a 0.69M solution of toluenesulfonic acid in acetic acid was added. The mixture was heated to reflux for 24 hours. After cooling to room temperature, 50 mL ethyl acetate was added, and the resulting solution washed with 40 mL of 2M sodium hydroxide. The organic layer was dried over magnesium sulfate, filtered, concentrated, and purified by silica gel chromatography (eluting with 1/19/80 concentrated ammonium hydroxide/ethanol/chloroform), affording 21 (106.9 mg, 64%) as a yellow solid. ¹H NMR (MeOD) δ 8.44 (s, 1H), 8.37 (s, 1H), 8.24 (s, 1H), 7.63 (d, J=8.7 Hz, 1H), 7.37 (dd, J=1.5, 8.7 Hz, 1H), 3.90 (s, 2H), 2.77 (q, J=7.2 Hz, 2H) 2.64 (br s, 4H), 2.34 (s, 3H), 1.86 (br s, 4H), 1.20 (t, J=7.2 Hz, 3H). Anal. Calc. for C₂₃H₂₆N₆.0.5H₂O.0.1CHCl₃: C, 68.09; H, 6.70; N, 20.63. Found: C, 67.98; H, 6.86; N, 20.35.

### EXAMPLE 22

### {5-[3-(4,5-Diethyl-1H-imidazol-2-yl)-1H-indazol-5-yl}-4-methyl-pyridin-3-ylmethyl}-ethyl-amine

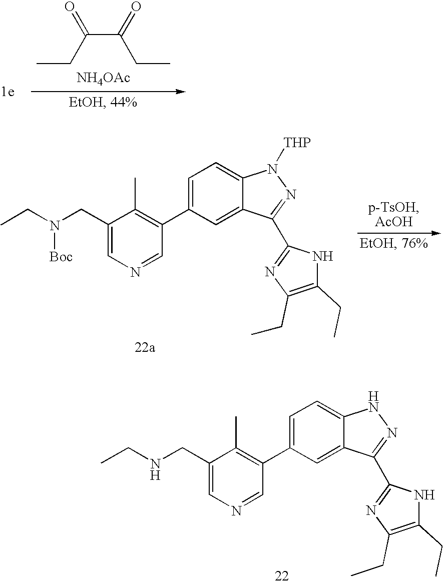

### {5-[3-(4,5-Diethyl-1H-imidazol-2-yl)-1-(tetrahydro-pyran-2-yl)-1H-indazol-5-yl]-4-methyl-pyridin-3-ylmethyl}-ethyl-carbamic acid tert-butyl ester (22a)

Compound 22a was prepared from aldehyde 1e (504.8 mg, 1.05 mmol), 3,4-hexanedione (132 mg, 1.16 mmol), and ammonium acetate (486 mg, 6.3 mmol) by a procedure similar to 21a. Purification by silica gel chromatography (eluting with 60-100% ethyl acetate in hexanes) afforded 22a (263.2 mg, 44%) as a yellow foam. ¹H NMR (DMSO-d₆) δ 12.17 (s, 1H), 8.36 (s, 1H), 8.30 (s, 2H), 7.83 (d, J=8.6 Hz, 1H), 7.43 (dd, J=1.5, 8.7 Hz, 1H), 5.93 (dd, J=2.1, 10.0 Hz, 1H), 4.51 (s, 2H), 3.93 (m, 1H), 3.80 (m, 1H), 3.19 (m, 2H), 2.56 (q, J=7.5 Hz, 2H), 2.46 (q, J=7.5 Hz, 2H), 2.17 (s, 3H), 2.04 (m, 2H), 1.80 (m, 1H), 1.61 (m, 2H), 1.40 (br s, 9H), 1.16 (t, J=7.5 Hz, 3H), 1.13 (t, J=7.5 Hz, 3H), 1.02 (t, J=7.0 Hz, 3H).

### {5-[3-(4,5-Diethyl-1H-imidazol-2-yl)-1H-indazol-5-yl]-4-methyl-pyridin-3-ylmethyl}-ethyl-amine (22)

Example 22 was prepared from 22a (235.6 mg, 0.411 mmol) by the same procedure used to make 21. Analogous chromatography conditions yielded 22 (121.4 mg, 76%) as a yellow foam. ¹H NMR (DMSO-d₆) δ 13.26 (s, 1H), 12.16 (s, 1H), 8.45 (s, 1H), 8.32 (s, 1H), 8.25 (s, 1H), 7.61 (d, J=8.5 Hz, 1H), 7.34 (dd, J=1.3, 8.5 Hz, 1H), 3.80 (s, 2H), 2.65 (q, J=7.2 Hz, 2H), 2.56 (m, 4H), 2.24 (s, 3H), 1.14 (m, 6H), 1.07 (t, J=7.0 Hz, 3H). Anal. Calc. for C₂₃H₂₈N₆.0.6H₂O: C, 69.18; H, 7.37; N, 21.05. Found: C, 69.37; H, 7.40; N, 20.72.

### EXAMPLE 23

### Ethyl-{5-[3-(4-ethyl-5-methyl-1H-imidazol-2-yl)-1H-indazol-5-yl]-4-methyl-pyridin-3-ylmethyl}-amine

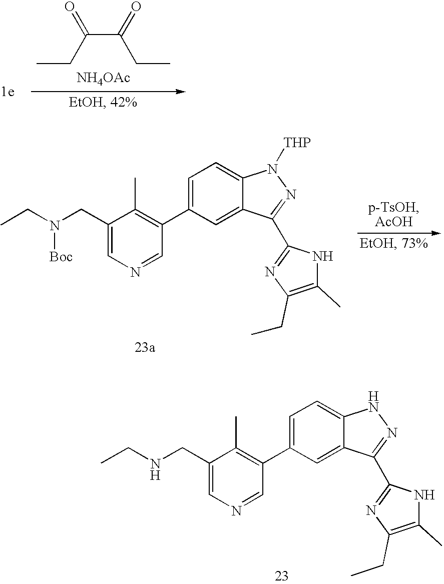

### Ethyl-{5-[3-(4-ethyl-5-methyl-1H-imidazol-2-yl)-1-(tetrahydro-pyran-2-yl)-1H-indazol-5-yl]-4-methyl-pyridin-3-ylmethyl}-carbamic acid tert-butyl ester (23a)

Compound 23a was prepared from aldehyde 1e (484.7 mg, 1.01 mmol), 2,3,-pentanedione (112 mg, 1.11 mmol), and ammonium acetate (468 mg, 6.08 mmol) by a procedure similar to 21a. Purification by silica gel chromatography (eluting with 70-100% ethyl acetate in hexanes) afforded 23a (237.4 mg, 42%) as a yellow foam. ¹H NMR (DMSO-d₆) δ 12.19 (s, 1H), 8.35 (s, 1H), 8.29 (s, 1H), 8.28 (s, 1H), 7.83 (d, J=8.7 Hz, 1H), 7.42 (dd, J=1.3, 8.7 Hz, 1H), 5.93 (dd, J=2.1, 10.0 Hz, 1H), 4.51 (s, 2H), 3.95 (m, 1H), 3.80 (m, 1H), 3.20 (m, 2H), 2.56 (m, 1H), 2.17 (s, 3H), 2.10 (m, 4H), 1.79 (m, 1H), 1.61 (m, 2H), 1.40 (br s, 9H), 1.14 (m, 3H), 1.03 (t, J=6.8 Hz, 3H).

### Ethyl-{5-[3-(4-ethyl-5-methyl-1H-imidazol-2-yl)-1H-indazol-5-yl]-4-methyl-pyridin-3-ylmethyl}-amine (23)

Example 23 was prepared from 23a (203.3 mg, 0.364 mmol) by the same procedure used to make 21. Analogous chromatography conditions yielded 23 (99.6 mg, 73%) as an off-white solid. ¹H NMR (DMSO-d₆) δ 13.25 (s, 1H), 12.17 (s, 1H), 8.46 (s, 1H), 8.32 (s, 1H), 8.32 (s, 1H), 8.24 (s, 1H), 7.61 (d, J=8.7 Hz, 1H), 7.34 (dd, J=1.5, 8.5 Hz, 1H), 3.83 (s, 2H), 2.68 (q, J=7.2 Hz, 2H) 2.51 (m, 2H), 2.24 (s, 3H), 2.11 (m, 3H), 1.14 (m, 3H), 1.09 (t, J=7.2 Hz, 3H). Anal. Calc. for C₂₂H₂₆N₆.0.4H₂O.0.1 Hexane: C, 69.45; H, 7.28; N, 21.53. Found: C, 69.38; H, 7.15; N, 21.49.

### EXAMPLE 24

### {5-[3-(4,5-Dimethyl-1H-imidazol-2-yl)-1H-indazol-5-yl]-4-methyl-pyridin-3-ylmethyl}-ethyl-amine

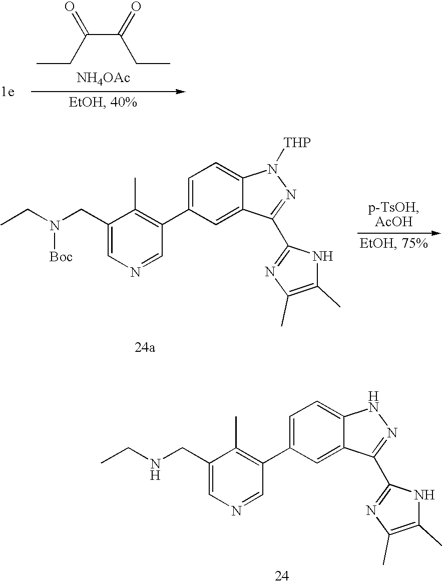

### {5-[3-(4,5-Dimethyl-1H-imidazol-2-yl)-1-(tetrahydro-pyran-2-yl)-1H-indazol-5-yl]-4-methyl-pyridin-3-ylmethyl}-ethyl-carbamic acid tert-butyl ester (24a)

Compound 24a was prepared from aldehyde 1e (499.4 mg, 1.04 mmol), 2,3-butanedione (98.8 mg, 1.15 mmol), and ammonium acetate (483 mg, 6.26 mmol) by a procedure similar to 21a. Purification by silica gel chromatography (eluting with 100% ethyl acetate) afforded 24a (225.6 mg, 40%) as a yellow foam. ¹H NMR (DMSO-d₆) δ 12.21 (s, 1H), 8.35 (s, 1H), 8.29 (s, 1H), 8.26 (s, 1H), 7.83 (d, J=8.9 Hz, 1H), 7.41 (dd, J=1.5, 8.7 Hz, 1H), 5.93 (dd, J=2.2, 10.1 Hz, 1H), 4.51 (s, 2H), 3.92 (m, 1H), 3.80 (m, 1H), 3.20 (m, 2H), 2.55 (m, 1H), 2.16 (s, 3H), 2.12 (m, 9H), 1.79 (m, 1H), 1.61 (m, 2H), 1.40 (br s, 9H), 1.04 (t, J=7.0 Hz, 3H).

### {5-[3-(4,5-Dimethyl-1H-imidazol-2-yl)-1H-indazol-5-yl]-4-methyl-pyridin-3-ylmethyl}-ethyl-amine (24)

Example 23 was prepared from 24a (209.5 mg, 0.385 mmol) by the same procedure used to make 21. Analogous chromatography conditions yielded 24 (104.2 mg, 75%) as an off-white solid. ¹H NMR (DMSO-d₆) δ 13.25 (s, 1H), 12.16 (s, 1H), 8.44 (s, 1H), 8.30 (s, 1H), 8.22 (s, 1H), 7.61 (d, J=8.5 Hz, 1H), 7.33 (dd, J=1.5, 8.5 Hz, 1H), 3.78 (s, 2H), 2.64 (q, J=7.0 Hz, 2H) 2.22 (s, 3H), 2.11 (br s, 6H), 1.07 (t, J=7.0 Hz, 3H). Anal. Calc. for C₂₁H₂₄N₆.0.4H₂O.0.2Hexane: C, 69.27; H, 7.23; N, 21.84. Found: C, 69.23; H, 7.01; N, 21.89.

### EXAMPLE 25

### (5-{3-[4-(4-Chloro-phenyl)-5-methyl-1H-imidazol-2-yl]-1H-indazol-5-yl}-4-methyl-pyridin-3-ylmethyl)-ethyl-amine

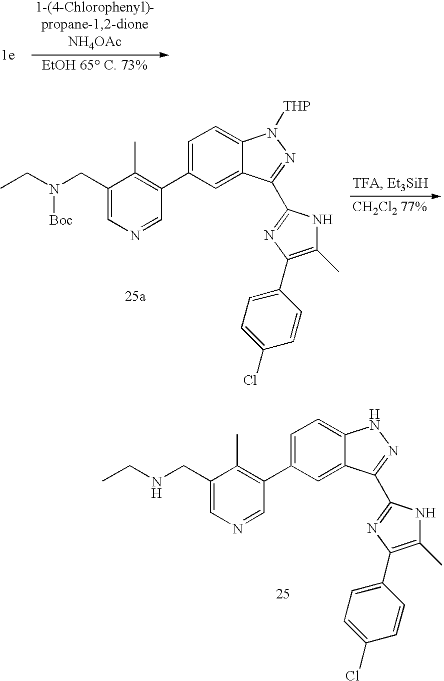

### {5-[3-[4-(4-Chloro-phenyl)-5-methyl-1H-imidazol-2-yl]-1-(tetrahydro-pyran-2-yl)-1H-indazol-5-yl]-4-methyl-pyridin-3-ylmethyl)ethyl-carbamic acid tert-butyl ester (25a)

Example 25a was prepared similarly to Example 19a using aldehyde 1e (300 mg, 0.626 mmol), ammonium acetate (242 mg, 3.14 mmol) and 1-(4-chlorophenyl)-propane-1,2-dione (120 mg, 0.661 mmol). Analogous chromatography conditions afforded 25a (293 mg, 73%) as a glass: 1H NMR (300 MHz, CHLOROFORM-D) δ ppm 8.49 (s, 1 H) 8.46 (s, 1 H) 8.34 (s, 1 H) 7.63 (d, J=8.48 Hz, 3 H) 7.30-7.40 (m, 3 H) 5.75 (dd, J=9.61, 2.26 Hz, 1 H) 4.02-4.15 (m, 1 H) 3.72-3.86 (m, 1 H) 3.25 (s, 2 H) 2.53-2.67 (m, J=27.32 Hz, 1 H) 2.49 (s, 3 H) 2.18-2.27 (m, 3 H) 2.08-2.16 (m, 3 H) 1.64-1.85 (m, 4 H) 1.39-1.53 (m, 9 H) 1.10 (t, J=7.06 Hz, 3 H).

### (5-{3-[4-(4-Chloro-phenyl)-5-methyl-1H-imidazol-2-yl]-1H-indazol-5-yl}-4-methyl-pyridin-3-ylmethyl)-ethyl-amine (25)

Example 25 was prepared similarly to Example 2. Analogous chromatography conditions afforded 25 (161 mg, 77%) as a clear glass: 1H NMR (300 MHz, MeOH) δ ppm 8.43 (s, 1 H) 8.35 (s, 1 H) 8.30 (s, 1 H) 7.63 (d, J=8.29 Hz, 3 H) 7.37 (d, J=8.10 Hz, 3 H) 3.86 (s, 2 H) 2.75 (q, J=6.97 Hz, 2 H) 2.46 (s, 3 H) 2.32 (s, 3 H) 1.17 (t, 3 H).

Anal. Calcd. for C₂₆H₂₅ClN₆.0.7 MeOH: C, 66.89; H, 5.85; N, 17.53; Cl, 7.40. Found: C, 67.02; H, 5.82; N, 17.41; Cl, 7.19.

### EXAMPLE 26

### (5-{3-[4-(3,5-Dichloro-phenyl)-5-methyl-1H-imidazol-2-yl]-1H-indazol-5-yl}-4-methyl-pyridin-3-ylmethyl)-ethyl-amine

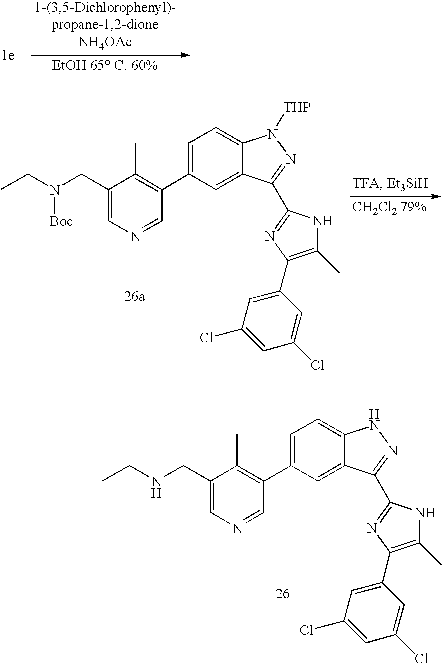

### {5-[3-[4-(3,5-Dichloro-phenyl)-5-methyl-1H-imidazol-2-yl]-1-(tetrahydro-pyran-2-yl)-1H-indazol-5-yl]-4-methyl-pyridin-3-ylmethyl}-ethyl-carbamic acid tert-butyl ester (26a)

Example 26a was prepared similarly to Example 19a using aldehyde 1e (300 mg, 0.626 mmol), ammonium acetate (242 mg, 3.14 mmol) and 1-(3,5-dichlorophenyl)-propane-1,2-dione (136 mg, 0.633 mmol). Analogous chromatography conditions afforded 26a (255 mg, 60%) as a glass: 1H NMR (300 MHz, CHLOROFORM-D) δ ppm 9.87 (s, 1 H) 8.46 (s, 2 H) 8.36 (s, 1 H) 7.61-7.70 (m, 3 H) 7.35 (dd, J=8.67, 1.51 Hz, 1 H) 7.21 (s, 1 H) 5.77 (dd, J=9.70, 2.35 Hz, 1 H) 4.55 (s, 2 H) 4.04-4.16 (m, 1 H) 3.74-3.89 (m, J=3.01 Hz, 1 H) 3.18-3.36 (m, J=6.97 Hz, 2 H) 2.55 (s, 3 H) 2.52-2.71 (m, 1 H) 2.22 (s, 3 H) 2.06-2.27 (m, 1 H) 1.64-1.89 (m, 4 H) 1.47 (s, 9 H) 1.10 (t, J=6.97 Hz, 3 H).

### (5-{3-[4-(3,5-Dichloro-phenyl)-5-methyl-1H-imidazol-2-yl]-1H-indazol-5-yl}-4-methyl-pyridin-3-ylmethyl)-ethyl-amine (26)

Example 26 was prepared similarly to Example 2. Analogous chromatography conditions afforded 26 (146 mg, 79%) as a clear glass: 1H NMR (300 MHz, MeOH) δ ppm 8.45 (s, 1 H) 8.38 (s, 1 H) 8.31 (s, 1 H) 7.60-7.70 (m, 3 H) 7.40 (d, J=8.67 Hz, 1 H) 7.27-7.31 (m, 1 H) 3.89-3.97 (m, 2 H) 2.78 (q, 2 H) 2.51 (s, 3 H) 2.36 (s, 3 H) 1.20 (t, J=7.06 Hz, 3 H).

Anal. Calcd. for C₂₆H₂₄Cl₂N₆.0.2 MeOH: C, 63.21; H, 5.02; N, 16.88: Cl, 14.24. Found: C, 63.13; H, 4.98; N, 16.91; Cl, 14.22.

### EXAMPLE 27

### Ethyl-(4-methyl-5-{3-[5-methyl-4-(3-trifluoromethyl-phenyl)-1H-imidazol-2-yl]-1H-indazol-5-y}-pyridin-3-ylmethyl)-amine

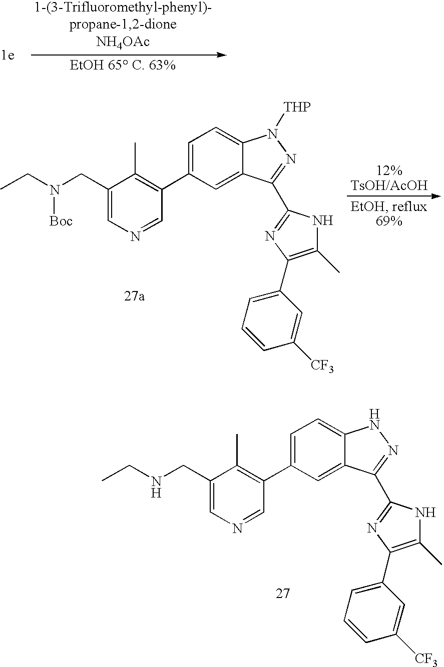

### Ethyl-{4-methyl-5-[3-[5-methyl-4-(3-trifluoromethyl-phenyl)-1H-imidazol-2-yl]-1-(tetrahydro-pyran-2-yl)-1H-indazol-5-yl]-pyridin-3-ylmethyl}-carbamic acid tert-butyl ester (27a)

Example 27a was prepared similarly to Example 19a using aldehyde 1e (302 mg, 0.630 mmol), ammonium acetate (252 mg, 3.27 mmol) and 1-(3-trifluoromethyl-phenyl)-propane-1,2-dione (140 mg, 0.648 mmol). Analogous chromatography conditions afforded 27a (268 mg, 63%) as a glass: 1H NMR (300 MHz, CHLOROFORM-D) δ ppm 8.51 (s, 1 H) 8.46 (s, 1 H) 8.34 (s, 1 H) 7.93 (s, 2 H) 7.61-7.68 (m, 1 H) 7.43-7.56 (m, 3 H) 7.34 (dd, J=8.57, 1.22 Hz, 1 H) 5.76 (dd, J=9.61, 2.26 Hz, 1 H) 4.54 (s, 2 H) 4.02-4.13 (m, 1 H) 3.72-3.85 (m, 1 H) 3.26 (s, 2 H) 2.52-2.67 (m, 1 H) 2.50-2.56 (m, 3 H) 2.24 (s, 3 H) 2.06-2.20 (m, 2 H) 1.66-1.84 (m, 3 H) 1.47 (s, 9 H) 1.10 (t, J=6.97 Hz, 3 H).

### Ethyl-(4-methyl-5-{3-[5-methyl-4-(3-trifluoromethyl-phenyl)-1H-imidazol-2-yl]-1H-indazol-5-yl}-pyridin-3-ylmethyl)-amine (27)

The imidazole 27a (268 mg, 0.397 mmol) was dissolved in EtOH (7 ml) and 12% TsOH in acetic acid solution. The reaction mixture was heated to reflux for 16 hr. The crude mixture was poured into 2N NaOH solution (75 ml) and extracted with 20% isopropanol in chloroform solution (2×50 ml) and EtOAc (2×50 ml). The combined organic layers were dried over magnesium sulfate, filtered and concentrated to a crude oil. Analogous chromatography to conditions for Example 2 afforded 27 (134 mg, 69%) as a clear glass: 1H NMR (300 MHz, MeOH) δ ppm 8.45 (s, 1 H) 8.38 (s, 1 H) 8.32-8.35 (m, 1 H) 7.92-8.03 (m, 2 H) 7.53-7.72 (m, J=8.67 Hz, 3 H) 7.41 (dd, J=8.57, 1.60 Hz, 1 H) 3.90 (s, 2 H) 2.76 (q, 2 H) 2.52 (s, 3 H) 2.36 (s, 3 H) 1.20 (t, J=7.16 Hz, 3 H).

Anal. Calcd. for C₂₇H₂₅F₃N₆.0.1 iPOH.0.2 CHCl₃: C, 63.47; H, 5.04; N, 16.15: F, 10.95. Found: C, 63.48; H, 5.07; N, 16.14; F, 10.68.

### EXAMPLE 28

### Ethyl-(4-methyl-5-{3-[5-methyl-4-(4-trifluoromethyl-phenyl)-1H-imidazol-2-yl]-1H-indazol-5-yl}-pyridin-3-ylmethyl)-amine

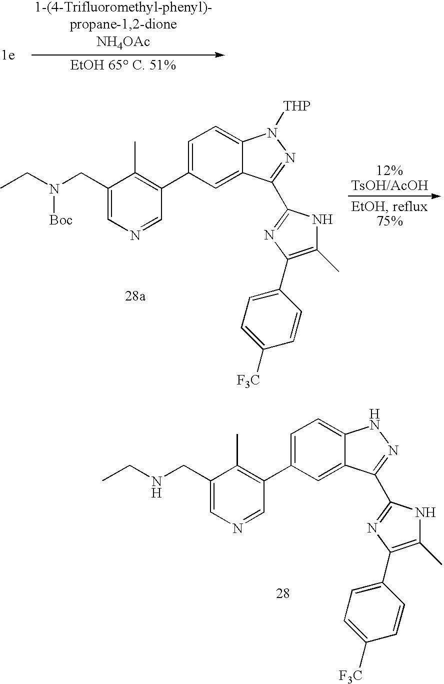

### Ethyl-{4-methyl-5-[3-[5-methyl-4-(4-trifluoromethyl-phenyl)-1H-imidazol-2-yl]-1-(tetrahydro-pyran-2-yl)-1H-indazol-5-yl]-pyridin-3-ylmethyl)carbamic acid tert-butyl ester (28a)

Example 28a was prepared similarly to Example 19a using aldehyde 1e (310 mg, 0.647 mmol), ammonium acetate (246 mg, 3.19 mmol) and 1-(4-trifluoromethyl-phenyl)-propane-1,2-dione (142 mg, 0.657 mmol). Analogous chromatography conditions afforded 28a (268 mg, 63%) as a glass: 1H NMR (300 MHz, CHLOROFORM-D) δ ppm 8.51 (d, J=0.75 Hz, 1 H) 8.46 (s, 1 H) 8.35 (s, 1 H) 7.78-7.89 (m, J=6.97 Hz, 2 H) 7.59-7.69 (m, 3 H) 7.33 (dd, J=8.67, 1.32 Hz, 1 H) 5.76 (d, J=7.91 Hz, 1 H) 4.53 (s, 2 H) 4.01-4.15 (m, 1 H) 3.73-3.86 (m, 1 H) 3.27 (s, 2 H) 2.53 (s, 3 H) 2.50-2.69 (m, 1 H) 2.22 (s, 3 H) 2.04-2.25 (m, 2 H) 1.64-1.86 (m, 3 H) 1.47 (s, 9 H) 1.11 (t, J=6.97 Hz, 3 H).

### Ethyl-(4-methyl-5-{3-[5-methyl-4-(4-trifluoromethyl-phenyl)-1H-imidazol-2-yl]-1H-indazol-5-yl}-pyridin-3-ylmethyl)-amine (28)

Example 28 was prepared similarly to Example 27. Analogous chromatography conditions afforded 28 (122 mg, 75%) as a clear glass: 1H NMR (300 MHz, MeOH) δ ppm 8.44 (s, 1 H) 8.36 (s, 1 H) 8.32 (s, 1 H) 7.86 (d, J=8.10 Hz, 2 H) 7.61-7.74 (m, 3 H) 7.38 (dd, J=8.67, 1.51 Hz, 1 H) 3.87 (s, 2 H) 2.75 (q, J=7.16 Hz, 2 H) 2.53 (s, 3 H) 2.33 (s, 3 H) 1.19 (t, J=7.16 Hz, 3 H).

Anal. Calcd. for C₂₇H₂₅F₃N₆.0.1 MeOH.0.3 H₂O: C, 65.21; H, 5.25; N, 16.84: F, 11.42. Found: C, 65.24; H, 5.19; N, 16.80; F, 11.34.

### EXAMPLE 29

### Ethyl-{4-methyl-5-[3-(5-methyl-4-phenyl-1H-imidazol-2-yl)-1H-indazol-5-yl]-pyridin-3-ylmethyl}-amine

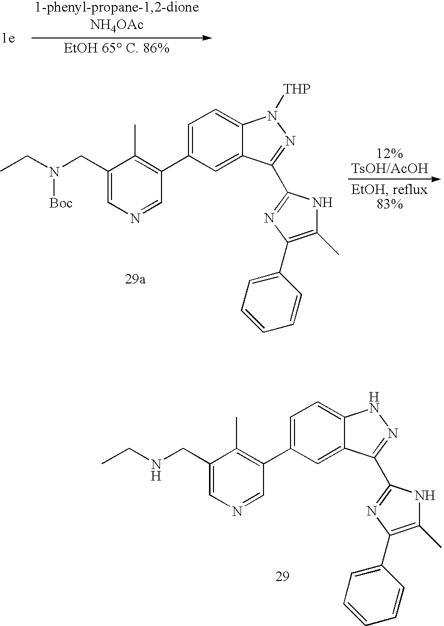

### Ethyl-{4-methyl-5-[3-(5-methyl-4-phenyl-1H-imidazol-2-yl)-1-(tetrahydro-pyran-2-yl)-1H-indazol-5-yl]-pyridin-3-ylmethyl}-carbamic acid tert-butyl ester (29a)

Example 29a was prepared similarly to Example 19a using aldehyde 1e (400 mg, 0.835 mmol), ammonium acetate (361 mg, 4.69 mmol) and 1-phenyl-propane-1,2-dione (120 ul, 0.893 mmol). Analogous chromatography conditions afforded 29a (437 mg, 86%) as a glass: 1H NMR (300 MHz, CHLOROFORM-D) δ ppm 8.52 (s, 1 H) 8.46 (s, 1 H) 8.34 (s, 1 H) 7.61-7.71 (m, 3 H) 7.41 (t, J=7.54 Hz, 2 H) 7.27-7.37 (m, 2 H) 5.76 (d, 1 H) 4.54 (s, 2 H) 4.05-4.17 (m, 1 H) 3.73-3.87 (m, 1 H) 3.26 (s, 2 H) 2.53-2.68 (m, 1 H) 2.50-2.57 (m, 3 H) 2.23 (s, 3 H) 2.06-2.21 (m, 2 H) 1.65-1.87 (m, 3 H) 1.47 (s, 9 H) 1.07-1.15 (m, 3 H).

### Ethyl-{4-methyl-5-[3-(5-methyl-4-phenyl-1H-imidazol-2-yl)-1H-indazol-5-yl]-pyridin-3-ylmethyl}-amine (29)

Example 29 was prepared similarly to Example 27. Analogous chromatography conditions afforded 29 (252 mg, 83%) as a clear glass: 1H NMR (300 MHz, DMSO-D6) δ ppm 13.40 (s, 1 H) 12.66 (s, 1 H) 8.45 (s, 1 H) 8.33-8.38 (m, 2 H) 7.64-7.78 (m, 3 H) 7.33-7.43 (m, 3 H) 7.20 (d, J=7.35 Hz, 1 H) 3.77 (s, 2 H) 2.63 (q, J=7.16 Hz, 2 H) 2.50-2.53 (m, 3 H) 2.25-2.30 (m, 3 H) 1.07 (t, J=7.06 Hz, 3 H).

Anal. Calcd. for C₂₆H₂₆N₆.0.6 H₂O.0.15 MeOH.0.03 CHCl₃: C, 71.18; H, 6.35; N, 19.03. Found: C, 71.20; H, 6.32; N, 19.02.

### EXAMPLE 30

### Ethyl-{4-methyl-5-[3-(4-phenyl-1H-imidazol-2-yl)-1H-indazol-5-yl]-pyridin-3-ylmethyl}-amine

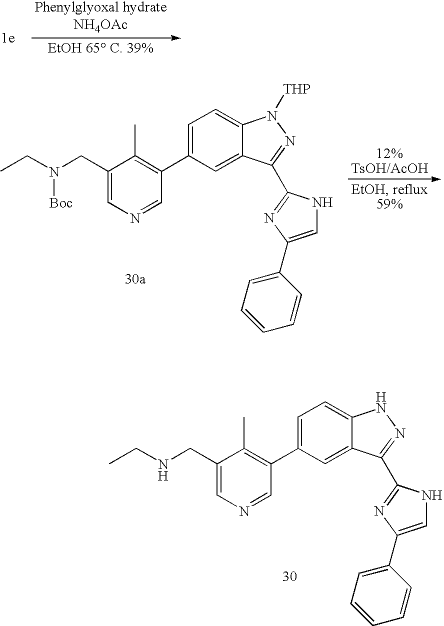

### Ethyl-{4-methyl-5-[3-(4-phenyl-1H-imidazol-2-yl)-1-(tetrahydro-pyran-2-yl)-1H-indazol-5-yl]-pyridin-3-ylmethyl}-carbamic acid tert-butyl ester (30a)

Example 30a was prepared similarly to Example 19a using aldehyde 1e (401 mg, 0.837 mmol), ammonium acetate (325 mg, 4.22 mmol) and phenylglyoxal hydrate (128 mg, 0.836 mmol). Analogous chromatography conditions afforded 30a (191 mg, 39%) as a glass: 1H NMR (300 MHz, CHLOROFORM-D) δ ppm 8.54 (s, 1 H) 8.47 (s, 1 H) 8.35 (s, 1 H) 7.72-7.81 (m, J=8.10 Hz, 2 H) 7.65 (d, J=8.67 Hz, 1 H) 7.31-7.43 (m, 4 H) 7.22-7.29 (m, 1 H) 5.77 (dd, J=9.51, 2.35 Hz, 1 H) 4.54 (s, 2 H) 4.03-4.15 (m, 1 H) 3.74-3.86 (m, 1 H) 3.26 (s, 2 H) 2.53-2.68 (m, 1 H) 2.22-2.26 (m, 3 H) 2.07-2.20 (m, 2 H) 1.67-1.86 (m, 3 H) 1.48 (s, 9 H) 1.10 (t, 3 H).

### Ethyl-{4-methyl-5-[3-(4-phenyl-1H-imidazol-2-yl)-1H-indazol-5-yl]-pyridin-3-ylmethyl}-amine (26)

Example 30 was prepared similarly to Example 27. Analogous chromatography conditions afforded 30 (79 mg, 59%) as a clear glass: 1H NMR (300 MHz, DMSO-D6) δ ppm 13.45 (s, 1 H) 12.84 (s, 1 H) 8.46 (s, 1 H) 8.27-8.42 (m, 2 H) 7.86 (d, J=7.91 Hz, 2 H) 7.65-7.74 (m, 2 H) 7.30-7.45 (m, 3 H) 7.13-7.22 (m, 1 H) 3.79 (s, 2 H) 2.64 (q, J=7.16 Hz, 2 H) 2.28 (s, 3 H) 1.08 (t, J=6.97 Hz, 3 H).

Anal. Calcd. for C₂₅H₂₄N₆.0.05 H₂O.0.4 EtOAc: C, 71.85; H, 6.19; N, 18.90. Found: C, 71.88; H, 6.17; N, 18.91.

### EXAMPLE 31

### Ethyl-(5-{3-[4-(4-fluoro-phenyl)-1H-imidazol-2-yl]-1H-indazol-5-yl}-4-methyl-pyridin-3-ylmethyl)-amine

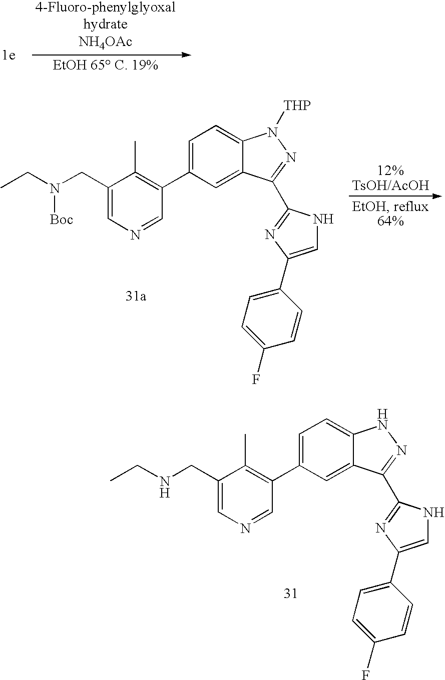

### Ethyl-{5-[3-[4-(4-fluoro-phenyl)-1H-imidazol-2-yl]-1-(tetrahydro-pyran-2-yl)-1H-indazol-5-yl]-4-methyl-pyridin-3-ylmethyl}-carbamic acid tert-butyl ester (31a)

Example 31a was prepared similarly to Example 19a using aldehyde 1e (755 mg, 1.576 mmol), ammonium acetate (755 mg, 7.86 mmol) and 4-fluoro-phenylglyoxal hydrate (800 mg, 4.706 mmol) in EtOH (20 ml). Analogous chromatography conditions afforded 31a (185 mg, 19%) as a glass: 1H NMR (300 MHz, CHLOROFORM-D) δ ppm 8.53 (s, 1 H) 8.47 (s, 1 H) 8.35 (s, 1 H) 7.76 (s, 2 H) 7.63-7.69 (m, 2 H) 7.34 (s, 1 H) 7.05 (t, J=8.19 Hz, 2 H) 5.76 (d, J=8.67 Hz, 1 H) 4.54 (s, 2 H) 4.03-4.11 (m, 1 H) 3.79 (t, J=10.46 Hz, 1 H) 3.23-3.33 (m, J=3.39 Hz, 2 H) 2.53-2.67 (m, J=10.74 Hz, 1 H) 2.22 (s, 3 H) 2.06-2.18 (m, J=10.17 Hz, 2 H) 1.66-1.82 (m, 3 H) 1.47 (s, 9 H) 1.11 (t, J=6.97 Hz, 3 H).

### Ethyl-(5-{3-[4-(4-fluoro-phenyl)-1H-imidazol-2-yl]-1H-indazol-5-yl}-4-methyl-pyridin-3-ylmethyl)-amine (31)

Example 31 was prepared similarly to Example 27. Analogous chromatography conditions afforded 31 (82 mg, 64%) as a clear glass: 1H NMR (400 MHz, MeOD) δ ppm 8.41 (s, 1 H) 8.33 (d, J=8.08 Hz, 2 H) 7.79 (dd, J=8.08, 5.56 Hz, 2 H) 7.62 (d, J=8.59 Hz, 1 H) 7.48 (s, 1 H) 7.36 (dd, J=8.59, 1.26 Hz, 1 H) 7.07 (t, J=8.72 Hz, 2 H) 3.83 (s, 2 H) 2.69-2.74 (m, 2 H) 2.30 (s, 3 H) 1.17 (t, J=7.20 Hz, 3 H).

Anal. Calcd. for C₂₅H₂₃FN₆.0.35 MeOH.0.03 CHCl₃: C, 68.44; H, 5.53; N, 18.84. Found: C, 68.49; H, 5.53; N, 18.64.

### EXAMPLE 32

### Ethyl-(5-{3-[4-(3-fluoro-phenyl)-1H-imidazol-2-yl]-1H-indazol-5-yl}-4-methyl-pyridin-3-ylmethyl)-amine

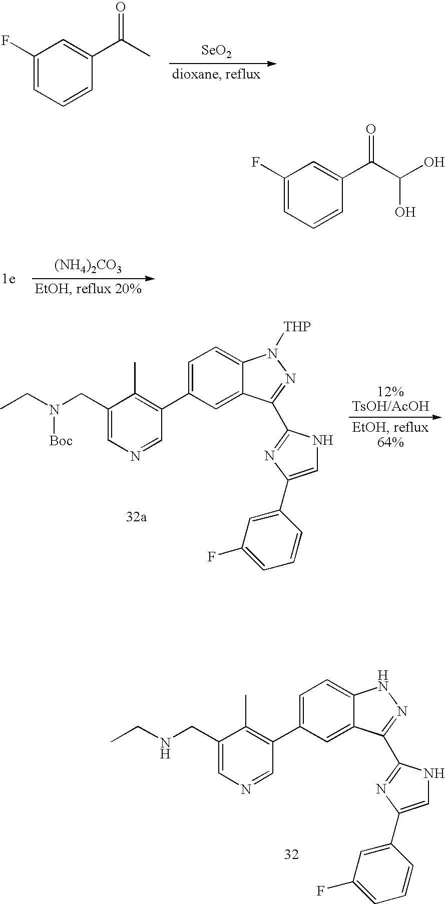

### Ethyl-{5-[3-[4-(3-fluoro-phenyl)-1H-imidazol-2-yl]-1-(tetrahydro-pyran-2-yl)-1H-indazol-5-yl]-4-methyl-pyridin-3-ylmethyl}-carbamic acid tert-butyl ester (32a)

3-Fluoro-acetophenone (275 ul, 2.24 mmol) was dissolved in 1,4-dioxane (15 ml). To this solution was added selenium dioxide (275 mg, 2.48 mmol) and the mixture heated to reflux over 16 hr. The reaction mixture was filtered through celite eluting with EtOAc (75 ml) and then the solvents removed in vacuo. The resulting crude glyoxal was dissolved in EtOH (15 ml) along with aldehyde 1e (745 mg, 1.56 mmol). Finally, ammonium carbonate (755 mg, 7.86 mmol) was added and the reaction mixture heated to reflux for 16 hr. The mixture was then poured into EtOAc (100 ml) and washed with brine (3×50 ml). The aqueous layer was reextracted with EtOAc (2×50 ml). The combined organics were dried over magnesium sulfate, filtered and concentrated to a crude oil. Purification by silica gel chromatography (eluting with 40-100% ethyl acetate in hexanes) afforded 32a (189 mg, 20%) as a clear glass: 1H NMR (400 MHz, CHLOROFORM-D) δ ppm 8.53 (s, 1 H) 8.46 (s, 1 H) 8.36 (s, 1 H) 7.65 (d, J=8.59 Hz, 1 H) 7.57 (s, 1 H) 7.51 (s, 1 H) 7.41 (s, 1 H) 7.29-7.37 (m, 2 H) 6.88-6.95 (m, 1 H) 5.73-5.81 (m, 1 H) 4.54 (s, 2 H) 4.03-4.12 (m, 1 H) 3.74-3.84 (m, 1 H) 3.27 (s, 2 H) 2.54-2.68 (m, 1 H) 2.23 (s, 3 H) 2.07-2.20 (m, 2 H) 1.66-1.84 (m, 3 H) 1.47 (s, 9 H) 1.11 (t, J=6.95 Hz, 3 H).

### Ethyl-(5-{3-[4-(3-fluoro-phenyl)-1H-imidazol-2-yl]-1H-indazol-5-yl}-4-methyl-pyridin-3-ylmethyl)-amine (32)

Example 32 was prepared similarly to Example 27. Analogous chromatography conditions afforded 32 (95 mg, 72%) as a clear glass: 1H NMR (300 MHz, DMSO-D6) δ ppm 13.49 (s, 1 H) 12.94 (s, 1 H) 8.45 (s, 1 H) 8.34-8.40 (m, 2 H) 7.84 (s,1 H) 7.62-7.72 (m, J=8.67 Hz, 3 H) 7.35-7.44 (m, J=8.10 Hz, 2 H) 6.99 (t, J=8.38 Hz, 1 H) 3.78 (s, 2 H) 2.63 (q, J=6.59 Hz, 2 H) 2.28 (s, 3 H) 1.07 (t, J=6.97 Hz, 3 H).

Anal. Calcd. for C₂₅H₂₃FN₆.0.25 MeOH.0.1 H₂O: C, 68.20; H, 5.44; N, 18.83. Found: C, 68.13; H, 5.45; N, 18.82.

### EXAMPLE 33

### (5-{3-[4-(3,4-Dimethoxy-phenyl)-1H-imidazol-2-yl]-1H-indazol-5-yl}-4-methyl-pyridin-3-ylmethyl)-ethyl-amine

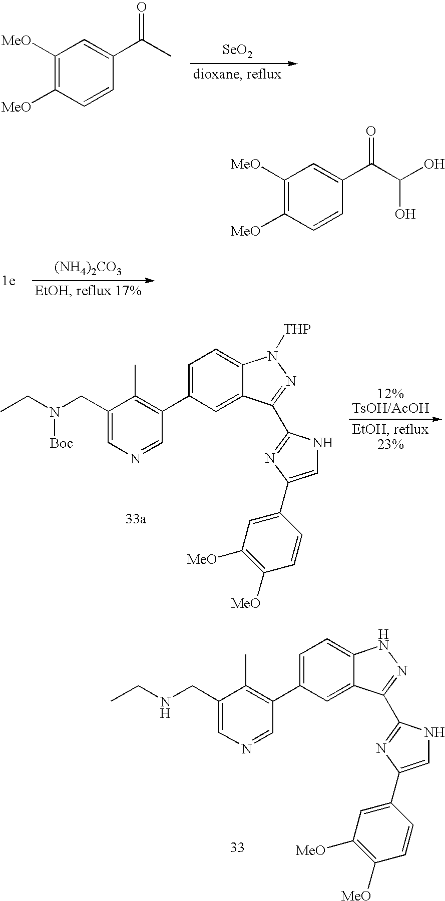

### {5-[3-[4-(3,4-Dimethoxy-phenyl)-1H-imidazol-2-yl]-1-(tetrahydro-pyran-2-yl)-1H-indazol-5-yl]-4-methyl-pyridin-3-ylmethyl}-ethyl-carbamic acid tert-butyl ester (33a)

Example 33a was prepared similarly to Example 32a using 3,4-dimethoxyacetophenone (405 mg, 2.25 mmol), aldehyde 1e (750 mg, 1.57 mmol) and ammonium carbonate (755 mg, 7.86 mmol). Analogous chromatography conditions afforded 33a (170 mg, 17%) as a glass: 1H NMR (400 MHz, CHLOROFORM-D) δ ppm 8.52 (s, 1 H) 8.46 (s, 1 H) 8.34 (s,1 H) 7.61-7.71 (m, 2 H) 7.42-7.48 (m, 1 H) 7.31-7.36 (m, 1 H) 6.90 (d, J=8.34 Hz, 1 H) 5.76 (dd, J=9.47, 2.15 Hz, 1 H) 4.53 (s, 2 H) 4.05-4.11 (m, 2 H) 3.92-3.94 (m, 3 H) 3.89 (s, 3 H), 3.76-3.82 (m, J=7.83 Hz, 1 H) 3.25 (s, 2 H) 2.53-2.68 (m, J=9.35 Hz, 1 H) 2.24 (s, 3 H) 2.05-2.21 (m, 2 H) 1.66-1.84 (m, 3 H) 1.47 (s, 9 H) 1.11 (t, J=6.82 Hz, 3 H).

### (5-{3-[4-(3,4-Dimethoxy-phenyl)-1H-imidazol-2-yl]-1H-indazol-5-yl}-4-methyl-pyridin-3-ylmethyl)-ethyl-amine (33)

Example 33 was prepared similarly to Example 27. Analogous chromatography conditions afforded 33 (28 mg, 23%) as a clear glass: 1H NMR (300 MHz, MeOH) δ ppm 8.48 (s, 1 H) 8.42 (s, 1 H) 8.32 (s, 1 H) 7.68 (d, J=8.67 Hz, 1 H) 7.32-7.47 (m, 4 H) 6.97 (d, J=8.29 Hz, 1 H) 4.01 (s, 2 H) 3.88-3.92 (m, 3 H) 3.82-3.86 (m, 3 H) 2.81-2.92 (m, 2 H) 2.37 (s, 3 H) 1.25 (t, J=7.16 Hz, 3 H).

Anal. Calcd. for C₂₇H₂₈N₆O₂.0.27 CHCl₃.1.8 H₂O: C, 61.58; H, 6.00; N, 15.64. Found: C, 61.42; H, 6.02; N, 15.76.

### EXAMPLE 34

### Ethyl-(5-{3-[4-(4-methoxy-phenyl)-1H-imidazol-2-yl]-1H-indazol-5-yl}-4-methyl-pyridin-3-ylmethyl)-amine

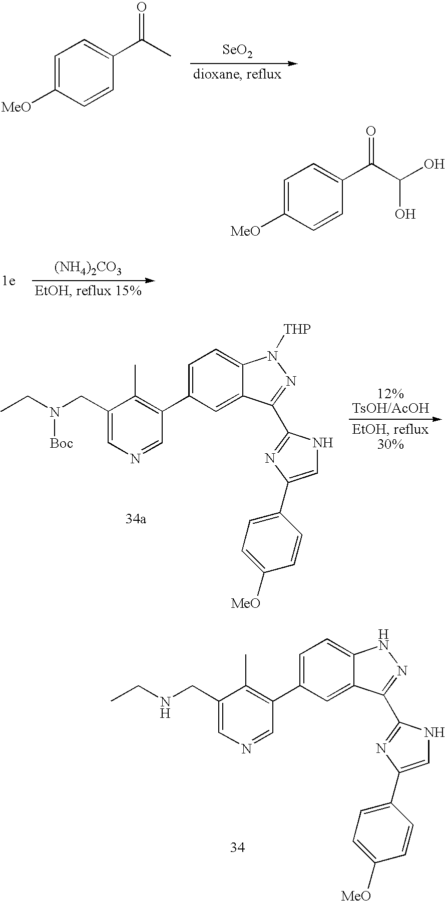

### Ethyl-{5-[3-[4-(4-methoxy-phenyl)-1H-imidazol-2-yl]-1-(tetrahydro-pyran-2-yl)-1H-indazol-5-yl]-4-methyl-pyridin-3-ylmethyl}-carbamic acid tert-butyl ester (34a)

Example 34a was prepared similarly to Example 32a using 4-methoxyacetophenone (338 mg, 2.25 mmol), aldehyde 1e (755 mg, 1.58 mmol) and ammonium carbonate (755 mg, 7.86 mmol). Analogous chromatography conditions afforded 34a (150 mg, 15%) as a glass: 1H NMR (400 MHz, CHLOROFORM-D) δ ppm 8.53 (s, 1 H) 8.46 (s, 1 H) 8.31-8.39 (m, 1 H) 7.62-7.71 (m, 3 H) 7.31-7.34 (m, J=3.54, 3.54 Hz, 2 H) 6.93 (d, J=8.84 Hz, 2 H) 5.77 (dd, J=9.47, 2.40 Hz, 1 H) 4.54 (s, 2 H) 4.01-4.10 (m, 1 H) 3.81-3.84 (m, 3 H) 3.76-3.84 (m, 1 H) 3.26 (s, 2 H) 2.56-2.67 (m, 1 H) 2.25 (s, 3 H) 2.08-2.21 (m, 2 H) 1.67-1.85 (m, 3 H) 1.48 (s, 9 H) 1.12 (t, J=6.95 Hz, 3 H).

### Ethyl-(5-{3-[4-(4-methoxy-phenyl)-1H-imidazol-2-yl]-1H-indazol-5-yl}-4-methyl-pyridin-3-ylmethyl)-amine (34)

Example 34 was prepared similarly to Example 27. HPLC chromatography conditions (0.5% TFA/CH₃CN and 0.1% TFA/H₂O) afforded 34 (32 mg, 30%) as a clear glass: 1H NMR (300 MHz, MeOH) δ ppm 8.63 (d, J=8.85 Hz, 2 H) 8.06 (d, J=8.85 Hz, 2 H) 7.73 (dd, J=12.15, 8.76 Hz, 3 H) 7.62 (s, 1 H) 7.04 (d, J=9.04 Hz, 2 H) 4.46 (s, 2 H) 3.89 (s, 3 H) 3.23-3.28 (m, 2 H) 2.49 (s, 3 H) 1.35-1.42 (m, 3 H).

Anal. Calcd. for C₂₆H₂₆N₆O.1.0 MeOH.2.0 TFA.1.6 H₂O: C, 51.18; H, 4.88; N, 11.55. Found: C, 51.14; H, 4.90; N, 11.59.

### EXAMPLE 35

### Ethyl-(5-{3-[4-(3-methoxy-phenyl)-1H-imidazol-2-yl]-1H-indazol-5-yl}-4-methyl-pyridin-3-ylmethyl)-amine

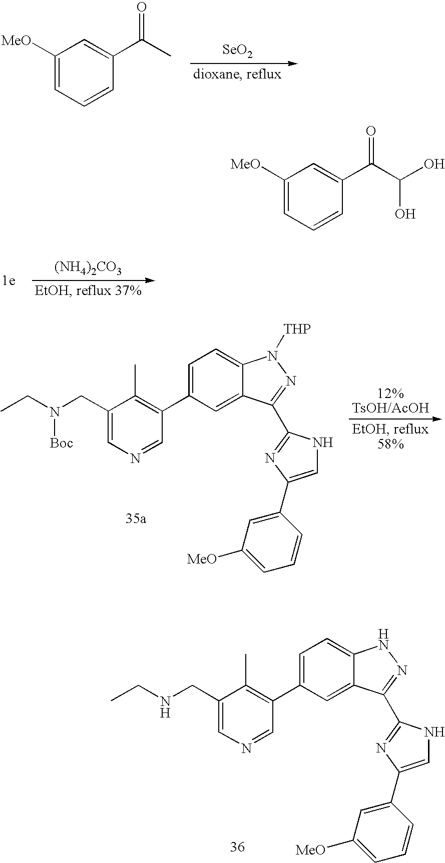

### Ethyl-{5-[3-[4-(3-methoxy-phenyl)-1H-imidazol-2-yl]-1-(tetrahydro-pyran-2-yl)-1H-indazol-5-yl]-4-methyl-pyridin-3-ylmethyl}-carbamic acid tert-butyl ester (35a)

Example 35a was prepared similarly to Example 32a using 3-methoxyacetophenone (310 ul, 2.26 mmol), aldehyde 1e (751 mg, 1.57 mmol) and ammonium carbonate (755 mg, 7.86 mmol). Analogous chromatography conditions afforded 35a (268 mg, 37%) as a glass: 1H NMR (400 MHz, CHLOROFORM-D) δ ppm 8.52 (s, 1 H) 8.46 (s, 1 H) 8.35 (s, 1 H) 7.64-7.69 (m, 1 H) 7.41-7.46 (m, 2 H) 7.27-7.36 (m, 3 H) 6.77-6.84 (m, 1 H) 5.77 (dd, J=9.47, 2.40 Hz, 1 H) 4.54 (s, 2 H) 4.04-4.12 (m, 1 H) 3.84 (s, 3 H) 3.76-3.87 (m, 1 H) 3.27 (s, 2 H) 2.55-2.69 (m, 1 H) 2.25 (s, 3 H) 2.07-2.19 (m, 2 H) 1.67-1.84 (m, 3 H) 1.48 (s, 9 H) 1.12 (t, J=7.07 Hz, 3 H).

### Ethyl-(5-{3-[4-(3-methoxy-phenyl)-1H-imidazol-2-yl]-1H-indazol-5-yl}-4-methyl-pyridin-3-ylmethyl)-amine (35)

Example 35 was prepared similarly to Example 27. Analogous chromatography conditions afforded 35 (132 mg, 58%) as a clear glass: 1H NMR (400 MHz, MeOD) δ ppm 8.42 (s, 1 H) 8.36 (s, 1 H) 8.32 (s, 1 H) 7.64 (d, J=8.59 Hz, 1 H) 7.52 (s, 1 H) 7.37 (t, J=7.33 Hz, 3 H) 7.27 (t, J=7.83 Hz, 1 H) 6.80 (d, J=7.07 Hz, 1 H) 3.85 (s, 2 H) 3.81 (s, 3 H) 2.74 (q, J=7.07 Hz, 2 H) 2.32 (s, 3 H) 1.18 (t, J=7.20 Hz, 3 H).

Anal. Calcd. for C₂₆H₂₆N₆O.0.1 CHCl₃.0.1 H₂O: C, 69.31; H, 5.86; N, 18.58. Found: C, 69.34; H, 5.87; N, 18.61.

### EXAMPLE 36

### 4-Methyl-5-[3-(4,5,6,7-tetrahydro-1H-benzoimidazol-2-yl)-1H-indazol-5-yl]-pyridin-3-ylamine

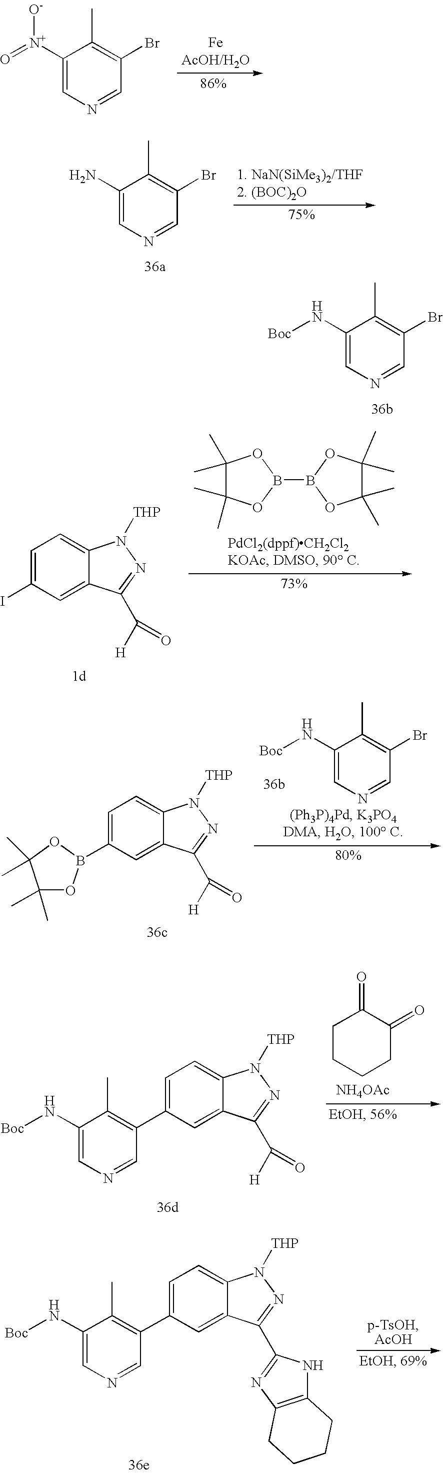

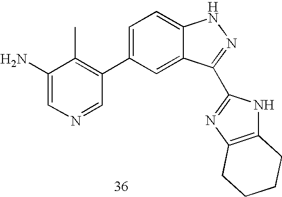

### 5-Bromo-4-methyl-pyridin-3-ylamine (36a)

3-Bromo-4-methyl-5-nitro-pyridine [Prepared as described in: Prokopov, A. A.; Yakhontov, L. N. Chem. Hetero. Compd. (Engl. Transl.), 1979, 15, 76-78.] (21.73 g, 100 mmol) was dissolved in 200 mL glacial acetic acid and 50 mL deionized water. Iron powder (16.78 g, 300 mmol) was added in small portions over one hour, slowly enough to keep the internal temperature below 45° C. Stirring was continued at room temperature for 45 minutes. The reaction mixture was diluted with 200 mL ethyl acetate and 500 mL 5M sodium hydroxide. The resulting emulsion was filtered through Celite, the layers separated, and the aqueous layer back-extracted with 200 mL ethyl acetate. The combined organic layers were dried over magnesium sulfate, filtered, and concentrated. Purification of the crude product by silica gel chromatography (eluting with 80% ethyl acetate in hexanes) afforded amine 36a (16.16 g, 86%) as a white solid. ¹H NMR (DMSO-d₆) δ 7.84 (s, 1H), 7.82 (s, 1H), 5.46 (br s, 2H), 2.14 (s, 3H).

### (5-Bromo-4-methyl-pyridin-3-yl)-carbamic acid tert-butyl ester (36b)

Amine 36a (6.60 g, 35.3 mmol) was dissolved in 375 mL anhydrous tetrahydrofuran at room temperature. A solution of sodium bis(trimethylsilyl) amide (1.0 M in THF, 77.6 mL) was added dropwise over 5 minutes, and stirring was continued at room temperature for 15 minutes. Solid di-tert-butyl dicarbonate (8.47 g, 38.8 mmol) was added in one portion, and stirring continued at room temperature for 1 hour, 10 minutes. The mixture was quenched with 150 mL deionized water and extracted with ethyl acetate (2×300 mL). The organic extracts were dried over magnesium sulfate, filtered and concentrated to a red-brown solid. This was purified by silica gel chromatography (eluting with 50% ethyl acetate in hexanes), affording 36b (7.65 g, 75%) as a yellow solid. ¹H NMR (DMSO-d₆) δ 9.05 (s, 1H), 8.46 (d, J=1.9 Hz, 1H), 8.40 (d, J=1.7 Hz, 1H), 2.27 (s, 3H), 1.45 (s, 9H). Anal. Calc. for C₁₁H₁₅BrN₂O₂: C, 46.01; H, 5.27; N, 9.76; Br, 27.83. Found: C, 46.12; H, 5.30; N, 9.69; Br, 27.66.

### 1-(Tetrahydro-pyran-2-yl)-5-(4,4,5,5-tetramethyl-[1,3,2]dioxaborolan-2-yl)-1H-indazole-3-carbaldehyde (36c)

As in the synthesis of 1e, iodo-indazole 1a (18.24 g, 51.2 mmol) was treated with bis(pinacolato)diboron (14.3 g, 56.3 mmol), potassium acetate (25.1 g, 256 mmol), and [1,1′-bis(diphenylphosphino)-ferrocene]dichloropalladium(II) complex with dichloromethane (1.04 g, 1.28 mmol), but in 500 mL dimethylsulfoxide as solvent. After heating at 90° C. for 2 hours and cooling to room temperature, the mixture was diluted with 500 mL ethyl acetate and washed with deionized water (2×300 mL). The combined aqueous washes were back-extracted with ethyl acetate (2×300 mL). All the organic extracts were combined, dried over sodium sulfate, filtered, and concentrated to a crude red-black solid. Silica gel chromatography (eluting with a gradient of 10-20% tert-butyl methyl ether in a mixture of 3/1 hexanes/dichloromethane) yielded a pink solid (12.23 g), which contained boronic ester 36c contaminated with pinacol. This material was triturated from cyclohexane and dried to give pure 36c (10.64 g, 58%) as a white powder. The cyclohexane solubles from the trituration were combined with impure, product-containing fractions from the column, concentrated, and repurified as above to give a second crop of 36c (2.72 g, 15%) as a white powder. ¹H NMR (CDCl₃) δ 10.26 (s, 1H), 8.80 (s, 1H), 7.86 (d, J=8.6 Hz, 1H), 7.62 (d, J=8.6 Hz, 1H), 5.83 (dd, J=8.7, 2.9 Hz, 1H), 4.00 (m, 1H), 3.78 (m, 1H), 2.57 (m, 1H), 2.13 (m, 2H), 1.78 (m, 3 H), 1.36 (s, 12H).

### {5-[3-Formyl-1-(tetrahydro-pyran-2-yl)-1H-indazol-5-yl]-4-methyl-pyridin-3-yl}-carbamic acid tert-butyl ester (36d)

Boronic ester 36c (5.46 g, 15.33 mmol), bromopyridine 36b (4.40 g, 15.33 mmol), and potassium phosphate (4.88 g, 23.0 mmol) were dissolved in 153 mL N,N-dimethylacetamide and 15.3 ml deionized water. The solution was degassed as described in the procedure for 1e, tetrakis(triphenylphosphine) palladium (0) (0.88 g, 0.766 mmol) was added, and the solution degassed again. The solution was heated to 100° C. for 2 hours. After cooling to room temperature, 100 mL deionized water and 100 mL saturated aqueous sodium bicarbonate solution were added. The mixture was extracted with ethyl acetate (3×200 mL), and the combined organic extracts were dried over magnesium sulfate, filtered, and concentrated. The brown oil obtained was purified by silica gel chromatography (50-100% ethyl acetate in hexanes), followed by trituration of the columned product from chloroform/hexanes. After drying, 36d (5.36 g, 80%) was obtained as a white powder. ¹H NMR (DMSO-d₆) δ 10.21 (s, 1H), 8.97 (s, 1H), 8.47 (s, 1H), 8.23 (s, 1H), 8.04 (s, 1H), 8.02 (d, J=9.8 Hz, 1H), 7.55 (dd, J=1.5, 8.7 Hz, 1H), 6.12 (dd, J=2.0, 9.2 Hz, 1H), 3.90 (m, 2H), 2.42 (m, 1H), 2.10 (s, 3H), 2.09 (m, 2H), 1.80 (m, 1H), 1.63 (m, 2H), 1.47 (s, 9H). Anal. Calc. for C₂₄H₂₈N₄O₄.0.06 CHCl₃: C, 65.13; H, 6.38; N, 12.63. Found: C, 65.39; H, 6.30; N, 12.35.

### {4-Methyl-5-[3-(4,5,6,7-tetrahydro-1H-benzoimidazol-2-yl)-1-(tetrahydro-pyran-2-yl)-1H-indazol-5-yl]-pyridin-3-yl}-carbamic acid tert-butyl ester (36e)

By the same procedure used to make 21a, aldehyde 36d (834.5 mg, 1.91 mmol) was cyclized with 1,2-cyclohexanedione (225 mg, 2.01 mmol) in the presence of ammonium acetate (884 mg, 11.5 mmol), affording, after analogous purification, 36e (564.4 mg, 56%) as a yellow powder. ¹H NMR (DMSO-d₆) δ 12.24 (s, 1H), 8.95 (s, 1H), 8.46 (s, 1H), 8.27 (s, 1H), 8.22 (s, 1H), 7.84 (d, J=8.7 Hz, 1H), 7.41 (dd, J=1.1, 8.5 Hz, 1H), 5.93 (d, J=8.1 Hz, 1H), 3.92 (m, 1H), 3.79 (m, 1H), 2.54 (m, 4H), 2.10 (s, 3H), 2.05 (m, 2H), 1.75 (m, 5H), 1.61 (m, 2H), 1.47 (s, 9H). Anal. Calc. for C₃₀H₃₆N₆O₃.0.3 EtOAc.0.3 H₂O: C, 66.86; H, 7.01; N, 15.00. Found: C, 66.63; H, 6.79; N, 14.85.

### 4-Methyl-5-[3-(4,5,6,7-tetrahydro-1H-benzoimidazol-2-yl)-1H-indazol-5-yl]-pyridin-3-ylamine (36)

Example 36 was prepared from 36e (404.2 mg, 0.765 mmol) by the same procedure used to make 21. Analogous chromatography conditions yielded 36 (181.3 mg, 69%) as a yellow solid: ¹H NMR (MeOD) δ 8.21 (s, 1H), 7.94 (s, 1H), 7.79 (s, 1H), 7.60 (d, J=8.7 Hz, 1H), 7.35 (dd, J=1.5, 8.7 Hz, 1H), 2.64 (br s, 4H), 2.11 (s, 3H), 1.86 (br s, 4H). Anal. Calc. for C₂₀H₂₀N₆.0.5CHCl₃.0.2MeOH: C, 60.56; H, 5.23; N, 20.47. Found: C, 60.62; H, 5.26; N, 20.41.

### EXAMPLE 37

### 5-[3-(4,5-Diethyl-1H-imidazol-2-yl)-1H-indazol-5-yl]-4-methyl-pyridin-3-ylamine

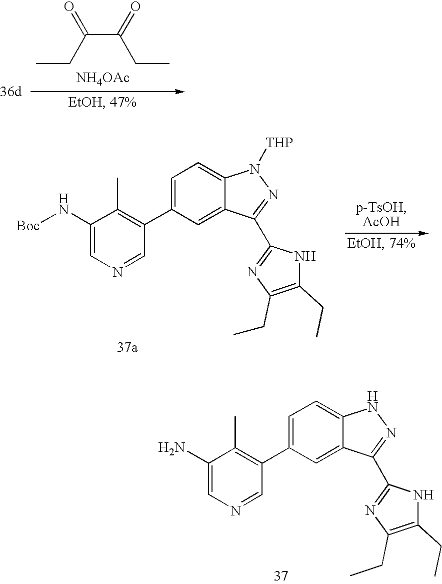

### {5-[3-(4,5-Diethyl-1H-imidazol-2-yl)-1-(tetrahydro-pyran-2-yl)-1H-indazol-5-yl]-4-methyl-pyridin-3-yl}-carbamic acid tert-butyl ester (37a)

Compound 37a was prepared from aldehyde 36d (574.5 mg, 1.32 mmol), 3,4-hexanedione (165 mg, 1.45 mmol), and ammonium acetate (609 mg, 7.9 mmol) by a procedure similar to 21a. Purification by silica gel chromatography (eluting with 60-100% ethyl acetate in hexanes) afforded 37a (329.2 mg, 47%) as a yellow powder. ¹H NMR (DMSO-d₆) δ 12.19 (s, 1H), 8.95 (s, 1H), 8.46 (s, 1H), 8.32 (s, 1H), 8.24 (s, 1H), 7.83 (d, J=8.7 Hz, 1H), 7.43 (dd, J=1.5, 8.7 Hz, 1H), 5.93 (dd, J=2.1, 10.2 Hz, 1H), 3.93 (m, 1H), 3.80 (m, 1H), 2.53 (m, 4H), 2.12 (s, 3H), 2.04 (m, 2H), 1.79 (m, 1H), 1.61 (m, 2H), 1.47 (s, 9H), 1.15 (t, J≦7.5 Hz, 6H).

### 5-[3-(4,5-Diethyl-1H-imidazol-2-yl)-1H-indazol-5-yl]-4-methyl-pyridin-3-ylamine (37)

Example 37 was prepared from 37a (251.1 mg, 0.473 mmol) by the same procedure used to make 21. Analogous chromatography conditions yielded 37 (120.9 mg, 74%) as a yellow foam. ¹H NMR (DMSO-d₆) δ 13.23 (s, 1H), 12.26 (s, 1H), 8.23 (s, 1H), 7.95 (s, 1H), 7.67 (s, 1H), 7.58 (d, J=8.5 Hz, 1H), 7.32 (dd, J=1.5, 8.5 Hz, 1H), 5.21 (br s, 2H), 2.53 (m, 4H), 1.98 (s, 3H), 1.15 (t, J=7.5 Hz, 6H). Anal. Calc. for C₂₀H₂₂N₆.0.3EtOH0.1CH₂Cl₂: C, 67.42; H, 6.56; N, 22.79. Found: C, 67.33; H, 6.58; N, 22.86.

### EXAMPLE 38

### 5-{3-[4-(3-Fluoro-phenyl)-1H-imidazol-2-yl]-1H-indazol-5-yl}-4-methyl-pyridin-3-ylamine

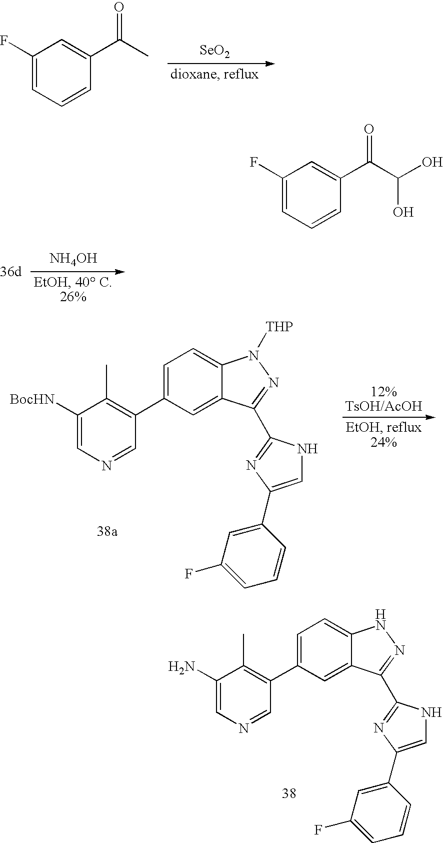

### {5-[3-[4-(3-Fluoro-phenyl)-1H-imidazol-2-yl]-1-(tetrahydro-pyran-2-yl)-1H-indazol-5-yl]-4-methyl-pyridin-3-yl}-carbamic acid tert-butyl ester (38a)

3-Fluoro-acetophenone (275 ul, 2.24 mmol) was dissolved in 1,4-dioxane (15 ml). To this solution was added selenium dioxide (275 mg, 2.48 mmol) and the mixture heated to reflux over 16 hr. The reaction mixture was filtered through celite eluting with EtOAc (75 ml) and then the solvents removed in vacuo. The resulting crude glyoxal was dissolved in EtOH (15 ml) along with aldehyde 36d (365 mg, 0.836 mmol). Finally, ammonium hydroxide solution (2.5 ml)) was added and the reaction mixture heated to 40° C. for 16 hr. The mixture was then poured into EtOAc (100 ml) and washed with brine (3×50 ml). The aqueous layer was reextracted with EtOAc (2×50 ml). The combined organics were dried over magnesium sulfate, filtered and concentrated to a crude oil. Purification by silica gel chromatography (eluting with 50-100% ethyl acetate in hexanes) afforded 38a (122 mg, 26%) as a brown solid: LC/MS (M+H=427).

### 5-{3-[4-(3-Fluoro-phenyl)-1H-imidazol-2-yl]-1H-indazol-5-yl}-4-methyl-pyridin-3-ylamine (38)

Example 38 was prepared similarly to Example 21. Analogous chromatography conditions afforded 38 (20 mg, 24%) as a yellow solid: 1H NMR (300 MHz, DMSO-D6) δ ppm 13.48 (bs, 1 H) 12.96 (bs, 1 H) 8.34 (s, 1 H) 7.97 (s, 1 H) 7.83 (s, 1 H) 7.61-7.76 (m, 4 H) 7.34-7.48 (m, J=7.72, 7.72 Hz, 2 H) 7.00 (t, J=7.63 Hz, 1 H) 5.23 (bs, 2 H) 2.01 (s, 3 H)

Anal. Calc. for C₂₂H₁₇FN₆.1.05 iPOH.0.2 CHCl₃: C, 64.65; H, 5.32; N, 17.66. Found: C, 64.59; H, 5.47; N, 17.82.

### 4-Methyl-5-[3-(4-phenyl-1H-imidazol-2-yl)-1H-indazol-5-yl]-pyridin-3-ylamine

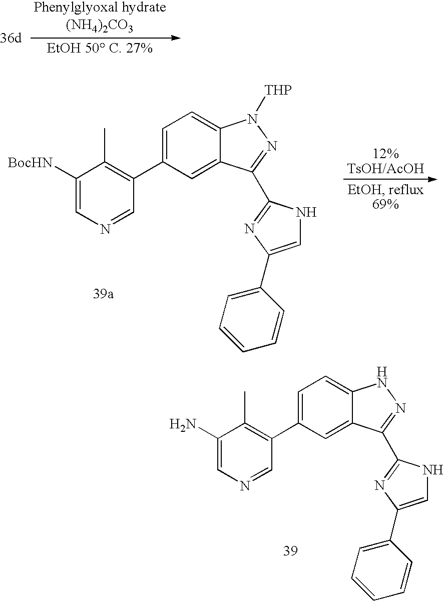

### {4-Methyl-5-[3-(4-phenyl-1H-imidazol-2-yl)-1-(tetrahydro-pyran-2-yl)-1H-indazol-5-yl]-pyridin-3-yl)carbamic acid tert-butyl ester (39a)

Example 39a was prepared similarly to Example 32a using phenylglyoxal (784 mg, 5.23 mmol), aldehyde 36d (748 mg, 1.72 mmol) and ammonium carbonate (826 mg, 8.60 mmol). Analogous chromatography conditions afforded 39a (256 mg, 27%) as a brown glass: 1H NMR (400 MHz, CHLOROFORM-D) δ ppm 8.88 (s, 1 H) 8.53 (s, 1 H) 8.31 (s, 1 H) 7.75-7.81 (m, J=6.32 Hz, 1 H) 7.63 (d, J=8.59 Hz, 1 H) 7.30-7.43 (m, 4 H) 7.21-7.24 (m, 1 H) 6.47 (s, 1 H) 5.75 (dd, J=9.47, 2.40 Hz, 1 H) 4.04-4.11 (m, 1 H) 3.72-3.82 (m, 1 H) 2.52-2.64 (m, 1 H) 2.17 (s, 3 H) 2.05-2.20 (m, 2 H) 1.62-1.83 (m, 3 H) 1.52 (s, 9 H).

### 4-Methyl-5-[3-(4-phenyl-1H-imidazol-2-yl)-1H-indazol-5-yl]-pyridin-3-ylamine (39)

Example 39 was prepared similarly to Example 21. HPLC chromatography conditions (0.5% TFA/CH₃CN and 0.1% TFA/H₂O) afforded 39 (97 mg, 57%) as a yellow solid: 1H NMR (400 MHz, MeOD) δ ppm 8.31 (s, 1 H) 7.97 (s, 1 H) 7.77-7.86 (m, 3 H) 7.65 (d, J=8.59 Hz, 1 H) 7.53 (s, 1 H) 7.38 (t, J=7.96 Hz, 3 H) 7.23 (t, J=7.33 Hz, 1 H) 2.14 (s, 3 H).

Anal. Calc. for C₂₂H₁₈N₆.1.4 DMSO.0.8 H₂O.0.3 TFA: C, 58.19; H, 5.44; N, 16.03. Found: C, 58.18; H, 5.38; N, 15.89.

### EXAMPLE 40

### 5-{3-[4-(4-Fluoro-phenyl)-1H-imidazol-2-yl]-1H-indazol-5-yl}-4-methyl-pyridin-3-ylamine

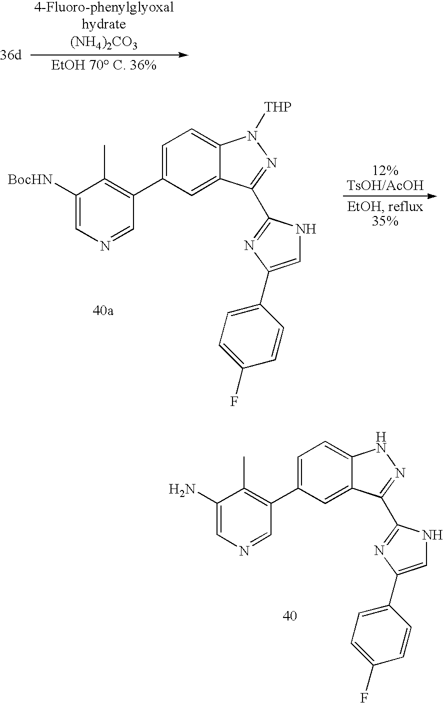

### {5-[3-[4-(4-Fluoro-phenyl)-1H-imidazol-2-yl]-1-(tetrahydro-pyran-2-yl)-1H-indazol-5-yl]-4-methyl-pyridin-3-yl}-carbamic acid tert-butyl ester (40a)

Example 40a was prepared similarly to Example 32a using 4-fluorophenylglyoxal (881 mg, 5.18 mmol), aldehyde 36d (750 mg, 1.72 mmol) and ammonium carbonate (826 mg, 8.60 mmol). Analogous chromatography conditions afforded 40a (355 mg, 36%) as a brown glass: 1H NMR (300 MHz, CHLOROFORM-D) δ ppm 9.01 (s, 1 H) 8.52 (s, 1 H) 8.32 (s, 1 H) 7.75-7.81 (m, J=7.91, 5.84 Hz, 2 H) 7.64-7.69 (m, 1 H) 7.37 (s, 1 H) 7.31 (dd, J=8.76, 1.41 Hz, 1 H) 7.01-7.11 (m, 2 H) 6.57 (s, 1 H) 5.71-5.81 (m, 1 H) 4.09 (d, J=10.36 Hz, 1 H) 3.73-3.85 (m, 1 H) 2.51-2.69 (m, 1 H) 2.24 (s, 3 H) 2.05-2.26 (m, 2 H) 1.67-1.82 (m, J=15.73, 10.08 Hz, 3 H) 1.54 (s, 9 H).

### 5-{3-[4-(4-Fluoro-phenyl)-1H-imidazol-2-yl]-1H-indazol-5-yl}-4-methyl-pyridin-3-ylamine (40)

Example 40 was prepared similarly to Example 21. Analogous chromatography conditions afforded 40 (83 mg, 35%) as a yellow solid: 1H NMR (300 MHz, MeOH) δ ppm 8.30 (s, 1 H) 7.96 (s, 1 H) 7.81 (bs, 3 H) 7.64 (d, J=8.67 Hz, 1 H) 7.50 (s, 1 H) 7.36-7.43 (m, 1 H) 7.05-7.17 (m, J=8.57, 8.57 Hz, 3 H) 2.13 (s, 3 H).

Anal. Calc. for C₂₂H₁₇FN₆.0.14 tBuOH.0.3 iPOH.0.11 CHCl₃: C, 66.27; H, 4.87; N, 19.56. Found: C, 66.29; H, 4.78; N, 19.43.

### EXAMPLE 41

### 5-(5-Methoxymethyl-4-methyl-pyridin-3-yl)-3-(4,5,6,7-tetrahydro-1H-benzoimidazol-2-yl)-1H-indazole

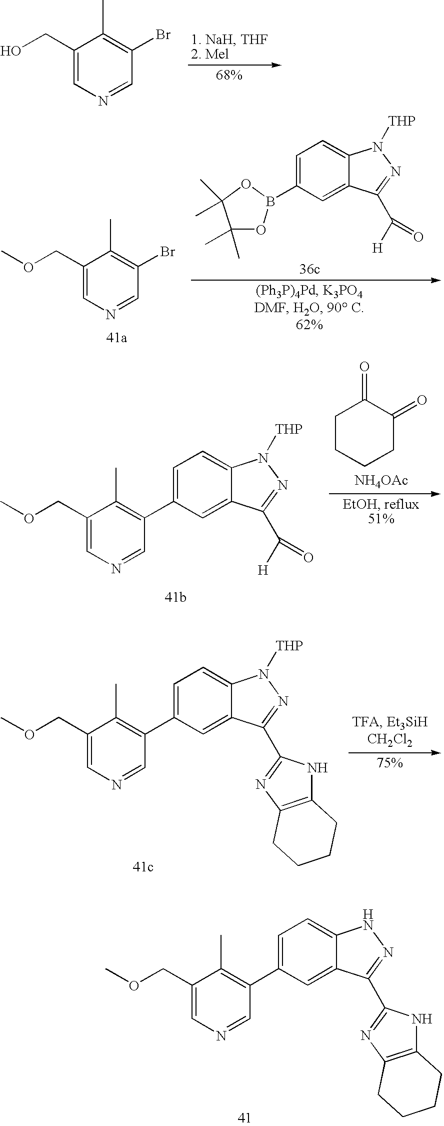

### 3-Bromo-5-methoxymethyl-4-methyl-pyridine (41a)

A solution of (5-Bromo-4-methyl-pyridin-3-yl)-methanol (5.01 g, 24.8 mmol, in 25 mL THF) [for the preparation of this compound see: Reich, S. R.; Bleckman, T. M.; Kephart, S. E.; Romines, W. H.; Wallace, M. B., U.S. Pat. No. 6,555,539, Apr. 29, 2003.] was added dropwise to a suspension of sodium hydride (95% dry, 655 mg, 27.3 mmol) in 50 mL THF over 5 minutes. Stirring was continued at room temperature for 1 hour, then iodomethane (3.87 g, 27.3 mmol) was added in one portion. After 4 more hours at room temperature, the reaction was quenched with 10 mL deionized water and 20 mL saturated aqueous sodium bicarbonate solution. The mixture was extracted with ethyl acetate (3×50 mL). The combined organic extracts were dried over magnesium sulfate, filtered, concentrated, and purified by silica gel chromatography (eluting with 20-80% ethyl acetate in hexanes) to give ether 41a (3.64 g, 68%) as a yellow oil. ¹H NMR (CDCl₃) δ 8.60 (s, 1H), 8.35 (s, 1H), 4.46 (s, 2H), 3.39 (s, 3H), 2.41 (s, 3H). Anal. Calc. for C₈H₁₀BrNO: C, 44.47; H, 4.66; N, 6.48; Br, 36.98. Found: C, 44.58; H, 4.87; N, 6.32; Br, 36.74.

### 5-(5-Methoxymethyl-4-methyl-pyridin-3-yl)-1-(tetrahydro-pyran-2-yl)-1H-indazole-3-carbaldehyde (41b)

By the same procedure used to make 36d, boronic ester 36c (5.00 g, 14.0 mmol) was coupled with bromide 41a (3.03 g, 14.0 mmol) in the presence of potassium phosphate (4.46 g, 21 mmol) and tetrakis(triphenylphosphine) palladium (0) (809 mg, 0.7 mmol) in N,N-dimethylformamide (140 mL) and water (20 mL). After workup and purification analogous to 36d, aldehyde 41b (3.21 g, 62%) was obtained as a white powder. ¹H NMR (CDCl₃) δ 10.26 (s, 1H), 8.49 (s, 1H), 8.42 (s, 1H), 8.23 (s, 1H), 7.75 (d, J=8.6 Hz, 1H), 7.40 (dd, J=1.5, 8.6 Hz, 1H), 5.88 (dd, J=3.0, 9.1 Hz, 1H), 4.53 (s, 2H), 4.04 (m, 1H), 3.81 (m, 1H), 3.44 (s, 3H), 2.60 (m, 1H), 2.25 (s, 3H), 2.19 (m, 2H), 2.05 (m, 1H), 1.80 (m, 3H). Anal. Calc. for C₂₁H₂₃N₃O₃.0.2H₂O: C, 68.35; H, 6.39; N, 11.39. Found: C, 68.25; H, 6.30; N, 11.30.

### 5-(5-Methoxymethyl-4-methyl-pyridin-3-yl)-3-(4,5,6,7-tetrahydro-1H-benzoimidazol-2-yl)-1-(tetrahydro-pyran-2-yl)-1H-indazole (41c)

By the same procedure used to make 21a, aldehyde 41b (847.2 mg, 2.32 mmol) was cyclized with 1,2-cyclohexanedione (273 mg, 2.43 mmol) in the presence of ammonium acetate (1.07 g, 13.9 mmol), affording, after analogous purification, 41c (584.0 mg, 51%) as a yellow foam. ¹H NMR (DMSO-d₆) δ 12.22 (s, 1H), 8.47 (s, 1H), 8.38 (s, 1H), 8.26 (s, 1H), 7.83 (d, J=8.8 Hz, 1H), 7.43 (dd, J=1.5, 8.6 Hz, 1H), 5.93 (dd, J=2.3, 9.9 Hz, 1H), 4.54 (s, 2H), 3.92 (m, 1H), 3.78 (m, 1H), 3.36 (s, 3H), 2.57 (m, 2H), 2.20 (s, 3H), 2.05 (m, 2H), 1.75 (m, 5H), 1.61 (m, 2H). Anal. Calc. for C₂₇H₃₁N₅O₂.0.3 EtOAc.0.1 cyclohexane: C, 70.25; H, 7.08; N, 14.22. Found: C, 70.04; H, 7.13; N, 14.29.

### 5-(5-Methoxymethyl-4-methyl-pyridin-3-yl)-3-(4,5,6,7-tetrahydro-1H-benzoimidazol-2-yl)-1H-indazole (41)

Compound 41c (518.5 mg, 1.133 mmol) was dissolved in 4.36 mL dichloromethane and treated with triethyl silane (329 mg, 2.83 mmol) and trifluoroacetic acid (4.36 mL). After stirring for 20 hours at room temperature, the solvents were removed in vacuo. The residue was partitioned between 25 mL ethyl acetate and 25 mL 2N sodium hydroxide solution. The organic layer was dried over magnesium sulfate, filtered, concentrated, and purified by silica gel chromatography (eluting with 5-10% [5% concentrated ammonium hydroxide in ethanol] in chloroform), affording 41 (359.7 mg, 75%) as a white foam. ¹H NMR (MeOD) δ 8.42 (s, 1H), 8.40 (s, 1H), 8.25 (s, 1H), 7.63 (d, J=8.6 Hz, 1H), 7.37 (dd, J=1.5, 8.6 Hz, 1H), 4.59 (s, 2H), 3.45 (s, 3H), 2.64 (br s, 4H), 2.31 (s, 3H), 1.86 (br s, 4H). Anal. Calc. for C₂₂H₂₃N₅O.0.3H₂O.0.5 cyclohexane: C, 71.33; H, 7.09; N, 16.64. Found: C, 71.05; H, 7.03; N, 16.62.

### EXAMPLE 42

### 3-(1H-Imidazol-2-yl)-5-(5-methoxymethyl-4-methyl-pyridin-3-yl)-1H-indazole

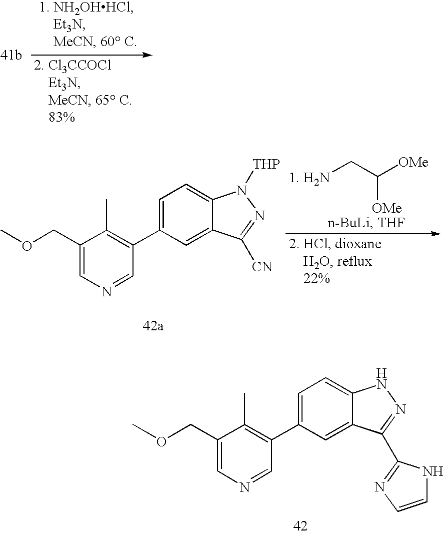

### 5-(5-Methoxymethyl-4-methyl-pyridin-3-yl)-1-(tetrahydro-pyran-2-yl)-1H-indazole-3-carbonitrile (42a)

By the same procedure used to make intermediate 1g, aldehyde 41b (1.0455 g, 2.86 mmol) was converted into nitrile 42a (932.1 mg, 83%), a white foam. ¹H NMR (DMSO-d₆) δ 8.48 (s, 1H), 8.41 (s, 1H), 8.07 (d, J=8.8 Hz, 1H), 7.88 (s, 1H), 7.61 (dd, J=1.5, 8.8 Hz, 1H), 6.11 (dd, J=2.5, 9.3 Hz, 1H), 4.53 (s, 2H), 3.87 (m, 1H), 3.82 (m, 1H), 3.35 (s, 3H), 2.35 (m, 1H), 2.21 (s, 3H), 2.05 (m, 2H), 1.77 (m, 1H), 1.62 (m, 2H).

### 3-(1H-Imidazol-2-yl)-5-(5-methoxymethyl-4-methyl-pyridin-3-yl)-1H-indazole (42)

Nitrile 42a (928.1 mg, 2.56 mmol) was converted into imidazole 42 by the procedure used to convert nitrile 1g into example 1 (example 1, method B). The crude product was purified by silica gel chromatography (eluting with 1/19/80 concentrated ammonium hydroxide/ethanol/chloroform), affording 42 (189.1 mg, 22%) as a pinkish solid. ¹H NMR (DMSO-d₆) δ 13.38 (s, 1H), 12.69 (s, 1H), 8.46 (s, 1H), 8.39 (s, 1H), 8.28 (s, 1H), 7.66 (d, J=8.6 Hz, 1H), 7.39 (dd, J=1.5, 8.6 Hz, 1H), 7.19 (br s, 1H), 7.06 (br s, 1H), 4.54 (s, 2H), 3.36 (s, 3H), 2.22 (s, 3H).

Anal. Calc. for C₁₈H₁₇N₅O.0.1 H₂O.0.1 MeOH.0.1 CHCl₃: C, 65.00; H, 5.31; N, 20.83. Found: C, 65.35; H, 5.54; N, 20.43.

### EXAMPLE 43

### 5-(5-Ethoxymethyl-4-methyl-pyridin-3-yl)-3-(4,5,6,7-tetrahydro-1H-benzoimidazol-2-yl)-1H-indazole

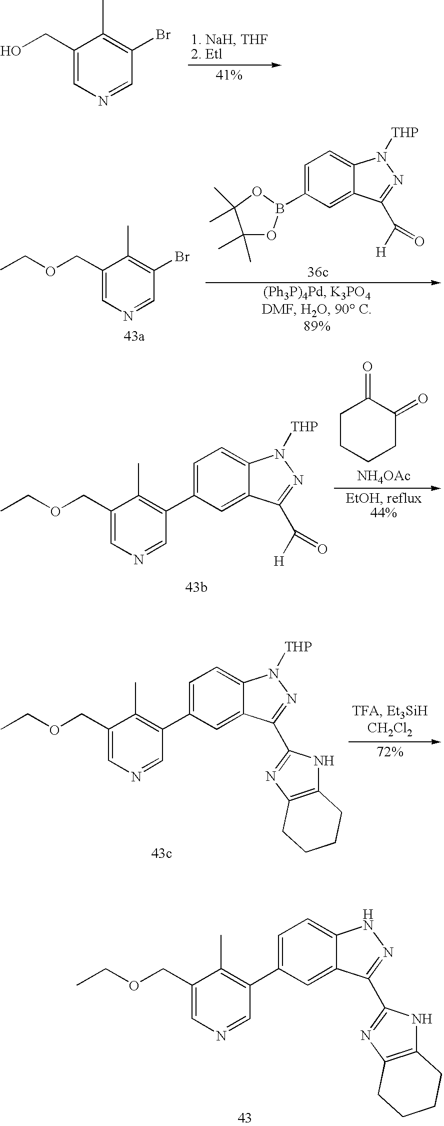

### 3-Bromo-5-ethoxymethyl-4-methyl-pyridine (43a)

Compound 43a was prepared similarly to 41a, using (5-Bromo-4-methyl-pyridin-3-yl)-methanol (5.03 g, 24.9 mmol), sodium hydride (95% dry, 657 mg, 27.4 mmol) and iodoethane (4.27 g, 27.4 mmol). Analogous chromatography conditions gave ethyl ether 43a (2.35 g, 41%) as a yellow oil. ¹H NMR (CDCl₃) δ 8.59 (s, 1H), 8.36 (s, 1H), 4.50 (s, 2H), 3.55 (q, J=6.8 Hz, 2H), 2.42 (s, 3H), 1.24 (t, J=6.8 Hz, 3H). Anal. Calc. for C₉H₁₂BrNO: C, 46.98; H, 5.26; N, 6.09; Br, 34.73. Found: C, 46.92; H, 5.38; N, 6.02; Br, 34.79.

### 5-(5-Ethoxymethyl-4-methyl-pyridin-3-yl)-1-(tetrahydro-pyran-2-yl)-1H-indazole-3-carbaldehyde (43b)

By the same procedure used to make 36d, boronic ester 36c (1.47 g, 4.12 mmol) was coupled with bromide 43a (947.3 mg, 4.12 mmol) in the presence of potassium phosphate (1.31 g, 6.18 mmol) and tetrakis(triphenylphosphine) palladium (0) (237.5 mg, 0.21 mmol) in N,N-dimethyacetamide (41.2 mL) and water (5.8 mL). After workup and purification analogous to 36d, aldehyde 43b (1.44 g, 89%) was obtained as a yellow foam. ¹H NMR (DMSO-d₆) δ 10.21 (s, 1H), 8.48 (s, 1H), 8.38 (s, 1H), 8.05 (s, 1H), 8.02 (d, J=8.8 Hz, 1H), 7.57 (dd, J=1.3, 8.6 Hz, 1H), 6.12 (dd, J=2.3, 9.3 Hz, 1H), 4.57 (s, 2H), 3.91 (m, 1H), 3.82 (m, 1H), 3.56 (q, J=7.1 Hz, 2H), 2.20 (s, 3H), 2.08 (m, 2H), 1.80 (m, 1H), 1.63 (m, 2H), 1.18 (t, J=7.0 Hz, 3H). Anal. Calc. for C₂₂H₂₅N₃O₃.0.2H₂O.0.1EtOAc: C, 68.65; H, 6.74; N, 10.72. Found: C, 68.74; H, 6.55; N, 10.54.

### 5-(5-Ethoxymethyl-4-methyl-pyridin-3-yl)-3-(4,5,6,7-tetrahydro-1H-benzoimidazol-2-yl)-1-(tetrahydro-pyran-2-yl)-1H-indazole (43c)

By the same procedure used to make 21a, aldehyde 43b (1.22 g, 3.21 mmol) was cyclized with 1,2-cyclohexanedione (387 mg, 3.37 mmol) in the presence of ammonium acetate (1.48 g, 19.3 mmol), affording, after analogous purification, 43c (729.9 mg, 44%) as a yellow foam. ¹H NMR (DMSO-d₆) δ 12.22 (s, 1H), 8.47 (s, 1H), 8.37 (s, 1H), 8.26 (s, 1H), 7.83 (d, J=8.6 Hz, 1H), 7.43 (dd, J=1.5, 8.6 Hz, 1H), 5.93 (dd, J=2.3, 9.9 Hz, 1H), 4.57 (s, 2H), 3.92 (m, 1H), 3.77 (m, 1H), 3.56 (q, J=6.8 Hz, 2H), 2.57 (m, 2H), 2.20 (s, 3H), 2.05 (m, 2H), 1.75 (m, 5H), 1.61 (m, 2H), 1.18 (t, J=6.9 Hz, 3H). Anal. Calc. for C₂₈H₃₃N₅O₂.0.2 EtOAc.0.3 cyclohexane: C, 71.44; H, 7.48; N, 13.61. Found: C, 71.12; H, 7.37; N, 13.30.

### 5-(5-Ethoxymethyl-4-methyl-pyridin-3-yl)-3-(4,5,6,7-tetrahydro-1H-benzoimidazol-2-yl)-1H-indazole (43)

Example 43 was prepared in the same manner as example 41. Compound 43c (666.8 mg, 1.414 mmol) yielded, after analogous chromatography, 43 (414.4 mg, 72%), a white foam. ¹H NMR (MeOD) δ 8.43 (s, 1H), 8.40 (s, 1H), 8.25 (s, 1H), 7.63 (d, J=8.6 Hz, 1H), 7.38 (dd, J=1.8, 8.6 Hz, 1H), 4.64 (s, 2H), 3.64 (q, J=7.1 Hz, 2H), 2.64 (br s, 4H), 2.33 (s, 3H), 1.86 (br s, 4H), 1.26 (t, J=7.0 Hz, 3H). Anal. Calc. for C₂₃H₂₅N₅O.0.2H₂O.0.2 EtOAc: C, 69.94; H, 6.66; N, 17.14. Found: C, 69.87; H, 6.62; N, 17.28.

### EXAMPLE 44

### 5-(5-Ethoxymethyl-4-methyl-pyridin-3-yl)-3-(1H-imidazol-2-yl)-1H-indazole

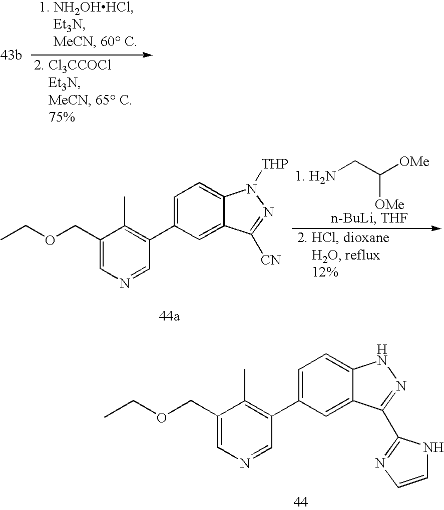

### 5-(5-Ethoxymethyl-4-methyl-pyridin-3-yl)-1-(tetrahydro-pyran-2-yl)-1H-indazole-3-carbonitrile (44a)

By the same procedure used to make intermediate 1g, aldehyde 43b (1.365 g, 3.60 mmol) was converted into nitrile 44a (1.13 g, 75%), a white foam. ¹H NMR (DMSO-d₆) δ 8.48 (s, 1H), 8.40 (s, 1H), 8.07 (d, J=8.8 Hz, 1H), 7.88 (s, 1H), 7.61 (dd, J=1.3, 8.6 Hz, 1H), 6.11 (dd, J=2.5, 9.1 Hz, 1H), 4.56 (s, 2H), 3.87 (m, 1H), 3.80 (m, 1H), 3.55 (q, J=7.1 Hz, 2H), 2.35 (m, 1H), 2.21 (s, 3H), 2.07 (m, 2H), 1.78 (m, 1H), 1.62 (m, 2H), 1.17 (t, J=7.1 Hz, 3H).

### 5-(5-Ethoxymethyl-4-methyl-pyridin-3-yl)-3-(1H-imidazol-2-yl)-1H-indazole (44)

Nitrile 44a (1.13 g, 3.00 mmol) was converted into imidazole 44 by the procedure used to convert nitrile 1g into compound 1 (example 1, method B). The crude product was purified by silica gel chromatography (eluting with 1/19/80 concentrated ammonium hydroxide/ethanol/chloroform), affording 44 (123.1 mg, 12%) as a pinkish solid. ¹H NMR (DMSO-d₆) δ 13.38 (s, 1H), 12.68 (s, 1H), 8.46 (s, 1H), 8.38 (s, 1H), 8.28 (s, 1H), 7.66 (d, J=8.6 Hz, 1H), 7.39 (dd, J=1.3, 8.6 Hz, 1H), 7.19 (s, 1H), 7.06 (s, 1H), 4.57 (s, 2H), 3.56 (q, J=6.8 Hz, 2H), 2.22 (s, 3H), 1.18 (t, J=7.1 Hz, 3H). Anal. Calc. for C₁₉H₁₉N₅O.0.05 H₂O.0.2 EtOAc: C, 67.58; H, 5.93; N, 19.90. Found: C, 67.88; H, 5.94; N, 19.64.

### EXAMPLE 45

### Method A: {5-[3-(1H-Imidazol-2-yl)-1H-indazol-5-yl]-4-methyl-pyridin-3-ylmethyl}-methyl-amine

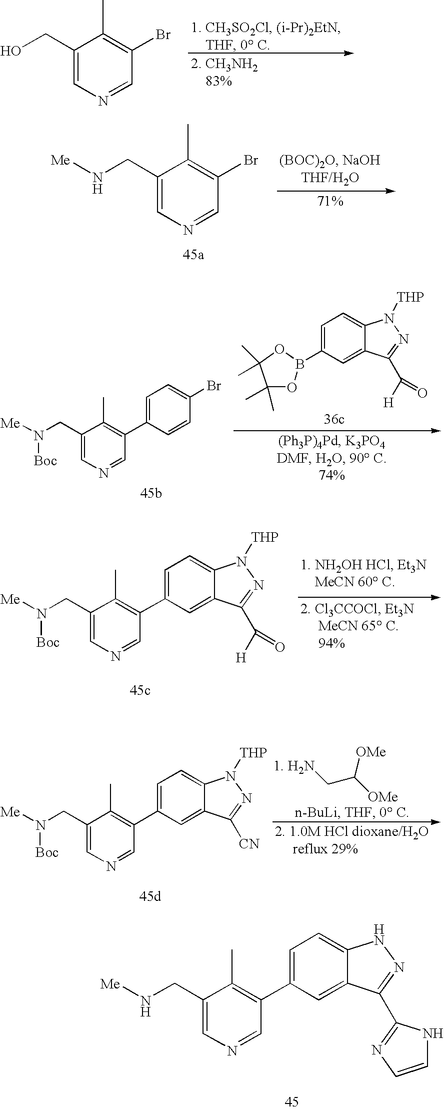

### (5-Bromo-4-methyl-pyridin-3-ylmethyl)-methyl-amine (45a)

(5-Bromo-4-methyl-pyridin-3-yl)-methanol (4.90 g, 24.25 mmol) [for the preparation of this compound see: Reich, S. R.; Bleckman, T. M.; Kephart, S. E.; Romines, W. H.; Wallace, M. B., U.S. Pat. No. 6,555,539, Apr. 29, 2003.] was dissolved in dry THF (500 mL). Diisopropyl ethyl amine (8.87 mL, 50.9 mmol) was added, and the solution cooled to 0° C. in an ice-salt bath. Methanesulfonyl chloride (4.74 mL, 61.2 mmol) was added dropwise over 2-3 minutes. Stirring was continued for 1 hour at 0° C., then methylamine gas (large excess) was bubbled into the solution via a fritted gas dispersion tube for 30 minutes. Stirring was continued at room temperature for 18 hours. The reaction mixture was partitioned between 500 mL ethyl acetate and 100 mL deionized water. The organic layer was washed with 100 mL saturated aqueous sodium chloride, dried over magnesium sulfate, filtered and concentrated in vacuo to a crude yellow oil (6.85 g). Purification by silica gel chromatography (eluting with 1/9/90 concentrated ammonium hydroxide/ethanol/dichloromethane) affords amine 45a (4.3178 g, 83%) as a yellow liquid. ¹H NMR (CDCl₃) δ 8.54 (s, 1H), 8.31 (s, 1H), 3.74 (s, 2H), 2.45 (s, 3H), 2.42 (s, 3H).

### (5-Bromo4-methyl-pyridin-3-ylmethyl)-methyl-carbamic acid tert-butyl ester (45b)

Amine 45a (5.65 g, 26.3 mmol) was dissolved in THF (500 mL). Di-tert-butyl dicarbonate (8.03 g, 36.8 mmol) and 1.0 M NaOH solution (78.9 mL, 78.9 mmol) were added. Stirring was continued at room temperature for 18 hours, 40 minutes. The mixture was partitioned between 200 mL deionized water and 200 mL ethyl acetate. The organic layer was washed with saturated aqueous sodium chloride (200 mL), dried over magnesium sulfate, filtered, and concentrated to give a crude colorless oil. Silica gel chromatography (eluting with 40-80% ethyl acetate in hexanes) afforded pure 45b (5.85 g, 71%) as a pale yellow oil. ¹H NMR (CDCl₃) δ 8.59 (s, 1H), 8.22 (s, 1H), 4.49 (br s, 2H), 2.77 (s, 3H), 2.38 (s, 3H), 1.47 (s, 9H).

Anal. Calc. for C₁₃H₁₉BrN₂O₂: C, 49.54; H, 6.08; N, 8.89; Br, 25.35. Found: C, 49.68; H, 6.04; N, 8.88; Br, 25.19.

### {5-[3-Formyl-1-(tetrahydro-pyran-2-yl)-1H-indazol-5-yl]-4-methyl-pyridin-3-ylmethyl}-methyl-carbamic acid tert-butyl ester (45c)

Boronic ester 36c (4.04 g, 11.36 mmol), bromopyridine 45b (3.58 g, 11.36 mmol), and potassium phosphate (3.62 g, 17.4 mmol) were dissolved in 114 mL N,N-dimethylacetamide and 12.0 ml deionized water. The solution was degassed as described in the procedure for 1e, tetrakis(triphenylphosphine) palladium (0) (0.96 g, 0.83 mmol) was added, and the solution degassed again. The solution was heated to 90° C. for 4.5 hours. After cooling to room temperature, 100 mL deionized water and 100 mL saturated aqueous sodium bicarbonate solution were added. The mixture was extracted with ethyl acetate (2×100 mL), and the combined organic extracts were dried over magnesium sulfate, filtered, and concentrated. The orange oil thus obtained was purified by silica gel chromatography (50-100% ethyl acetate in hexanes), to give 45c (3.92 g, 74%) as a yellow foam. ¹H NMR (CDCl₃) δ 10.26 (s, 1H), 8.40 (s, 1H), 8.35 (s, 1H), 8.23 (s, 1H), 7.76 (d, J=8.7 Hz, 1H), 7.39 (dd, J=1.5, 8.7 Hz, 1H), 5.89 (dd, J=2.8, 8.9 Hz, 1H), 4.55 (br s, 2H), 4.03 (m, 1H), 3.81 (m, 1H), 2.85 (s, 3H), 2.59 (m, 1H), 2.19 (s, 3H), 2.18 (m, 2H), 1.80 (m, 3H), 1.48 (s, 9H).

### {5-[3-Cyano-1-(tetrahydro-pyran-2-yl)-1H-indazol-5-yl]-4-methyl-pyridin-3-ylmethyl}-methyl-carbamic acid tert-butyl ester (45d)

Example 45d was prepared in a similar manner to Example 1g, Method B, using aldehyde 45c (1.0 g, 2.153 mmol), hydroxylamine hydrochloride (159 mg, 2.288 mmol) and triethylamine (320 ul, 2.30 mmol) in acetonitrile (25 ml) for the first step. The second step used trichloroacetylchloride (365 ul, 3.269 mmol) and triethylamine (640 ul, 4.60 mmol). Analogous chromatography gave 45d (933 mg, 94%) as a white solid: 1H NMR (300 MHz, CHLOROFORM-D) δ ppm 8.36 (d, J=9.80 Hz, 2 H) 7.81 (d, J=8.67 Hz, 1 H) 7.73 (s, 1 H) 7.40 (dd, J=8.67, 1.51 Hz, 1 H) 5.85 (dd, J=8.29, 2.64 Hz, 1 H) 4.53 (s, 2 H) 3.91-4.02 (m, 1 H) 3.71-3.83 (m, 1 H) 2.84 (s, 3 H) 2.45-2.59 (m, 1 H) 2.17 (s, 3 H) 2.10-2.23 (m, 2 H) 1.65-1.83 (m, 3 H) 1.46 (s, 9 H).

### {5-[3-(1H-Imidazol-2-yl)-1H-indazol-5-yl]-4-methyl-pyridin-3-ylmethyl}-methyl-amine (45)

Example 45, Method A, was prepared in a similar manner to Example 1, Method B, using nitrile 45d (933 mg, 2.024 mmol). A 2.5 M solution of n-butyllithium in hexanes (4.1 ml, 10.25 mmole) was added under argon to a solution of aminoacetaldehyde dimethyl acetal (1.1 ml, 10.11 mmole) in THF (10 ml) at −78° C. After stirring for a further 30 minutes at −78° C., a portion (8 ml, 5.392 mmole) of this lithium 2,2-dimethoxyethylamide solution was added to a solution nitrile 45d (933 mg, 2.024 mmole) in THF (20 ml). The resultant solution was stirred, under argon, at 0° for 2 hours, then quenched by addition of 50% aqueous CH₃OH (4 ml). The volatiles were removed by concentration, in vacuo, and the residue obtained was dissolved in 4.0 M HCl in 1,4-dioxane (10 ml). This solution was diluted with water (10 ml), then heated at reflux overnight. Reaction work-up was analogous to Example 1, Method B. HPLC chromatography conditions (0.5% TFA/CH3CN and 0.1% TFA/H₂O) and recrystallization from hot ethanol afforded compound 45 (189 mg, 29%) as a white solid: 1H NMR (400 MHz, MeOD) δ ppm 8.61 (s, 1 H) 8.56 (s, 1 H) 8.20 (s, 1 H) 7.84 (d, J=8.59 Hz, 1 H) 7.62 (s, 2 H) 7.53 (dd, J=8.84, 1.26 Hz, 1 H) 4.44 (s, 2 H) 2.87 (s, 3 H) 2.42 (s, 3 H).

Anal. Calcd. for C₁₈H₁₈N₆*2.0 TFA: C, 48.35; H, 3.69; N, 15.38; F, 20.86. Found: C, 48.20; H, 3.72; N, 15.27; F, 20.70.

### EXAMPLE 45

### Method B: {5-[3-(1H-Imidazol-2-yl)-1H-indazol-5-yl]-4-methyl-pyridin-3-ylmethyl}-methyl-amine

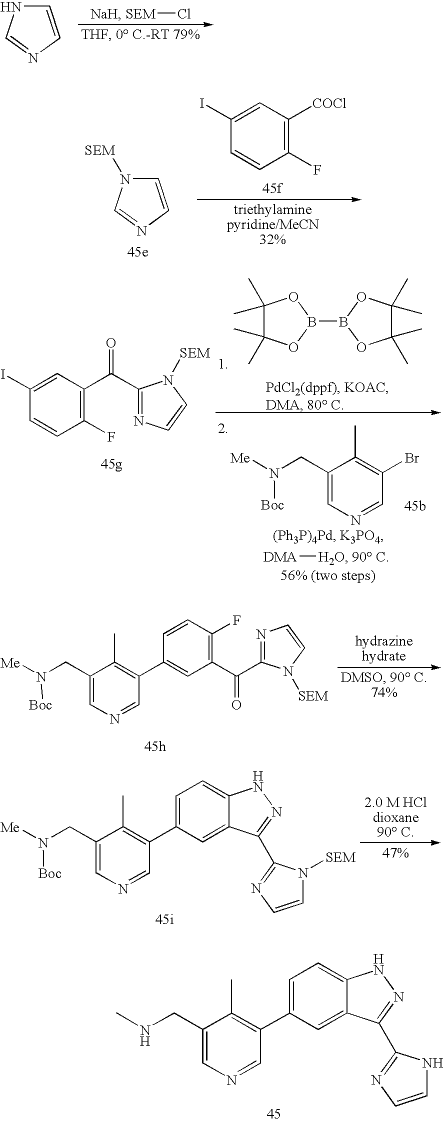

### 1-(2-Trimethylsilanyl-ethoxymethyl)-1H-imidazole (45e)

A suspension of 95% NaH (1.17 g, 48.6 mmol) in anhydrous THF (150 ml) was cooled to 0° C. via ice bath. In a separate flask was dissolved imidazole (3.01 g, 44.3 mmol) in THF (100 ml). The imidazole solution was added dropwise to the NaH suspension over 30 minutes. After an additional 30 minutes, SEM-Cl (9.75 ml, 55.07 mmol) was added. The reaction mixture stirred for 16 hr at ambient temperature. The mixture was then poured into NaHCO₃ solution (200 ml) and then extracted with EtOAc (3×100 ml). The combined organics were dried over magnesium sulfate, filtered and concentrated to a crude oil. Purification by silica gel chromatography (eluting with 80-100% ethyl acetate in hexanes) afforded 45e (6.93 g, 79%) as a clear oil: 1H NMR (300 MHz, CHLOROFORM-D) δ ppm 7.58 (s, 1 H) 7.05 (d, J=16.77 Hz, 2 H) 5.25 (s, 2 H) 3.40-3.50 (m, 2 H) 0.82-0.93 (m, 2 H) −0.04 (s, 9 H).

### 2-Fluoro-5-iodobenzoyl chloride (45f)

A suspension of the acid (5.37 g, 20.19 mmol) in SOCl₂ (40.82 g, 25 mL, 343 mmol) was refluxed for 22h. The solvents were removed by rotary evaporation and the residue partitioned between EtOAc and saturated aqueous Na₂CO₃. The organic extracts were dried over MgSO₄ and concentrated yielding 45f (5.36 g, 93%) as a pale pink solid which was used in the next step without further purification: ¹H NMR (CDCl₃) 6.96 (t, J=6.0 Hz, 1H), 7.90-7.94 (m, 1H), 8.35 (d, J=3.0 Hz, 1H).

### (2-Fluoro-5-iodo-phenyl)-[1-(2-trimethylsilanyl-ethoxymethyl)-1H-imidazol-2-yl]-methanone (45g)

In a flask was dissolved protected imidazole 45e (3.77 g, 18.94 mmol) in dry pyridine (100 ml) and triethylamine (20 ml, 143.8 mmol). In an additional funnel fixed to the flask was dissolved acid chloride 45f (5.37 g, 18.89 mmol) in anhydrous acetonitrile (40 ml). The acid chloride solution was added dropwise over 30 minutes at room temperature and the reaction mixture allowed to stir for 20 hr. The solvents were removed in vacuo and the crude dissolved in EtOAc (150 ml). The solution was washed with 2N NaOH solution (3×75 ml). The aqueous layers were reextracted with EtOAc (2×50 ml). The combined organics were dried over magnesium sulfate, filtered and concentrated to a crude oil. Purification by silica gel chromatography (eluting with 10-100% ethyl acetate in hexanes) afforded 45g (2.66 g, 32%) as a clear gum: 1H NMR (300 MHz, CHLOROFORM-D) δ ppm 7.95 (dd, J=6.22, 2.26 Hz, 1 H) 7.73-7.83 (m, 1 H) 7.39 (s, 1 H) 7.27 (s, 1 H) 6.92 (t, J=9.14 Hz, 1 H) 5.83 (s, 2 H) 3.56-3.66 (m, 2 H) 0.89-0.99 (m, 2 H) −0.03 (s, 9 H).

### 4(5-{4-Fluoro-3-[1-(2-trimethylsilanyl-ethoxymethyl)-1H-imidazole-2-carbonyl]-phenyl}-methyl-pyridin-3-ylmethyl)-methyl-carbamic acid tert-butyl ester (45h)

Example 45h was formed in a similar manner as Example 1e using the biarylketone 45g (1.02 g, 2.287 mmol), bis(pinacolato)diboron (610 mg, 2.40 mmol), potassium acetate (672 mg, 6.86 mmol) and [1,1′-bis(diphenylphosphino)-ferrocene] dichloropalladium(II)complex with dichloromethane (65 mg, 0.08 mmol) in N,N-dimethylacetamide (20 mL) in the first step. Bromopyridine 45b (755 mg, 2.404 mmol), potassium phosphate (727 mg, 3.431 mmol), tetrakis(triphenylphosphine) palladium (0) (80 mg, 0.07 mmol), deionized water (5 mL) and N,N-dimethylacetamide (5 mL) were added for the second step. Analogous chromatography conditions afforded 45h (713 mg, 56%) as a yellow oil: 1H NMR (300 MHz, CHLOROFORM-D) δ ppm 8.39 (s, 1 H) 8.31 (s, 1 H) 7.63 (s, 1 H) 7.37-7.47 (m, J=12.25 Hz, 2 H) 7.25 (s, 2 H) 5.85 (s, 2 H) 4.51 (s, 2 H) 3.56-3.68 (m, J=5.09 Hz, 2 H) 2.81 (s, 3 H) 2.21 (s, 3 H) 1.46 (s, 9 H) 0.89-1.00 (m, J=6.22 Hz, 2 H) −0.04 (s, 9 H).

### Methyl-(4-methyl-5-{3-[1-(2-trimethylsilanyl-ethoxymethyl)-1H-imidazol-2-yl]-1H-indazol-5-yl}-pyridin-3-ylmethyl)-carbamic acid tert-butyl ester (45i)

The biarylketone 45h (713 mg, 1.287 mmol) and hydrazine hydrate (400 ul, 12.86 mmol) were dissolved in DMSO (14 ml). The reaction mixture was heated to 90° C. for 4 hr. After cooling, the reaction mixture was poured into EtOAc (100 ml) and washed with water (2×50 ml) and brine (2×50 ml). The aqueous layers were reextracted with EtOAc (2×50 ml). The combined organics were dried over magnesium sulfate, filtered and concentrated to a crude oil. Purification by silica gel chromatography (eluting with 50-100% ethyl acetate in hexanes) afforded indazole 45i (525 mg, 74%) as a yellow glass: 1H NMR (300 MHz, CHLOROFORM-D) δ ppm 10.58 (s, 1 H) 8.51 (d, J=10.93 Hz, 2 H) 8.38 (s, 1 H) 7.21-7.33 (m, 4 H) 6.00 (s, 2 H) 4.58 (s, 2 H) 3.58-3.70 (m, 2 H) 2.88 (s, 3 H) 2.24 (s, 3 H) 1.52 (s, 9 H) 0.87-1.01 (m, 2 H) −0.06 (s, 9 H).

### {5-[3-(1H-Imidazol-2-yl)-1H-indazol-5-yl]-4-methyl-pyridin-3-ylmethyl}-methyl-amine (45)

The indazole 45i (500 mg, 0.912 mmol) was dissolved in dioxane (5 ml). To this was added 2N HCl in dioxane solution (5 ml). The reaction mixture was heated to 90° C. for 16 hr. The solvent was removed in vacuo and the crude was dissolved in 20% iPOH in chloroform solution (75 ml). The solution was washed with 2N NaOH solution (3×40 ml) and brine (2×40 ml). The aqueous layers were reextracted with 20% iPOH in chloroform solution (2×50 ml). The combined organics were dried over magnesium sulfate, filtered and concentrated to a crude oil. Purification by silica gel chromatography {eluting with 15% (5% concentrate ammonium hydroxide in ethanol) in chloroform afforded a crude paste. Recrystalliztion from hot ethanol gave indazole 45 (136 mg, 47%) as a white powder: 1H NMR (400 MHz, MeOD) δ ppm 8.61 (s, 1 H) 8.56 (s, 1 H) 8.20 (s, 1 H) 7.84 (d, J=8.59 Hz, 1 H) 7.62 (s, 2 H) 7.53 (dd, J=8.84, 1.26 Hz, 1 H) 4.44 (s, 2 H) 2.87 (s, 3 H) 2.42 (s, 3 H).

Anal. Calcd. for C₁₈H₁₈N₆.0.3 EtOH.0.3 H₂O: C, 64.90; H, 5.89; N, 24.42. Found: C, 64.78; H, 5.76; N, 24.63.

### EXAMPLE 46

### Ethyl-{4-ethyl-5-[3-(4,5,6,7-tetrahydro-1H-benzoimidazol-2-yl)-1H-indazol-5-yl]-pyridin-3-ylmethyl}-amine

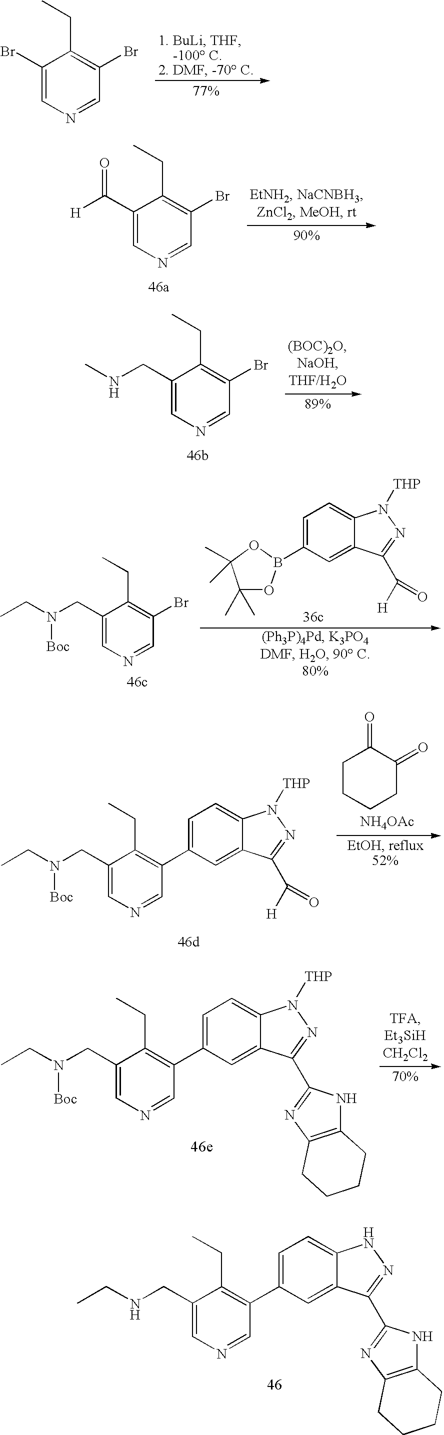

### 5-Bromo-4-ethyl-pyridine-3-carbaldehyde (46a)

A solution of 3,5-Dibromo-4-ethyl-pyridine (26.52 g, 100 mmol) [for the preparation of this compound see: Reich, S. R.; Bleckman, T. M.; Kephart, S. E.; Romines, W. H.; Wallace, M. B., U.S. Pat. No. 6,555,539, Apr. 29, 2003.] in 1.0 L anhydrous tetrahydrofuran was cooled to an internal temperature of 98° C. (methanol/N₂). n-Butyllithium (2.5 M in hexanes, 44 mL) was added dropwise over 10 minutes, slowly enough to keep the internal reaction temperature below 90° C. After 10 additional minutes, N,N-dimethylformamide (14.6 g, 200 mmol) was added. The mixture was allowed to warm to 65° C. over 1 hour, then the temperature was maintained at 70° C. for a second hour. The cooling bath was removed and the solution (at 10° C.) was quenched with 150 mL saturated aqueous ammonium chloride and 75 mL deionized water. Tetrahydrofuran was removed in vacuo, and the aqueous residue extracted with 500 mL ethyl acetate. The organic layer was washed with brine, dried over sodium sulfate, filtered, concentrated, and purified by silica gel chromatography (eluting with 20-50% ethyl acetate in hexanes), affording aldehyde 46a (16.51 g, 77%) as a pale yellow oil which crystallized on standing. ¹H NMR (CDCl₃) δ 10.24 (s, 1H), 8.84 (s, 1H), 8.83 (s, 1H), 3.22 (q, J=7.5 Hz, 2H), 1.22 (t, J=7.5 Hz, 3H).

### (5-Bromo-4-ethyl-pyridin-3-ylmethyl)-ethyl-amine (46b)

Reductive amination of 46a (5.80 g, 27.1 mmol) by the same procedure used to make 1a afforded, after analogous chromatography, 46b (5.91 g, 90%) as a cloudy yellow oil. ¹H NMR (CDCl₃) δ 8.54 (s, 1H), 8.36 (s, 1H), 3.80 (s, 2H), 2.86 (q, J=7.6 Hz, 2H), 2.70 (q, J=7.0 Hz, 2H), 1.18 (t, J=7.6 Hz, 3H), 1.13 (t, J=7.1 Hz, 3H). Anal. Calc. for C₁₀H₁₅BrN₂: C, 49.40; H, 6.22; N, 11.52; Br, 32.86. Found: C, 49.23; H, 6.14; N, 11.45; Br, 32.66.

### (5-Bromo-4-ethyl-pyridin-3-ylmethyl)-ethyl-carbamic acid tert-butyl ester (46c)

By an analogous procedure to 1b, amine 46b (5.89 g, 24.2 mmol) was converted to intermediate 46c (7.55 g, 89%), a colorless oil. ¹H NMR (CDCl₃) δ 8.56 (s, 1H), 8.22 (s, 1H), 4.49 (s, 2H), 3.15 (br s, 2H), 2.80 (q, J=7.6 Hz, 2H), 1.44 (s, 9H), 1.12 (t, J=7.6 Hz, 3H), 1.05 (t, J=7.0 Hz, 3H). Anal. Calc. for C₁₅H₂₃BrN₂O₂.0.3H₂O: C, 51.67; H, 6.82; N, 8.03; Br, 22.92. Found: C, 51.57; H, 6.71; N, 7.91; Br, 22.76.

### Ethyl-{4-ethyl-5-[3-formyl-1-(tetrahydro-pyran-2-yl)-1H-indazol-5-yl]-pyridin-3-ylmethyl}-carbamic acid tert-butyl ester (46d)

Following a similar procedure to 36d, bromide 46c (10.84 g, 31.6 mmol) was coupled with boronic ester 36c (11.25 g, 31.6 mmol), yielding, after column chromatography and trituration for cyclohexane, aldehyde 46d (12.53 g, 80%) as an off-white powder. ¹H NMR (DMSO-d₆) δ 10.20 (s, 1H), 8.33 (s, 1H), 8.29 (s, 1H), 8.03 (s, 1H), 8.02 (d, J=9.8 Hz, 1H), 7.54 (dd, J=1.3, 8.6 Hz, 1H), 6.12 (dd, J=2.5, 9.4 Hz, 1H), 4.55 (s, 2H), 3.91 (m, 1H), 3.82 (m, 1H), 3.21 (br s, 2H), 2.58 (q, J=7.6 Hz, 2H), 2.43 (m, 1H), 2.09 (m, 2H), 1.80 (m, 1H), 1.63 (m, 2H), 1.38 (br s, 9H), 1.04 (t, J=7.0 Hz, 3H), 0.87 (t, J=7.0 Hz, 3H). Anal. Calc. for C₂₈H₃₆N₄O₄.0.1H₂O: C, 68.02; H, 7.38; N, 11.33. Found: C, 68.02; H, 7.24; N, 11.06.

### Ethyl-{4-ethyl-5-[3-(4,5,6,7-tetrahydro-1H-benzoimidazol-2-yl)-1-(tetrahydro-pyran-2-yl)-1H-indazol-5-yl]-pyridin-3-ylmethyl}-carbamic acid tert-butyl ester (46e)

By the same procedure used to make 21a, aldehyde 46d (1.20 g, 2.44 mmol) was cyclized with 1,2-cyclohexanedione (287 mg, 2.56 mmol) in the presence of ammonium acetate (1.13 g, 14.6 mmol), affording, after analogous purification, 46e (802.5 mg, 52%) as a yellow foam. ¹H NMR (DMSO-d₆) δ 12.22 (s, 1H), 8.32 (s, 1H), 8.30 (s, 1H), 8.27 (s, 1H), 7.83 (d, J=8.6 Hz, 1H), 7.41 (dd, J=1.3, 8.6 Hz, 1H), 5.93 (dd, J=2.3, 9.8 Hz, 1H), 4.55 (s, 2H), 3.93 (m, 1H), 3.77 (m, 1H), 3.21 (m, 2H), 2.57 (m, 5H), 2.05 (m, 2H), 1.74 (m, 5H), 1.61 (m, 2H), 1.40 (s, 9H), 1.05 (t, J=7.2 Hz, 3H), 0.88 (t, J=7.0 Hz, 3H). Anal. Calc. for C₃₄H₄₄N₆O₃.0.5 EtOAc: C, 68.76; H, 7.69; N, 13.37. Found: C, 68.73; H, 7.36; N, 13.46.

### Ethyl-{4-ethyl-5-[3-(4,5,6,7-tetrahydro-1H-benzoimidazol-2-yl)-1H-indazol-5-yl]-pyridin-3-ylmethyl}-amine (46)

Example 46 was prepared in the same manner as example 41. Compound 46e (699.3 mg, 1.18 mmol) yielded, after workup and purification, white solid 46 (340.2 mg, 70%). ¹H NMR (MeOD) δ 8.49 (s, 1H), 8.30 (s, 1H), 8.24 (s, 1H), 7.63 (d, J=8.6 Hz, 1H), 7.38 (dd, J=1.8, 8.6 Hz, 1H), 3.91 (s, 2H), 2.80 (q, J=7.1 Hz, 2H), 2.76 (q, J=7.3 Hz, 2H), 2.64 (br s, 4H), 1.86 (br s, 4H), 1.20 (t, J=7.1 Hz, 3H), 0.97 (t, J=7.6 Hz, 3H). Anal. Calc. for C₂₄H₂₈N₆.0.2 H₂O.0.15 EtOH: C, 71.00; H, 7.19; N, 20.45. Found: C, 70.93; H, 7.13; N, 20.17.

### EXAMPLE 47

### Ethyl-{4-ethyl-5-[3-(1H-imidazol-2-yl)-1H-indazol-5-yl]-pyridin-3-ylmethyl}-amine

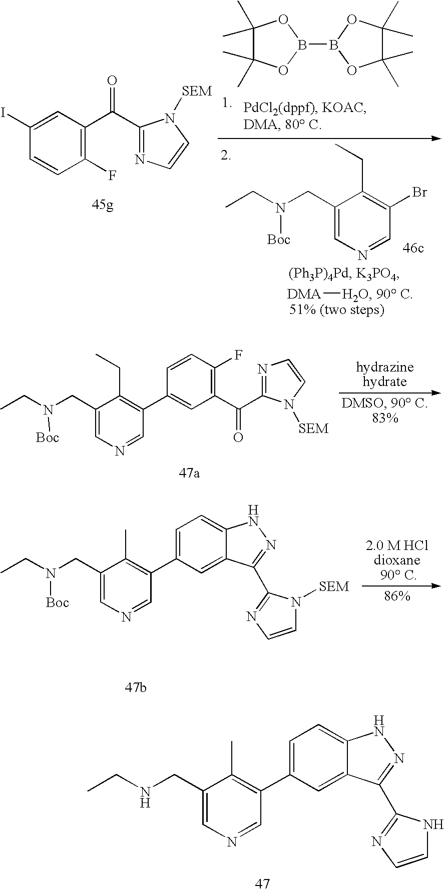

### Ethyl-(4-ethyl-5-{4-fluoro-3-[1-(2-trimethylsilanyl-ethoxymethyl)-1H-imidazole-2-carbonyl]-phenyl}-pyridin-3-ylmethyl)-carbamic acid tert-butyl ester (47a)

Example 47a was formed in a similar manner as Example 1e using the biarylketone 45g (1.02 g, 2.287 mmol), bis(pinacolato)diboron (610 mg, 2.40 mmol), potassium acetate (672 mg, 6.86 mmol) and [1,1′-bis(diphenylphosphino)-ferrocene] dichloropalladium(II)complex with dichloromethane (60 mg, 0.07 mmol) in N,N-dimethylacetamide (20 mL) in the first step. Bromopyridine 46c (821 mg, 2.401 mmol), potassium phosphate (727 mg, 3.431 mmol), tetrakis(triphenylphosphine) palladium (0) (80 mg, 0.07 mmol), deionized water (5 mL) and N,N-dimethylacetamide (5 mL) were added for the second step. Analogous chromatography conditions afforded 47a (684 mg, 51%) as a yellow oil: 1H NMR (300 MHz, CHLOROFORM-D) δ ppm 8.35 (s, 1 H) 8.34 (s, 1 H) 7.64 (dd, J=6.50, 2.35 Hz, 1 H) 7.40-7.46 (m, 1 H) 7.39 (d, J=0.94 Hz, 1 H) 7.26 (d, J=0.94 Hz, 1 H) 7.19-7.24 (m, 1 H) 5.85 (s, 2 H) 4.55 (s, 2 H) 3.57-3.66 (m, 2 H) 3.24 (bs, 2 H) 2.64 (q, J=7.54 Hz, 2 H) 1.46 (s, 9 H) 1.09 (t, J=6.97 Hz, 3 H) 0.90-1.02 (m, 5 H) −0.04 (s, 9 H).

### Ethyl-(4-ethyl-5-{3-[1-(2-trimethylsilanyl-ethoxymethyl)-1H-imidazol-2-yl]-1H-indazol-5-yl}-pyridin-3-ylmethyl)-carbamic acid tert-butyl ester (47b)

Example 47b was prepared in a similar manner as Example 45i, Method B using the biarylketone compound 47a (684 mg, 1.175 mmol) and hydrazine hydrate (365 ul, 11.75 mmol) in DMSO (14 ml). Analogous chromatography conditions afforded 47b (560 mg, 83%) as a yellow powder: 1H NMR (300 MHz, CHLOROFORM-D) δ ppm 8.50 (s, 1 H) 8.39 (s, 1 H) 8.36 (s, 1 H) 7.54 (d, J=8.48 Hz, 1 H) 7.35 (dd, J=8.57, 1.41 Hz, 1 H) 7.23 (d, J=1.13 Hz, 1 H) 7.19 (d, J=1.13 Hz, 1 H) 5.96 (s, 2 H) 4.58 (s, 2 H) 3.53-3.65 (m, 2 H) 3.25 (bs, 2 H) 2.64 (q, J=7.54 Hz, 2 H) 1.47 (s, 9 H) 1.12 (t, J=6.88 Hz, 3 H) 0.85-1.00 (m, 5 H) −0.11 (s, 9 H).

### Ethyl-{4-ethyl-5-[3-(1H-imidazol-2-yl)-1H-indazol-5-yl]-pyridin-3-ylmethyl}-amine (47)

Example 47 was prepared in a similar manner as Example 45, Method B using the indazole compound 47b (530 mg, 0.920 mol) in 2N HCl solution (5 ml). Analogous chromatography conditions afforded 47 (275 mg, 86%) as a yellow powder: 1H NMR (300 MHz, MeOH) δ ppm 8.49 (s, 1 H) 8.28 (d, J=11.87 Hz, 2 H) 7.57-7.71 (m, J=6.22 Hz, 1 H) 7.32-7.46 (m, J=5.46 Hz, 1 H) 7.17 (bs, 2 H) 3.89 (s, 2 H) 2.76 (bs, 4 H) 1.18 (bs, 3 H) 0.96 (bs, 3 H).

Anal. Calcd. for C₂₀H₂₂N₆.0.15 EtOH.0.1 HCl: C, 68.30; H, 6.49; N, 23.54. Found: C, 68.33; H, 6.52; N, 23.57.

### EXAMPLE 48

### {5-[3-(1H-Imidazol-2-yl)-1H-indazol-5-yl]-4-methyl-pyridin-3-ylmethyl}-isopropyl-amine

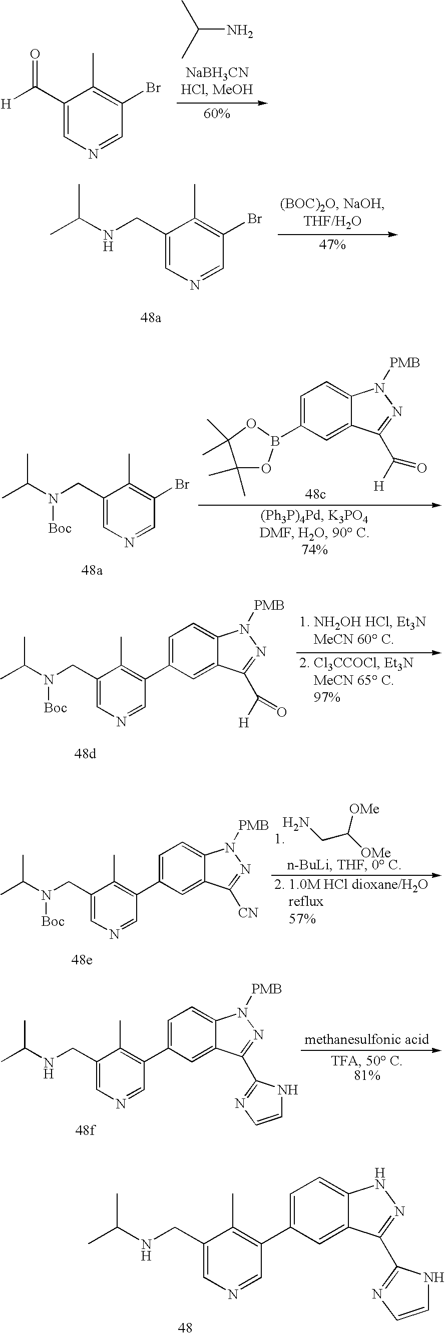

### (5-Bromo-4-methyl-pyridin-3-ylmethyl)-isopropyl-amine (48a)

Isopropyl amine (8.87 g, 150 mmol) was added to a solution of 5-Bromo-4-methyl-pyridine-3-carbaldehyde (5.00 g, 25.0 mmol) [for the preparation of this compound see: Reich, S. R.; Bleckman, T. M.; Kephart, S. E.; Romines, W. H.; Wallace, M. B., U.S. Pat. No. 6,555,539, Apr. 29, 2003.] in 25 mL methanol. The mixture was cooled to 0° C. and a solution of HCl in dioxane (4.0M, 18.75 mL) was added, causing fuming. Solid sodium cyanoborohydride (943 mg, 15 mmol) was added in one portion, the cooling bath removed, and the solution stirred at room temperature for 18 hours. The reaction was quenched with 10 mL 3M aqueous HCl, and the solvents removed in vacuo. The remaining acidic solution was extracted with diethyl ether (2×50 mL). The ether extracts were discarded, and the aqueous layer treated with solid sodium hydroxide to bring the pH up to 10. This basic solution was extracted with ethyl acetate (3×100 mL). The combined ethyl acetate extracts were dried over magnesium sulfate, filtered, concentrated, and purified by silica gel chromatography (eluting with 1/19/80 concentrated ammonium hydroxide/ethanol/ethyl acetate), to give amine 48a (3.62 g, 60%) as a yellow liquid. ¹H NMR (CDCl₃) δ 8.55 (s, 1H), 8.34 (s, 1H), 3.78 (s, 2H), 2.86 (quint, J=6.2 Hz, 1H) 2.45 (s, 3H), 1.26 (br s, 1H), 1.11 (d, J=6.2 Hz, 6H). Anal. Calc. for C₁₀H₁₅BrN₂: C, 49.40; H, 6.22; N, 11.52; Br, 32.86. Found: C, 49.24; H, 6.15; N, 11.50; Br, 32.72.

### (5-Bromo-4-methyl-pyridin-3-ylmethyl)-isopropyl-carbamic acid tert-butyl ester (48b)

Amine 48a (3.25 g, 13.37 mmol) was dissolved in THF (300 mL). Di-tert-butyl dicarbonate (3.21 g, 14.7 mmol) and 1.0 M NaOH solution (33.4 mL) were added. Stirring was continued at room temperature for 6.5 hours. The solvents were removed in vacuo, and the residue partitioned between 50 mL deionized water and 200 mL ethyl acetate. The organic layer was washed with saturated aqueous sodium chloride (100 mL), dried over magnesium sulfate, filtered, and concentrated. Silica gel chromatography (eluting with 30-80% ethyl acetate in hexanes) afforded pure 48b (2.15 g, 47%) as a colorless oil, which crystallized on standing. ¹H NMR (DMSO-d₆) δ 8.55 (s, 1H), 8.14 (s, 1H), 4.36 (s, 2H), 4.14 (br s, 1H), 2.36 (s, 3H), 1.32 (s, 9H), 1.07 (d, J=6.2 Hz, 6H). Anal. Calc. for C₁₅H₂₃BrN₂O₂: C, 52.49; H, 6.75; N, 8.16; Br, 23.28. Found: C, 52.21; H, 6.79; N, 7.69; Br, 22.99.

### {5-[3-Formyl-1-(4-methoxy-benzyl)-1H-indazol-5-yl]-4-methyl-pyridin-3-ylmethyl}-isopropyl-carbamic acid tert-butyl ester (48d)

Bromide 48b (1.54 g, 4.49 mmol) and potassium phosphate (1.43 g, 6.73 mL) were added to a solution of 1-(4-Methoxy-benzyl)-5-(4,4,5,5-tetramethyl-[1,3,2]dioxaborolan-2-yl)-1H-indazole-3-carbaldehyde 48c [1.76 g, 4.49 mmol, for the preparation of this compound see: Reich, S. R.; Bleckman, T. M.; Kephart, S. E.; Romines, W. H.; Wallace, M. B., U.S. Pat. No. 6,555,539, Apr. 29, 2003.] in 38 mL N,N-dimethylacetamide. The solution was degassed as described in the procedure for 1e, tetrakis(triphenylphosphine) palladium (0) (518 mg, 0.45 mmol) was added, and the solution degassed again. The solution was heated to 85° C. for 2 hours. After cooling to room temperature, the mixture was partitioned between 50 mL deionized water and 100 mL ethyl acetate. The organic layer was washed with brine, dried over magnesium sulfate, filtered and concentrated in vacuo. The crude red oil thus obtained was purified by silica gel chromatography

(eluting with 50-100% ethyl acetate in hexanes), yielding a yellow foam (2.31 g) which contained both 48d and pinacol. A second silica gel chromatography (eluting with 5% methanol in dichloromethane) afforded pure 48d (1.94 g, 82%) as a pale yellow foam. ¹H NMR (DMSO-d₆) δ 10.19 (S, 1H), 8.29 (s, 1H), 8.24 (s, 1H), 8.05 (m, 2H), 7.50 (dd, J=1.1, 8.9 Hz, 1H), 7.36 (d, J=8.7 Hz, 2H), 6.91 (d, J=8.7 Hz, 2H), 5.80 (s, 2H), 4.38 (s, 2H), 4.18 (br s, 1H), 3.71 (s, 3H), 2.16 (s, 3H), 1.35 (s, 9H), 1.11 (d, J=6.8 Hz, 6H). Anal. Calc. for C₃₁H₃₆N₄O₄: C, 70.43; H, 6.86; N, 10.60. Found: C, 70.17; H, 6.89; N, 10.29.

### {5-[3-Cyano-1-(4-methoxy-benzyl)-1H-indazol-5-yl]-4-methyl-pyridin-3-ylmethyl}-isopropyl-carbamic acid tert-butyl ester (48e)

Example 48e was prepared in a similar manner to Example 1g, Method B, using aldehyde 48d (1.024 g, 1.937 mmol), hydroxylamine hydrochloride (140 mg, 2.014 mmol) and triethylamine (280 ul, 2.013 mmol) in acetonitrile (25 ml) for the first step. The second step used trichloroacetylchloride (320 ul, 2.866 mmol) and triethylamine (560 ul, 4.026 mmol). Analogous chromatography gave 48e (985 mg, 97%) as a light yellow solid: 1H NMR (300 MHz, CHLOROFORM-D) δ ppm 8.39 (s, 1 H) 8.33 (s, 1 H) 7.73 (s, 1 H) 7.55 (d, J=8.67 Hz, 1 H) 7.35 (d, J=8.85 Hz, 1 H) 7.26 (d, J=8.10 Hz, 2 H) 6.87 (d, J=8.48 Hz, 2 H) 5.62 (s, 2 H) 4.40 (s, 2 H) 4.04-4.19 (m, J=7.03, 7.03, 7.03 Hz, 1 H) 3.78 (s, 3 H) 2.18 (s, 3 H), 1.44 (s, 9 H) 1.17 (d, J=6.78 Hz, 6 H).

### {5-[3-(1H-Imidazol-2-yl)-1-(4-methoxy-benzyl)-1H-indazol-5-yl]4-methyl-pyridin-3-ylmethyl}-isopropyl-amine (48f)

Example 48f was prepared in a similar manner to Example 1, Method B, using nitrile 48e (985 mg, 1.876 mmol). A 2.5 M solution of n-butyllithium in hexanes (4.1 ml, 10.25 mmole) was added under argon to a solution of aminoacetaldehyde dimethyl acetal (1.1 ml, 10.11 mmole) in THF (10 ml) at −78° C. After stirring for a further 30 minutes at −78° C., a portion (7 ml, 4.725 mmole) of this lithium 2,2-dimethoxyethylamide solution was added to a solution nitrile 48e (985 mg, 1.876 mmol) in THF (20 ml). The resultant solution was stirred, under argon, at 0° for 2 hours, then quenched by addition of 50% aqueous CH₃OH (4 ml). The volatiles were removed by concentration, in vacuo, and the residue obtained was dissolved in 4.0 M HCl in 1,4-dioxane (10 ml). This solution was diluted with water (10 ml) and then heated at reflux overnight. Reaction work-up and chromatography were analogous to Example 1, Method B, and afforded compound 48f (499 mg, 57%) as a brown solid: 1H NMR (300 MHz, MeOH) δ ppm 8.42 (s,1 H) 8.32 (s,1 H) 8.26 (s, 1 H) 7.59 (d, J=8.67 Hz, 1 H) 7.32 (dd, J=8.67, 1.51 Hz, 1 H) 7.23 (d, J=8.48 Hz, 2 H) 7.17 (s, 2 H) 6.81 (d, J=8.67 Hz, 2 H) 5.60 (s, 2 H), 3.83 (s, 2 H) 3.69 (s, 3 H) 2.84-2.94 (m, 1 H) 2.29 (s, 3 H) 1.15 (d, J=6.22 Hz, 6 H).

### {5-[3-(1H-Imidazol-2-yl)-1H-indazol-5-yl]-4-methyl-pyridin-3-ylmethyl}-isopropyl-amine (48)

In a flask was dissolved the PMB-protected indazole 48f (499 mg, 1.071 mmol) in TFA (10 ml) and CF₃SO₃H (2.5 ml). The reaction mixture was heated to 50° C. for 3 hr, cooled, poured into 38% ammonium hydroxide solution (20 ml) and water (10 ml), then extracted with EtOAc (3×75 ml). The organics were combined and washed with 2N NaOH solution (2×50 ml), dried over magnesium sulfate, filtered and concentrated to a crude oil. Purification by silica gel chromatography {eluting with 15% (5% concentrate ammonium hydroxide in ethanol) in chloroform afforded 48 (300 mg, 81%) as an off-white powder: 1H NMR (300 MHz, MeOH) δ ppm 8.54 (s, 1 H) 8.48 (s, 1 H) 8.27 (s, 1 H) 7.69 (d, J=8.67 Hz, 1 H) 7.40 (d, J=8.48 Hz, 1 H) 7.19 (s, 2 H) 4.25 (s, 2 H) 3.34-3.47 (m, J=12.72, 6.31 Hz, 1 H) 2.41 (s, 3 H) 1.37 (d, J=6.41 Hz, 6 H).

Anal. Calcd. for C₂₀H₂₂N₆.0.8 TFA.0.4 CHCl₃.0.9 H₂O: C, 52.67; H, 5.02; N, 16.75; F, 9.01. Found: C, 52.30; H, 4.99; N, 17.10; F, 8.93.

### EXAMPLE 49

### 2-(5-{5-[(ethylamino)methyl]-4-methylpyridin-3-yl}-1H-indazol-3-yl)-N-(pyridin-3-ylmethyl)-1H-imidazole-5-carboxamide

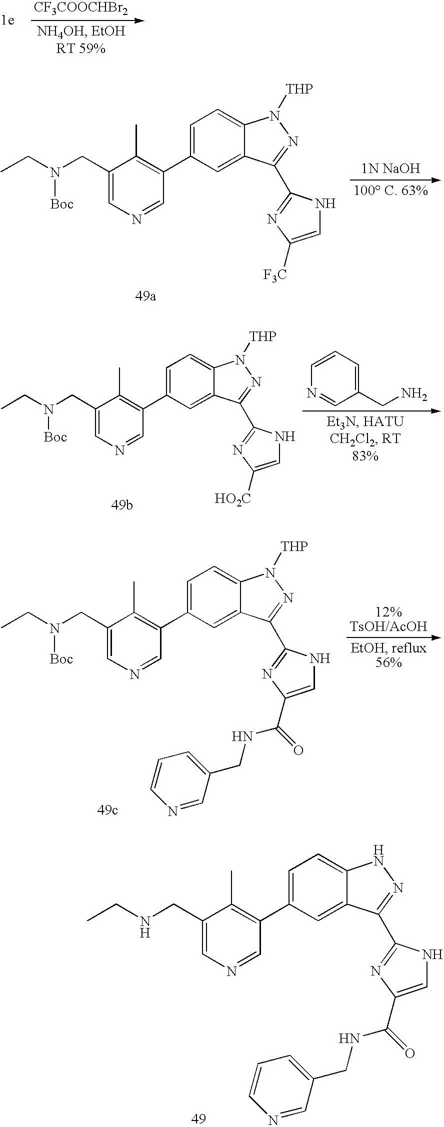 tert-Butyl ethyl[(4-methyl-5-{1-(tetrahydro-2H-pyran-2-yl)-3-[5-(trifluoromethyl)-1H-imidazol-2-yl]-1H-indazol-5-yl}pyridin-3-yl)methyl]carbamate (49a)

A clear solution of NaOAc (1.032 g, 12.53 mmol) and dibromotrifluoroacetone (1.669 g, 6.292 mmol) in 27 mL: H₂O was heated at 100° C. for 50 min. After cooling a solution of the aldehyde 1e (1.00 g, 2.089 mmol) in 40 mL EtOH and 8 mL concentrated NH₄OH was added to the reaction mixture (Ref: J. Med. Chem., 1979, vol. 22 no.6, p687-694). The reaction mixture was stirred at rt for 24h. The solvents were removed by rotary evaporation and the residue partitioned between EtOAc and saturated aqueous Na₂CO₃. The organic extracts were dried over MgSO₄ and concentrated. The crude compound was purified by silica gel chromatography (eluting with a gradient of 0% to 1% MeOH in CHCl₃), yielding 49a (0.720 g, 59%) as a pale yellow solid: ¹H NMR (DMSO-d₆) 613.46 (s, 1 H), 8.37 (s,1 H), 8.31 (s, 1H), 8.19 (s, 1H), 7.92 (t, J=9.0 Hz, 2H), 7.49 (d, J=6.0 Hz, 1H), 6.01 (d, J=9.0 Hz, 1H), 4.51 (s, 2H), 3.93-3.97 (m, 1H), 3.76-3.85 (m, 1H), 3.20 (q, J=6.0 Hz, 2H), 2.16 (s, 3H), 2.01-2.14 (m, 2H), 1.76-1.88 (m,1H), 1.59-1.65 (m, 2H), 1.40 (s, 9H), 1.23 (s, 1H) 1.03 (t, J=6.0 Hz, 3H); M+H⁺ 585.

### 1b 2-[5-(5-{[(tert-butoxycarbonyl)(ethyl)amino]methyl}-4-methylpyridin-3-yl)-1-(tetrahydro-2H-pyran-2-yl)-1H-indazol-3-yl]-1H-imidazole-5-carboxylic acid (49b)

A yellow suspension of 49a (0.580 g, 0.993 mmol) in 30 mL 1 N NaOH was heated at 100° C. for 2 h. The reaction mixture was then diluted with H₂O and washed with EtOAc. The aqueous layer was brought to pH 5 with 1N HCl and extracted with EtOAc several times. The combined organic extracts were dried over MgSO₄ and concentrated to give 49b (0.347 g, 63%) as a yellow solid, which was used in the next step without further purification: ¹H NMR (DMSO-d₆) 11.85 (br s, 1H), 8.38 (s, 1H), 8.29 (s, 2H), 7.90 (d, J=9.0 Hz, 1H), 7.81 (s, 1H), 7.46 (d, J=9.0 Hz, 1H), 6.00 (d, J=9.0 Hz, 1H), 4.51 (s, 2H), 3.93-4.00 (m, 1H), 3.78-3.85 (m, 1H), 3.20 (q, J=9.0 Hz, 2H), 2.16 (s, 3H), 2.01-2.12 (m, 3H), 1.76-1.87 (m, 1H), 1.62 (br s, 2H), 1.40 (s, 9H), 1.03 (t, J=9.0 Hz, 3H); M+H⁺ 561

### tert-Butyl ethyl({4-methyl-5-[3-(5-{[(pyridin-3-ylmethyl)amino]carbonyl}-1H-imidazol-2-yl)-1-(tetrahydro-2H-pyran-2-yl)-1H-indazol-5-yl]pyridin-3-yl}methyl)carbamate (49c)

To a clear greenish yellow solution of 49b (0.300 g, 0.536 mmol) in 10 ml CH₂Cl₂ was added Et₃N (0.090 mL, 0.065 g, 0.643 mmol), 3-(aminomethyl) pyridine (0.066 mL, 0.070 g, 0.643 mmol), and HATU (0.224 g, 0.590 mmol). The reaction mixture was stirred at rt for 2 h. The solvent was removed by rotary evaporation and 2 mL MeOH was added to the residue followed by H₂O when 49c (0.287 g, 82%) crashes out as a yellow solid which was collected by filtration and dried. This product was used in the next step without further purification: ¹H NMR (DMSO-d₆) δ 13.18 (br s, 1H), 8.76 (t, J=6.0 Hz, 1H), 8.29-8.65 (m, 5H), 7.90 (d, J=9.0 Hz, 1H), 7.79 (d, J=9.0 Hz, 1H), 7.71 (s, 1H), 7.41-7.47 (m, 2H), 5.99 (d, J=9.0 Hz, 1H), 4.47-4.51 (m, 4H), 3.93-3.98 (m, 1H), 3.72-3.82 (m, 1H), 3.22 (q, J=6.0 Hz, 2H), 2.68-2.71 (m, 1H), 2.18 (s, 3H), 2.00-2.14 (m, 2H), 1.79 (br s, 1H), 1.62 (br s, 2H), 1.41 (s, 9H), 1.05 (t, J=6.0 Hz, 3H); M+H⁺ 651.

### 2-(5-{5-[(ethylamino)methyl]-4-methylpyridin-3-yl}-1H-indazol-3-yl)-N-(pyridin-3-ylmethyl)-1H-imidazole-5-carboxamide (49)

A solution of 49c (0.263 g, 0.405 mmol) in 25 mL 10% aqueous EtOH and 0.69M TsOH in AcOH (12 mL, 8.10 mmol) was refluxed for 21 h. The solvent was removed by rotary evaporation and the residue was taken up in 5 mL H₂O, made basic to pH 12 with 50% wt NaOH and extracted several times with 20% iPrOH in CHCl₃. The organic extracts were dried over MgSO₄ and concentrated. The crude compound was purified by silica gel chromatography (eluting with a gradient of 0% to 30% MeOH saturated with NH₃ in CHCl₃), yielding 49 as a pale yellow hygroscopic solid. The hygroscopic solid was dissolved in 10 mL MeOH with PS-Trisamine resin (30 mg) and stirred for 18 h at rt. The resin was filtered and the filtrate concentrated to give 1 (0.092 g, 49%) as yellow crystalline solid: ¹H NMR (DMSO-d₆) δ 13.57 (br s, 1H), 13.16 (br s, 1H), 8.68 (t, J=6.0 Hz, 1H), 8.58 (s, 1H), 8.41-8.51 (m, 4H), 7.67-7.72 (m, 3H), 7.46 (d, J=9.0 Hz, 2H), 7.29-7.37 (m, 2H), 7.10 (d, J=9.0 Hz, 2H), 4.46 (d, J=6.0 Hz, 2H), 4.22 (s, 2H), 3.05 (q, J=6.0 Hz, 2H), 2.29 (s, 3H), 2.27 (s, 3H), 1.22 (t, J=6.0 Hz, 3H); M+H⁺ 467; Anal. (C₂₆H₂₆N₈O₄.1TsOH.2.5 H₂O) C, H, N.

### EXAMPLE 50

### N-cyclohexyl-2-(5-{5-[(ethylamino)methyl]-4-methylpyridin-3-yl}-1H-indazol-3-yl)-1H-imidazole-5-carboxamide

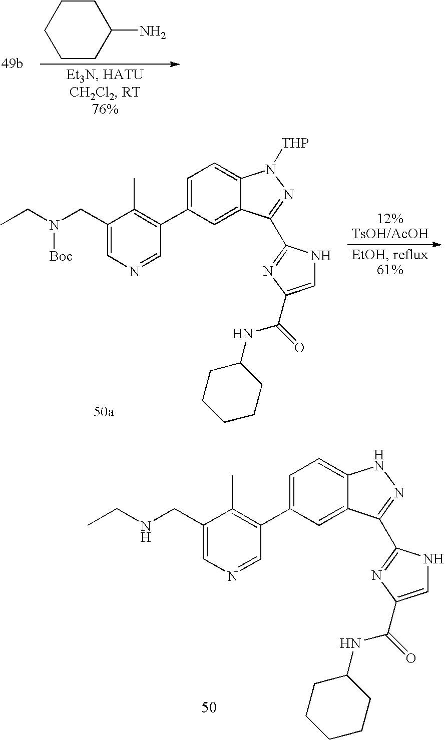

### tert-Butyl ({5-[3-{5-[(cyclohexylamino)carbonyl]-1H-imidazol-2-yl}-1-(tetrahydro-2H-pyran-2-yl)-1H-indazol-5-yl]-4-methylpyridin-3-yl}methyl)ethylcarbamate (50a)

By the same procedure used to synthesize intermediate 49c, acid 49b (0.250 g, 0.446 mmol), Et₃N (0.14 mL, 0.99 g, 0.981 mmol), cyclohexylamine (0.11 mL, 0.097 g, 0.981 mmol), HATU (0.373 g, 0.981 mmol) in 10 mL CH₂Cl₂ were considered affording 50a (0.217 g, 76%) as yellow solid: ¹H NMR (DMSO-d₆) δ 13.11 (br s, 1H), 8.40-8.41 (m, 2H), 8.30 (s, 1H), 7.90 d, J=9.0 Hz, 1H), 7.61-7.66 (m, 2H), 7.48 (dd, J=3.0 Hz, J=9.0 Hz, 1H), 5.99 (d, J=6.0 Hz, 1H), 4.51 (s, 2H), 3.93-3.97 (m, 1H), 3.67-3.84 (m, 2H), 3.24 (q, J=6.0 Hz, 2H), 2.20 (s, 3H), 1.98-2.08 (m, 2H), 1.53-1.67 (m, 8H), 1.41 (s, 9H), 1.06-1.35 (m, 6H), 1.04 (t, J=6.0 Hz, 3H); M+H⁺ 642.

### N-cyclohexyl-2-(5-{5-[(ethylamino)methyl]-4-methylpyridin-3-yl}-1H-indazol-3-yl)-1H-imidazole-5-carboxamide (50)

In the same manner as the deprotection of 49c, intermediate 50a (0.187 g, 0.292 mmol) was converted to the title compound 50 (0.081 g, 61%), as a yellow crystalline solid: ¹H NMR (DMSO-d₆) δ 13.52 (br s, 1H), 8.43 (s, 1H), 8.39 (s, 1H), 8.35 (s, 1H), 7.68 (d, J=9.0 Hz, 1H), 7.63 (s, 1H), 7.61 (d, J=9.0 Hz, 1H), 7.40 (dd, J=3.0 Hz, J=6.0 Hz, 1H), 3.77 (s, 2H), 3.67-3.73 (m,1H), 2.63 (q, J=6.0 Hz, 2H), 2.28 (s, 3H), 2.21-2.27 (m,1H), 1.72-1.79 (m, 2H), 1.64-1.70 (m, 2H), 1.50-1.60 (m, 1H), 1.20-1.39 (m, 4H), 1.07 (t J=6.0 Hz, 2H); M+H⁺458; Anal. (C₂₆H₃₁N₇O.1.25 H₂O) C, H, N.

### EXAMPLE 51

### N-[(1R)-2,3-dihydro-1H-inden-1-yl]-2-(5-[5-[(ethylamino)methyl]-4-methylpyridin-3-yl)1H-indazol-3-yl)-1H-imidazole-5-carboxamide

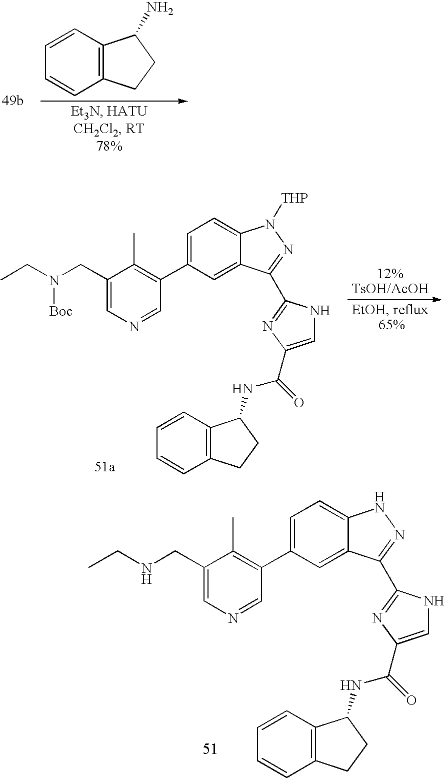

### tert-Butyl ({5-[3-(5-{[(1R)-2,3-dihydro-1H-inden-1-ylamino]carbonyl}-1H-imidazol-2-yl)-1-(tetrahydro-2H-pyran-2-yl)-1H-indazol-5-yl]-4-methylpyridin-3-yl}methyl)ethylcarbamate (51a)

By the same procedure used to synthesize intermediate 49c, acid 49b (0.270 g, 0.482 mmol), Et₃N (0.21 mL, 0.156 g, 1.54 mmol), (R)-1-aminoindane (0.20 mL,0.205 g, 1.54 mmol), HATU (0.403 g, 1.06 mmol) in 10 mL CH₂Cl₂ were considered affording 51a (0.254 g, 78%) as yellow solid: ¹H NMR (DMSO-d₆) δ 13.14 (s,1H), 8.45 (s,1H), 8.35(s,1H), 8.25-8.27 (m, 2H), 7.89 (d, J=9.0 Hz, 1H), 7.75 (s, 1H), 7.43 (d. J=9.0 Hz, 1H), 7.14-7.21 (m, 4H), 5.98 (d, J=9.0 Hz, 1H), 5.52 (q, J=9.0 Hz, 2H), 4.46 (s, 2h), 4.09-4.11 (m,1 h), 3.93-3.97 (m, 1H), 3.76-3.84 (m, 1H), 2.77-2.97 (m, 3H), 2.34-2.44 (m, 1H), 2.15 (s, 3H), 2.01-2.08 (m, 3H), 3.17 (q, J=6.0 Hz, 2H), 1.62 (br s, 2H), 1.40 Z(s, 9H), 1.03 (t, J=6.0 Hz, 3 H); M+H⁺676

### N-[(1R)-2,3-dihydro-1H-inden-1-yl]-2-(5-{5-[(ethylamino)methyl]-4-methylpyridin-3-yl}-1H-indazol-3-yl)-1H-imidazole-5-carboxamide (51)

In the same manner as the deprotection of 49c, intermediate 51 a (0.226 g, 0.335 mmol) was converted to the title compound 51 (0.106 g, 65%), as a yellow solid: ¹H NMR (DMSO-d₆) δ 13.51 (br s, 1H),8.40-8.41 (m, 2H), 8.30 (s,1H), 8.24 (d, J=9.0 Hz, 1H), 7.72 (s, 1H), 7.66 (d, J=9.0 Hz, 1H), 7.37 (dd, J=3.0 Hz, J=9.0 Hz, 1H), 7.12-7.24 (m, 4H), 5.51 (q, J=6.0 Hz, 1H), 3.72 (s, 2H), 2.76-2.96 (m, 2H), 2.61 (q, J=6.0 Hz, 2H), 2.35-2.42 (m, 1H), 2.21 (s, 3H), 2.00-2.07 (m, 1H), 1.05 (t, J=6.0 Hz, 3H); M+H⁺ 492; Anal. (C₂₉H₂₉N₇O.1.75 H₂O) C, H, N.

### EXAMPLE 52

### 2-(5-{5-[(ethylamino)methyl]-4-methylpyridin-3-yl}1H-indazol-3-yl)-N-(pyridin-2-ylmethyl)-1H-imidazole-5-carboxamide

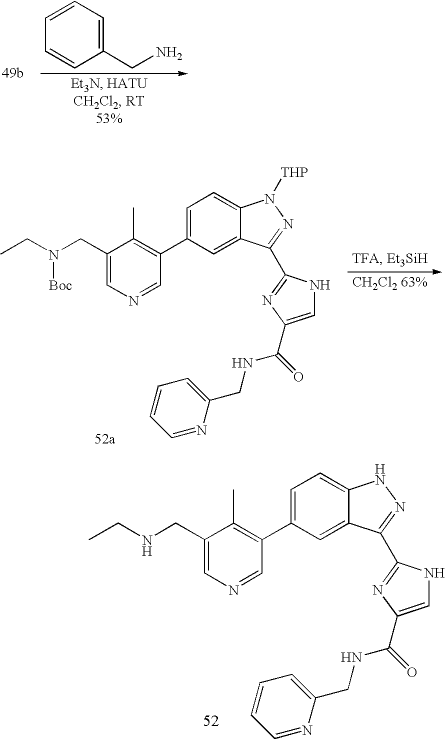

### tert-Butyl ethyl({4-methyl-5-[3-(5-{[(pyridin-2-ylmethyl)amino]carbonyl}-1H-imidazol-2-yl)-1-(tetrahydro-2H-pyran-2-yl)-1H-indazol-5-yl]pyridin-3-yl}methyl)carbamate (52a)

By the same procedure used to synthesize intermediate 49c, acid 49b (0.250 g, 0.446 mmol), Et₃N (0.12 mL, 0.090 g, 0.892 mmol), 2-(aminomethyl)pyridine (0.092 mL, 0.096 g, 0.892 mmol), HATU (0.187 g, 0.491 mmol) in 10 mL CH₂Cl₂ were considered affording 52a (0.155 g, 53%) as yellow solid: ¹H NMR (DMSO-d₆) δ 13.20 (s, 1H), 8.75 (d, J=6.0 Hz, 1H), 8.51 (s, 1H), 8.40 (s,1H), 8.35 (d, J=3.0 Hz, 1H), 8.28 (s, 1H), 7.90 (d, J=9.0 Hz, 1H), 7.71-7.75 (m, 2H), 7.46 (d, J=9.0 Hz, 1H), 7.30 (d, J=9.0 Hz, 1H), 7.21 (t, J=9.0 Hz, 1H), 6.00 (d, J=9.0.Hz, 1H), 4.54 (d, J=6.0 Hz, 2H), 4.49 (s, 2H), 3.94-3.97 (m, 1H), 3.75-3.87 (m, 1H), 3.18 (q, J=6.0 Hz, 2H), 2.54-2.64 (m, 1H), 2.18 (s, 3H), 2.04-2.13 (m, 2H), 1.75-1.89 (m, 1H), 1.63 (s, 2H), 1,40 (s, 9H), 1.03 (t, J=6.0 Hz, 3H); M+H⁺ 651.

### 2-(5-{5-[(ethylamino)methyl]-4-methylpyridin-3-yl}-1H-indazol-3-yl)-N-(pyridin-2-ylmethyl)1H-imidazole-5-carboxamide (52)

A clear yellow solution of 52a (0.143 g, 0.220 mmol)in 9 mL CH₂Cl₂, 1 mL TFA,and 0.1 mL Et₃SiH was stirred at rt for 22 h. The solvent was removed by rotary evaporation and the residue was taken up in 5 mL H₂O, made basic to pH 12 with 50% wt NaOH and extracted several times with 20% iPrOH in CHCl₃. The organic extracts were dried over MgSO₄ and concentrated. The crude compound was purified by silica gel chromatography (eluting with a gradient of 0% to 15% MeOH saturated with NH₃ in CHCl₃), yielding 52 (0.065 g, 63%), as a yellow solid: ¹H NMR (DMSO-d₆) δ 13.53 (s,1H),8.72-8.76 (m,1H), 8.44-8.48 (m,2H), 8.32-8.36 (m, 2H), 7.67-7.75 (m, 3H), 7.39 (d, J=9.0 Hz, 1H), 7.30 (d, J=9.0 Hz, 1H), 7.22 (t, J=6.0 Hz, 1H), 4.55 (d, J=3.0 Hz, 2H), 3.74 (s, 2H), 2.58 (q, J=6.0 Hz, 2H), 2.25 (s, 3H), 1.03 (t, J=6.0Hz, 3H); M+H⁺ 467; Anal. (C₂₆H₂₆N₈O.1.25H₂O.0.65 iPrOH.0.1 TFA) C, H, N.

### EXAMPLE 53

### N-[(1S)-2,3-dihydro-1H-inden-1-yl]-2-(5-{5-[(ethylamino)methyl]-4-methylpyridin-3-yl]1H-indazol-3-yl)-1H-imidazole-5-carboxamide

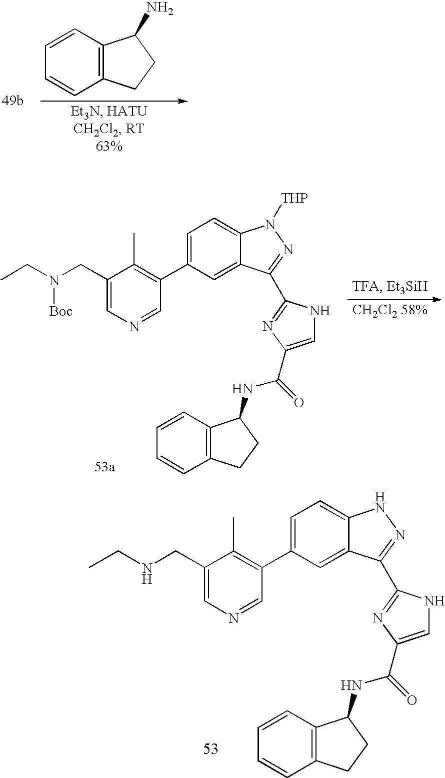

### tert-butyl ({5-[3-(5-{[(1S)-2,3-dihydro-1H-inden-1-ylamino]carbonyl}-1H-imidazol-2-yl)-1-(tetrahydro-2H-pyran-2-yl)-1H-indazol-5-yl]-4-methylpyridin-3-yl}methyl)ethylcarbamate (53a)

By the same procedure used to synthesize intermediate 49c, acid 49b (0.250 g, 0.446 mmol), Et₃N (0.12 mL, 0.090 g, 0.892 mmol), (S)-aminoindane (0.115 mL, 0.119 g, 0.892 mmol), HATU (0.187 g, 0.491 mmol) in 10 mL CH₂Cl₂ were considered affording 53a (0.191 g, 63%) as yellow solid: ¹H NMR (DMSO-d₆) δ 13.14 (s,1H), 8.44 (s, 1H), 8.35 (s, 1H), 8.24 (s, 1H), 7.88 (d, J=9.0 Hz, 1H), 7.74 (s, 1H), 7.44 (d, J=9.0 Hz, 1H), 7.15-7.23 (m, 5H), 5.98 (d, J=9.0 Hz, 1H), 5.53 (q, J=6.0 Hz, 1H), 4.46 (d, J=6.0 Hz, 2H), 3.93-3.96 (m, 1H), 3.76-3.83 (m, 1H), 3.19 (q, J=6.0 Hz, 2H), 2.75-2.97 (m, 2H), 2.34-2.45 (m, 2H), 2.15 (s, 3H), 2.00-2.11 (m, 4H), 1.62 (s, 2H), 1.40 (s, 9H), 1.03 (t, J=6.0 Hz, 3H); M+H⁺ 676.

### N-[(1S)-2,3-dihydro-1H-inden-1-yl]-2-(5-{5-[(ethylamino)methyl]-4-methylpyridin-3-yl]-1H-indazol-3-yl)-1H-imidazole-5-carboxamide (53)

In the same manner as the deprotection of 52a, intermediate 53a (0.180 g, 0.267 mmol) was converted to the title compound 53 (0.076 g, 58%), as an orange solid: ¹H NMR (DMSO-d₆) δ 13.52 (s, 1H), 8.42-8.45 (m, 2H), 8.32 (s, 1H), 8.24 (d, J=6.0 Hz, 1H), 7.66-7.73 (m, 2H), 7.36 (d, J=9.0 Hz, 1H), 7.15-7.22 (m, 4H), 5.47-5.56 (m, 1H), 3.79 (s, 2H), 2.79-2.98 (m, 2H), 2,66 (q, J=6.0 Hz, 2H), 2.34-2.43 (s,1H), 2.23 (s, 3H), 1.98-2.09 (m, 1H), 1.08 (t, J=6.0 Hz, 3H); M+H⁺ 492; Anal. (C₂₉H₂₉N₇O.0.1 TFA 1.75 H₂O) C, H, N.

### EXAMPLE 54

### N-[2-(dimethylamino)ethyl]-2-(5-{5-[(ethylamino)methyl]-4-methylpyridin-3-yl}-1H-indazol-3-yl)-1H-imidazole-5-carboxamide

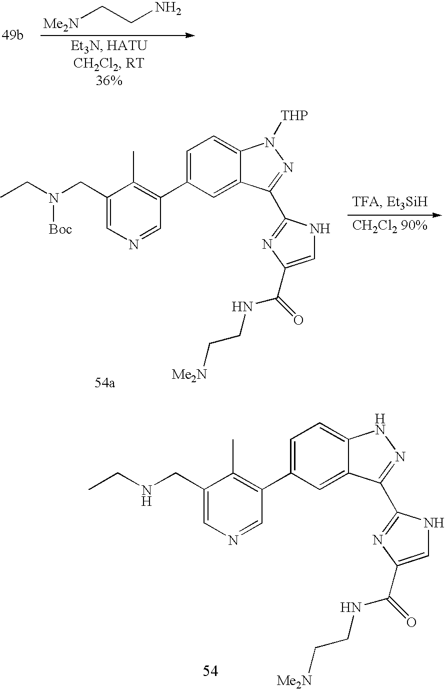

### tert-Butyl ({5-[3-[5-({[2-(dimethylamino)ethyl]amino}carbonyl)-1H-imidazol-2-yl]-1-(tetrahydro-2H-pyran-2-yl)-1H-indazol-5-yl]-4-methylpyridin-3-yl}methyl)ethylcarbamate (54a)

By the same procedure used to synthesize intermediate 49c, acid 49b (0.250 g, 0.446 mmol), Et₃N (0.12 mL, 0.090 g, 0.892 mmol), N,N-dimethylethylenediamine (0.098 mL, 0.079 g, 0.892 mmol), HATU (0.187 g, 0.491 mmol) in 10 mL CH₂Cl₂ were considered affording 54a (0.102 g, 36%) as yellow solid: ¹H NMR (DMSO-d₆) δ 13.16 (s, 1H), 8.40 (d, J=6.0 Hz, 2H), 8.29 (s, 1H), 7.99 (br s, 1H), 7.91 (d, J=9.0 Hz, 1H), 7.65 (s, 1H), 7.46 (dd, J=3.0 Hz, J=6.0 Hz, 1H), 5.99 (d, J=9.0 Hz, 1H), 4.51 (s, 2H), 3.93-3.98 (m, 1H), 3.77-3.82 (m, 1H), 3.32 (s, 4H), 3.20 (q J=6.0 Hz, 2H), 2.39-2.43 (m, 1H), 2.18 (s, 6H), 2.15 (s, 3H), 2.02-2.08 (m, 2H), 1.77-1.89 (m, 1H), 1.62 (s, 2H), 1.41 (s, 9H), 1.05 (t, J=6.0 Hz, 3H); M+H⁺631.

### N-[2-(dimethylamino)ethyl]-2-(5-{5-[(ethylamino)methyl]-4-methylpyridin-3-yl}-1H-indazol-3-yl)-1H-imidazole-5-carboxamide (54)

In the same manner as the deprotection of 52a, intermediate 54a (0.094 g, 0.149 mmol) was converted to the title compound 54 (0.062 g, 94%), as a yellow solid: ¹H NMR (DMSO-d₆) δ 13.57 (s, 1H), 8.52 (s, 1H), 8.40 (d, J=9.0 Hz, 2H), 7.99 (s, 1H), 7.64-7.72 (m, 2H), (d, J=9.0 Hz, 1H), 4.01 (s, 2H), 3.41 (s, 4H), 2.83 (q, J=6.0 Hz, 2H), 2.29 (s, 3H), 2.20 (s, 6H), 1.16 (t, J=6.0 Hz, 3H); M+H⁺ 447; Anal. (C₂₄H₃₀N₈O 1 TFA 2 H₂O) C, H, N.

### EXAMPLE 55

### N-[(4-methyl-5-{3-[5-(morpholin-4-ylcarbonyl)-1H-imidazol-2-yl]-1H-indazol-5-yl}pyridin-3-yl)methyl]ethanamine

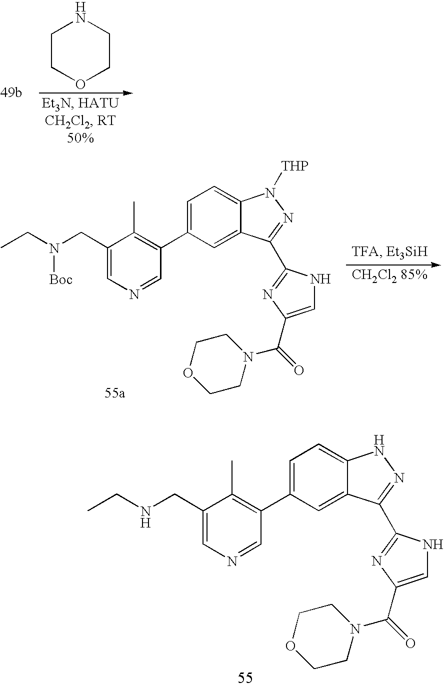

### tert-Butyl ethyl({4-methyl-5-[3-[5-(morpholin-4-ylcarbonyl)-1H-imidazol-2-yl]-1-(tetrahydro-2H-pyran-2-yl)-1H-indazol-5-yl]pyridin-3-yl}methyl)carbamate (55a)

By the same procedure used to synthesize intermediate 49c, acid 49b (0.250 g, 0.446 mmol), Et₃N (0.14 mL, 0.099 g, 0.981 mmol), morpholine (0.085 mL, 0.085 g, 0.981 mmol), HATU (0.373 g, 0.981 mmol) in 10 mL CH₂Cl₂ were considered affording 55a (0.140 g, 50%) as yellow solid: ¹H NMR (DMSO-d₆) δ 13.27 (s, 1H), 8.39 (s, 1H), 8.31 (s, 1H), 8.22 (s, 1H), 7.92 (d, J=6.0 Hz, 1H), 7.69 (d, J=3.0 Hz, 1H), 7.50 (d, J=6.0 Hz, 1H), 6.00 (d, J=6.0 Hz, 1H), 4.52 (s, 2H), 4.28 (br s, 1H), 3.94-3.97 (m, 1H), 3.77-3.82 (m, 1H), 3.58 (s, 6H), 3.21 (s, 2H), 2.49-2.51 (m, 1H), 2.20 (s, 3H), 2.03-2.12 (m, 2H), 1.79-1.82 (m, 1H), 1.62 (s, 2H), 1.40 (s, 9H), 1.02 (t, J=6.0 Hz, 3H), 0.85 (t, J=6.0 Hz, 1H); M+H⁺ 630.

### N-[(4-methyl-5-{3-[5-(morpholin-4-ylcarbonyl)-1H-imidazol-2-yl]-1H-indazol-5-yl}pyridin-3-yl)methyl]ethanamine (55)

In the same manner as the deprotection of 52a, intermediate 55a (0.130 g, 0.207 mmol) was converted to the title compound 7 (0.078 g, 85%), as a pale yellow solid: ¹H NMR (DMSO-d₆) δ 13.54 (s, 1H), 8.48 (s, 1H), 8.38 (s, 1H), 8.21 (s, 1H), 7.67-7.72 (m, 2H), 7.43 (d, J=6.0 Hz, 1H), 4.28-4.37 (m, 1H), 3.91 (s, 2H), 3.60 (br s, 5H), 3.41-3.50 (m, 1H), 2.75 (q, J=6.0 Hz, 2H), 2.30 (s, 3H), 1.11 (t, J=6.0 Hz, 3H), 1.04 (t, J=6.0 Hz, 1H); M+H⁺ 446; Anal. (C₂₄H₂₇N₇O₂ 0.4 TFA 0.5 H₂O) C, H, N.

### EXAMPLE 56

### N,N-diethyl-2-(5-{5-[(ethylamino)methyl]-4-methylpyridin-3-yl}-1H-indazol-3-yl)-1H-imidazole-5-carboxamide

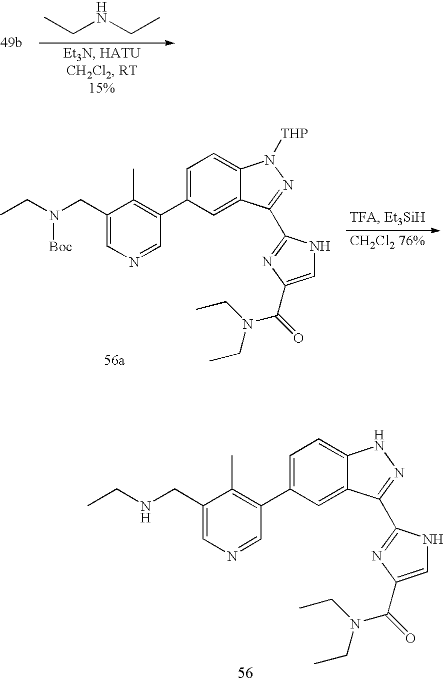

### tert-Butyl ({5-[3-{5-[(diethylamino)carbonyl]-1H-imidazol-2-yl}-1-(tetrahydro-2H-pyran-2-yl)-1H-indazol-5-yl]-4-methylpyridin-3-yl}methyl)ethylcarbamate (56a)

By the same procedure used to synthesize intermediate 49c, acid 49b (0.250 g, 0.446 mmol), Et₃N (0.14 mL, 0.099 g, 0.981 mmol), diethylamine (0.102 mL, 0.072 g, 0.981 mmol), HATU (0.373 g, 0.981 mmol) in 10 mL CH₂Cl₂ were considered affording 56a (0.042 g, 15%) as yellow solid: ¹H NMR (DMSO-d₆) δ 13.20 (s, 1H), 8.37 (s, 1H), 8.30 (s, 2H), 7.92 (d, J=6.0 Hz, 1H), 7.65 (s, 1H), 7.49 (d, J=6.0 Hz, 1H) 6.00 (d, J=6.0 Hz, 1H), 4.51 (s, 2H), 3.94-3.97 (m, 3H), 3.79-3.83 (m, 1H), 3.28-3.37 (m, 2H), 3.19 (q, J=6.0 Hz, 2H), 2.46-2.52 (m, 1H), 6H), 1.02 (t, J=6.0 Hz, 3H); M+H⁺ 616.

### N,N-diethyl-2-(5-{5-[(ethylamino)methyl]-4-methylpyridin-3-yl}-1H-indazol-3-yl)-1H-imidazole-5-carboxamide (56)

In the same manner as the deprotection of 52a, intermediate 56a (0.042 g, 0.068 mmol) was converted to the title compound 56 (0.022 g, 76%), as a pale yellow solid: ¹H NMR (DMSO-d₆) δ 13.55 (s, 1H), 13.16 (s, 1H), 8.55 (s, 1H), 8.44 (s, 1H), 8.30 (s, 1H), 7.72 (d, J=9.0 Hz, 1H), 7.63 (s, 1H), 7.42 (d, J=9.0 Hz, 1H), 4.14 (s, 2H), 3.92 (br s, 2H), 3.33-3.41 (m, 2H), 2.95-2.98 (m, 2H), 2.32 (s, 3H), 1.12-1.22 (m, 9H); M+H⁺ 432; Anal. (C₂₄H₂₉H₇O 0.9 TFA 0.75 H₂O) C, H, N.

### EXAMPLE 57

### N-{[4-methyl-5-(3-[5-[(4-methylpiperazin-1-yl)carbonyl]-1H-imidazol-2-yl}-1H-indazol-5-yl)pyridin-3-yl]methyl}ethanamine

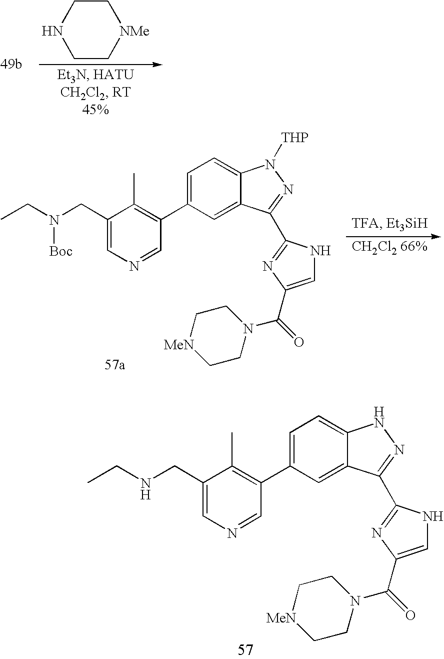

### tert-Butyl ethyl({4-methyl-5-[3-{5-[(4-methylpiperazin-1-yl)carbonyl]-1H-imidazol-2-yl}-1-(tetrahydro-2H-pyran-2-yl)-1H-indazol-5-yl]pyridin-3-yl}methyl)carbamate (57a)

By the same procedure used to synthesize intermediate 49c, acid 49b (0.250 g, 0.446 mmol), Et₃N (0.14 mL, 0.099 g, 0.981 mmol), 1-methylpiperazine (0.11 mL, 0.098 g, 0.981 mmol), HATU (0.373 g, 0.981 mmol) in 10 mL CH₂Cl₂ were considered affording 57a (0.130 g, 45%) as yellow solid: ¹H NMR (DMSO-d₆) δ 13.25 (s, 1H), 8.39 (s, 1H), 8.30 (s, 1H), 8.26 (s, 1H), 7.91 (d, J=9.0 Hz, 1H), 7.66 (s, 1H), 7.50 (d, J=9.0 Hz, 1H), 5.99 (d, J=9.0 Hz, 1H), 4.52 (s, 2H), 4.08-4.26 (m, 2H), 3.94-3.97 (m,1H), 3.76-3.82 (m, 1H), 3.51-3.61 (m, 2H), 3.20 (q, J=6.0 Hz, 2H), 2.52-2.55 (m, 1H), 2.31 (br s, 4H), 2.20 (s, 3H), 2.13 (s, 3H), 2.02-2.07 (m, 2H), 1.74-1.86 (m, 1H), 1.62 (br s, 2H), 1.40 (br s, 9H), 1.02 (t, J=6.0 Hz, 3H); M+H⁺643.

### N-{[4-methyl-5-(3-{5-[(4-methylpiperazin-1-yl)carbonyl]-1H-imidazol-2-yl}-1H-indazol-5-yl)pyridin-3-yl]methyl}ethanamine (57)

In the same manner as the deprotection of 52a, intermediate 57a (0.112 g, 0.174 mmol) was converted to the title compound 57 (0.053 g, 66%), as a white solid: ¹H NMR (DMSO-d₆) δ 13.52 (s, 1H), 8.45 (s, 1H), 8.35 (s 1H) 8.25 (s, 1H) 7.70 (d, J=9.0 Hz, 1H), 7.64 (s, 1H) 7.44 (d, J=9.0 Hz, 1H), 4.12-4.33 (m, 2H, 3.80 (s, 2H), 3.50-3.66 (m, 2H), 2.65 (q, J=6.0 Hz, 2H), 2.29 (s, 7H), 2.13 (s, 3H), 1.07 (t, J=6.0 Hz, 3H). HRMS [M+H]⁺ calcd. 459.2616; found 459.2631. Anal. (C₂₅H₃₀N₈O 1.1H₂O 0.4 EtOAc) C, H, N.

### EXAMPLE 58

### N-cyclopentyl-2-(5-{5-[(ethylamino)methyl]-4-methylpyridin-3-yl}-1H-indazol-3-yl)-1H-imidazole-5-carboxamide

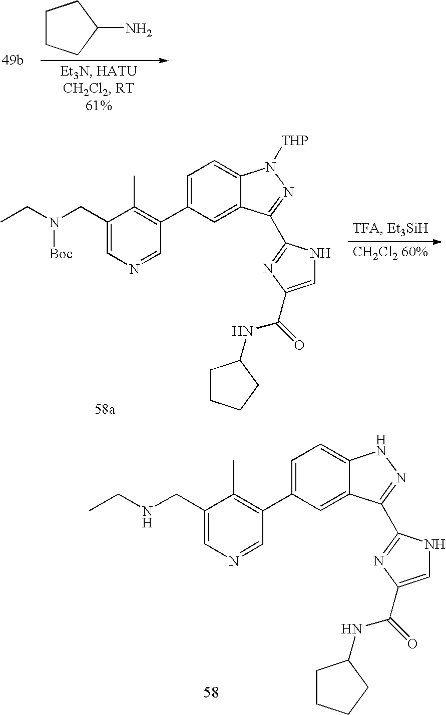

### tert-Butyl({5-[3-{5-[(cyclopentylamino)carbonyl]-1H-imidazol-2-yl}-1-(tetrahydro-2H-pyran-2-yl)-1H-indazol-5-yl]-4-methylpyridin-3-yl}methyl)ethylcarbamate (58a)

By the same procedure used to synthesize intermediate 49c, acid 49b (0.250 g, 0.446 mmol), Et₃N (0.14 mL, 0.099 g, 0.981 mmol), cyclopentylamine (0.097 mL, 0.084 g, 0.981 mmol), HATU (0.373 g, 0.981 mmol) in 10 mL CH₂Cl₂ were considered affording 58a (0.170 g, 61%) as a pale yellow solid: ¹H NMR (DMSO-d₆) δ13.10 (s, 1H), 8.43 (s, 1H), 8.40 (s, 1H), 8.29 (s, 1H), 7.90 (d, J=9.0 Hz, 1H), 7.72 (d, J=3.0 Hz, 1H), 7.66 (s, 1H), 7.46 (d, J=9.0 Hz, 1H), 5.99 (d, J=9.0 Hz, 1H), 4.51 (s, 2H), 4.12-4.22 (m, 1H), 3.93-3.97 (m, 1H), 3.75-3.84 (m, 1H), 3.20 (q, J=6.0 Hz, 2H), 2.50-2.59 (m,1H), 2.20(s, 3H), 2.02-2.08 (m, 2H), 1.80-1.86 (m, 3H), 1.63 (br s, 4H), 1.50 (br s, 4H), 1.41 (br s, 9H), 1.04 (t, J=6.0 Hz, 3H); M+H⁺628.

### N-cyclopentyl-2-(5-{5-[(ethylamino)methyl]-4-methylpyridin-3-yl}-1H-indazol-3-yl)-1H-imidazole-5-carboxamide (58)

In the same manner as the deprotection of 52a, intermediate 58a (0.148 g, 0.236 mmol) was converted to the title compound 58 (0.063 g, 60%), as a pale yellow solid: ¹H NMR (DMSO-d₆) δ13.52 (s, 1H), 8.44 (s, 1H), 8.40 (s, 1H), 8.35 (s, 1H), 7.70 (d, J=6.0 Hz, 1H), 7.63 (s, 1H), 7.40 (dd, J=3.0 Hz, J=6.0 Hz, 1H), 4.12-4.22 (m, 1H), 3.78 (s, 2H), 2.64 (q, J=6.0 Hz, 2H), 2.28 (s, 3H), 2.25-2.27 (m, 1H), 1.80-1.90 (m, 2H), 1.60-1.70 (m, 2H), 1.50-1.57 (m, 4H), 1.07 (t, J=6.0 Hz, 3H). HRMS [M+H]⁺ calcd. 444.2507; found 444.2502. Anal. (C₂₅H₂₉N₇O 1.5H₂O) C, H, N.

### EXAMPLE 59

### N-{[4-methyl-5-(3-{5-[(4-phenylpiperazin-1-yl)carbonyl]-1H-imidazol-2-yl}-1H-indazol-5-yl)pyridin-3-yl]methyl}ethanamine

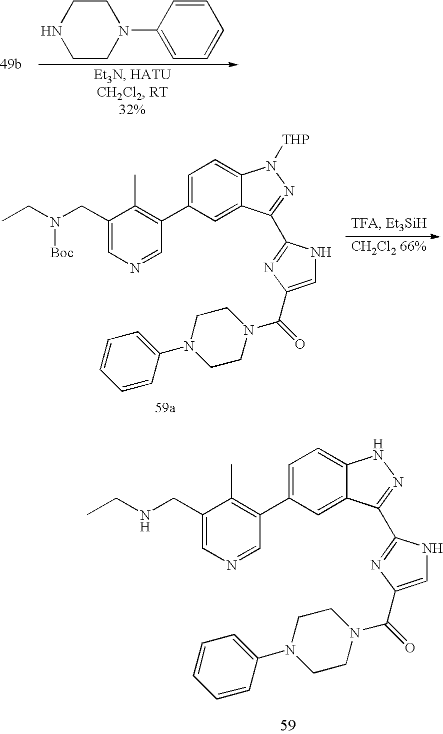

### tert-Butyl ethyl({4-methyl-5-[3-{5-[(4-phenylpiperazin-1-yl)carbonyl]-1H-imidazol-2-yl}-1-(tetrahydro-2H-pyran-2-yl)-1H-indazol-5-yl]pyridin-3-yl}methyl)carbamate (59a)

By the same procedure used to synthesize intermediate 49c, acid 49b (0.250 g, 0.446 mmol), Et₃N (0.14 mL, 0.099 g, 0.981 mmol), 1-phenylpiperazine (0.15 mL, 0.159 g, 0.981 mmol), HATU (0.373 g, 0.981 mmol) in 10 mL CH₂Cl₂ were considered affording 59a (0.100 g, 32%) as a pale yellow solid: ¹H NMR (DMSO-d₆) δ 13.29 (s,1H), 8.46 (s, 1H), 8.35 (s, 1H),8.32 (s, 1H), 7.92 (d, J=9.0 Hz, 1H), 7.71 (s, 1H), 7.53 (d, J=9.0 Hz, 1H), 7.18-7.23 (m,2H), 6.77-6.86 (m, 3H), 6.00 (d, J=9.0 Hz, 1H), 4.50 (s, 2H), 3.94-3.98 (m, 1H), 3.71-3.82(m, 2H), 3.30-3.32 (m, 4H), 3.13 (br s, 6H), 2.22 (s, 3H), 2.02-2.11 (m, 2H), 1.74-1.90 (m, 1H), 1.63 br s, 2H), 1.39 (br s, 9H), 0.96 (t, J=6.0 Hz, 3H); M+H⁺ 705.

### N-{[4-methyl-5-(3-{5-[(4-phenylpiperazin-1-yl)carbonyl]-1H-imidazol-2-yl}-1H-indazol-5-yl)pyridin-3-yl]methyl}ethanamine (59)

In the same manner as the deprotection of 52a, intermediate 59a (0.079 g, 0.112 mmol) was converted to the title compound 59 (0.038 g, 66%), as a pale yellow solid: ¹H NMR (DMSO-d₆) δ 13.53 (s, 1H), 8.49 (s, 1H), 8.41 (s, 1H), 8.30 (s, 1H), 7.70 (d, J=9.0 Hz, 2H), 7.45 (d, J=6.0 Hz, 1H), 7.22 (t, J=9.0 Hz, 2H), 6.78-6.86 (m, 3H), 3.79 (s, 2H), 3.30-3.32 (m,4H), 3.15 (br s, 4H), 2.60 (q, J=6.0 Hz, 2H), 2.30 (s, 3H), 1.03 (t, J=6.0 Hz, 3H). HRMS [M+H]⁺ calcd. 521.2772; found 521.2755. Anal. (C₃₀H₃₂N₈O 1 H₂O) C, H, N.

### EXAMPLE 60

### N-({5-[3-(5-{[(2R,6S)-2,6-dimethylmorpholin-4-yl]carbonyl}-1H-imidazol-2-yl)-1H-indazol-5-yl]-4-methylpyridin-3-yl}methyl)ethanamine

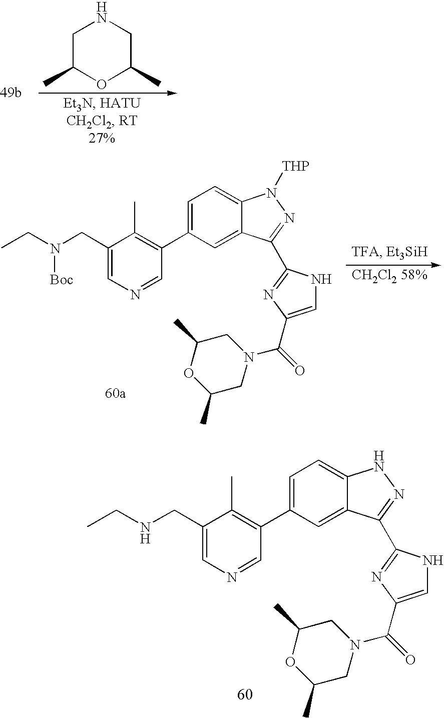

### tert-Butyl ({5-[3-(5-{[(2R,6S)-2,6-dimethylmorpholin-4-yl]carbonyl}-1H-imidazol-2-yl)-1-(tetrahydro-2H-pyran-2-yl)-1H-indazol-5-yl]-4-methylpyridin-3-yl}methyl)ethylcarbamate (60a)

By the same procedure used to synthesize intermediate 49c, acid 49b (0.250 g, 0.446 mmol), Et₃N (0.14 mL, 0.099 g, 0.981 mmol), cis-2,6-dimethylmorpholine (0.121 mL, 0.113 g, 0.981 mmol), HATU (0.373 g, 0.981 mmol) in 10 mL CH₂Cl₂ were considered affording 60a (0.080 g, 27%) as a pale yellow solid: ¹H NMR (DMSO-d₆) δ 13.29 (s,1H), 8.36 (s,1H), 8.30 (s, 1H), 8.20 (s, 1H), 7.92 (d, J=9.0 Hz, 1H), 7.70 (d, J=3.0 Hz, 1H), 7.48 (d, J=9.0 Hz, 1H), 6.00 (d, J=6.0 Hz, 1H), 5.42-5.57 (m, 1H), 4.51 (s, 2H), 4.23-4.42 (m, 1H), 3.93-4.00 (m, 1H), 3.76-3.84 (m,1H), 3.51 (br s, 2H), 3.15-3.25 (m, 2H), 2.17 (s, 3H), 2.03-2.10 (m, 2H), 1.73-1.84 (m, 1H), 1.58-1.67 (m, 3H), 1.40 (br s, 9H), 1.23 (br s, 3H), 1.11 (br s, 3H), 1.03 (t, J=6.0Hz, 3H), 0.73 (br s, 2H); M+H⁺ 658.

### N-({5-[3-(5-{[(2R,6S)-2.6-dimethylmorpholin-4-yl]carbonyl}-1H-imidazol-2-yl)-1H-inadazol-5-yl]- 4-methylpyridin-3-yl}methyl)ethanamine (60)

In the same manner as the deprotection of 52a, intermediate 60a (0.070 g, 0.106 mmol) was converted to the title compound 60 (0.029 g, 58%), as a pale yellow solid: ¹H NMR (DMSO-d₆) δ 13.54 (s, 1H), 8.44 (s, 1H), 8.31 (s, 1H), 8.19 (s, 1H), 7.68-7.71 (m, 2H), 7.42 (d, J=9.0 Hz, 1H), 5.48-5.69 (m, 1H), 4.22-4.42 (m, 1H), 3.78 (s, 2H), 3.48-3.55 (m, 2H), 2.61 (q, J=6.0 Hz, 2H), 2.25 (s, 3H), 1.07 (t, J=6.0 Hz, 9H), 0.72-0.84 (m, 2H). HRMS [M+H]⁺ calcd. 474.2612; found 474.2617. Anal. (C₂₆H₃₁N₇O₂ 0.75 H₂O 0.3 EtOAc) C, H, N.

### EXAMPLE 61

### N-[(4-methyl-5-{3-[5-(pyrrolidin-1-ylcarbonyl)-1H-imidazol-2-yl]-1H-indazol-5-yl}pyridin-3-yl)methyl]ethanamine

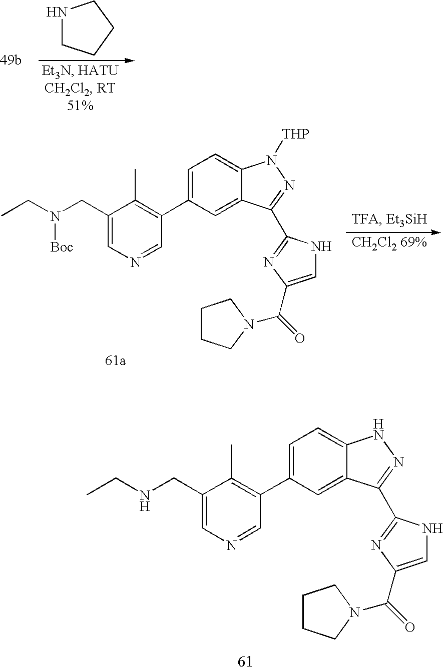

### tert-Butyl ethyl({4-methyl-5-[3-[5-(pyrrolidin-1-ylcarbonyl)-1H-imidazol-2-yl]-1-(tetrahydro-2H-pyran-2-yl)-1H-indazol-5-yl]pyridin-3-yl}methyl)carbamate (61a)

By the same procedure used to synthesize intermediate 49c, acid 49b (0.250 g, 0.446 mmol), Et₃N (0.14 mL, 0.099 g, 0.981 mmol), pyrrolidine (0.082 mL, 0.070 g, 0.981 mmol), HATU (0.373 g, 0.981 mmol) in 10 mL CH₂Cl₂ were considered affording 61a (0.140 g, 51%) as a pale yellow solid: ¹H NMR (DMSO-d₆) δ13.21 (s, 1H), 8.39 (s, 1H), 8.30 (br s, 2H), 7.92 (d, J=9.0 Hz, 1H), 7.67 (d, J=3.0 Hz, 1H), 7.50 (d, J=9.0 Hz, 1H), 6.00 (d, J=9.0 Hz, 1H), 4.51 (s, 2H), 4.03 (t, J=6.0 Hz, 2H), 3.93-3.98 (m, 1H), 3.78-3.82 (m, 1H), 3.35 (t, J=6.0 Hz, 2H), 3.20 (q, J=6.0 Hz, 2H), 2.22 (s, 3H), 2.03-2.07 (m, 2H), 1.76-1.85 (m, 5H), 1.62 (br s, 3H), 1.40 (br s, 9H), 1.02 (t, J=6.0 Hz, 3H); M+H⁺ 614.

### N-[(4-methyl-5-{3-[5-(pyrrolidin-1-ylcarbonyl)-1H-imidazol-2-yl]-1H-indazol-5yl}pyridin-3-yl)methyl]ethanamine (61)

In the same manner as the deprotection of 52a, intermediate 61a (0.123 g, 0.201 mmol) was converted to the title compound 61 (0.059 g, 69%), as a pale yellow crystalline solid: ¹H NMR (DMSO-d₆) δ 13.49 (s, 1H), 8.43 (s, 1H), 8.35 (s, 1H), 8.31 (s, 1H) 7.69 (d, J=9.0 Hz, 1H), 7.65 (s, 1H), 7.43 (d, J=9.0 Hz, 1H), 4.02-4.07 (m, 2H), 3.78 (s, 2H), 3.45-3.48 (m, 2H), 2.64 (q, J=6.0 Hz, 2H), 2.31 (s, 3H), 1.76-1.90 (m, 4H), 1.07 (t, J=6.0 Hz, 3H). HRMS [M+H]⁺ calcd. 430.2350; found 430.2343. Anal. (C₂₄H₂₇N₇O 0.15 H₂O 0.5 EtOAc) C, H, N.

### EXAMPLE 62

### N-[(4-methyl-5-{3-[5-(piperidin-1-ylcarbonyl)-1H-imidazol-2-yl]-1H-indadzol-5-yl}pyridin-3-yl)methyl]ethanamine

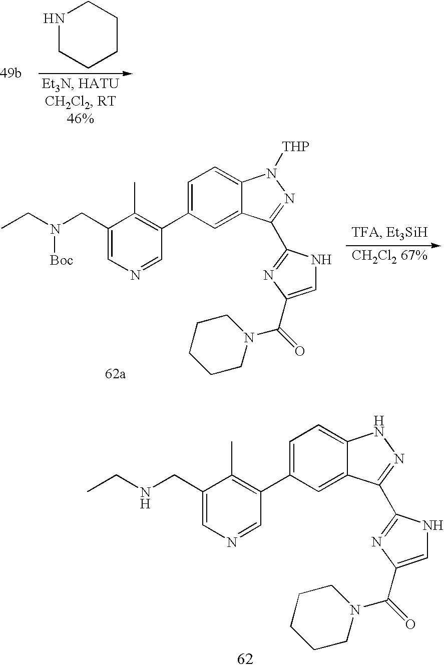

### tert-Butyl ethyl({4-methyl-5-[3-[5-(piperidin-1-ylcarbonyl)-1H-imidazol-2-yl]-1-(tetrahydro-2H-pyran-2-yl)-1H-indazol-5-yl]pyridin-3-yl}methyl)carbamate (62a)

By the same procedure used to synthesize intermediate 49c, acid 49b (0.250 g, 0.446 mmol), diisopropylethylamine (0.17 mL, 0.127 g, 0.981 mmol), piperidine (0.097 mL, 0.084 g, 0.981 mmol), HATU (0.373 g, 0.981 mmol) in 10 mL CH₂Cl₂ were considered affording 62a (0.129 g, 46%) as a yellow solid: ¹H NMR (DMSO-d₆) δ13.18 (s, 1H), 8.37 (s, 1H), 8.31 (s, 1H), 8.27 (s, 1H), 7.91 (d, J=6.0Hz, 1H), 7.62 (s, 1H), 7.49 (d, J=6.0 Hz, 1H), 5.99 (d, J=6.0 Hz, 1H), 4.51 (s, 2H), 4.14 (br s, 2H), 3.93-3.97 (m, 1H), 3.77-3.81 (m, 1H), 3.51 (m, 2H), 3.19 (m, 2H), 2.49-2.51 (m, 1H), 2.20 (s, 3H), 2.04-2.12 (m, 2H), 1.81-1.97 (m, 1H), 1.57-1.62 (m, 4H), 1.48 (s, 4H), 1.40(s, 9H), 1.02 (t, J=6.0 Hz, 3H); M+H⁺ 628.

### N-[(4-methyl-5-{3-[5-(piperidin-1-ylcarbonyl)-1H-imidazol-2-yl]-1H-indazol-5-yl}pyridin-3-yl)methyl]ethanamine (62)

In the same manner as the deprotection of 52a, intermediate 62a (0.120 g, 0.191 mmol) was converted to the title compound 62 (0.057 g, 67%), as a pale yellow solid: ¹H NMR (DMSO-d₆) δ 13.50 (s, 1H), 13.14 (br s, 1H), 8.42 (s, 1H), 8.33 (s, 1H), 8.26 (s, 1H), 7.69 (d, J=6.0Hz, 1H), 7.60 (s, 1H), 7.42 (d, J=6.0 Hz, 1H), 4.16 (br s, 2H), 3.76 (s, 2H), 3.55 (br s, 2H), 2.61 (q, J=6.0 Hz, 2H), 2.28 (s, 3H), 1.59 (br s, 2H), 1.50 (br s, 4H), 1.06 (t, J=6.0 Hz, 3H).

HRMS [M+H]^{+ calcd.} 444.2507; found 444.2500. Anal. (C₂₅H₂₉N₇O 1 H₂O) C, H, N.

### EXAMPLE 63

### N-ethyl-2-(5-{5-[(ethylamino)methyl]-4-methylpyridin-3-yl}-1H-indazol-3-yl)-1H-imidazole-5-carboxamide

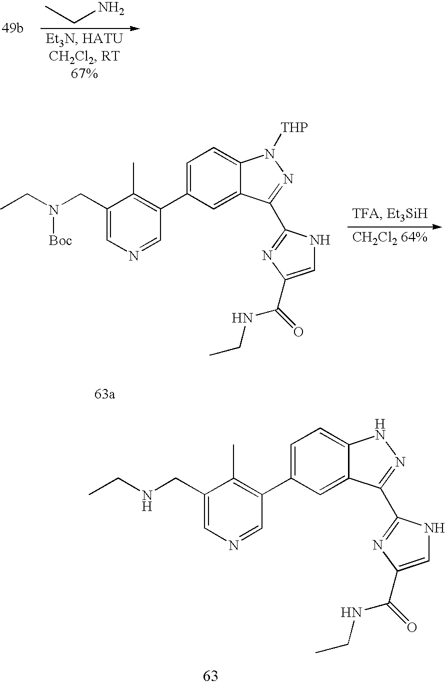

### tert-Butyl ethyl({5-[3-{5-[(ethylamino)carbonyl]-1H-imidazol-2-yl}1-(tetrahydro-2H-pyran-2-yl)-1H-indazol-5-yl]-4-methylpyridin-3-yl}methyl)carbamate (63a)

By the same procedure used to synthesize intermediate 49c, acid 49b (0.250 g, 0.446 mmol), diisopropylethylamine (0.17 mL, 0.127 g, 0.981 mmol), 2M ethylamine in THF (0.491 mL, 0.981 mmol), HATU (0.373 g, 0.981 mmol) in 10 mL CH₂Cl₂ were considered affording 63a (0.176 g, 67%) as a yellow solid: ¹H NMR (DMSO-d₆) δ13.07 (s, 1H), 8.46 (s, 1H), 8.39 (s, 1H), 8.29 (s, 1H), 8.07 (t, J=6.0 Hz, 1H), 7.89 (d, J=6.0 Hz, 1H), 7.63 (s, 1H), 7.44 (d, J=6.0 Hz, 1H), 5.99 (d, J=6.0 Hz, 1H), 4.51 (s, 2H), 3.93-3.96 (m, 1H), 3.78-3.82 (m, 1H), 3.21-3.25 (m, 4H), 2.53-2.57 (m, 1H), 2,18 (s, 3H), 2.03-2.12 (m, 2H), 1.78-1.84 (m, 1H), 1.62 (m, 2H), 1.41 (s, 9H), 1.07 (t, J=6.0 Hz, 6H); M+H⁺588.

### N-ethyl-2-(5-{5-[(ethylamino)methyl]-4-methylpyridin-3-yl}-1H-indazol-3-yl)-1H-imidazole-5-carboxamide (63)

In the same manner as the deprotection of 52a, intermediate 63a (0.156 g, 0.266 mmol) was converted to the title compound 63 (0.068 g, 64%), as a pale yellow solid: ¹H NMR (DMSO-d₆) δ 13.51 (s, 1H), 8.44 (d, J=3.0 Hz, 2H), 8.34 (s, 1H), 8.06 (t, J=6.0 Hz, 1H), 7.67 (d, J=6.0 Hz, 1H), 7.60 (s, 1H), 7.37 (d, J=6.0 Hz, 1H), 3.77 (s, 2H), 3.24 (q, J=6.0 Hz, 2H), 2.63 (q, J=6.0 Hz, 2H), 2.25 (s, 3H), 1.07 (t, J=6.0 Hz, 6H). HRMS [M+H]⁺ calcd. 404.2194; found 404.2194. Anal. (C₂₂H₂₅N₇O 1.25 H₂O 0.1 EtOAc) C, H, N.

### EXAMPLE 64

### 2-(5-{5-[(ethylamino)methyl]-4-methylpyridin-3-yl}-1H-indazol-3-yl)-N-isopropyl-1H-imidazole-5-carboxamide

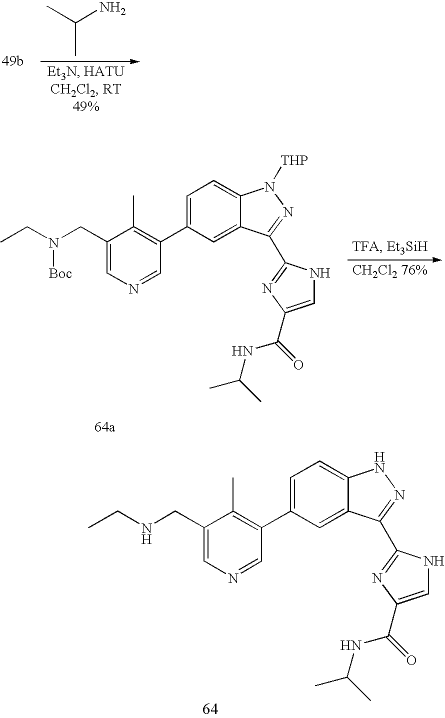

### tert-Butyl ethyl({5-[3-{5-[(isopropylamino)carbonyl]-1H-imidazol-2-yl}-1-(tetrahydro-2H-pyran-2-yl)-1H-indazol-5-yl]-4-methylpyridin-3-yl}methyl)carbamate (64a)

By the same procedure used to synthesize intermediate 49c, acid 49b (0.250 g, 0.446 mmol), diisopropylethylamine (0.17 mL, 0.127 g, 0.981 mmol), isopropylamine (0.084 mL, 0.058 g, 0.981 mmol), HATU (0.373 g, 0.981 mmol) in 10 mL CH₂Cl₂ were considered affording 64a (0.130 g, 49%) as a yellow solid: ¹H NMR (DMSO-d₆) δ13.08 (s, 1H), 8.42 (s, 1H), 8.39 (s, 1H), 8.29 (s, 1H), 7.90 (d, J=6.0 Hz, 1H), 7.61-7.65 (m, 2H), 7.47 (d, J=6.0 Hz, 1H), 6.00 (d, J=6.0 Hz, 1H), 4.51 (s, 2H), 4.03-4.10 (m, 1H), 3.93-3.96 (m, 1H), 3.75-3.83 (m, 1H), 3.20 (q, J=6.0 Hz, 2H), 2.52-2.59 (m, 1H), 2.20 (s, 3H), 2.03-2.12 (m, 2H), 1.80 (br s, 1H), 1.62 (s, 2H), 1.41 (s, 9H), 1.13-1.18 (m, 6H), 1.04 (t, J=6.0 H, 3H); M+H⁺ 602.

### 2-(5-{5-[(ethylamino)methyl]-4-methylpyridin-3-yl}-1H-indazol-3-yl)-N-isopropyl-1H-imidazole-5-carboxamide (64)

In the same manner as the deprotection of 52a, intermediate 64a (0.110 g, 0.183 mmol) was converted to the title compound 64 (0.058 g, 76%), as a pale yellow solid: ¹H NMR (DMSO-d₆) δ 13.52 (s, 1H), 8.43 (s, 1H), 8.41 (s 1H), 8.34 (s, 1H), 7.68 (d, J=6.0 Hz, 1H), 7.61-7.63 (m, 2H), 7.40 (dd, J=3.0 Hz, J=6.0 Hz, 1H), 4.01-4.08 (m, 1H), 3.77 (s, 2H), 2.63 (q, J=6.0 Hz, 2H), 2.27 (s, 3H), 1.12-1.18 (m, 6H), 1.07 (t, J=6.0 Hz 3H). HRMS [M+H]⁺ calcd. 418.2350; found 418.2358. Anal. (C₂₃H₂₇N₇O 1.25 H₂O 0.1 EtOAc) C, H, N.

### EXAMPLE 65

### 2-(5-{5-[(ethylamino)methyl]-4-methylpyridin-3-yl}-1H-indazol-3-yl)-N-(2-methoxyethyl)-1H-imidazole-5-carboxamide

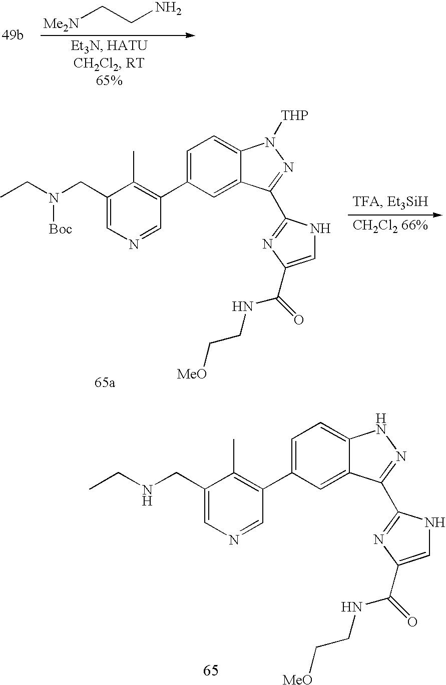

### tert-Butyl ethyl({5-[3-(5-{[(2-methoxyethyl)amino]carbonyl)}-1H-imidazol-2-yl)-1-(tetrahydro-2H-pyran-2-yl)-1H-indazol-5-yl]-4-methylpyridin-3-yl}methyl)carbamate (65a)

By the same procedure used to synthesize intermediate 49c, acid 49b (0.250 g, 0.446 mmol), diisopropylethylamine (0.17 mL, 0.127 g, 0.981 mmol), 2-methoxyethylamine (0.086 mL, 0.074 g, 0.981 mmol), HATU (0.373 g, 0.981 mmol) in 10 mL CH₂Cl₂ were considered affording 65a (0.180 g, 65%) as a yellow solid: ¹H NMR (DMSO-d₆) δ13.13 (s, 1H), 8.42 (s, 1H), 8.39 (s, 1H), 8.28 (s, 1H), 7.97 (t, J=6.0 Hz, 1H), 7.90 (d, J=6.0 Hz, 1H), 7.66 (s, 1H), 7.45 (d, J=6.0 Hz, 1H), 6.00 (d, J=6.0 Hz, 1H), 4.51 (s, 2H), 3.93-3.96 (m, 1H), 3.77-3.82 (m,1H), 3.38-3.41 (m, 4H), 3.20-3.22 (m, 5H), 2.51-2.62 (m, 1H), 2.19 (s, 3H), 2.04-2.08 (m, 2H), 1.78-1.84 (m, 1H), 1.62 (s, 2H), 1.41 (s, 9H), 1.05 (t, J=6.0 Hz, 3H); M+H⁺ 618.

### 2-(5-{5-[(ethylamino)methyl]-4-methylpyridin-3-yl}-1H-indazol-3-yl)-N-(2-methoxyethyl)-1H-imidazole-5-carboxamide (65)

In the same manner as the deprotection of 52a, intermediate 65a (0.170 g, 0.276 mmol) was converted to the title compound 65 (0.079 g, 66%), as a pale yellow solid: ¹H NMR (DMSO-d₆) δ 13.52 (s, 1H), 8.44 (s, 1H), 8.40 (s, 1H), 8.34 (s, 1H), 7.96 (t, J=6.0 Hz, 1H), 7.68 (d, J=6.0 Hz, 1H), 7.63 (s, 1H), 7.38 (dd, J=3.0 Hz, J=6.0 Hz, 1H), 3.77 (s, 2H), 3.38-3.42 (m, 4H), 3.21 (s, 3H), 2.62 (q, J=6.0 Hz, 2H), 2.26 (s, 3H), 1.07 (t, J=6.0 Hz, 3H). (HRMS [M+H]⁺ calcd. 434.2299; found 434.2298. Anal. (C₂₃H₂₇N₇O₂ 1H₂O) C, H, N.

### EXAMPLE 66

### N-{[5-(3-{5-[(4-benzylpiperazin-1-yl)carbonyl]-1H-imidazol-2-yl}-1H-indazol-5-yl)-4-methylpyridin-3-yl]methyl}ethanamine

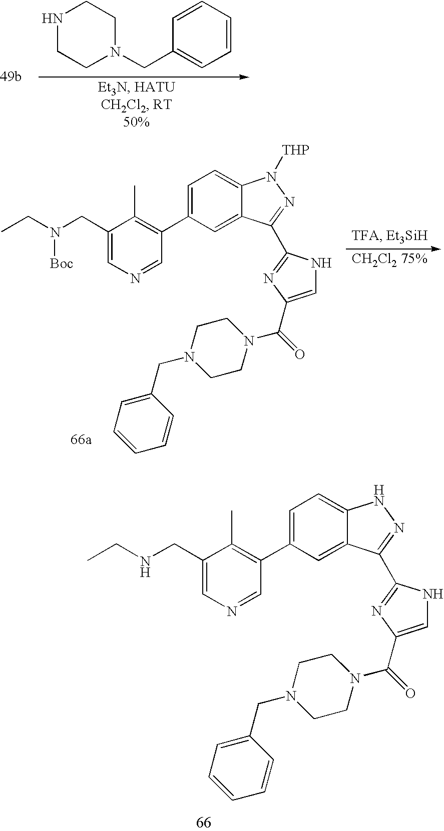

### tert-Butyl ({5-[3-{5-[(4-benzylpiperazin-1-yl)carbonyl]-1H-imidazol-2-yl}-1-(tetrahydro-2H-pyran-2-yl)-1H-indazol-5-yl]-4-methylpyridin-3-yl}methyl)ethylcarbamate (66a)

By the same procedure used to synthesize intermediate 49c, acid 49b (0.250 g, 0.446 mmol), diisopropylethylamine (0.17 mL, 0.127 g, 0.981 mmol), 1-benzylpiperazine (0.171 mL, 0.173 g, 0.981 mmol), HATU (0.373 g, 0.981 mmol) in 10 mL CH₂Cl₂ were considered affording 66a (0.160 g, 50%) as a pale yellow solid: ¹H NMR (DMSO-d₆) δ13.23 (s, 1H), 8.40 (s, 1H), 8.30 (s, 1H), 8.25 (s, 1H), 7.91 (d, J=6.0 Hz, 1H), 7.65 (s, 1H), 7.50 (d, J=6.0 Hz, 1H), 7.21-7.32 (m, 5H), 5.99 (d, J=6.0 Hz, 1H), 4.50 (s, 2H), 4.14-4.30 (m, 2H), 3.94-3.96 (m, 1H), 3.79-3.83 (m, 1H), 3.50-3.62 (m, 2H), 3.42 (s, 2H), 3.20 (br s, 2H), 2.51-2.53 (m, 1H), 3.79-2.20 (s, 3H), 2.02-2.12 (m, 2H), 1.79-1.83 (m, 1H), 1.62(s, 2H), 1.39 (br s, 9H), 1.00 (t, J=6.0 Hz, 3H); M+H⁺ 719.

### N-{[5-(3-{5-[(4-benzylpiperazin-1-yl)carbonyl]-1H-imidazol-2-yl}-1H-indazol-5-yl)-4-methylpyridin-3-yl]methyl}ethanamine (66)

In the same manner as the deprotection of 52a, intermediate 66a (0.140 g, 0.195 mmol) was converted to the title compound 66 (0.078 g, 75%), as a pale yellow solid: ¹H NMR (DMSO-d₆) δ 13.51 (br s, 1H), 13.19 (br s, 1H), 8.44 (s, 1H), 8.35 (s, 1H), 8.24 (s, 1H), 7.69 (d, J=6.0 Hz, 1H), 7.63 (s, 1H), 7.43 (d, J=6.0 Hz, 1H), 7.22-7.30 (m, 5H), 4.25 (br s, 2H), 3.75 (s, 2H), 3.56 (br s, 2H), 3.43 (s, 2H), 2.60 (q, J=6.0 Hz, 2H), 2.38 (br s, 4H), 2.28 (s, 3H), 1.05 (t, J=6.0 Hz, 3H). HRMS [M+H]+ calcd. 535.2929; found 535.2908. Anal. (C₃₁H₃₄N₈O 1 H₂O) C, H, N.

### EXAMPLE 67

### N-{[4-methyl-5-(3-{5-[(4-phenyl-3,6-dihydropyridin-1(2H)-yl)carbonyl]-1H-imidazol-2-yl}-1H-indazol-5-yl)pyridin-3-yl]methyl}ethanamine

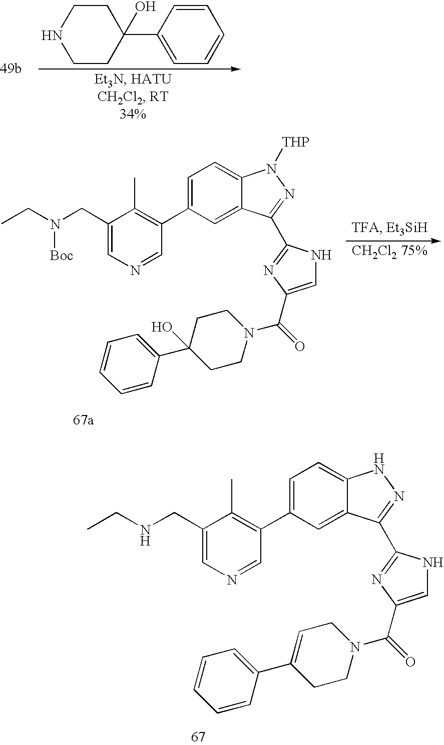

### tert-Butyl ethyl({5-[3-{5-[(4-hydroxy-4-phenylpiperidin-1-yl)carbonyl]-1H-imidazol-2-yl}-1-(tetrahydro-2H-pyran-2-yl)-1H-indazol-5-yl]-4-methylpyridin-3-yl}methyl)carbamate (67a)

By the same procedure used to synthesize intermediate 49c, acid 49b (0.250 g, 0.446 mmol), diisopropylethylamine (0.17 mL, 0.127 g, 0.981 mmol), 4-hydroxy-4-phenyl-piperidine (0.174 g, 0.981 mmol), HATU (0.373 g, 0.981 mmol) in 10 mL CH₂Cl₂ were considered affording 67a (0.110 g, 34%) as a pale yellow solid: ¹H NMR (DMSO-d₆) δ13.20 (s, 1H), 8.34 (s, 1H), 8.24 (d, J=3.0 Hz, 2H), 7.91 (d, J=6.0 Hz, 1H), 7.67 (s, 1H), 7.46 (d, J=6.0 Hz, 1H), 7.39 (d, J=3.0 Hz, 2H), 7.27 (t, J=6.0 Hz, 2H), 7.19 (t, J=6.0 Hz, 1H), 5.99 (d, J=6.0 Hz, 1H), 5.21-5.31 (m, 1H), 5.10 (s, 1H), 4.40 (s, 3H), 3.94-3.97 (m, 1H), 3.77-3.86 (m, 1H), 3.42-3.57 (m, 1H), 3.11-3.20 (m, 3H), 2.53-2.58 (m, 1H), 2.14 (s, 3H), 2.02-2.09 (m, 1H), 1.75-1.89 (m, 3H), 1.62 (br s, 5H), 1.40 (br s, 9H), 0.98 (t, J=6.0 Hz, 3H); M+H⁺ 720.

### N-{[4-methyl-5-(3-{5-[(4-phenyl-3,6-dihydropyridin-1(2H)-yl)carbonyl]-1H-imidazol-2-yl}-1H-indazol-5-yl)pyridin-3-yl]methyl}ethanamine (67)

In the same manner as the deprotection of 52a, intermediate 67a (0.090 g, 0.125 mmol) was converted to the title compound 67 (0.057 g, 85%), as a pale yellow solid: ¹H NMR (DMSO-d₆) δ 13.53 (s, 1H), 13.25 (br s, 1H), 8.48 (s, 1H), 8.41 (s, 1H), 8.36 (s, 1H), 7.22-7.68 (m, 2H), 7.45 (d, J=6.0 Hz, 1H), 7.35-7.26 (m, 5H), 4.39 (s, 1H), 4.21 (s, 1H), 3.74 (s, 2H), 2.45-2.54 (m, 7H), 2.29 (s, 3H), 1.00 (t, J=6.0 Hz, 3H).

HRMS [M+H]+ calcd. 518.2663; found 518.2660. Anal. (C₃₁H₃₁N₇O 1.0 H₂O) C, H, N.

### EXAMPLE 68

### N-[(5-{3-[5-(1,3-dihydro-2H-isoindol-2-ylcarbonyl)-1H-imidazol-2-yl]-1H-inadazol-5-yl}4-methylpyridin-3-yl)methyl]ethanamine

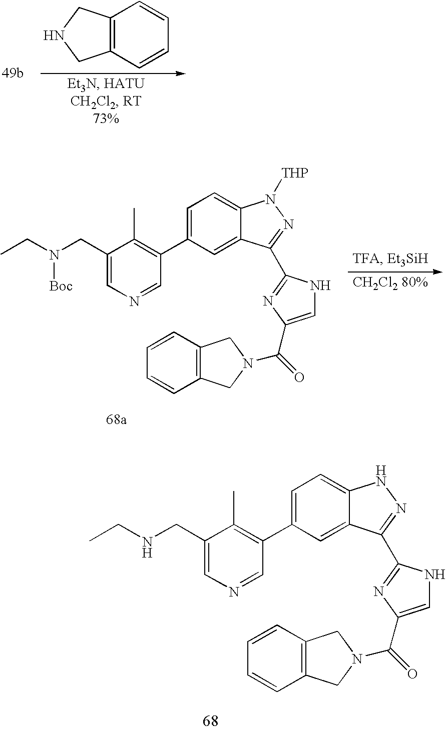

### tert-Butyl ({5-[3-[5-(1,3-dihydro-2H-isoindol-2-ylcarbonyl)-1H-imidazol-2-yl]-1-(tetrahydro-2H-pyran-2-yl)-1H-indazol-5-yl]-4-methylpyridin-3-yl}methyl)ethylcarbamate (68a)

By the same procedure used to synthesize intermediate 49c, acid 49b (0.250 g, 0.446 mmol), diisopropylethylamine (0.17 mL, 0.127 g, 0.981 mmol), isoindoline 0.11 mL, 0.117 g, 0.981 mmol), HATU (0.373 g, 0.981 mmol) in 10 mL CH₂Cl₂ were considered affording 68a (0.216 g, 73%) as a pale yellow solid: ¹H NMR (DMSO-d₆) δ13.36 (s, 1H), 8.49 (s, 1H), 8.46 (s, 1H), 8.34 (s, 1H), 7.94 (d, J=6.0 Hz, 1H), 7.81 (s, 1H), 7.55 (d, J=6.0 Hz, 1H), 7.39 (d, J=6.0 Hz, 1H), 7.24-7.30 (m, 2H),7.02 (d, J=3.0 Hz, 1H), 6.01 (d, J=6.0 Hz, 1H), 5.48 (s, 2H), 4.85 (s, 2H), 4.52 (s, 2H), 3.95-3.99 (m, 1H), 3.78-3.84 (m, 1H), 3.19 (br s, 2H), (br s, 2H), 2.52-2.56 (m, 1H), 2.25 (s, 3H), 2.05-2.13 (m, 2H), 1.78-1.88 (m, 1H), 1.63 (br s, 2H), 1.40 (br s, 9H), 1.01 (t, J=6.0 Hz, 3H); M+H⁺ 662.

### N-[(5-{3-[5-(1,3-dihydro-2H-isoindol-2-ylcarbonyl)-1H-imidazol-2-yl]-1H-indazol-5-yl}-4-methylpyridin-3-yl)methyl]ethanamine (68)

In the same manner as the deprotection of 52a, intermediate 68a (0.186 g, 0.281 mmol) was converted to the title compound 68 (0.107 g, 80%), as a pale buff solid: ¹H NMR (DMSO-d₆) δ 13.53 (s, 1H), 8.48 (s, 1H), 8.44 (s, 2H), 7.79 (s, 1H), 7.72 (d, J=6.0 Hz, 1H), 7.47 (d, J=6.0 Hz, 1H), 7.39 (d, J=6.0 Hz, 1H), 7.24-7.32 (m, 2H), 7.07 (d, J=6.0 Hz, 1H), 5.50 (s, 2H), 4.85 (s, 2H), 3.77 (s, 2H), 2.60 (q, J=6.0 Hz, 2H), 2.34 (s, 3H), 1.03 (t, J=6.0 Hz, 3H).

HRMS [M+H]+ calcd. 478.2350; found 478.2344. Anal. (C₂₈H₂₇N₇O 1.25 H₂O) C, H, N.

### EXAMPLE 69

### N-({5-[3-(5-{[4-(2,4-difluorophenyl)piperazin-1-yl]carbonyl}-1H-imidazol-2-yl)-1H-indazol-5-yl]-4-methylpyridin-3-yl}methyl)ethanamine

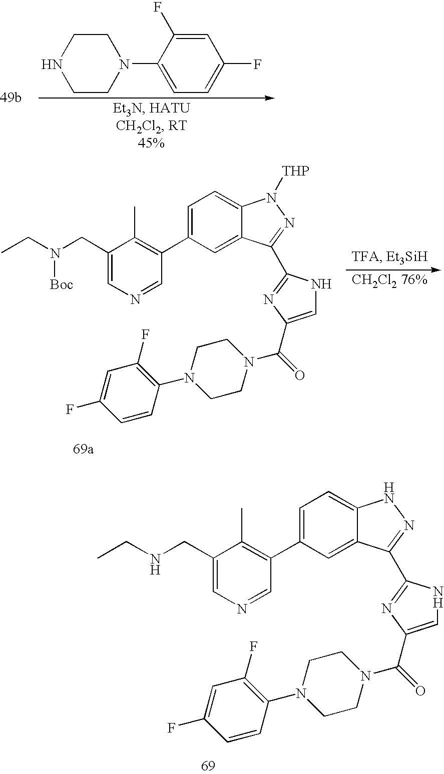

### tert-Butyl ({5-[3-(5-{[4-(2,4-difluorophenyl)piperazin-1-yl]carbonyl}-1H-imidazol-2-yl)-1-(tetrahydro-2H-pyran-2-yl)-1H-indazol-5-yl]-4-methylpyridin-3-yl}methyl)ethylcarbamate (69a)

By the same procedure used to synthesize intermediate 49c, acid 49b (0.250 g, 0.446 mmol), diisopropylethylamine (0.17 mL, 0.127 g, 0.981 mmol), 3,4-difluoro-1-phenylpiperazine (0.194 g, 0.981 mmol), HATU (0.373 g, 0.981 mmol) in 10 mL CH₂Cl₂ were considered affording 69a (0.216 g, 73%) as a pale yellow solid: ¹H NMR (DMSO-d₆) δ 13.29 (s, 1H), 8.42 (s, 1H), 8.29 (s, 2H), 7.92 (d, J=6.0 Hz, 1H), 7.70 (s,1H), 7.51 (d, J=6.0 Hz, 1H), 7.20 (t, J=6.0 Hz, 1H), 6.96 (d, J=6.0 Hz, 2H), 6.00 (d, J=6.0 Hz,1H), 4.48 (s, 2H), 4.33-4.46 (m, 2H), 3.94-4.00 (m, 1H), 3.76-3.83 (m, 3H), 3.14 (br s, 2H), 2.96 (br s, 4H), 2.52-2.58 (m, 1H), 2.20 (m, 3H), 2.04-2.13 (m, 2H), 1.74-1.87 (m, 1H), 1.63 (br s, 2H), 1.39 (s, 9H), 0.96 (t, J=6.0 Hz, 3H); M+H⁺ 741.

### N-({5-[3-(5-{[4-(2,4-difluorophenyl)piperazin-1-yl]carbonyl}-1H-imidazol-2-yl)-1H-indazol-5-yl]-4-methylpyridin-3-yl}methyl)ethanamine (69)

In the same manner as the deprotection of 52a, intermediate 69a (0.140 g, 0.189 mmol) was converted to the title compound 69 (0.080 g, 76%), as a pale yellow solid: ¹H NMR (DMSO-d₆) δ 13.52 (br s, 1H), 13.22 (br s, 1H), 8.43 (s, 1H), 8.36 (s, 1H), 8.28 (s, 1H), 7.69-7.71 (m, 2H), 7.44 (d, J=6.0 Hz, 1H), 7.00-7.22 (m,1H), 6.91-7.00 (m, 2H), 4.38-4.50 (m, 1H), 3.74-3.79 (m, 1H), 3.71 (s, 2H), 2.97 (br s, 4H), 2.49-2.58 (m, 4H), 2.27 (s, 3H), 1.01 (t, J=6.0 Hz, 3H). HRMS [M+H]^{+ calcd.} 557.2584; found 557.2560. Anal. (C₃₀H₃₀N₈OF₂ 0.5 H₂O 0.1 EtOAc) C, H, N.

### EXAMPLE 70

### N-{[4-methyl-5-(3-{5-[(4-phenoxypiperidin-1-yl)carbonyl]-1H-imidazol-2-yl}-1H-indazol-5-yl)pyridin-3-yl]methyl}ethanamine

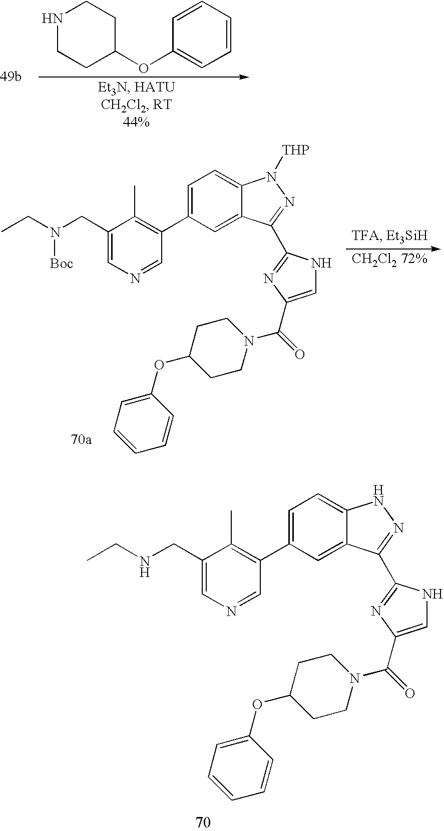

### tert-Butyl ethyl({4-methyl-5-[3-{5-[(4-phenoxypiperdin-1-yl)carbonyl]-1H-imidazol-2yl}-1-(tetrahydro-2H-pyran-2-yl)-1H-indazol-5-yl]pyridin-3-yl}methyl)carbamate (70a)

By the same procedure used to synthesize intermediate 49c, acid 49b (0.250 g, 0.446 mmol), diisopropylethylamine (0.34 mL, 0.254 g, 1.962 mmol), 4-phenoxy-piperidine (0.209 g, 0.981 mmol), HATU (0.373 g, 0.981 mmol) in 10 mL CH₂Cl₂ were considered affording 70a (0.140 g, 44%) as a pale yellow solid: 1H NMR (DMSO-d₆) δ 13.24 (s,1H), 8.38 (s,1H), 8.26 (s, 2H), 7.92 (d, J=9.0 Hz, 1H), 7.67 (s, 1H), 7.49 (d, J=6.0 Hz, 1H), 7.27 (t, J=6.0 Hz, 2H), 6.90-6.94 (m, 3H), 6.00 (d, J=6.0 Hz,1H), 4.60-4.70 (m, 2H), 4.47 (s, 2H), 3.94-3.97 (m, 3H), 3.77-3.81 (m, 1H), 3.28-3.36 (m, 1H), 3.16 (br s, 2H), 2.53-2.57 (m, 1H), 2.19 (s, 3H), 2.03-2.11 (m, 2H), 1.94 (br s, 2H), 1.76-1.84 (m, 1H), 1.62 (br s, 4H), 1.39 (br s, 9H), 0.96 (t, J=6.0 Hz, 3H); M+H⁺ 720.

### N-{[4-methyl-5-(3-{5-[(4-phenoxypiperidin-1-yl)carbonyl]-1H-imidazol-2-yl}-1H-indazol-5-yl)pyridin-3-yl]methyl}ethanamine (70)

In the same manner as the deprotection of 52a, intermediate 70a (0.130 g, 0.181 mmol) was converted to the title compound 70 (0.070 g, 72%), as a pale yellow solid: ¹H NMR (DMSO-d₆) δ 13.51 (s, 1H), 13.16 (s, 1H), 8.39 (s, 1H), 8.32 (s, 1H), 8.25 (s, 1H), 7.65-7.79 (m, 2H), 7.42 (d, J=6.0 Hz, 1H), 7.28 (t. J=6.0 Hz, 2H), 6.91-6.97 (m, 3H), 4.66-4.73 (m, 1H), 4.60-4.63 (m,1H), 3.90-4.06 (m, 2H), 3.70 (s, 2H), 3.40-3.46 (m, 1H), 2.57 (q, J=6.0 Hz, 2H), 2.26 (s, 3H), 1.92-1.98 (m, 2H), 1.61 (br s, 2H), 1.02 (t J=6.0 Hz, 3H). HRMS [M+H]⁺ calcd. 536.2769; found 536.2774. Anal. (C₃₁H₃₃N₇O₂ 0.75 H₂O) C, H, N.

### EXAMPLE 71

### N-[(4-methyl-5-{3-[5-(octahydroisoguinolin-2(1H)-ylcarbonyl)-1H-imidazol-2-yl]-1H-indazol-5-yl}pyridin-3-yl)methyl]ethanamine

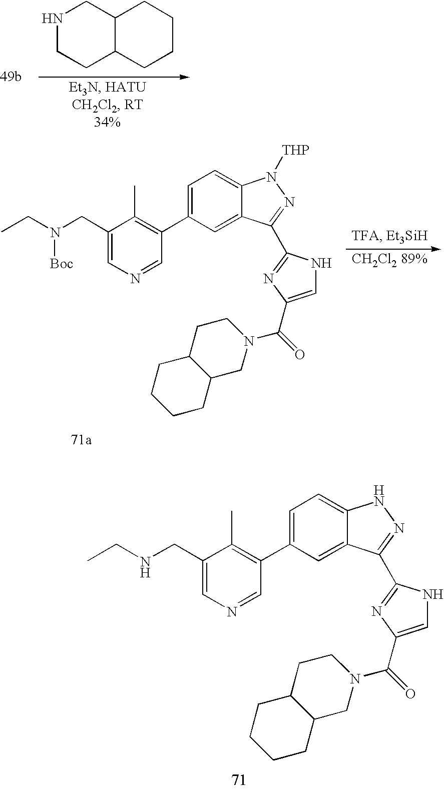

### tert-Butyl ethyl({4-methyl-5-[3-[5-(octahydroisoquinolin-2(1H)-ylcarbonyl)-1H-imidazol-2-yl]-1-(tetrahydro-2H-pyran-2-yl)-1H-indazol-5-yl]pyridin-3-yl}methyl)carbamate (71a)

By the same procedure used to synthesize intermediate 49c, acid 49b (0.250 g, 0.446 mmol), diisopropylethylamine (0.34 mL, 0.254 g, 1.962 mmol), perhydroisoquinoline (0.15 mL, 0.136 g, 0.981 mmol), HATU (0.373 g, 0.981 mmol) in 10 mL CH₂Cl₂ were considered affording 71a (0.104 g, 34%) as a yellow solid: ¹H NMR (DMSO-d₆) δ 13.19 (s, 1H), 8.35 (s,1H), 8.29 (d, J=3.0 Hz, 2H), 7.91 (d, J=6.0 Hz, 1H), 7.62 (d, J=3.0 Hz, 1H), 7.48 (d, J=6.0 Hz, 1H), 5.99 (d, J=6.0 Hz, 1H), 4.51 (s, 2H), 3.94-3.97 (m, 1H), 3.77-3.83 (m, 1H), 3.20 (q, J=6.0 Hz, 2H), 2.44-2.56 (m, 5H), 2.17 (s, 3H), 2.03-2.07 (m, 2H), 1.73-1.85 (m, 2H), 1.62 (s, 3H), 1.52-1.55 (m, 2H), 1.40 (br s, 11H), 1.22 (br s, 3H), 1.03 (br s, 6H); M+H⁺ 682.

### N-[(4-methyl-5-{3-[5-(octahydroisoquinolin-2(1H)-ylcarbonyl)-1H-imidazol-2-yl]-1H-indazol-5-yl}pyridin-3-yl)methyl]ethanamine (71)

In the same manner as the deprotection of 52a, intermediate 71a (0.090 g, 0.132 mmol) was converted to the title compound 71 (0.059 g, 89%), as a pale yellow solid: ¹H NMR (DMSO-d₆) δ 13.50 (s, 1H), 13.14 (br s, 1H), 8.43 (s,1H), 8.22-8.31 (m, 2H), 7.69 (d, J=9.0 Hz, 1H), 7.60 (d, J=6.0 Hz, 1H), 7.41 (d, J=6.0 Hz, 1H), 3.76 (s, 2H), 2.61 (q, J=6.0 Hz, 2H), 2.49 (br s, 4H), 2.25 (s, 4H), 1.82 (br s, 1H), 1.66 (br s, 1H), 1.51-1.54 (m, 2H), 1.40 (br s, 2H), 1.22 (br s, 3H), 1.06 (t, J=6.0 Hz, 5 H). HRMS [M+H]⁺ calcd. 498.2976; found 498.2980.

Anal. (C₂₉H₃₅N₇O 0.9 H₂O 0.2 MeOH) C, H, N.

### EXAMPLE 72

### N-2-adamantyl-2-(5-{5-[(ethylamino)methyl]-4-methylpyridin-3-yl}-1H-indazol-3-yl)-1H-imidazole-5-carboxamide

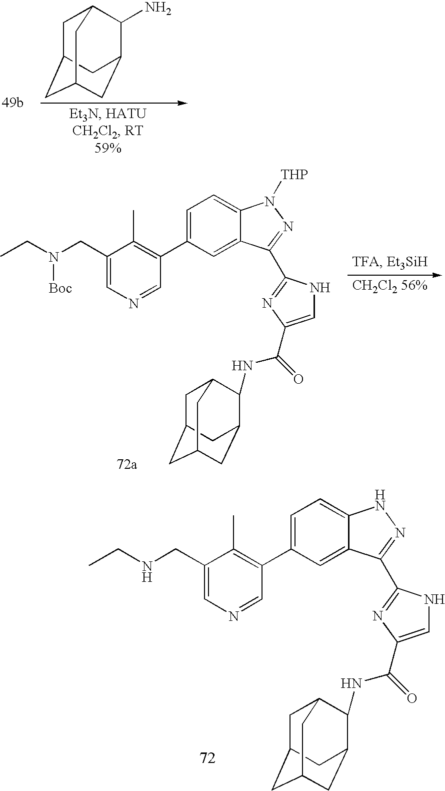

### tert-Butyl ({5-[3-{5-[(2-adamantylamino)carbonyl]-1H-imidazol-2-yl}-1-(tetrahydro-2H-pyran-2-yl)-1H-indazol-5-yl]-4-methylpyridin-3-yl}methyl)ethylcarbamate (72a)

By the same procedure used to synthesize intermediate 49c, acid 49b (0.250 g, 0.446 mmol), diisopropylethylamine (0.34 mL, 0.254 g, 1.962 mmol), adamantine hydrochloride (0.184 g, 0.981 mmol), HATU (0.373 g, 0.981 mmol) in 10 mL CH₂Cl₂ were considered affording 72a (0.183 g, 59%) as a yellow solid: ¹H NMR (DMSO-d₆) δ 13.28 (s, 1H), 8.38 (d, J=6.0 Hz, 1H), 8.28 (s, 1H), 7.93 (d, J=6.0 Hz, 1H), 7.72 (d, J=3.0 Hz, 1H), 7.70 (s, 1H), 7.51 (d, J=6.0 Hz, 1H), 7.40 (d, J=3.0 Hz,1H), 6.00 (d, J=6.0 Hz,1H), 4.50 (s, 2H), 3.94-4.01 (m, 1H), 3.77-3.83 (m, 1H), 3.19 (br s, 2H), 2.53-2.57 (m, 1H), 2.21 (s, 3H), 2.03-2.12 (m, 3H), 1.90-1.98 (m, 2H), 1.62-1.86 (m, 13H), 1.53-1.56 (m, 2H), 1.40 (br s, 9H), 1.03 (t, J=6.0 Hz, 3H); M+H⁺ 694.

### N-2-adamantyl-2-(5-{5-[(ethylamino)methyl]-4-methylpyridin-3-yl}-1H-indazol-3-yl)-1H-imidazole-5-carboxamide (72).

In the same manner as the deprotection of 52a, intermediate 72a (0.159 g, 0.229 mmol) was converted to the title compound 72 (0.066 g, 56%), as a whitesolid: ¹H NMR (DMSO-d₆) δ 13.55 (s, 1H), 8.43 (s, 1H), 8.34 (d, J=3.0 Hz, 2H), 7.68-7.76 (m, 3H), 7.45 (dd, J=3.0 Hz, J=6.0 Hz, 1H), 3.75 (s, 2H), 2.60 (q, J=6.0 Hz, 2H), 2.30 (s, 3H), 1.70-1.87 (m, 13H), 1.55-1.58 (m, 2H), 1.06 (t, J=6.0 Hz, 3H). HRMS [M+H]⁺ calcd. 510.2976; found 510.2984. Anal. (C₃₀H₃₅N₇O 1.25 H₂O) C, H, N.

### EXAMPLE 73

### N-[(3S)-1-azabicyclo[2.2.2]oct-3-yl]-2-(5-{5-](ethylamino)methyl]-4-methylpyridin-3-yl}-1H-indazol-3-yl)-1H-imidazole-5-carboxamide

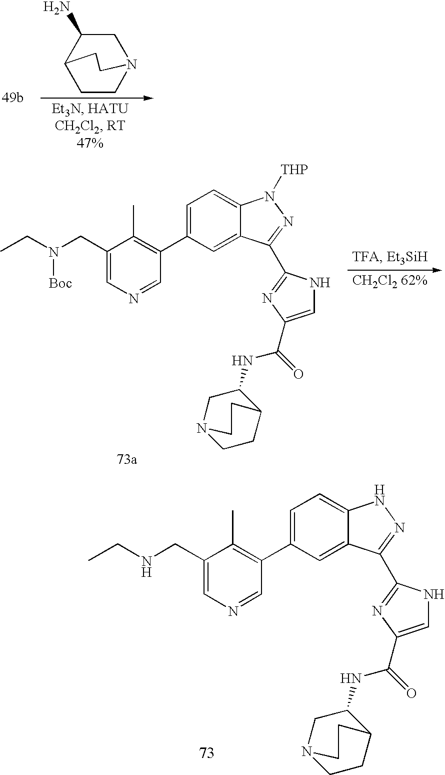

### tert-Butyl ({5-[3-(5-{[(3S)-1-azabicyclo[2.2.2]oct-3-ylamino]carbonyl}-1H-imidazol-2-yl)-1-(tetrahydro-2H-pyran-2-yl)-1H-indazol-5-yl]-4-methylpyridin-3-yl}methyl)ethylcarbamate (73a)

By the same procedure used to synthesize intermediate 49c, acid 49b (0.250 g, 0.446 mmol), diisopropylethylamine (0.51 mL, 0.380 g, 2.943 mmol), 3-aminoquinuclidine dihydrochloride (0.195 g, 0.981 mmol), HATU (0.373 g, 0.981 mmol) in 10 mL CH₂Cl₂ were considered affording 73a (0.140 g, 47%) as a pale yellow solid: ¹H NMR (DMSO-d₆) δ 13.21 (s, 1H), 8.42 (d, J=9.0 Hz, 2H), 8.29 (s, 1H), 8.24 (d, J=3.0 Hz, 1H), 7.91 (d, J=6.0 Hz, 1H), 7.79 (s, 1H), 7.49 (d, J=9.0 Hz, 1H), 6.00 (d, J=6.0 Hz, 1H), 4.51 (s, 2H), 4.27-4.31 (m, 1H), 3.94-3.97 (m, 1H), 3.77-3.84 (m, 1H), 3.53-3.59 (m, 2H), 3.10-3.27 (m, 6H), 2.49-2.52 (m, 1H), 2.21 (s, 3H), 2.04-2.17 (m, 4H), 1.77-1.86 (m, 3H), 1.62 (br s, 3H), 1.42 (br s, 9H), 1.04 (t, J=6.0 Hz, 3H); M+H⁺ 669.

### N-[(3S)-1-azabicyclo[2.2.2]oct-3-yl]-2-(5-{5-[(ethylamino)methyl]-4-methylpyridin-3-yl}-1H-indazol-3-yl)-1H-imidazole-5-carboxamide (73)

In the same manner as the deprotection of 52a, intermediate 73a (0.129 g, 0.193 mmol) was converted to the title compound 73 (0.058 g, 62%), as a pale yellow solid: ¹H NMR (DMSO-d₆) δ 13.57 (s, 1H), 8.49 (s, 1H), 8.41 (s, 2H), 8.01 (d, J=6.0 Hz, 1H), 7.69-7.73 (m, 2H), 7.42 (d, J=6.0 Hz, 1H), 4.12 (br s, 1H), 3.92 (s, 2H), 3.40 (t J=6.0 Hz, 2H), 2.89-3.10 (m, 4H), 2.77 (q, J=6.0 Hz, 2H), 2.30 (s, 3H), 2.02 (br s, 1H), 1.85-1.92 (m, 1H), 1.74 (br s, 2H), 1.50-1.56 (m, 1H), 1.13 (t, J=6.0 Hz, 3H). HRMS [M+H]⁺ calcd. 485.2772; found 485.2773.

Anal. (C₂₇H₃₂N₈O 1 TFA 1.25 H₂O) C, H, N.

### EXAMPLE 74

### N-[(5-{3-[5-(azepan-1-ylcarbonyl)-1H-imidazol-2-yl]-1H-indazol-5-yl}-4-methylpyridin-3-yl)methyl]ethanamine

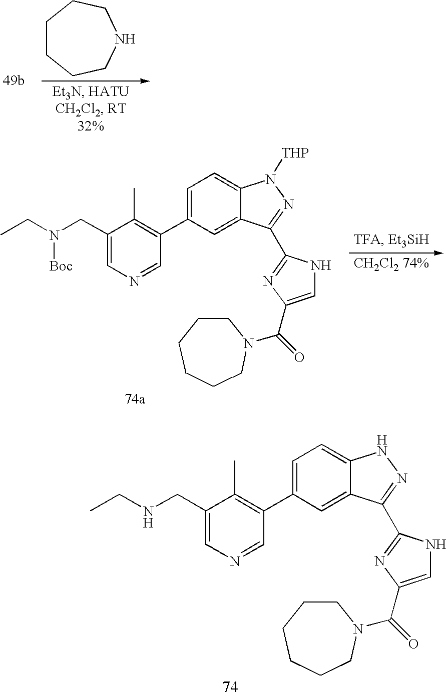

### tert-Butyl ({5-[3-[5-(azepan-1-ylcarbonyl)-1H-imidazol-2-yl]-1-(tetrahydro-2H-pyran-2-yl)-1H-indazol-5-yl]-4-methylpyridin-3-yl}methyl)ethylcarbamate (74a)

By the same procedure used to synthesize intermediate 74c, acid 74b (0.250 g, 0.446 mmol), diisopropylethylamine (0.17 mL, 0.127 g, 0.981 mmol), azepane (0.097 g, 0.981 mmol), HATU (0.373 g, 0.981 mmol) in 10 mL CH₂Cl₂ were considered affording 74a (0.092 g, 32%) as a yellow solid: ¹H NMR (DMSO-d₆) δ 13.19 (s, 1H), 8.37 (s, 1H), 8.30 (d, J=3.0 Hz, 2H), 7.91 (d, J=6.0 Hz, 1H), 7.64 (s, 1H), 7.49 (d, J=6.0 Hz,1H), 6.00 (d, J=6.0 Hz, 1H), 4.51 (s, 2H), 4.06 (t, J=6.0 Hz, 2H), 3.93-3.97 (m, 1H), 3.78-3.83 (m, 1H), 3.52 (t, J=6.0 Hz, 2H), 3.19 (br s, 2H), 2.52-2.56 (m,1H), 2.19 (s, 3H), 2.03-2.13 (m, 2H), 1.61-1.74 (m, 7H), 1.40-1.47 (m, 13H), 1.02 (t, J=6.0 Hz, 3H); M+H⁺ 642.

### N-[(5-{3-[5-(azepan-1-ylcarbonyl)-1H-imidazol-2-yl]-1H-indazol-5-yl}-4-methylpyridin-3-yl)methyl]ethanamine (74)

In the same manner as the deprotection of 52a, intermediate 74a (0.080 g, 0.125 mmol) was converted to the title compound 74 (0.042 g, 74%), as a pale yellow solid: ¹H NMR (DMSO-d₆) δ 13.49 (s, 1H), 13.13 (br s, 1H), 8.42 (s, 1H), 8.32 (s, 1H), 8.29 (s, 1H), 7.69 (d, J=6.0 Hz, 1H), 7.61 (s, 1H), 7.42 (d, J=6.0 Hz, 1H), 4.08 (t, J=6.0 Hz, 2H), 3.76 (s, 2H), 3.52 (t, J=6.0 Hz, 2H), 2.60 (q, J=6.0 Hz, 2H), 2.28 (s, 3H), 1.76 (br s, 2H), 1.68 (br s, 2H), 1.49 (br s, 4H), 1.06 (t, J=6.0 Hz, 3H). HRMS [M+H]⁺ calcd. 458.2663; found 458.2659. Anal. (C₂₆H₃₁N₇O 0.8 H₂O 0.05 EtOAc) C, H, N.

### EXAMPLE 75

### 2-(5-{5-[(ethylamino)methyl]-4-methylpyridin-3-yl}-1H-indazol-3-yl)-N-[(3S)-2-oxoazepan-3-yl]-1H-imidazole-5-carboxamide

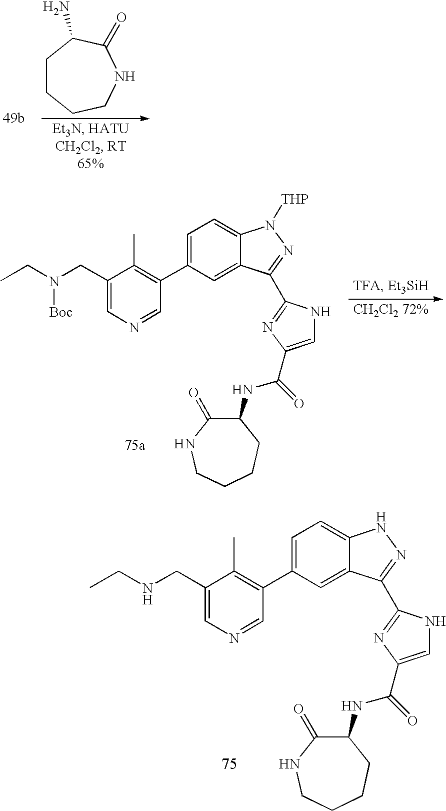

### tert-butyl ethyl({4-methyl-5-[3-{5-[(methylamino)carbonyl]-1H-imidazol-2-yl}-1-(tetrahydro-2H-pyran-2-yl)-1H-indazol-5-yl]pyridin-3-yl}methyl)carbamate-azepan-2-one (75a)

By the same procedure used to synthesize intermediate 49c, acid 49b (0.250 g, 0.446 mmol), diisopropylethylamine (0.17 mL, 0.127 g, 0.981 mmol), (S)-3-amino-azepan-2-one (0.126 g, 0.981 mmol), HATU (0.373 g, 0.981 mmol) in 10 mL CH₂Cl₂ were considered affording 75a (0.193 g, 65%) as a pale buff solid: ¹H NMR (DMSO-d₆) δ 13.22 (s, 1H), 8.39 (s, 1H), 8.34 (s, 1H), 8.27-8.28 (m, 2H), 7.93 (d, J=9.0 Hz, 2H), 7.69 (d, J=3.0 Hz, 1H), 7.50 (dd, J=3.0 Hz, J=6.0 Hz, 1H), 6.00 (d, J=6.0 Hz, 1H), 4.53 (s, 2H), 4.47-4.51 (m, 1H), 3.94-3.98 (m, 1H), 3.77-3.83 (m, 1H), 3.19-3.25 (m, 3H), 3.01-3.11 (m, 1H), 2.49-2.56 (m, 1H), 2,25 (s, 3H), 1.98-2.12 (m, 3H), 1.68-1.91 (m, 4H), 1.62 (br s, 3H), 1.41 (br s, 10 H), 1.05 (t, J=6.0 Hz, 3H); M+H⁺ 671.

### 2-(5-{5-[(ethylamino)methyl]-4-methylpyridin-3-yl}-1H-indazol-3-yl)-N-[(3S)-2-ozoazepan-3-yl]-1H-imidazole-5-carboxamide (75)

In the same manner as the deprotection of 52a, intermediate 75a (0.174 g, 0.260 mmol) was converted to the title compound 75 (0.091 g, 72%), as a pale yellow crystalline solid: ¹H NMR (DMSO-d₆) δ 13.56 (s, 1H), 8.44 (s, 1H), 8.35 (s, 1H), 8.31 (s, 1H), 8.26 (d, J=6.0 Hz, 1H), 7.93 (t, J=6.0 Hz, 1H), 7.70 (d, J=6.0 Hz, 1H), 7.67 (s, 1H), 7.42 (dd, J=6.0 Hz, 1H), 4.47-4.51 (m, 1H), 3.79 (s, 2H), 3.15-3.22 (m, 1H), 3.02-3.09 (m, 1H), 2.64 (q, J=6.0 Hz, 2H), 2.32 (s, 3H), 1.99-2.02 (m, 1H), 1.88-1.91 (m, 1H), 1,68-1.77 (m, 2H), 1.34-1.43 (m, 1H), 1.18-1.24 (m, 1H), 1.07 (t, J=6.0 Hz, 3H). HRMS [M+H]^{+ calcd.} 487.2565; found 487.2559. Anal. (C₂₆H₃₀N₈O₂₂ 1.0 H₂O 0.1 EtOAc) C, H, N.

### EXAMPLE 76

### 2-(5-55 5-[(ethylamino)methyl]-4-methylpyridin-3-yl}-1H-indazol-3-yl)-N-piperidin-4-yl-1H-imidazole-5-carboxamide

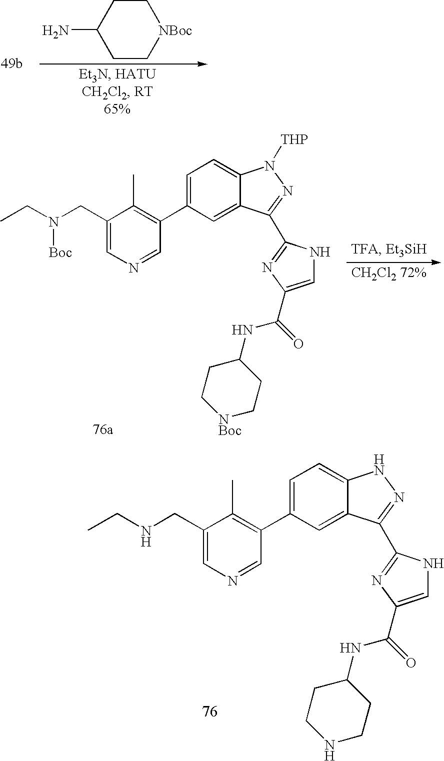

### tert-Butyl 4-[({2-[5-(5-{[(tert-butoxycarbonyl)(ethyl)amino]methyl}-4-methylpyridin-3-yl-1-(tetrahydro-2H-pyran-2-yl)-1H-indazol-3-yl]-1H-imidazol-5-yl}carbonyl)amino]piperidine-1-carboxylate (76a)

By the same procedure used to synthesize intermediate 49c, acid 49b (0.250 g, 0.446 mmol), diisopropylethylamine (0.17 mL, 0.127 g, 0.981 mmol), N-Boc-4-aminopiperidine (0.196 g, 0.981 mmol), HATU (0.373 g, 0.981 mmol) in 10 mL CH₂Cl₂ were considered affording 76a (0.229 g, 69%) as a yellow solid: ¹H NMR (DMSO-d₆) δ 13.10 (s, 1H), 8.44(s, 1H), 8.39 (s, 1H), 8.28 (s, 1H), 7.90 (d, J=6.0 Hz, 1H), 7.83 (d, J=6.0 Hz, 1H), 7.68 (s, 1H), 7.46 (dd, J=3.0 Hz, J=6.0 Hz, 1H), 5.99 (d, J=6.0 Hz, 1H), 4.51 (s, 2H), 3.93 (br s, 4H), 3.77-3.83 (m, 2H), 3.21 (q, J=6.0 Hz, 2H), 2.79 (br s, 2H), 2.49-2.52 (m, 1H), 2.19 (s, 3H), 2.03-2.11 (s, 2H), 1.77-1.82 (m, 2H), 1.62-1.72 (m, 4H), 1.41 (s, 9H), 1.38 (s, 9H), 1.05 (t, J=6.0 Hz, 3H); M+H⁺ 743.

### 2-(5-{5-[(ethylamino)methyl]-4-methylpyridin-3-yl}-1H-indazol-3-yl)-N-piperidin-4-yl-1H-imidazole-5-carboxamide (76)

In the same manner as the deprotection of 52a, intermediate 76a (0.210 g, 0.283 mmol) was converted to the title compound 76 (0.096 g, 74%), as a pale yellow crystalline solid: ¹H NMR (DMSO-d₆) δ 13.60 (s, 1H), 13.13 (s, 1H), 8.59 (s, 1H), 8.49 (s, 1H), 8.37 (s, 1H), 8.00 (d, J=6.0 Hz, 1H), 7.71 (d, J=6.0 Hz, 2H), 7.37 (d, J=6.0 Hz, 1H), 4.22 (br s, 2H), 3.95-4.06 (m, 1H), 3.27-3.31 (m, 2H), 2.97-3.05 (m, 4H), 2.30 (s, 3H), 1.90-1.96 (m, 2H), 1.73-1.79 (m, 2H), 1.23 (t, J=6.0 Hz, 3H). HRMS [M+H]⁺ calcd. 459.2616; found 459.2620. Anal. (C₂₅H₃₀N₈O 2.2 TFA 1.4 H₂O) C, H, N.

### EXAMPLE 77

### N-8-azabicyclo[3.2.1]oct-3-yl-2-(5-{5-[(ethylamino)methyl]-4-methylpyridin-3-yl}-1H-indazol-3-yl)-1H-imidazole-5-carboxamide

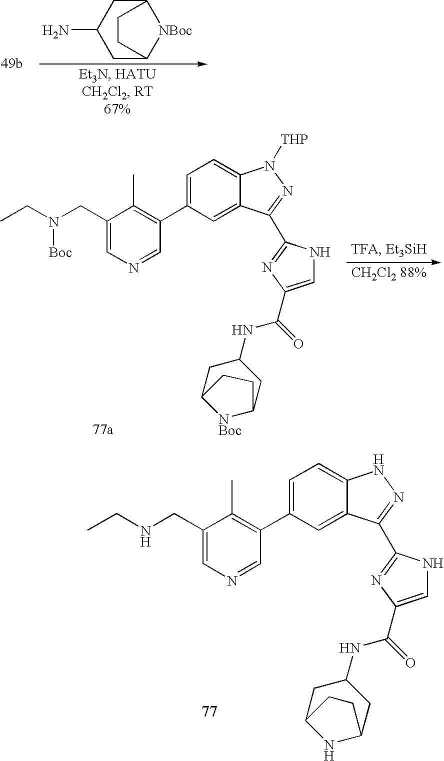

### tert-Butyl 3-[({2-[5-(5-{[(tert-butoxycarbonyl)(ethyl)amino]methyl}-4-methylpyridin-3-yl)-1-(tetrahydro-2H-pyran-2-yl)-1H-indazol-3-yl]-1H-imidazol-5-yl)carbonyl)amino]-8-azabicyclo[3.2.1]octane-8-carboxylate (77a)

By the same procedure used to synthesize intermediate 49c, acid 49b (0.250 g, 0.446 mmol), diisopropylethylamine (0.17 mL, 0.127 g, 0.981 mmol), N-Boc-tropaneamine (0.222 g, 0.981 mmol), HATU (0.373 g, 0.981 mmol) in 10 mL CH₂Cl₂ were considered affording 77a (0.229 g, 67%) as a yellow solid: ¹H NMR (DMSO-d₆) δ 13.30 (s, 1H), 8.35 (s, 2H), 8.30 (s, 1H), 7.95 (d, J=6.0 Hz, 1H), 7.92 (d, J=6.0 Hz, 1H), 7.69 (s, 1H), 7.48 (dd, J=3.0 Hz, J=6.0 Hz, 1H), 6.00 (d, J=6.0 Hz, 1H), 4.51 (s, 2H), 4.13 (q, J=6.0 Hz, 1H), 3.98 (s, 2H), 3.72-3.83 (m, 1H), 3.18 (br s, 2H), 2.52-2.57 (m, 1H), 2.17 (s, 3H), 1.88-2.04 (m, 8H), 1.57-1.68 (m, 6H), 1.40 (br s, 9H), 1.38 (br s 9H), 1.03 (t, J=6.0 Hz, 3H); M+H⁺ 769.

### N-8-azabicyclo[3.2.1]oct-3-yl-2-(5-{5-[(ethylamino)methyl]-4-methylpyridin-3-yl}-1H-indazol-3-yl)-1H-imidazole-5-carboxamide (77)

In the same manner as the deprotection of 52a, intermediate 77a (0.210 g, 0.283 mmol) was converted to the title compound 77 (0.116 g, 88%), as a pale yellow crystalline solid: ¹H NMR (DMSO-d₆) δ 13.57 (s, 1H), 8.46 (s, 1H), 8.37 (d, J=6.0 Hz, 2H), 7.95 (d, J=6.0 Hz, 1H), 7.81 (d, J=6.0 Hz, 1H), 7.70 (d, J=6.0 Hz, 2H), 7.42 (d, J=6.0 Hz, 1H), 4.06-4.12 (m, 1H), 3.84 (s, 2H), 3.70-3.76 (m, 1H), 2.70 (q, J=6.0 Hz, 2H), 2.27 (s, 3H), 2.12-2.20 (m, 3H), 1.98-2.01 (m, 2H), 1.86-1.89 (m, 2H), 1.74-1.77 (m, 2H), 1.10 (t, J=6.0 Hz, 3H). HRMS [M+H]⁺ calcd. 485.2772; found 485.2785. Anal. (C₂₇H₃₂N₈O 1.5 TFA 1.75 H₂O 0.6 EtOAc) C, H, N.

### EXAMPLE 78

### N-({5-[3-(5-{[4-(2-methoxyethyl)piperazin-1-yl]carbonyl}-1H-imidazol-2-yl)-1H-indazol-5-yl]-4-methylpyridin-3-yl}methyl)ethanamine

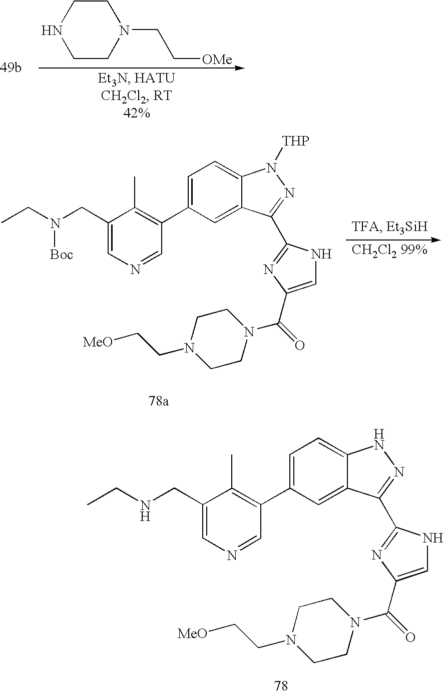

### tert-Butyl ethyl({5-[3-(5-{[4-(2-methoxyethyl)piperazin-1-yl]carbonyl}-1H-imidazol-2yl)-1-(tetrahydro-2H-pyran-2-yl)-1H-indazol-5-yl]-4-methylpyridin-3-yl}methyl)carbamate (78a)

By the same procedure used to synthesize intermediate 49c, acid 49b (0.250g, 0.446 mmol), diisopropylethylamine (0.17 mL, 0.127 g, 0.981 mmol), 1-(2-methoxyethyl)piperazine (0.141 g, 0.981 mmol), HATU (0.373 g, 0.981 mmol) in 10 mL CH₂Cl₂ were considered affording 78a (0.130 g, 42%) as a yellow crystalline solid: ¹H NMR (DMSO-d₆) δ 13.23 (s, 1H), 8.39(s, 1H), 8.30 (s, 1H), 8.26 (s, 1H), 7.91 (d, J=6.0 Hz, 1H), 7.65 (s, 1H), 7.51 (dd, J=3.0 Hz, J=6.0 Hz, 1H), 5.99 (d, J=6.0 Hz, 1H), 4.52 (s, 2H), 4.13-4.29 (m, 1H), 3.94-3.97(m, 1H), 3.77-3.83 (m, 1H), 3.56 (br s, 2H), 3.39 (t, J=6.0 Hz, 2H), 3.21 (s, 3H), 2.54-2.57 (m, 2H), 2.40-2.43 (m, 6H), 2.21 (s, 3H), 1.98-2.12 (m, 2H), 1.77-1.82 (m, 1H), 1.62 (s, 2H), 1.40 (br s, 9H), 1.23 (s, 2H), 1.02 (t, J=6.0 Hz, 3H); M+H⁺ 687.

### N-({5-[3-(5-{[4-(2-methoxyethyl)piperazin-1-yl]carbonyl}-1H-imidazol-2-yl)-1H-indazol-5-yl]-4-methylpyridin-3-yl}methyl)ethanamine (78)

In the same manner as the deprotection of 52a, intermediate 78a (0.210 g, 0.283 mmol) was converted to the title compound 78 (0.090 g, 99%), as a pale yellow crystalline solid: ¹H NMR (DMSO-d₆) δ13.55 (s, 1H), 13.20 (br s, 1H), 8.59 (s, 1H), 8.48 (s, 1H), 8.26 (s, 1H), 7.72 (d, J=6.0 Hz, 1H), 7.63 (s, 1H), 7.43 (d, J=6.0 Hz, 1H), 4.23 (s, 2H), 3.41 (t, J=6.0 Hz, 2H), 3.23 (s, 3H), 3.04 (q, J=6.0 Hz, 2H), 2.54 (s, 2H), 2.39-2.44 (m, 8H), 2.34 (s, 3H), 1.23 (t, J=6.0 Hz, 3H). HRMS [M+H]⁺ calcd. 503.2878; found 503.2873. Anal. (C₂₇H₃₄N₈O₂ 1.0 TFA 1.0 H₂O) C, H, N.

### EXAMPLE 79

### N-({4-methyl-5-[3-(5-}[4-(pyrimidin-2-ylmethyl)azepan-1-yl]carbonyl}-1H-imidazol-2-yl)-1H-indazol-5-yl]pyridin-3-yl}methyl)ethanamine

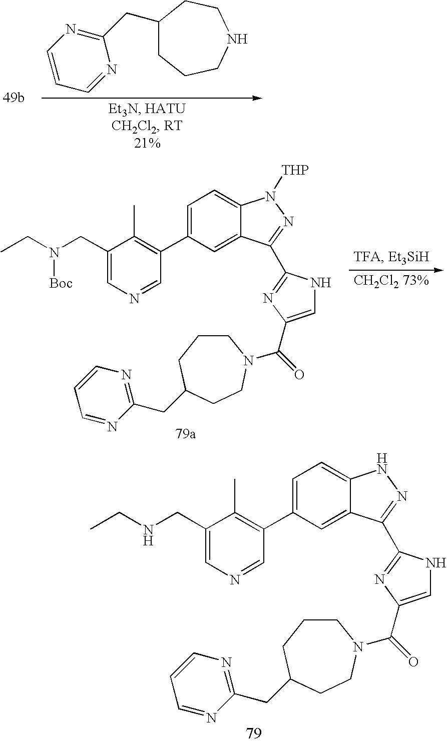

### tert-Butyl ethyl({4-methyl-5-[3-(5-{[4-(pyrimidin-2-ylmethyl)azepan-1-yl]carbonyl}-1H-imidazol-2-yl)-1-(tetrahydro-2H-pyran-2-yl)-1H-indazol-5-yl]pyridin-3-yl}methyl)carbamate (79a)

By the same procedure used to synthesize intermediate 49c, acid 49b (0.250 g, 0.446 mmol), diisopropylethylamine (0.17 mL, 0.127 g, 0.981 mmol), 4-pyrimidin-2-ylmethyl-azepane (0.187 g, 0.981 mmol), HATU (0.373 g, 0.981 mmol) in 10 mL CH₂Cl₂ were considered affording 79a (0.068 g, 21%) as a yellow solid: ¹H NMR (DMSO-d₆) δ 13.20 (s, 1H), 8.67 (d, J=6.0 Hz, 1H), 8.61 (d, J=6.0 Hz, 1H), 8.35 (d, J=6.0 Hz, 1H), 8.27 (s, 1H), 8.21 (s, 1H), 7.91 (d, J=6.0 Hz, 1H), 7.65 (s, 1H), 7.46-7.49 (m, 1H), 7.27 (tt, J=6.0 Hz, J=18 Hz, 1H), 5.99 (d, J=6.0 Hz, 1H), 4.46 (q, J=6.0 Hz, 2H), 4.27-4.43 (m, 1H), 3.94-3.97 (m, 1H), 3.77-3.83 (m, 2H), 3.42-3.57 (m, 3H), 3.10-3.16 (m, 2H), 2.77-2.79 (m, 1H), 2.49-2.53 (m, 1H), 2.17 (s, 3H), 2.03-2.12 (m, 4H), 1.75-1.80 (m, 4H), 1.62 (br s, 3H), 1.51-1.54 (m, 1H), 1.39 (br s, 9H), 0.99 (t, J=−6.0 Hz, 3H); M+H⁺ 734.

### N-({4-methyl-5-[3-(5-{[4-(pyrimidin-2-ylmethyl)azepan-1-yl]carbonyl}-1H-imidazol-2-yl)-1H-indazol-5-yl]pyridin-3-yl}methyl)ethanamine (79)

In the same manner as the deprotection of 52a, intermediate 79a (0.060 g, 0.082 mmol) was converted to the title compound 79 (0.033 g, 73%), as a pale yellow solid: ¹H NMR (DMSO-d₆) δ63.49 (s, 1H), 13.15 (br s, 1H), 8.68 (d, J=6.0 Hz, 1H), 8.63 (d, J=6.0 Hz, 1H), 8.40 (s, 1H), 8.27-8.33 (m, 2H), 7.68 (d, J=9.0 Hz, 1H), 7.63 (s, 1H), 7.40 (t, J=6.0 Hz, 1H), 7.28 (tt, J=6.0 Hz, J=15 Hz, 1H), 4.28-4.44 (m, 1H), 3.90-3.94 (m, 1H), 3.72 (s, 2H), 3.42-3.60 (m, 2H), 2.78-2.80 (m, 1H), 2.51-2.58 (m, 3H), 2.24 (s, 3H), 2.07-2.17 (m, 1H), 1.76-1.90 (m, 2H), 1.43-1.59 (m, 3H), 1.22-1.29 (m, 1H), 1.04 (t, J=6.0 Hz, 3H); HRMS [M+H]⁺ calcd. 550.3038; found 550.3039. Anal. (C₃₁H₃₅N₉O 1.25 H₂O 0.25 EtOAc) C, H, N.

### EXAMPLE 80

### N-(2-cyclohex-1-en-1-ylethyl)-2-(5-{5-[(ethylamino)methyl]-4-methylpyridin-3-yl}-1H-indazol-3-yl)-1H-imidazole-5-carboxamide

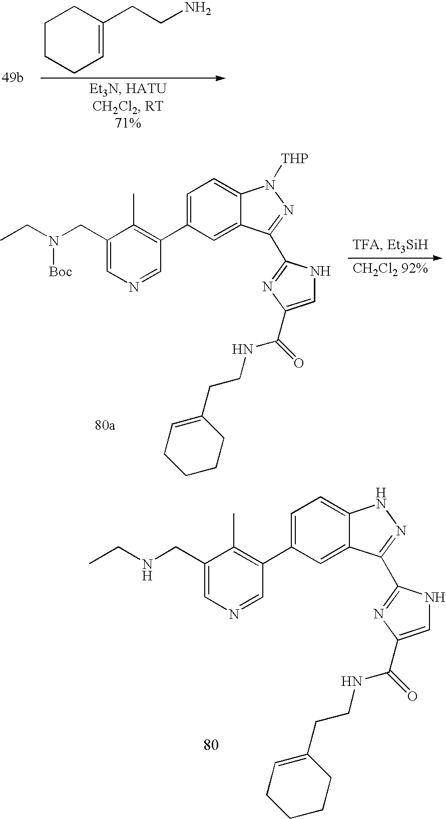

### tert-Butyl ({5-[3-(5-{[(2-cyclohex-1-en-1-ylethyl)amino]carbonyl}-1H-imidazol-2-yl)-1-(tetrahydro-2H-pyran-2-yl)-1H-indazol-5-yl]-4-methylpyridin-3-yl}methyl)ethylcarbamate (80a)

By the same procedure used to synthesize intermediate 49c, acid 49b (0.250 g, 0.446 mmol), diisopropylethylamine (0.17 mL, 0.127 g, 0.981 mmol), 2-(1-cyclohexenyl)ethylamine (0.123 g, 0.981 mmol), HATU (0.373 g, 0.981 mmol) in 10 mL CH₂Cl₂ were considered affording 80a (0.212 g, 71%) as a yellow solid: ¹H NMR (DMSO-d₆) δ 13.12 (s, 1H), 8.38 (s, 2H), 8.29 (s, 1H), 7.90 (d, J=6.0 Hz, 1H), 7.78 (s, 1H), 7.63 (s, 1H), 7.44 (dd, J=3.0 Hz, J=6.0 Hz, 1H), 5.99 (d, J=6.0 Hz, 1H), 5.35 (s, 1H), 4.51 (s, 2H), 3.92-3.96 (m, 1H), 3.77-3.83 (m, 1H), 3.20 (q, J=6.0 Hz, 2H), 2.88 (s, 2H), 2.18 (s, 3H), 2.03-2.11 (m, 4H), 1.88 (br s, 3H), 1,73 (s, 3H), 1.47 (br s, 2H), 1.33-1.44 (m, 13H), 1.06 (t, J=6.0 Hz, 3H); M+H⁺ 668.

### N-(2-cyclohex-1-en-1-ylethyl)-2-(5-{5-[(ethylamino)methyl]-4-methylpyridin-3-yl}-1H-indazol-3-yl)-1H-imidazole-5-carboxamide (80)

In the same manner as the deprotection of 52a, intermediate 80a (0.188 g, 0.282 mmol) was converted to the title compound 80 (0.126 g, 92%), as a yellow solid: ¹H NMR (DMSO-d₆) δ13.45 (s, 1H), 12.92 (br s, 1H), 8.44 (s, 1H), 8.30 (s, 2H), 7.66 (d, J=6.0 Hz, 1H), 7.43 (s, 1H), 7.37 (dd, J=3.0 Hz, J=6.0 Hz, 1H), 3.77 (s, 2H), 3.40-3.44 (m, 2H), 2.62 (q, J=6.0 Hz, 2H), 2.23 (s, 3H), 1.60-1.75 (m, 6H), 1.36-1.54 (m, 5H), 1.06 (t, J=6.0 Hz, 3H). HRMS [M+H]⁺ calcd. 484.2820; found 484.2828. Anal. (C₂₈H₃₃N₇O 0.1 TFA 0.75 H₂O) C, H, N.

### EXAMPLE 81

### N-benzyl-N-[2-(dimethylamino)ethyl]-2-(5-{5-[(ethylamino)methyl]-4-methylpyridin-3-yl}-1H-indazol-3-yl)-1H-imidazole-5-carboxamide

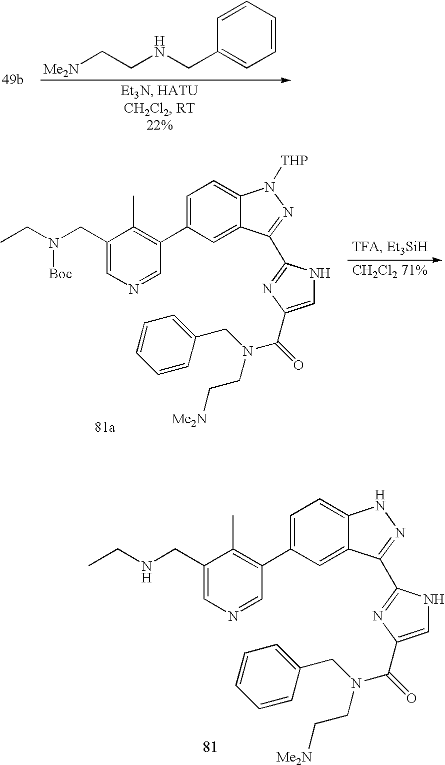

### tert-Butyl({5-[3-[5-({benzyl[2-(dimethylamino)ethyl]amino}carbonyl)-1H-imidazol-2-yl]-1-(tetrahydro-2H-pyran-2-yl)-1H-indazol-5-yl]-4-methylpyridin-3-yl}methyl)ethylcarbamate (81a)

By the same procedure used to synthesize intermediate 49c, acid 49b (0.250 g, 0.446 mmol), diisopropylethylamine (0.17 mL, 0.127 g, 0.981 mmol), N′-benzyl-N,N-dimethylethylenediamine (0.175 g, 0.981 mmol), HATU (0.373 g, 0.981 mmol) in 10 mL CH₂Cl₂ were considered affording 81a (0.072 g, 22%) as an oil: ¹H NMR (DMSO-d₆) δ 8.35 (d, J=6.0 Hz, 1H), 7.90 (d, J=6.0 Hz, 1H), 7.34-7.19 (m, 8H), 6.96-6.89 (m, 1H), 6.01-5.98 (m, 1H), 4.50 (d, J=6.0 Hz, 2H), 4.01-3.94 (m, 1H), 3.83-3.72 (m, 1H), 3.70 (s, 2H), 2.5-2.49 (m, 3H), 2.34-2.31 (t, J=6.0 Hz, 3H), 2.15-2.09 (m, 1OH), 1.97 (s, 2H), 1.75 (s, 1H), 1.62 (s, 2H), 1.42 (s, 9H), 1.01 (t, J=6.0 Hz, 3H).

M+H⁺ 6721.

### N-benzyl-N-[2-(dimethylamino)ethyl]-2-(5-{5-[(ethylamino)methyl]-4-methylpyridin-3-yl}-1H-indazol-3-yl)-1H-imidazole-5-carboxamide (81)

In the same manner as the deprotection of 52a, intermediate 81a (0.069 g, 0.096 mmol) was converted to the title compound 81 (0.051 g, 71%), as a yellow solid: ¹H NMR (DMSO-d₆) δ-HRMS [M+H]⁺ calcd. 537.3085; found 537.3108. Anal. (C₃₁H₃₆N₈O 0.25 H₂O 0.1 iPrOH) C, H, N.

### EXAMPLE 82

### N-(1-benzylpyrrolidin-3-yl)-2-(5-{5-[(ethylamino)methyl-4-methylpyridin-3-yl}-1H-indazol-3-yl)-N-methyl-1H-imidazole-5-carboxamide

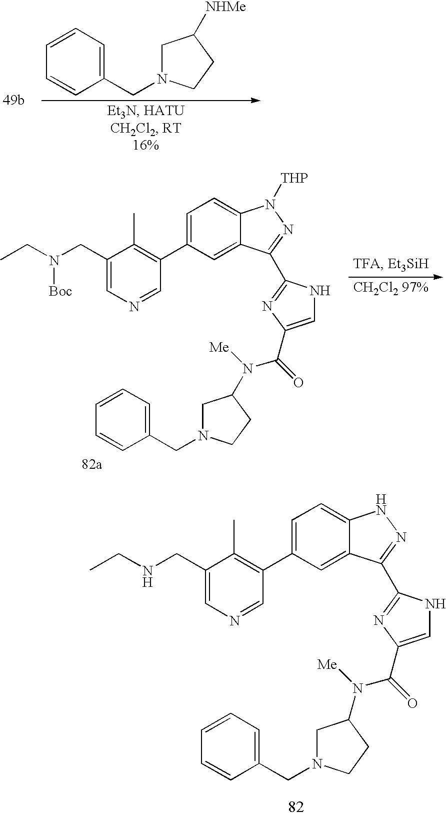

### tert-Butyl ({5-[3-(5-{[(1-benzylpyrrolidin-3-yl)(methyl)amino]carbonyl}-1H-imidazol-2-yl)-1-(tetrahydro-2H-pyran-2-yl)-1H-indazol-5-yl]-4-methylpyridin-3-yl}methyl)ethylcarbamate (82a)

By the same procedure used to synthesize intermediate 49c, acid 49b (0.250 g, 0.446 mmol), diisopropylethylamine (0.17 mL, 0.127 g, 0.981 mmol), 1-benzyl-30-(methylamino)pyrrolidine (0.186 g, 0.981 mmol), HATU (0.373 g, 0.981 mmol) in 10 mL CH₂Cl₂ were considered affording 82a (0.051 g, 16%) as a yellow solid: ¹H NMR (DMSO-d₆) δ 8.40 (s, 1H), 8.30 (br s, 2H), 7.92 (d, J=6.0 Hz, 1H), 7.63 (s, 1H), 7.49 (d, J=6.0 Hz, 1H), 7.17-7.31 (m, 5H), 5.99 (d, J=6.0 Hz, 1H), 4.46 (s, 2H), 3.94-3.97 (m, 1H), 3.77-3.83 (m, 1H), 3.34-3.36 (m, 1H), 3.07-3.17 (m, 2H), 2.90 (s, 2H), 2.65-2.68 (m, 2H), 2.45-2.51 (m, 4H), 2.19 (s, 3H), 2.03-2.12 (m, 4H), 1.75-1.83 (m, 2H), 1.62 (br s, 2H), 1.39 (br s, 9H), 1.20-1.28 (m, 1H), 0.98 (t, J=6.0 Hz, 3H); M+H⁺ 733.

### N-(1-benzylpyrrolidin-3-yl)-2-(5-{5-[(ethylamino)methyl]-4-methylpyridin-3-yl}-1H-indazol-3yl)-N-methyl-1H-imidazole-5-carboxamide (82)

In the same manner as the deprotection of 52a, intermediate 82a (0.045 g, 0.061 mmol) was converted to the title compound 82 (0.032 g, 97%), as a pale yellow solid: ¹H NMR (DMSO-d₆) δ 13.51 (s, 1H), 13.21 (s, 1H), 8.44 (s, 1H), 8.35 (s, 1H), 8.28 (s, 1H), 7.69 (d, J=6.0 Hz, 1H), 7.61 (s, 1H), 7.41 (d, J=6.0 Hz, 1H), 7.17-7.29 (m, 5H), 3.72(s, 2H), 2.90 (br s, 2H), 2.64-2.71 (m, 2H), 2.54-2.58 (m, 3H), 2.38-2.45 (m, 3H), 2.26 (s, 3H), 2.03-2.12 (m, 1H), 1.89-1.95 (m, 1H), 1.78-1.83 (m, 1H), 1.23 (s, 1H), 1.02 (t, J=6.0 Hz, 3H); HRMS [M+H³⁰ calcd. 549.3085; found 549.3092. Anal. (C₃₂H₃₆N₈O 0.25 H₂O 0.6 EtOAc) C, H, N.

### EXAMPLE 83

### N-({4-methyl-5-[3-(5-{[4-(pyridin-2-ylmethyl)piperazin-1-yl]carbonyl}-1H-imidazol-2-yl)-1H-indazol-5-yl]pyridin-3-yl}methyl)ethanamine

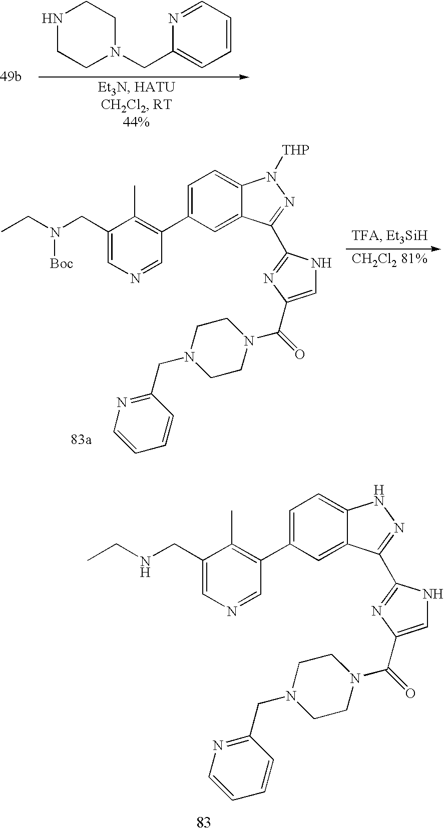

### tert-Butyl ethyl({4-methyl-5-[3-(5-{[4-(pyridin-2-ylmethyl)piperazin-1-yl]carbonyl}-1H-imidazol-2-yl)-1-(tetrahydro-2H-pyran-2-yl)-1H-indazol-5-yl]pyridin-3-yl}methyl)carbamate (83a)

By the same procedure used to synthesize intermediate 49c, acid 49b (0.250 g, 0.446 mmol), diisopropylethylamine (0.17 mL, 0.127 g, 0.981 mmol), 1-[(2-pyridyl)-methyl]-piperazine (0.174 g, 0.981 mmol), HATU (0.373 g, 0.981 mmol) in 10 mL CH₂Cl₂ were considered affording 83a (0.140 g, 44%) as a yellow solid: ¹H NMR (DMSO-d₆) δ 13.23 (s, 1H), 8.46 (d, J=3.0 Hz, 1H), 8.38 (s, 1H), 8.27 (s, 1H), 8.25 (s, 1H), 7.91 (d, J=6.0 Hz, 1H), 7.73 (t, J=6.0 Hz, 1H), 7.66 (s, 1H), 7.50 (dd, J=3.0 Hz, J=6.0 Hz, 1H), 7.40 (d, J=6.0 Hz, 1H)I 7.23-7.26 (m, 1H), 5.99 (dd, J=3.0 Hz, J=6.0 Jz, 1H), 4.50 (s, 2H), 4.26 (s, br s, 2H), 3.94-3.97 (m, 1H), 3.77-3.83 (m, 1H), 3.58-3.64 (m, 2H), 3.55 (s, 2H), 3.19 (s, 2H), 2.49-2.54 (m, 1H), 2.43 (s, 4H), 2.20 (s, 3H), 2.03-2.12 (m, 2H), 1.75-1.84 (m, 1H), 1.62 (br s, 2H), 1.38 (br s, 9H), 0.99 (t, J=6.0 Hz, 3H); M+H⁺ 720.

### N-({4-methyl-5-[3-(5-{[4-(pyridin-2-ylmethyl)piperazin-1-yl]carbonyl}-1H-imidazol-2yl)-1H-indazol-5-yl]pyridin-3-yl}methyl)ethanamine (83)

In the same manner as the deprotection of 52a, intermediate 83a (0.119 g, 0.166 mmol) was converted to the title compound 83 (0.072 g, 81%), as a pale yellow solid: ¹H NMR (DMSO-d₆) δ 13.51 (s, 1H), 13.20 (br s, 1H), 8.47 (d, J=6.0 Hz, 1H), 8.41 (s, 1H), 8.33 (s, 1H),8.23 (s, 1H),7.75 (t, J=6.0 Hz, 1H), 7.69 (d, J=6.0 Hz, 1H), 7.64 (s, 1H), 7.43 (dd, J=3.0 Hz, J=6.0 Hz, 1H), 7.39 (d, J=6.0 Hz, 1H), 7.24-7.27 (m, 1H), 4.30 (br s, 2H), 3.74 (s, 2H), 3.56-3.67 (m, 2H), 3.56 (s, 2H), 2.60 (q, J=6.0 Hz, 2H), 2.44 (br s, 4H), 2.28 (s, 3H), 1.05 (t, J=6.0 Hz, 3H). HRMS [M+H]⁺ calcd. 536.2881; found 536.2875. Anal. (C₃₀H₃₃N₉O 1 H₂O) C, H, N.

### EXAMPLE 84

### 2-(5-{5-[(ethylamino)methyl]-4-methylpyridin-3-yl}-1H-indazol-3-yl)-N-methyl-N-(1-methylpyrrolidin-3-yl)-1H-imidazole-5-carboxamide

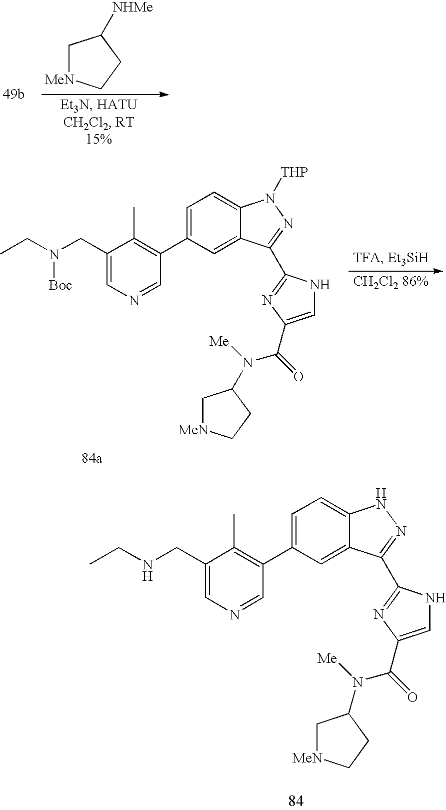

### tert-Butyl ethyl({4-methyl-5-[3-(5-{[methyl(1-methylpyrrolidin-3-yl)amino]carbonyl}-1H-imidazol-2-yl)-1-(tetrahydro-2H-pyran-2-yl)-1H-indazol-5-yl]pyridin-3-yl}methyl)carbamate (84a)

By the same procedure used to synthesize intermediate 49c, acid 49b (0.250 g, 0.446 mmol), diisopropylethylamine (0.17 mL, 0.127 g, 0.981 mmol), N,N′-dimethyl-3-aminopyrrolidine (0.112 g, 0.981 mmol), HATU (0.373 g, 0.981 mmol) in 10 mL CH₂Cl₂ were considered affording 84a (0.045 g, 15%) as a yellow solid: ¹H NMR (DMSO-d₆) δ 13.25 (s, 1H), 8.38 (s, 1H), 8.29 (s, 1H), 8.27 (s, 1H), 7.92 (d, J=6.0 Hz, 1H), 7.67 (s, 1H), 7.49 (d, J=6.0 Hz, 1H), 6.00 (d, J=6.0 Hz, 1H), 4.51 (s, 2H), 3.94-3.97 (m, 1H), 3.77-3.83 (m, 1H), 3.19 (s, 2H), 2.83-2.91 (m, 3H), 2.66-2.71 (m, 2H), 2.51-2.55 (m, 1H), 2.31-2.45 (m, 2H), 2.18 (s, 3H), 1.97-2.13 (m, 4H), 1.74-1.86 (m, 3H), 1.62 (br s, 2H), 1.41 (br s, 9H), 1.19-1.23 (m, 2H), 1.03 (t, J=6.0 Hz, 3H); M+H⁺ 657.

### 2-(5-{5-[(ethylamino)methyl]-4-methylpyridin-3-yl}-1H-indazol-3-yl)-N-methyl-N-(1-methylpyrrolidin-3-yl)-1H-imidazole-5-carboxamide (84)

In the same manner as the deprotection of 52a, intermediate 84a (0.040 g, 0.0609 mmol) was converted to the title compound 84 (0.025 g, 86%), as a pale yellow solid: ¹H NMR (DMSO-d₆) δ 13.51 (s, 1H), 8.43 (s, 1H), 8.32 (s, 1H), 8.27 (s, 1H), 7.69 (d, J=6.0 Hz, 1H), 7.61 (s, 1H), 7.40 (d, J=6.0 Hz, 1H), 3.75 (s, 2H), 2.88 (s, 2H), 2.57-2.64 (m, 5H), 2.37-2.42 (m, 1H), 2.25 (s, 3H), 2.19-2.21 (m, 1H), 2.00 (s, 3H), 1.86 (s, 2H), 1.74-1.81 (m, 1H), 1.06 (t, J=6.0 Hz, 3H). HRMS [M+H]⁺ calcd. 473.2772; found 473.2770. Anal. (C₂₆H₃₂N₈O 1.25 TFA 0.75 H₂O 0.5 EtOAc) C, H, N.

### EXAMPLE 85

### N-({4-methyl-5-[3-(5-{[4-(pyrimidin-2-ylmethyl)piperidin-1-yl]carbonyl}-1H-imidazol-2-yl)-1H-indazol-5-yl]pyridin-3-yl}methyl)ethanamine

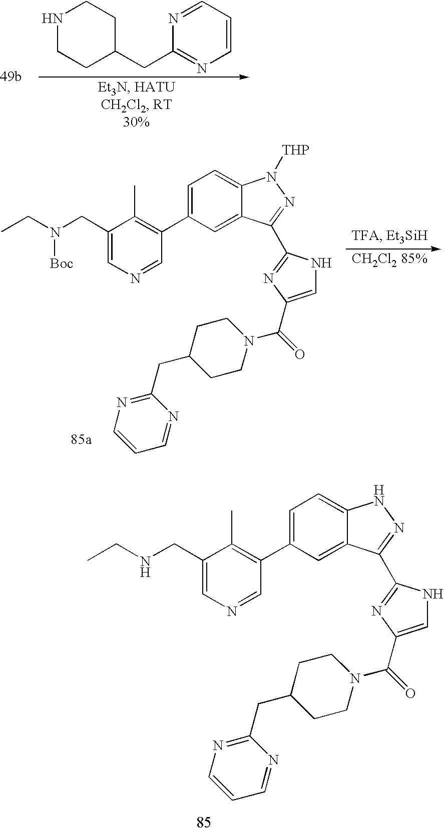

### tert-Butyl ethyl({4-methyl-5-[3-(5-{[4-(pyrimidin-2-ylmethyl)piperdin-1-yl]carbonyl}-1H-imidazol-2-yl)-1-(tetrahydro-2H-pyran-2-yl)-1H-indazol-5-yl]pyridin-3-yl}methyl)carbamate (85a)

By the same procedure used to synthesize intermediate 49c, acid 49b (0.250 g, 0.446 mmol), diisopropylethylamine (0.17 mL, 0.127 g, 0.981 mmol), 2-piperidin-40yl-methyl-pyrimidine (0.173 g, 0.981 mmol), HATU (0.373 g, 0.981 mmol) in 10 mL CH₂Cl₂ were considered affording 85a (0.095 g, 30%) as a yellow solid: ¹H NMR (DMSO-d₆) δ 13.19 (s, 1H), 8.70 (s, 1H), 8,69 (s, 1H), 8.37 (s, 1H), 8.27 (s, 2H), 7.91 (d, J=6.0 Hz, 1H), 7.63 (s, 1H), 7.49 (d, J=6.0 Hz, 1H), 7.32 (t, J=3.0 Hz, 1H),5.99 (d, J=6.0 Hz, 1H), 5.26 (br s, 1H), 4.50 (s, 2H), 4.43 (br s, 1H), 3.94-3.97 (m, 1H), 3.77-3.83 (m, 1H), 3.18 (s, 2H), 2.96-3.07 (br s, 1H), 2.63-2.73 (m, 3H), 2.49-2.54 (m, 1H), 2.17-2.19 (m, 4H), 2.03-2.10 (m, 2H), 1.75-1.85 (m, 1H), 1.56-1.62 (m, 5H), 1.38 (br s, 10H), 0.99 (t, J=6.0 Hz, 3H); M+H⁺ 720.

### N-({4-methyl-5-[3-(5-{[4-(pyrimidin-2-ylmethyl)piperidin-1-yl]carbonyl}-1H-imidazol-2-yl)-1H-indazol-5-yl]pyridin-3-yl}methyl)ethanamine (85)

In the same manner as the deprotection of 52a, intermediate 85a (0.040 g, 0.0609 mmol) was converted to the title compound 85 (0.052 g, 85%), as a pale yellow solid: ¹H NMR (DMSO-d₆) δ13.50 (s, 1H), 13.16 (br s, 1H), 8.71 (s, 1H), 8.70 (s, 1H), 8.42 (s, 1H), 8.32 (s, 1H), 8.25 (s, 1H), 7.69 (d, J=6.0 Hz, 1H), 7.61 (s, 1H), 7.42 (d, J=6.0 Hz, 1H), 7.33 (t, J=6.0 Hz, 1H), 5.22-5.36 (br s, 1H), 4.39-4.48 (br s, 1H), 3.78 (s, 2H), 3.02-3.15 (br s, 1H), 2.74-2.75 (m, 2H), 2.60-2.65 (m, 3H), 2.28 (s, 3H), 2.16-2.21 (m, 1H), 1.57-1.60 (m, 2H), 1.14-1.31 (m, 2H), 1.05 (t, J=6.0 Hz, 3H). HRMS [M+H]⁺ calcd. 536.2881; found 536.2881. Anal. (C₃₀H₃₃N₉O 2 H₂O) C, H, N.

### EXAMPLE 86

### 2-(5-{5-[(ethylamino)methyl]-4-methylpyridin-3-yl}-1H-indazol-3-yl)-1H-imidazole-5-carboxylic acid

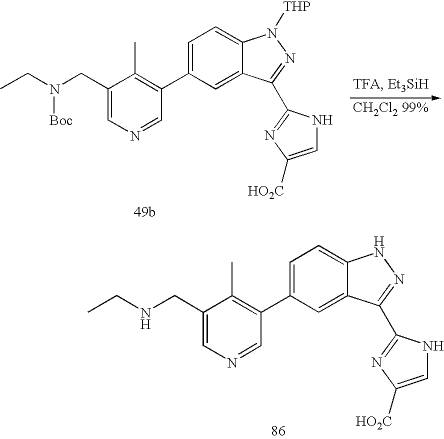

### 2-(5-{5-[(ethylamino)methyl]-4-methylpyridin-3-yl}-1H-indazol-3-yl)-1H-imidazole-5-carboxylic acid (86)

A clear yellow solution of 49b (0.100 g, 0.179 mmol)in 9 mL CH₂Cl₂, 1 mL TFA,and 0.1 mL Et₃SiH was stirred at rt for 48 h. The solvent was removed by rotary evaporation and on complete drying under vacuum yielded 86 (0.109 g, 99%), as a yellowish orange solid: ¹H NMR (DMSO-d₆) δ 13.64 (s, 1H), 8.72 (br s, 2H), 8.66 (s, 1H), 8.53 (s, 1H), 8.28 (s, 1H), 7.82 (s, 1H), 7.74 (d, J=6.0 Hz, 1H), 7.39 (d, J=6.0 Hz, 1H), 4.31-4.34 (m, 2H), 3.13 (q, J=6.0 Hz, 2H), 2.31 (s, 3H), 1.25 (t, J=6.0 Hz, 3H); HRMS [M+H]⁺ calcd. 377.1721; found 377.1723; Anal. (C₂₀H₂₀N₆O₂.1.5 H₂O 3.0 TFA) C, H, N.

### EXAMPLE 87

### N-({4-methyl-5-[3-(1,3-oxazol-5-yl)-1H-indazol-5-yl]pyridin-3-yl}methyl)ethanamine

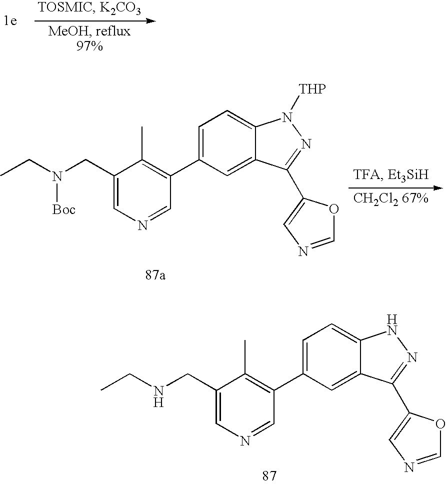

### tert-Butyl ethyl({4-methyl-5-[3-(1,3-oxazol-5-yl)-1-(tetrahydro-2H-pyran-2-yl)-1H-indazol-5-yl]pyridin-3-yl}methyl)carbamate (87a)

To a clear solution of the aldehyde 1e (0.453 g, 0.947 mmol) in 20 mL MeOH was added tosylmethyl isocyanide (0.185 g, 0.947 mmol) and K₂CO₃ (0.131 g, 0.947 mmol) at rt. The reaction mixture was refluxed for 2.5 h, and then stirred at rt for 18 h. The solvents were removed by rotary evaporation and the residue partitioned between EtOAc and H₂O. The organic extracts were dried over MgSO₄ and concentrated to give 87a (0.478 g, 97%) as an orange foam which was used in the next step without further purification. ¹H NMR (DMSO-d₆) δ 8.53 (s, 1H), 8.39 (s, 1H), 8.29 (s, 1H), 8.03 (s, 1H), 7.95 (s, 1H), 7.92 (d, J=6.0 Hz, 1H), 7.49 (d, J=6.0 Hz, 1H), 6.00 (d, J=6.0 Hz, 1H), 4.51 (s, 2H), 3.91-3.94 (m, 1H), 3.76-3.79 (m, 1H), 3.21 (q, J=6.0 Hz, 2H), 2.41-2.45 (m, 1H), 2.18 (s, 3H), 2.03-2.08 (m, 2H), 1.76-1.81 (m, 1H), 1.60-1.62 (m, 2H), 1.41 (s, 9H), 1.05 (t, J=6.0 Hz, 3H); M+H⁺ 518.

### N-({4-methyl-5-[3-(1,3-oxazol-5-yl)-1H-indazol-5-yl]pyridin-3-yl}methyl)ethanamine (87)

In the same manner as the deprotection of 2c, intermediate 87a (0.446 g, 0.863 mmol) was converted to the title compound 87 (0.082 g, 67%), as a pale yellow solid: ¹H NMR (DMSO-d₆) δ 13.61 (br s, 1H), 8.50 (s, 1H), 8.44 (s, 1H), 8.34 (s, 1H), 7.99 (s, 1H), 7.86 (s, 1H), 7.70 (d, J=6.0 Hz, 1H), 7.41 (d, J=6.0 Hz, 1H), 3.75 (s, 2H), 2.61 (q, J=6.0 Hz, 2H), 2.25 (s, 3H), 1.06 (t, J=6.0 Hz, 3H). HRMS [M+H]⁺ calcd. 334.1663; found 334.1655. Anal. (C₁₉H₁₉N₅O 0.5 H₂O 0.25 EtOAc) C, H, N.

### EXAMPLE 88

### N-({4-methyl-5-[3-(4-methyl-1,3-oxazol-5-yl)-1H-indazol-5-yl]pyridin-3-yl}methyl)ethanamine

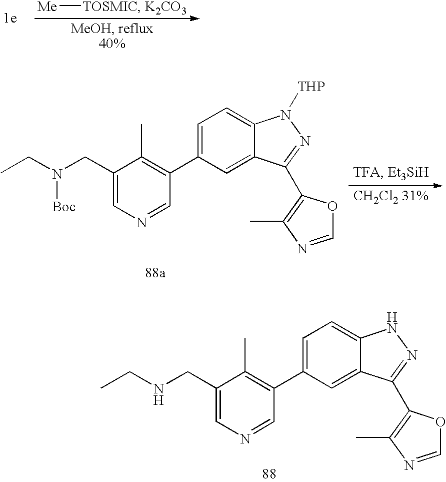

### tert-Butyl ethyl({4-methyl-5-[3-(4-methyl-1,3-oxazol-5-yl)-1-(tetrahydro-2H-pyran-2-yl)-1H-indazol-5-yl]pyridin-3-yl}methyl)carbamate (88a)

By the same procedure used to synthesize intermediate 87a , aldehyde 1e (1.541 g, 3.22 mmol), methyl tosmic (0.740 g, 3.542 mmol) and K₂CO₃ (0.489 g, 3.542 mmol) in 70 mL MeOH were considered affording 88a (0.640 g, 40%) as a yellow foam: ¹H NMR (DMSO-d₆) δ 8.41 (s, 1H), 8.37 (s, 1H), 8.28 (s, 1H), 7.93 (s, 1H), 7.91 (d, J=6.0 Hz, 1H), 7.48 (d, J=6.0 Hz, 1H), 6.01 (d, J=6.0 Hz, 1H), 4.50 (s, 2H), 3.89-3.91 (m, 1H), 3.76-3.82 (m, 1H), 3.20 (q, J=6.0 Hz, 2H), 2.45-2.54 (m, 4H), 2.17 (s, 3H), 2.06-2.11 (m, 2H), 1.75-1.84 (m, 1H), 1.62 (br s, 2H), 1.41 (s, 9H), 1.04 (t, J=6.0 Hz, 3H); M+H⁺ 532.

### N-({4-methyl-5-[3-(4-methyl-1,3-oxazol-5-yl)-1H-indazol-5-yl]pyridin-3-yl}methyl)ethanamine (88)

In the same manner as the deprotection of 2c, intermediate 88a (0.303 g, 0.571 mmol) was converted to the title compound 88 (0.051 g, 31%), as a pale yellow solid: ¹H NMR (DMSO-d₆) δ 8.45 (s, 1H), 8.32 (s, 1H), 8.24 (s, 1H), 8.02 (s, 1H), 7.68 (d, J=6.0 Hz, 1H), 7.41 (d, J=6.0 Hz, 1H), 3.89 (s, 2H), 2.77 (q, J=6.0 Hz, 2H), 2.54 (s, 3H), 2.34 (s, 3H), 1.20 (t, J=6.0 Hz, 3H). HRMS [M+H]⁺ calcd. 348.1819; found 348.1812. Anal. (C₂₀H₂₁N₅O 0.5 H₂O 0.20 EtOAc) C, H, N.

### EXAMPLE 89

### Ethyl-{4-methyl-5-[3-(5-methyl-2H-pyrazol-3-yl)-1H-indazol-5-yl]-pyridin-3-ylmethyl}-amine

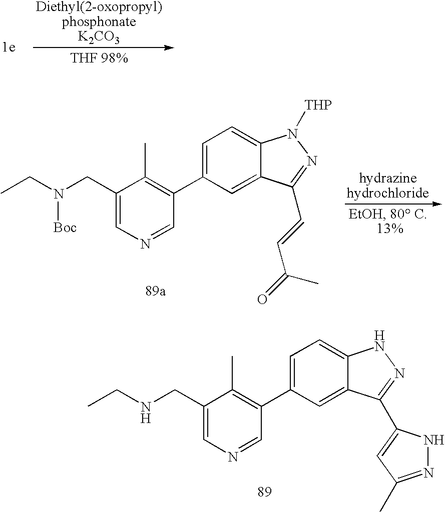

### Ethyl-{4-methyl-5-[3-(3-oxo-but-1-enyl)-1-(tetrahydro-pyran-2-yl)-1H-indazol-5-yl]-pyridin-3-ylmethyl}-carbamic acid tert-butyl ester (89a)

To a neat mixture of the aldehyde 1e (0.4 g, 0.84 mmol) and diethyl(2-oxopropyl)phosphonate (0.2 ul, 10 mmol) at RT was added an aqueous solution of potassium carbonate (231 mg, 1.67 mmol in 0.3 mL H₂O) followed by 0.5 mL THF. The resulting slurry was stirred vigourously at RT for 20 hr (Ref. Synthesis 1983, p 300-303.). The mixture was partitioned between ethyl acetate and water. The aqueous layer was extracted with ethyl acetate twice then the combined organics were washed with brine, dried (MgSO4), and concentrated to dryness. The resulting crisp foam 89a (0.42 g, 98%) was used without further purification: 1H NMR (400 MHz, ACETONITRILE-D3) δ ppm 8.39 (s, 1 H), 8.31 (s, 1 H), 7.99 (s, 1 H), 7.82 (d, J=16.42 Hz, 1 H), 7.80 (d, J=8.59 Hz, 1 H), 7.44 (dd, J=8.59, 1.52 Hz, 1 H), 7.06 (d, J=16.42 Hz, 1 H), 5.88 (dd, J=9.60, 2.27 Hz, 1 H), 4.51-4.57 (m, 2 H), 3.94-4.02 (m, 1 H), 3.75-3.86 (m, 1 H), 3.26 (q, J=7.07 Hz, 2 H), 2.44-2.59 (m, 1 H), 2.37 (s, 3 H), 2.20 (s, 3 H), 2.04-2.16 (m, 2 H), 1.61-1.87 (m, 3 H), 1.45 (s, 9 H), 1.08 (t, J=7.07 Hz, 3 H).

### Ethyl-{4-methyl-5-[3-(5-methyl-2H-pyrazol-3-yl)-1H-indazol-5-yl]-pyridin-3-ylmethyl}-amine (89)

To a solution of hydrazine hydrochloride (0.15 g, 2.18 mmol) and Iodine (0.55 g, 2.18 mmol) in EtOH (7 mL) was added a solution of the enone 89a (0.377 g, 0.727 mmol) in EtOH (3 mL) via pipette (Ref. Indian J. of Chem. 1999, p 250-127.). After stirring at 80° C. for 2 hr, the mixture was partitioned between aq. NaHSO₃ and EtOAc. The aqueous layer was basified to pH 8 with aq. NaHCO₃ and extracted with EtOAc (3×50 mL). The second set of extracts were dried (MgSO₄), reduced to minimum volume and the residue purified by radial chromatography using a gradient of 5 to 10% methanol (containing 10% NH₄OH) in CH₂Cl₂ as eluant to yield 34 mg (13%) of the desired product 89: 1H NMR (400 MHz, ACETONITRILE-D3) δ ppm 11.25 (s, 1 H), 8.43 (s, 1 H), 8.35 (s, 1 H), 8.17 (s, 1 H), J=8.59 Hz, 1 H), 7.36 (dd, J=8.59, 1.52 Hz, 1 H), 6.58 (s,1 H), 3.83 (s, 2 H), 2.70 (q, J=7.07 Hz, 2 H), 2.33 (s, 3 H), 2.28 (s, 3 H), 1.11 (t, J=7.07 Hz, 3 H).

Anal. Calcd for C₂₀H₂₂N₆.0.6 H₂O.0.4 EtOAc: C: 66.01; H: 6.78; N: 21.41. Found: C: 65.94; H: 6.64; N: 21.41.

### EXAMPLE 90

### Ethyl-{5-[3-(3H-imidazol-4-yl)-1H-indazol-5-yl]-4-methyl-pyridin-3-ylmethyl}amine

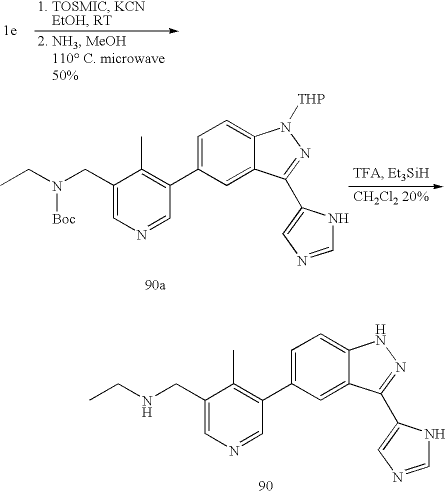

### tert-Butyl ethyl({5-[3-(1H-imidazol-5-yl)-1-(tetrahydro-2H-pyran-2-yl)-1H-indazol-5-yl]-4-methylpyridin-3-yl}methyl)carbamate (90a)

To a suspension of tert-butyl ethyl({5-[3-formyl-1-(tetrahydro-2H-pyran-2-yl)-1H-indazol-5-yl]-4-methylpyridin-3-yl}methyl)carbamate 1e (500 mg, 1.04 mmol) and tosylmethyl isocyanide (TosMIC, 204 mg, 1.04 mmol) in absolute ethanol was added sodium cyanide (5.1 mg, 0.104 mmol). The mixture was stirred at room temperature until a clear solution formed, then the solvent was removed in vacuo. The residue was dissolved in a solution of ammonia in methanol (˜7N, 5 mL) and heated to 110° C. for 15 min. in a microwave reactor. The mixture was concentrated and purified by silica gel chromatography (eluting with 2.5-10% MeOH/CH₂Cl₂) to afford 90a (270 mg, 50%) as a light brown solid. ¹H NMR (300 MHz, CDCl₃) δ 8.43 (s, 1 H), 8.35 (s, 1 H), 7.97-8.06 (m, J=1.13 Hz, 1 H), 7.80 (s, 1 H), 7.67 (s, 1 H), 7.63 (d, J=4.33 Hz, 1 H), 7.33 (d, J=9.04 Hz, 1 H), 5.76 (d, J=9.23 Hz, 1 H), 4.53 (s, 2 H), 4.11 (d, J=10.93 Hz, 1 H), 3.79 (t, J=9.42 Hz, 1 H), 3.27 (s, 2 H), 2.51-2.74 (m, J=10.36 Hz, 1 H), 2.21 (s, 2 H), 2.04-2.19 (m, 4 H), 1.62-1.87 (m, 3 H), 1.48 (s, 9 H), 1.10 (t, J=6.69 Hz, 3 H).

### N-({5-[3-(1H-imidazol-5-yl)-1H-indazol-5-yl]-4-methylpyridin-3-yl}methyl)ethanamine (90)

To a solution of tert-butyl ethyl({5-[3-(1H-imidazol-5-yl)-1-(tetrahydro-2H-pyran-2-yl)-1H-indazol-5-yl]-4-methylpyridin-3-yl}methyl)carbamate 90a (270 mg, 0.523 mmol) in CH₂Cl₂ (2.0 mL) were added triethylsilane (210 μL, 1.31 mmol) and trifluoroacetic acid (2.0 mL). The reaction was stirred at room temperature for 16 hours then concentrated in vacuo. The residue was diluted with EtOAc (50 mL), washed with 1N ammonium hydroxide (2×30 mL). The aqueous layer was back-extracted with EtOAc (50 mL). The combined organics were washed with brine, dried (MgSO₄), filtered, and concentrated in vacuo. The crude residue was purified by silica gel chromatography (eluting with 20% ammonia saturated methanol/CH₂Cl₂). The resulting powder was suspended in EtOAc (10 mL) and CH₂Cl₂ (20 mL) and filtered to remove excess silica gel to afford 90 (34.2 mg, 20%) as a yellow solid. ¹H NMR (300 MHz, CD₃OD) δ 8.35 (s, 1 H), 8.27 (s, 1 H), 7.99 (s, 1 H), 7.71 (s, 1 H), 7.54 (d, J=8.85 Hz, 1 H), 7.52 (s, 1 H), 7.29 (d, J=8.29 Hz, 1 H), 3.82 (s, 2 H), 2.70 (q, J=7.16 Hz, 2 H), 2.26 (s, 3 H), 1.91 (s, 2 H), 1.12 (t, J=7.06 Hz, 3 H).

Anal. Calcd for C₁₉H₂₀N₆.0.5(EtOAc).1.1(H₂O): C, 63.65; H, 6.66; N, 21.21. Found: C, 63.43; H, 6.35; N, 21.22.

### EXAMPLE 91

### Ethyl-{4-methyl-5-[3-(1H-tetrazol-5-yl)-1H-indazol-5-yl]-pyridin-3-ylmethyl}-amine

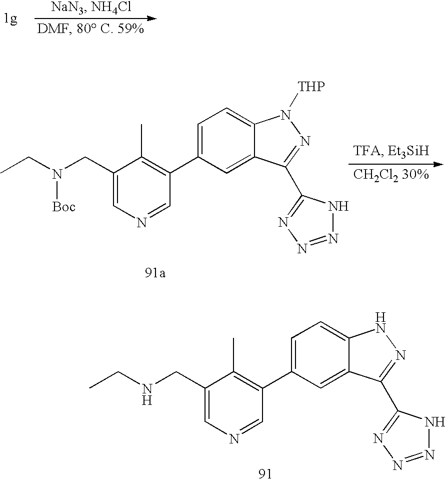

### Ethyl-{4-methyl-5-[1-(tetrahydro-pyran-2-yl)-3-(1H-tetrazol-5-yl)-1H-indazol-5-yl]-pyridin-3-ylmethyl}-carbamic acid tert-butyl ester (91a)

A solution of the nitrile 1 g (0.57 g, 1.2 mmol), ammonium chloride (0.128 g, 2.4 mmol), and sodium azide (0.156 g, 2.4 mmol) in DMF (8 mL) was heated at 80° C. for 17 hr. The mixture was poured into ice cold pH 7 phosphate buffer (100 mL) and extracted with ethyl acetate (3×50 mL). The combined organics were washed with water (1×25 ml), brine (1×25mL), dried (MgSO₄), then concentrated to dryness. The residue was purified by flash chromatography on silica gel using a gradient of 0% to 50% ethyl acetate in dichloromethane then 0% to 10% methanol in dichloromethane. The desired fractions were combined, concentrated to dryness, and the resulting residue triturated with TBME/hexanes to yield a white solid 91 a (0.36 g, 59%) that was collected by filtration: 1 H NMR (400 MHz, CHLOROFORM-D) δ ppm 8.48 (s, 1 H), 8.41 (s, 2 H), 7.75 (d, J=8.59 Hz, 1 H), 7.40 (d, J=8.59 Hz, 1 H), 5.83 (dd, J=9.22, 1.89 Hz, 1 H), 4.56 (s, 2 H), 3.94-4.10 (m, 1 H), 3.66-3.83 (m, 1 H), 3.28 (m, 2 H), 2.42-2.59 (m, 1 H), 2.24 (s, 3 H), 2.05-2.16 (m, 2 H), 1.61-1.82 (m, 3 H), 1.48 (s, 9 H), 1.12 (t, J=6.95 Hz, 3 H).

### Ethyl-{4-methyl-5-[3-(1H-tetrazol-5-yl)-1H-indazol-5-yl]-pyridin-3-ylmethyl}-amine (91)

To a solution of the indazole 91a (0.33 g, 0.64 mmol) and triethylsilane (0.2 mL) in dichloromethane (9 mL) was added trifluoroacetic acid (1 mL) at RT. After stirring at RT for 40 hours, the mixture was diluted with toluene and concentrated to dryness. The resulting residue was purified by reverse phase HPLC to yield 65 mg (30%) of the title compound 91: 1H NMR (400 MHz, DMSO-D6) δ ppm 13.20 (s,1 H), 8.58 (s,1 H), 8.46 (s,1 H), 8.34 (s, 1 H), 7.64 (d, J=8.59 Hz, 1 H), 7.33 (d, J=8.59 Hz, 1 H), 4.27 (s, 2 H), 3.10 (q, J=7.24 Hz, 2 H), 2.34 (s, 3 H), 1.25 (t, J=7.33 Hz, 3 H).

Anal. Calcd for C₁₇H₁₈N₈.0.8 H₂O: C: 58.54; H: 5.66; N: 32.13.Found: C: 58.43; H: 5.61; N: 31.96.

### EXAMPLE 92

### N-({4-methyl-5-[3-(4-methyl-1H-imidazol-2-yl)-1H-indazol-5-yl]pyridin-3-yl}methyl)ethanamine

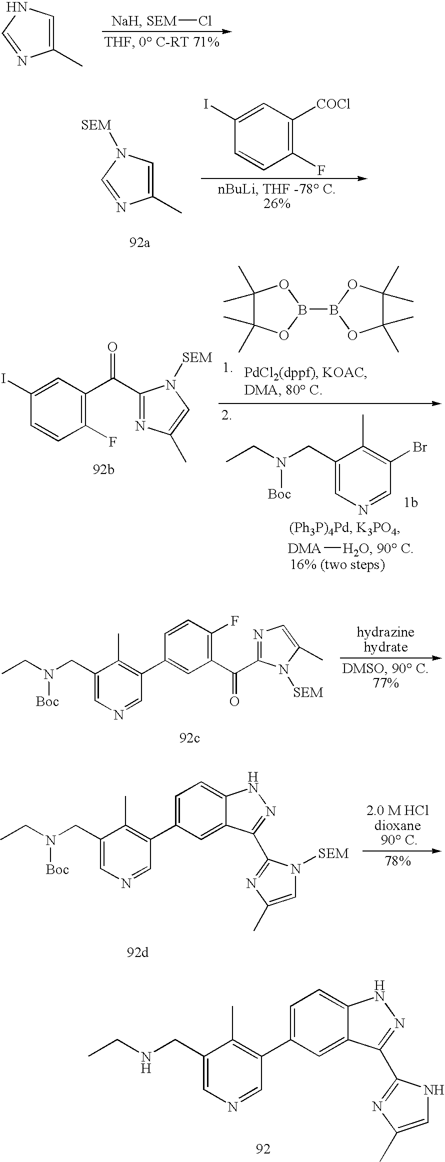

### 4-methyl-1-{[2-(trimethylsilyl)ethoxy]methyl}-1H-imidazole (92a)

To a solution of 4-methylimidazole (3.09 g, 37.68 mmol) in 100 mL THF was slowly added 95% NaH (0.995 g, 41.45 mmol) at 0° C. The suspension was stirred at 0° C. for 30 mins and then 2-(trimethylsilyl)ethoxymethyl chloride (7.853 g, 8.33 mL, 47.10 mmol) was added. The reaction mixture was stirred at rt for 21 h. Upon completion of reaction as indicated by TLC the mixture was partitioned between EtOAc and saturated aqueous NaHCO₃. The organic extracts were dried over MgSO₄ and concentrated. The crude compound was purified by silica gel chromatography (eluting with a gradient of 80% to 100% EtOAc in hexanes), yielding 92a (5.71 g, 71%) as a pale yellow oil: ¹H NMR (CDCl₃) mixture of tautomers δ 7.80 (d, J=6.0 Hz, 1H), 6.83 (s, 0.5H), 6.77 (s, 0.5H), 5.24 (d, J=3.0 Hz, 2H), 3.47 (t, J=6.0 Hz, 2H), 2.27 (s, 3H), 0.86-0.91 (m, 2H), 0.03 (s, 9H); M+H⁺ 214.

### (2-fluoro-5-iodophenyl)(4-methyl-1-{[2-(trimethylsilyl)ethoxy]methyl}-1H-imidazol-2-yl)methanone (92b)

To a solution of 92a (2.73 g, 12.88 mmol) in 50 mL THF was slowly added 2.5M nBuLi (6.2 mL, 15.46 mmol) at −78° C. The reaction was stirred at −78° C. for 30 mins and then 45 f (4.39 g, 8.33 mL, 15.46 mmol) in 50 mL THF was added dropwise. The reaction mixture was stirred at rt for 20 h. Upon completion of reaction as indicated by TLC the mixture was partitioned between EtOAc and H₂O. The organic extracts were dried over MgSO₄ and concentrated. The crude compound was purified by silica gel chromatography eluting with 20% EtOAc in hexanes, yielding 92b (1.563 g, 26%) as a yellow oil: ¹H NMR (CDCl₃) mixture of tautomers δ 8.19-8.21 (m, 0.29H), 7.95-7.97 (m, 0.52H), 7.87-7.89 (m, 0.38H), 7.74-7.80 (m, 1H), 7.14 (s, 0.5H), 7.04 (s, 0.5H), 6.87-6.94 (m, 1H), 5.87 (s, 1H), 5.77 (s, 1H), 3.57-3.62 (m, 2H), 2.38 (s, 1.5H), 2.30 (s, 1.5H), 0.87-0.98 (m, 2H), 0.04-0.02 (m, 9H); M+H⁺ 461.

### tert-Butyl ethyl[(5-{4-fluoro-3-[(4-methyl-1-{[2-(trimethylsilyl)ethoxy]methyl}-1H-imidazol-2-yl)carbonyl]phenyl}-4-methylpyridin-3-yl)methyl]carbamate (92c)

In a three necked flask was dissolved 92b (0.735 g, 1.594 mmol), bis(pinacolato)diborane (0.445 g, 1.753 mmol) and KOAc (0.469 g, 4.782 mmol) in 20 ml DMA. Nitrogen gas was continuously bubbled through the reaction mixture and PdCl₂(dppf).CH₂Cl₂ (0.039 g, 0.0478 mmol) was added. The reaction mixture was heated at 80-90° C. for 3 h and then a solution of the bromopyridine compound 1b (0.577 g, 1.753 mmol) in 10 ml DMA, K₃PO₄ (0.508 g, 2.391 mmol), 20 ml H₂O and (Ph₃P)₄Pd (0.055 g, 0.0478 mmol) was added. The resulting mixture was heated at 90-100° C. for 24 h. Upon completion of reaction as indicated by TLC the mixture was partitioned between EtOAc and H₂O. The organic extracts were dried over MgSO₄ and concentrated. The crude compound was purified by silica gel chromatography eluting with 30% to 100% EtOAc in hexanes, yielding 92c (0.149 g, 16%) as a yellow oil: ¹H NMR (CDCl₃) mixture of tautomers δ8.36 (s, 1H), 7.68-7.63 (m, 1H), 7.56-7.51 (m, 1H), 7.47-7.44 (m, 1H), 7.40-7.25 (m, 1H), 7.15 (s, 1H), 5.80 (m, 2H), 4.54 (s, 2H), 3.64-3.60 (m, 2H), 3.28 (s, 2H), 2.39-2.37 (m, 3H), 2.26-2.24 (m, 3H), 1.48 (s, 9H), 1.13 (s, 3H), 0.97-0.91 (m, 2H), −0.03 (s, 9H); M+H⁺ 583

### tert-Butyl ethyl({4-methyl-5-[3-(4-methyl-1-{[2-(trimethylsilyl)ethoxy]methyl}-1H-imidazol-2-yl)-1-(tetrahydro-2H-pyran-2-yl)-1H-indazol-5-yl]pyridin-3-yl}methyl)carbamate (92d)

To a clear yellow solution of 92c (0.149 g, 0.256 mmol) in 10 ml DMSO was added hydrazine hydrate (0.080 ml, 0.082 g, 2.56 mmol). The reaction mixture was stirred at 90-95° C. for 7 h then continued to stir at rt for 18 h. The reaction mixture was partitioned between EtOAc and 10% Na₂CO₃. The organic extracts were dried over MgSO₄ and concentrated. The crude compound was purified by silica gel chromatography eluting with 50% to 100% EtOAc in hexanes, yielding 92d (0.113 g, 77 %) as a yellow oil: ¹H NMR (CDCl₃) mixture of tautomers δ8.71 (s, 1H), 8.31 (s, 1H), 7.64-7.68 (m, 1H), 7.56-7.53 (m, 1H), 7.47-7.44 (m, 1H), 7.30-7.25 (m, 1H), 7.21 (s, 1H), 5.80 (s, 2H), 4.57 (s, 2H), 3.55-3.32 (m, 2H), 3.35-3.32 (m, 2H), 2.56-2.50 (m, 3H), 2.03 (s, 3H), 1.49 (s, 9H), 1.25 (t, J=6.0 Hz, 3H), 0.87-0.79 (m, 2H), −0.14 (m, 9H); M+H⁺ 577.

### N-({4-methyl-5-[3-(4-methyl-1H-imidazol-2-yl)-1H-indazol-5-yl]pyridin-3-yl}methyl)ethanamine (92)

Compound 92d (0.107 g, 0.186 mmol) was dissolved in 5 ml 1,4-dioxane and 5 ml 2N HCl. The reaction mixture was heated at 85-90° C. for 18h. The solvent was removed by rotary evaporation and the residue was taken up in 5 mL H₂O, made basic to pH 12 with 50% wt NaOH and extracted several times with 20% iPrOH in CHCl₃. The organic extracts were dried over MgSO₄ and concentrated. The crude compound was purified by silica gel chromatography (eluting with a gradient of 0% to 10% MeOH saturated with NH₃ in CHCl₃), yielding 92 (0.050 g, 78%), as a pale yellow solid: ¹H NMR (DMSO-d₆) δ13.29 (s, 1H), 12.44 (s, 1H), 8.44 (s, 1H), 8.30 (s, 1H), 8.24 (s, 1H), 7.63 (d, J=6.0 Hz, 1H), 7.35 (d, J=6.0 Hz, 1H), 6.73 (s, 1H), 3.76 (s, 2H), 2.62 (q, J=6.0 Hz, 2H), 2.23 (s, 3H), 2.16 (s, 3H), 1.06 (t, J=6.0 Hz, 3H); HRMS [M+H]⁺ calcd. 347.1979; found 347.1973; Anal. (C₂₀H₂₂N₆.0.75 H₂O.0.3 EtOAc) C, H, N.

### EXAMPLE 93

### N-methyl-1-{4-methyl-5-[3-(4-methyl-1H-imidazol-2-yl)-1H-indazol-5-yl]pyridin-3-yl}methanamine

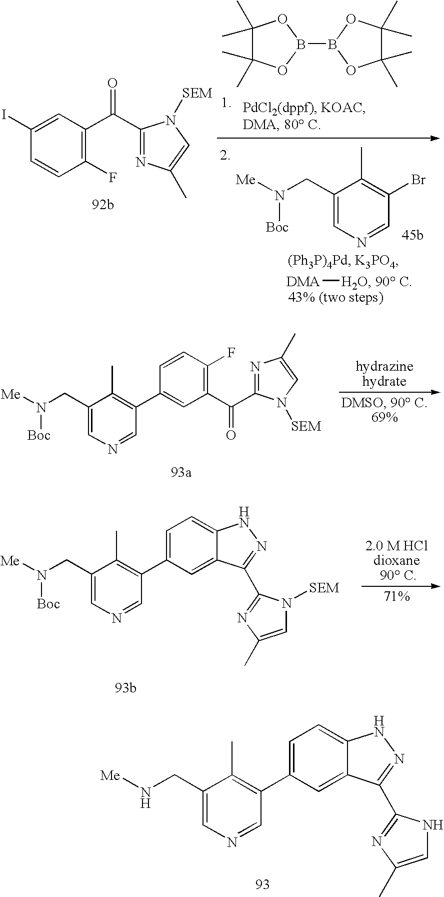

### tert-Butyl [(5-(4-fluoro-3-[(4-methyl-1-{[2-(trimethylsilyl)ethoxy]methyl}-1H-imidazol-2-yl)carbonyl]phenyl}-4-methylpyridin-3-yl)methyl]methylcarbamate (93a)

By the same procedure used to synthesize intermediate 92c, intermediate 92b (0.758 g, 1.644 mmol) was coupled to bromopyridine 45b (0.550 g, 1.753 mmol) to yield compound 93a (0.400 g, 43%), as a yellow oil: ¹H NMR (CDCl₃) δ 8.43 (s, 1H), 7.70-7.63 (m, 1H), 7.58-7.52 (m, 1H), 7.47-7.40 (m, 1H), 7.28-7.23 (m,1H), 7.13 (s, 1H), 5.79 (s, 2H), 3.63-3.59 (m, 2H), 2.87 (s, 2H), 2.38 (s, 3H), 2.26 (s, 3H), 2.03 (s, 3H), 1.47 (s, 9H), 0.96-0.89 (m, 2H), −0.03-0.05 (m, 9H); M+H⁺ 569

### tert-Butyl methyl({4-methyl-5-[3-(4-methyl-1-{[2-(trimethylsilyl)ethoxy]methyl}-1H-imidazol-2-yl)-1-(tetrahydro-2H-pyran-2-yl)-1H-indazol-5-yl]pyridin-3-yl}methyl)carbamate (93b)

By the same procedure used to synthesize intermediate 92d, intermediate 93a (0.400 g, 0.704 mmol) was converted to compound 93b (0.272 g, 69%), as a yellow oil: ¹H NMR (CDCl₃) δ 8.44 (s, 1H), 8.32 (s, 1H), 7.68-7.64 (m, 1H), 7.56-7.52 (m,1H), 7.48-7.43 (m, 1H), 7.24-7.18 (m, 1H), 7.00 (s, 1H), 5.74 (s, 2H), 3.46-3.35 (m, 2H), 2.86 (s, 2H), 2.41 (s, 3H), 2.23 (s, 3H), 2.03 (s, 3H), 1.48 (s, 9H), 0.82-0.70 (m, 2H), −0.16-0.22 (m, 9H); M+H⁺ 563

### N-methyl-1-{4-methyl-5-[3-(4-methyl-1H-imidazol-2-yl)-1H-indazol-5-yl]pyridin-3-yl}methanamine (93)

In the same manner as the deprotection of 92d, intermediate 93b (0.272 g, 0.484 mmol) was converted to the title compound 93(0.114 g, 71%), as a pale yellowsolid: ¹H NMR (DMSO-d₆) δ13.29 (s, 1H), 12.44 (s, 1H), 8.43 (s, 1H), 8.31 (s, 1H), 8.24 (s, 1H), 7.63 (d, J=6.0 Hz, 1H), 7.35 (d, J=6.0 Hz, 1H), 6.72 (s, 1H), 3.72 (s, 2H), 2.35 (s, 3H), 2.24-2.20 (m, 6H); HRMS [M+H]⁺ calcd. 333.1822; found 333.1822. Anal. (C₁₉H₂₀N₆ 0.75 H₂O 0.20 EtOAc) C, H,N.

### EXAMPLE 94

### 3-(1H-Imidazol-2-yl)-5-(1H-pyrrolo[2,3-c]pyridin-4-yl)-1H-indazole

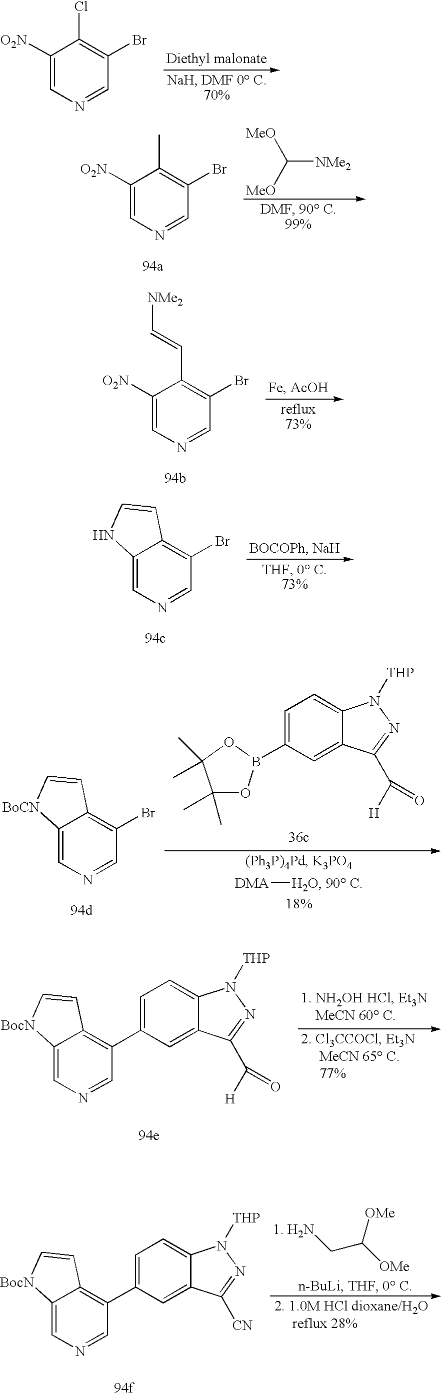

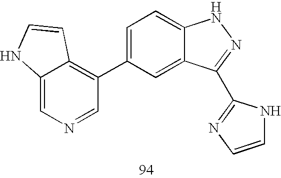

### 3-bromo-4-methyl-5-nitropyridine (94a)

Diethyl malonate (3.84 mL, 25.3 mmol) was slowly added to a suspension of sodium hydride (1.01 g of a 60% suspension in oil, 25.3 mmol) in DMF (15 mL) at 0° C. and stirred 30 min until gas evolution ceased. 3-bromo-4-chloro-5-nitropyridine (3.00 g, 12.6 mmol) was added slowly, and the dark reddish-brown solution was stirred at room temperature for 1 hour. The reaction was carefully quenched with water and acidified to pH 1 with 1N HCl. The aqueous mixture was extracted with EtOAc (2×150 mL). The combined organics were washed with water (100 mL) and brine, dried (Na₂SO₄), filtered, and concentrated in vacuo. The residue was diluted with 4N HCl (50 mL) and the solution refluxed 16 hours. The mixture was cooled in an ice bath and basified to pH 7 with 50% NaOH. The aqueous mixture was extracted with EtOAc (3×100 mL) and the combined organics were washed with brine, dried (MgSO₄), filtered and concentrated in vacuo to afford 94a (1.90 g, 70%) as a yellow solid. ¹H NMR (300 MHz, CDCl₃) δ 8.94 (s,1H), 8.97 (s,1H), 2.65 (s, 3H).

### 2-(3-bromo-5-nitropyridin-4-yl)-N,N-dimethylethylenamine (94b)

N,N-dimethylformamide dimethyl acetal (2.34 mL, 17.6 mmol) was added to a solution of 3-bromo-4-methyl-5-nitropyridine 94a (1.90 g, 8.80 mmol) in DMF (11 mL) and the red solution was heated at 90° C. for 90 min. The mixture was cooled and diluted with Et₂O (150 mL). The organic layer was washed with water (75 mL) and brine, dried (Na₂SO₄), filtered, and concentrated to afford 94b (2.38 g, 99%) as a blood-red solid that was used without further purification. ¹H NMR (300 MHz, CDCl₃) δ 8.54 (s, 1H), 8.51 (s, 1H), 7.20 (d, J=13.38 Hz, 1H), 5.20 (d, J=13.56 Hz, 1H), 2.98 (s, 6H).

### 4-bromo-1H-pyrrolo[2,3-c]pyridine (94c)

2-(3-bromo-5-nitropyridin-4-yl)-N,N-dimethylethylenamine 94b (2.38 g, 8.78 mmol) and iron powder (˜325 mesh, 2.45 g, 43.9 mmol) were stirred in acetic acid (25 mL). The red mixture was heated to reflux and turned grayish-green with a white precipitate. After refluxing for 45 min the reaction was cooled, diluted with EtOAc (150 mL), filtered through Celite and rinsed with additional EtOAc. The filtrate was slowly basified to pH 8 with saturated aqueous NaHCO₃, and the aqueous layer was extracted with EtOAc. The combined organics were washed with brine, dried (MgSO₄), filtered, and concentrated in vacuo. Silica gel chromatography (eluting with 5% MeOH in EtOAc) afforded 94c (1.26 g, 73%) as a yellow solid. ¹H NMR (300 MHz, CDCl₃) δ 8.96 (br s, 1H), 8.74 (s, 1H), 8.37 (s, 1H),7.46 (m, 1H), 6.66 (m,1H).

### tert-Butyl 4-bromo-1H-pyrrolo[2,3-c]pyridine-1-carboxylate (94d)

To a suspension of sodium hydride (307 mg of a 60% suspension in oil, 7.67 mmol) in anhydrous THF (5 mL) at 0° C. was slowly added 4-bromo-1H-pyrrolo[2,3-c]pyridine 94c (1.26 g, 6.39 mmol) in anhydrous THF (10 mL). The resulting solution was stirred until gas evolution ceased, about 10 min, before addition of tert-butylphenyl carbonate (1.30 mL, 7.03 mmol). The reaction was stirred 2 h at room temperature, quenched carefully with water, and partitioned between EtOAc (100 mL) and water (150 mL). The aqueous layer was extracted with EtOAc (2×50 mL) and the combined organics were washed with 1N NaOH (100 mL) and brine, dried (MgSO₄), filtered and concentrated in vacuo. Silica gel chromatography (eluting with 5% MeOH in EtOAc) afforded 94d (1.38 g, 73%) as a yellow solid. ¹H NMR (300 MHz, CDCl₃) δ 9.32 (s, 1H), 8.50 (s, 1H), 7.81 (d, J=3.58 Hz, 1H), 6.67 (d, J=3.01 Hz, 1H), 1.70 (s, 9H).

### tert-Butyl 4-(3-formyl-1-tetrahydro-2H-pyran-2-yl-1H-indazol-5-yl)-1H-pyrrolo[2,3-c]pyridine-1-carboxylate (94e)

A solution of tert-butyl 4-bromo-1H-pyrrolo[2,3-c]pyridine-1-carboxylate 94d (1.37 g, 4.61 mmol), 1-tetrahydro-2H-pyran-2-yl-5-(4,4,5,5-tetramethyl-1,3,2-dioxaborolan-2-yl)-1H-indazole-3-carbaldehyde 36c (1.78 g, 5.00 mmol), and potassium phosphate (1.47 g, 6.92 mmol) in N,N-dimethylacetamide (46 mL) and water (4.6 mL) was degassed by bubbling with N₂ for 30 min. Tetrakis(triphenylphosphine) palladium(0) (266 mg, 0.231 mmol) was added, and the mixture was degassed an additional 30 min before placing in a 90° C. oil bath. After 2 hours the reaction was removed from heat, diluted with EtOAc (150 mL), and washed with a mixture of water (100 mL) and saturated aqueous NaHCO₃ (100 mL). The aqueous layer was extracted with EtOAc (2×100 mL) and the combined organics were washed with brine, dried (MgSO₄), filtered, and concentrated in vacuo. The crude mixture was dissolved in anhydrous THF (20 mL) and added to a suspension of sodium hydride (307 mg of a 60% suspension in oil) in THF (10 mL) at 0° C. After 20 min stirring, tert-butylphenyl carbonate (1.0 mL, 5.4 mmol) was added and the mixture stirred 20 min before carefully quenching with water (50 mL). The aqueous layer was extracted with EtOAc (3×50 mL) and the combined organics were washed with bring, dried (MgSO₄), filtered, and concentrated in vacuo. Silica gel chromatography of the crude residue (eluting with a 10-80% EtOAc in hexanes gradient) afforded 94e (367 mg, 18%) as a yellow foam. ¹H NMR (300 MHz, CDCl₃) δ 10.30 (s, 1H), 9.42 (s, 1H), 8.56 (s, 1H), 8.54 (s, 1H), 7.83 (m, 2H), 7.74 (dd, J=1.70, 8.67 Hz, 1H), 6.78 (d, J=3.01 Hz, 1H), 5.91 (dd, J=2.64, 8.85 Hz, 1H), 4.05 (m, 1H), 3.83 (m, 1H), 2.62 (m, 2H), 2.23 (m, 2H), 1.84 (m, 2H), 1.73 (s, 9H).

### tert-Butyl 4-(3-cyano-1-tetrahydro-2H-pyran-2-yl-1H-indazol-5-yl)-1H-pyrrolo[2,3-c]pyridine-1-carboxylate (94f)

To a solution of tert-butyl 4-(3-formyl-1-tetrahydro-2H-pyran-2-yl-1H-indazol-5-yl)-1H-pyrrolo[2,3-c]pyridine-1-carboxylate 94e (223 mg, 0.500 mmol) in acetonitrile (5 mL) were added triethylamine (83 μL, 0.60 mmol) and hydroxylamine hydrochloride (38 mg, 0.55 mmol). The reaction mixture was placed in a 65° C. oil bath and stirred 2.5 hours before the addition of triethylamine (167 μL, 1.2 mmol) and trichloroacetyl chloride (78 μL, 0.70 mmol). After another 2 hours triethylamine (83 μL, 0.60 mmol) and trichloroacetyl chloride (67 μL, 0.60 mmol) were added. The reaction was stirred 20 min, removed from the oil bath and partitioned between EtOAc (20 mL) and a mixture of H₂O (10 mL) and brine (10 mL). The aqueous layer was extracted with EtOAc (2×20 mL). The combined organics were dried (Na₂SO₄), filtered, and concentrated in vacuo. Silica gel chromatography (eluting with a 5-25% ethyl acetate in dichloromethane gradient) afforded 94f (171 mg, 77%) as a pale orange foam. ¹H NMR (300 MHz, CDCl₃) δ 9.45 (s, 1H), 8.52 (s, 1H), 8.05 (s, 1H), 7.92 (d, J=3.20 Hz, 1H), 7.89 (d, J=0.75 Hz, 1H), 7.73 (dd, J=1.60, 8.76 Hz, 1H), 6.79 (d, J=3.77 Hz, 1H), 5.89 (dd, J=2.64, 8.29 Hz, 1H), 3.96 (m, 1H), 3.80 (m, 1H), 3.09 (m, 1H), 2.51 (m, 1H), 2.17 (m, 2H), 1.75 (m, 2H), 1.23 (s, 9H).

### 3-(1H-imidazol-2-yl)-5-(1H-pyrrolo[2,3-c]pyridin-4-yl)-1H-indazole (94)

To a solution of aminoacetaldehyde dimethyl acetal (158 μL, 1.45 mmol) in THF (1 mL) at −78° C. was added n-butyllithium (0.58 mL of a 2.5M solution in hexanes, 1.45 mmol) dropwise. The resulting orange solution was stirred 20 minutes. In a separate flask, tert-butyl 4-(3-cyano-1-tetrahydro-2H-pyran-2-yl-1H-indazol-5-yl)-1H-pyrrolo[2,3-c]pyridine-1-carboxylate 94f (171 mg, 0.386 mmol) was dissolved in 10 mL THF and cooled to 0° C. The lithium amide solution was added slowly and the mixture stirred 2 hours at 0° C. An additional solution of lithium amide was prepared and added to the reaction mixture which was stirred 15 minutes and quenched with a mixture of MeOH (0.5 mL) and H₂O (0.5 mL). The solvent was removed in vacuo and the residue was dissolved in HCl in dioxane (2 mL of a 4.0N solution) and H₂O (2 mL). The mixture was refluxed for 2 hours, cooled to room temperature, diluted with H₂O (10 mL), and washed was EtOAc (10 mL). The aqueous layer was basified to pH 9 with 1N NaOH and the solvent was removed in vacuo. Reverse phase prep HPLC (eluting with 5-30% CH₃CN in H₂O) afforded 94 (59 mg, 28%) as a white solid: ¹H NMR (300 MHz, CD₃OD) δ 9.09 (s, 1H), 8.61 (s, 1H), 8.43 (s, 1H), 8.28 (d, J=3.01 Hz, 1H), 7.99 (dd, J=1.31, 8.86 Hz, 1H), 7.94 (dd, J=0.75, 8.86 Hz, 1H), 7.19 (dd, J=0.75, 3.01 Hz, 1H).

Anal. Calcd for C₁₇H₁₂N₆.2(CF₃COOH).1.5(H₂O): C, 45.41; H, 3.09; F, 20.52; N, 15.13. Found: C, 45.55; H, 2.97; F, 19.17; N, 15.28.

### EXAMPLE 95

### 3-(3H-Imidazol-4-yl)-5-(1H-pyrrolo[2,3-c]pyridin-4-yl)-1H-indazole

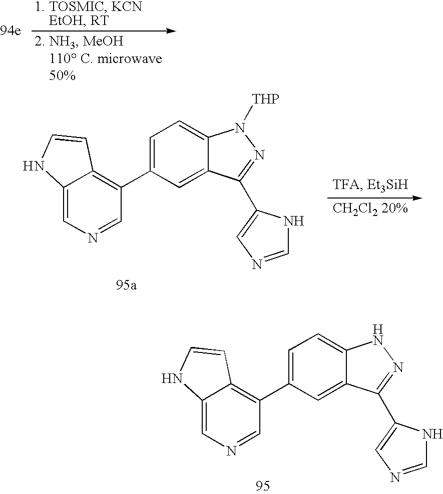

### 3-(1H-imidazol-5-yl)-5-(1H-pyrrolo[2,3-c]pyridin-4-yl)-1-(tetrahydro-2H-pyran-2-yl)-1H-indazole (95a)

The title compound was prepared as in Example 90a. tert-Butyl 4-(3-formyl-1-tetrahydro-2H-pyran-2-yl-1H-indazol-5-yl)-1H-pyrrolo[2,3-c]pyridine-1-carboxylate 94e (500 mg, 1.12 mmol), tosylmethyl isocyanide (219 mg, 1.12 mmol) and sodium cyanide (5.5 mg, 0.112 mmol) afforded 95a (230 mg, 53%) as a yellow foam. ¹H NMR (300 MHz, MeOH) δ 8.59 (s,1 H), 8.15 (s, 1 H), 7.75 (s, 1 H), 7.73 (d, J=0.94 Hz, 1 H), 7.56 (s, 1 H), 7.53 (d, J=3.20 Hz, 1 H), 6.69 (s,1 H), 5.80 (s,1 H), 5.39 (s, 1 H), 3.95 (s,1 H), 3.72-3.82 (m, 1 H), 2.53 (s, 1 H), 2.05 (s, 2 H), 1.80 (s, 1 H), 1.61 (s, 2 H).

### 3-(1H-imidazol-5-yl)-5-(1H-pyrrolo[2,3-c]pyridin-4-yl)-1H-indazole (95)

A solution of 3-(1H-imidazol-5-yl)-5-(1H-pyrrolo[2,3-c]pyridin-4-yl)-1-(tetrahydro-2H-pyran-2-yl)-1H-indazole 95a (230 mg, 0.597 mmol) and triethylsilane (0.24 mL, 1.49 mmol) in trifluoroacetic acid (2.3 mL) and CH₂Cl₂ (2.3 mL) was stirred 3 hours at room temperature. The mixture was concentrated in vacuo, neutralized with ammonia in MeOH, and concentrated. The crude material was purified by reverse phase prep HPLC (CH₃CN/H₂O with 0.1% TFA) to afford 95 (59 mg, 17%) as a white solid. ¹H NMR (300 MHz, MeOH) δ 8.98 (s, 1 H), 8.93 (d, J=0.94 Hz, 1 H), 8.35 (s, 1 H), 8.33 (s, 1 H), 8.16-8.19 (m, 2 H), 7.83 (dd, J=8.86, 1.51 Hz, 1 H), 7.78 (d, J=8.86 Hz, 1 H), 7.05 (dd, J=3.01, 0.75 Hz, 1 H).

Anal. Calcd for C₁₇H₁₂N₆.2(CF₃COOH).1.5(H₂O): C, 45.41; H, 3.09; F, 20.52; N, 15.13.

Found: C, 45.55; H, 2.97; F, 19.17; N, 15.28.

### EXAMPLE 96

### Ethyl-{4-methyl-5-[3-(2-pyridin-4-yl-3H-imidazol-4-yl)-1H-indazol-5-yl]-pyridin-3-ylmethyl}-amine

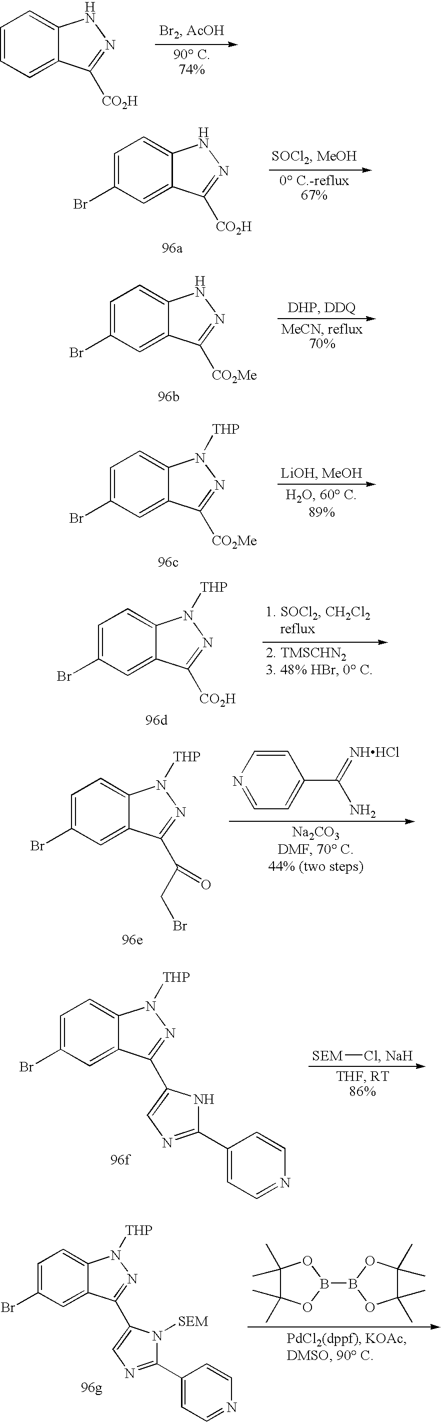

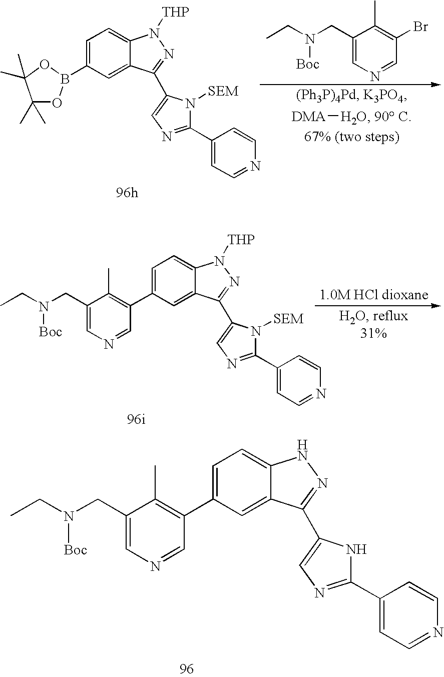

### 5-Bromo-1H-indazole-3-carboxylic acid (96a)

Indazole-3-carboxylic acid (8.57 g, 51.9 mmol) was suspended in glacial acetic acid (500 mL) in a 3-neck 1 L round-bottomed flask fitted with overhead stirrer. Upon heating to 90° C. the starting material went into solution. Bromine (5.3 mL, 104 mmol) was added in acetic acid (50 mL) via addition funnel. The orange mixture was stirred 16 h at 90° C., then cooled to 5° C. in an ice bath. The yellow precipitate was collected by vacuum filtration, washed with EtOAc and Et₂O, and dried under vacuum to afford 96a (9.24 g, 74%) as a pale yellow crystalline solid: ¹H NMR (300 MHz, DMSO-d6) δ 8.20 (d, J=1.88 Hz, 1H), 7.64 (d, J=8.86 Hz), 7.55 (dd, J=1.88, 8.86 Hz,1H).

### Methyl 5-bromo-1H-indazole-3-carboxylate (96b)

To suspension of 5-bromo-1H-indazole-3-carboxylic acid 96a (10.4 g, 43.1 mmol) in MeOH (200 mL) at 0° C. was slowly added thionyl chloride (15.7 mL, 216 mmol). The mixture was refluxed for 16 h and cooled to room temperature. Upon solvent removal a white precipitate formed and was collected by vacuum filtration. The filtrate was concentrated and the resulting precipitate was collected by vacuum filtration. The combined solids were dried under vacuum to afford 96b (7.38 g, 67%) as a white solid: ¹H NMR (300 MHz, DMSO-d6) δ 14.13 (br s, 1H), 8.20 (d, J=1.13 Hz, 1H), 7.66 (d, J=8.85 Hz), 7.56 (dd, J=1.70, 8.85 Hz), 3.92 (s, 3H).

### Methyl 5-bromo-1-tetrahydro-2H-pyran-2-yl-1H-indazole-3-carboxylate (96c)

To a solution of methyl 5-bromo-1H-indazole-3-carboxylate 96b (6.01 g, 23.6 mmol) in acetonitrile (250 mL) were added 3,4-dihydro-2H-pyran (3.22 mL, 35.3 mmol) and 2,3-dichloro-5,6-dicyano-1,4-benzoquinone (DDQ) (534 mg). The dark brown solution was refluxed 5 h, cooled to room temperature and concentrated in vacuo. Silica gel chromatography (eluting with dichloromethane) afforded 96c (5.56 g, 70%) as a yellow solid: ¹H NMR (300 MHz, DMSO-d6) δ 8.22 (d, J=1.51 Hz, 1H), 7.88 (d, J=9.04 Hz, 1H), 7.66 (dd, J=1.88, 9.04 Hz,1H), 6.01 (dd, J=2.26, 9.42 Hz), 3.93 (s, 3H), 3.86 (m,1H), 3.76 (m, 1H), 2.33 (m,1H), 2.02 (m, 2H), 1.75 (m,1H), 1.59 (m, 2H).

### 5-Bromo-1-tetrahydro-2H-pyran-2-yl-1H-indazole-3-carboxylic acid (96d)

A suspension of methyl 5-bromo-1-tetrahydro-2H-pyran-2-yl-1H-indazole-3-carboxylate 96c (2.54 g, 7.51 mmol) and LiOH.H₂O (473 mg, 11.3 mmol) in methanol (25 mL) and water (25 mL) was stirred at 60° C. for 2 h. The colorless solution was diluted with methanol (20 mL) and water (50 mL) and acidified to pH 1 with 1N HCl. The precipitate was collected by vacuum filtration and dried under high vacuum to afford 96d (2.16 g, 89%) as a white powder: ¹H NMR (300 MHz, CDCl₃) δ 8.41 (s, 1H), 7.62 (d, J=8.85 Hz, 1H), 7.54 (d, J=9.04 Hz, 1H), 5.82 (dd, J=2.07, 8.85 Hz, 1H), 3.99 (m, 1H), 3.75 (m, 1H), 2.51 (m, 2H), 2.18 (m, 2H), 1.74 (m, 2H).

### 2-Bromo-1-(5-bromo-1-tetrahydro-2H-pyran-2-yl-1H-indazol-3-yl)ethanone (96e)

Thionyl chloride (0.45 mL, 6.2 mmol) was added to a suspension of 5-bromo-1-tetrahydro-2H-pyran-2-yl-1H-indazole-3-carboxylic acid 96d (1.00 g, 3.08 mmol) in 1,2-dichloroethane (50 mL). The suspension was refluxed for 2 h, and the resulting orange solution was cooled to room temperature, concentrated in vacuo, redissolved in dichloromethane (25 mL) and hexanes (10 mL) and concentrated. The crude brown residue was dissolved in dichloromethane and cooled to 0° C. prior to the addition of (trimethylsilyl)diazomethane (4.62 mL of a 2.0M solution in hexanes, 9.24 mmol). The reaction mixture was warmed to room temperature, stirred 16 h and cooled again to 0° C. The careful addition of HBr (48%, 2 mL) was accompanied by gas evolution. After 30 min stirring the excess acid was neutralized with solid sodium carbonate. The mixture was dried over Na₂SO₄, filtered, and concentrated in vacuo to a brown foam 96e that was used without further purification: ¹H NMR (300 MHz, CDCl₃) δ 8.50 (m, 1H), 7.56 (d, J=0.75 Hz, 1H), 7.54 (d, J=1.70 Hz, 1H), 5.81 (dd, J=2.83, 8.29 Hz, 1H), 4.70 (dd, J=12.43, 19.78 Hz, 2H), 3.93 (m, 1H), 3.75 (m, 1H), 2.52 (m, 2H), 2.15 (m, 2H), 1.77 (m, 2H).

### 5-Bromo-3-(2-pyridin-4-yl-1H-imidazol-5-yl)-1-tetrahydro-2H-pyran-2-yl-1H-indazole (96f)

A solution of 2-bromo-1-(5-bromo-1-tetrahydro-2H-pyran-2-yl-1H-indazol-3-yl)ethanone 96e (1.24 g, 3.08 mmol), 4-amidinopyridine hydrochloride (507 mg, 3.22 mmol) and sodium carbonate (1.57 g, 14.8 mmol) in DMF (15 mL) was stirred 2 h at 70° C. The mixture was cooled to room temperature and partitioned between EtOAc (100 mL) and water (100 mL). The aqueous layer was acidified to pH 1 with 1N HCl; the layers were separated and the organic layer was extracted with 0.5N HCl (50 mL) and discarded. The combined aqueous layers were basified to pH 9 with 1N NaOH and extracted with EtOAc (3×100 mL). The combined organic layers were dried (Na₂SO₄), filtered, and concentrated in vacuo to afford 96f (575 mg, 44%) as a brown oil: ¹H NMR (300 MHz, CDCl₃) δ 8.70 (d, J=5.65 Hz, 2H), 8.01 (s, 2H), 7.83 (m, 1H), 7.73 (s, 1H), 7.49 (m, 2H), 5.69 (d, J=8.29 Hz, 1H), 4.08 (m, 1H), 3.77 (m, 1H), 2.54 (m, 2H), 2.09 (m, 2H), 1.77 (m, 2H).

### 5-Bromo-3-(2-pyridin-4-yl-1-{[2-(trimethylsilyl)ethoxy]methyl}-1H-imidazol-5-yl)-1-tetrahydro-2H-pyran-2-yl-1H-indazole (96g)

A solution of 5-bromo-3-(2-pyridin-4-yl-1H-imidazol-5-yl)-1-tetrahydro-2H-pyran-2-yl-1H-indazole 96f (960 mg, 2.26 mmol) in anhydrous THF (10 mL) was added dropwise to a suspension of sodium hydride (136 mg of a 60% suspension in oil, 3.39 mmol) in THF (20 mL) at 0° C. The mixture was stirred 15 min prior to the addition of 2-(trimethylsilyl)ethoxymethyl chloride (440 μL, 2.49 mmol). The reaction mixture was warmed to room temperature, stirred 30 min and diluted with EtOAc (150 mL). The organics were washed with water (100 mL) and the aqueous layer was back-extracted with EtOAc (100 mL). The combined organics were washed with 1N HCl (100 mL), saturated aqueous NaHCO₃ (100 mL) and brine; dried (Na₂SO₄); filtered; and concentrated in vacuo to afford 96g (1.08 g, 86%) as a dark reddish-brown foam: ¹H NMR (300 MHz, CDCl₃) δ 8.74 d, J=5.65 Hz, 2H), 8.61 (s, 1H), 7.93 (d, J=5.46 Hz, 2H), 7.73 (s, 1H), 7.49 (s, 2H), 5.71 (dd, J=1.98, 9.51 Hz, 1H), 5.40 (s, 2H), 4.09 (m, 1H), 3.73 (m, 3H), 2.56 (m, 2H), 2.15 (m, 2H), 0.99 (t, J=8.20 Hz, 2H), 0.85 (m, 2H), 0.01 (s, 9H).

### 3-(2-pyridin-4-yl-1-{[2-(trimethylsilyl)ethoxy]methyl}-1H-imidazol-5-yl)-1-tetrahydro-2H-pyran-2-yl-5-(4,4,5,5-tetramethyl-1,3,2-dioxaborolan-2-yl)-1H-indazole (96h)

A suspension of 5-bromo-3-(2-pyridin-4-yl-1-{[2-(trimethylsilyl)ethoxy]methyl}-1H-imidazol-5-yl)-1-tetrahydro-2H-pyran-2-yl-1H-indazole 96g (162 mg, 0.292 mmol), (bispinacolato)diboron (82 mg), 0.321 mmol), potassium acetate (143 mg, 1.46 mmol), and dichloro[1,1′-bis(diphenylphosphino)ferrocene]palladium (II) dichloromethane adduct (5.3 mg, 7.3 nmol) in DMSO (5 mL) was degassed by bubbling with N₂ for 20 min, placed in a 90° C. oil bath and stirred for 2 h. The reaction was removed from heat and diluted with EtOAc (100 mL). The organics were washed with water (50 mL) and brine, dried (Na₂SO₄), filtered, and concentrated in vacuo to afford 96 h. The crude residue was used without further purification.

### tert-Butyl ethyl({4-methyl-5-[3-(2-pyridin-4-yl-1-{[2-(trimethylsilyl)ethoxy]methyl}-1H-imidazol-5-yl)-1-tetrahydro-2H-pyran-2-yl-1H-indazol-5-yl]pyridin-3-yl}methyl)carbamate (96i)

A solution of tert-butyl 3-bromo-2-methylbenzyl(ethyl)carbamate 1b (96.1 mg, 0.292 mmol), 3-(2-pyridin-4-yl-1-{[2-(trimethylsilyl)ethoxy]methyl}-1H-imidazol-5-yl)-1-tetrahydro-2H-pyran-2-yl-5-(4,4,5,5-tetramethyl-1,3,2-dioxaborolan-2-yl)-1H-indazole 96h (176 mg, 0.292 mmol), and potassium phosphate (93.0 mg, 0.438 mmol) in H₂O (0.3 mL) and N,N-dimethylacetamide (3 mL) was degassed by bubbling with N₂ for 15 min. Tetrakis(triphenylphosphine)palladium(0) (16.9 mg, 0.0146 mmol) was added and the mixture degassed 10 min before heating to 90° C. for 2 hours. The mixture was cooled, diluted with EtOAc (20 mL) and washed with a mixture of sat. aq. NaHCO₃ (10 mL) and H₂O (10 mL). The aqueous layer was back-extracted with EtOAc (20 mL). The combined organics were extracted with 0.2N HCl (2×30 mL). The acidic aqueous layers were combined, basified to pH 10 with 1N NaOH, and extracted with EtOAc (2×30 mL). The organic layers were dried (Na₂SO₄), filtered, and concentrated in vacuo to afford 96i (142 mg, 67%) as a brown oil: ¹H NMR (300 MHz, CDCl₃) δ ppm 8.70 (d, J=5.27 Hz, 1 H), 8.46 (s,1 H), 8.35 (d, J=5.84 Hz, 1 H), 7.88 (d, J=5.27 Hz, 1 H), 7.75 (s,1 H), 7.67 (d, J=8.48 Hz, 1 H), 7.30 (d, J=8.67 Hz, 1 H), 5.79 (d, J=8.29 Hz, 1 H), 5.39 (s, 2 H), 4.53 (s, 2 H), 4.04 (m, 2H), 3.78 (m, 2H), 3.71 (t, J=8.01 Hz, 2 H), 2.63 (m, 2H), 2.22 (s, 3 H), 2.10 (m, 2H), 1.22 (s, 9 H), 1.10 (t, J=6.88 Hz, 3 H), 1.05-0.89 (m, 4H), 0.00 (s, 9 H).

### N-({4-methyl-5-[3-(2-pyridin-4-yl-1H-imidazol-5-yl)-1H-indazol-5-yl]pyridin-3-yl}methyl)ethanamine (96)

A solution of tert-butyl ethyl({4-methyl-5-[3-(2-pyridin-4-yl-1-{[2-(trimethylsilyl)ethoxy]methyl}-1H-imidazol-5-yl)-1-tetrahydro-2H-pyran-2-yl-1H-indazol-5-yl]pyridin-3-yl}methyl)carbamate 96i (142 mg, 0.196 mmol) in H₂O (1.5 mL) and HCl in dioxane (1.5 mL of a 4N solution) was heated to reflux for 3 hours. The mixture was removed from heat, diluted with H₂O (20 mL) and washed with EtOAc (10 mL). The organic layer was discarded and the aqueous layer was basified to pH 9 with 1N NaOH and extracted with a solution of 10% isopropanol in chloroform (5×10 mL). The combined organics were dried (Na₂SO₄), filtered, and concentrated in vacuo. Silica gel chromatography (eluting with 10% 7M NH3/MeOH in dichloromethane) afforded 96 (24 mg, 31%) as a yellow solid: ¹H NMR (300 MHz, CD₃OD) δ 8.61 (d, J=5.46 Hz, 2H), 8.47 (s, 1H), 8.39 (s, 1H), 7.99 (d, J=5.84 Hz, 2H), 7.82 (s, 1H), 7.67 (d, J=8.67 Hz, 1H), 7.41 (d, J=8.67 Hz, 1H), 3.95 (s, 2H), 2.82 (q, J=7.03 Hz, 2), 2.37 (s, 3H), 1.22 (t, J=7.16 Hz, 3H).

### EXAMPLE 97

### 5-(5-Ethylaminomethyl-4-methyl-pyridin-3-yl)-1H-indazole-3-carboxylic acid amide

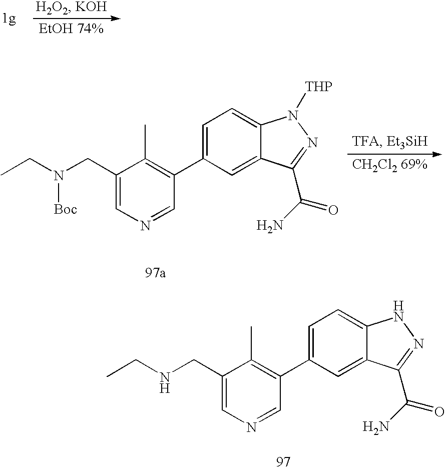

### {5-[3-Carbamoyl-1-(tetrahydro-pyran-2-yl)-1H-indazol-5-yl]-4-methyl-pyridin-3-ylmethyl}-ethyl-carbamic acid tert-butyl ester (97a)

To a clear yellow solution of 1 g (0.434 g, 0.914 mmol) in absolute 10 mL EtOH was added KOH (0.077 g, 1.371 mmol), followed by the dropwise addition of 50% H₂O₂ (0.87 ml, 1.026 g, 30.162 mmol) at rt. After the evolution of gas stopped the reaction mixture was heated at 60-70° C. for 45 mins. Upon completion of reaction as indicated by TLC, the reaction mixture was partitioned between EtOAc and H₂O. The organic extracts were dried over MgSO₄ and concentrated. The crude compound was purified by silica gel chromatography eluting with 0% to 3% MeOH in CHCl₃, yielding 97a (0.340 g, 76%) as a white solid: ¹H NMR (DMSO-d6) δ8.34 (s, 1H), 8.29 (s, 1H), 8.07 (s, 1H), 7.91 (d, J=6.0 Hz, 1H), 7.77 (s, 1H), 7.49 (s, 1H), 7.44 (d, J=6.0 Hz, 1H), 5.99 (d, J=6.0 Hz, 1H), 4.50 (s, 2H), 3.91-3.94 (m, 1H), 3.76-3.82 (m, 1H), 3.20 (q, J=6.0 Hz, 2H), 2.49-2.54 (m, 1H), 2.15 (s, 3H), 1.98-2.09 (m, 2H), 1.73-1.85 (m, 1H), 1.61 (s, 2H), 1.40 (s, 9H), 1.02 (t, J=6.0 Hz, 3H); M+H⁺ 494.

### 5-(5-Ethylaminomethyl-4-methyl-pyridin-3-yl)-1H-indazole-3-carboxylic acid amide (97)

In the same manner as the deprotection of 2c, intermediate 97a (0.340 g, 0.690 mmol) was converted to the title compound 97 (0.148 g, 69%), as a white solid: ¹H NMR (DMSO-d₆) δ 13.64 (br s, 1H), 8.43 (s, 1H), 8.28 (s, 1H), 8.06 (s, 1H), 7.78 (s, 1H), 7.69 (d, J=6.0 Hz, 1H), 7.38 (s, 1H), 7.37 (d, J=6.0 Hz, 1H), 3.75 (s, 2H), 2.60 (q, J=6.0 Hz, 2H), 2.22 (s, 3H), 1.06 (t, J=6.0 Hz, 3H). HRMS [M+H]⁺ calcd. 310.1663; found 310.1669. Anal. (C₁₇H₁₉N₅O 1.0 H₂O) C, H, N.

### EXAMPLE 98

### Ethyl-{4-methyl-5-[3-(4H-[1,2,4]triazol-3-yl)-1H-indazol-5-yl]-pyridin-3-ylmethyl}-amine

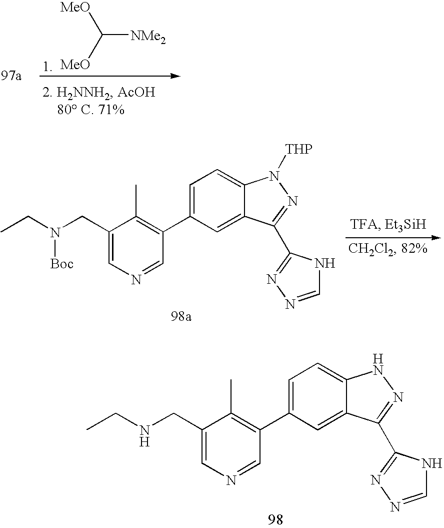

### Ethyl-{4-methyl-5-[1-(tetrahydro-pyran-2-yl)-3-(4H-[1,2,4]triazol-3-yl)-1H-indazol-5-yl]-pyridin-3-ylmethyl}-3-ylmethyl}-carbamic acid tert-butyl ester (98a)

A mixture of the amide 97a (0.36 g, 0.73 mmol) and DMF dimethyl acetal (5 mL) was stirred at 80° C. for 1 hr. The mixture was concentrated to dryness and the residue dissolved in acetic acid (5 mL). To the resulting solution was added hydrazine monohydrate(0.5 mL) and stirred at RT for 22 hr. The mixture was added dropwise to water (300 mL) and the resulting white precipitate was collected by filtration and washed with water. The solids were dissolved in ethyl acetate, dried (MgSO₄), and concentrated to dryness. The oily residue was taken up in acetonitrile and concentrated to dryness to yield the title compound as a crisp foam 98a (0.268 g, 71%) which was carded forward with out further purification: 1H NMR (400 MHz, ACETONITRILE-D3) δ ppm 8.38 (s, 1 H), 8.32 (s,1 H), 8.28 (dd, J=1.52, 0.76 Hz, 1 H), 8.25 (bs, 1 H), 7.81 (d, J=8.59 Hz, 1 H), 7.45 (dd, J=8.72, 1.64 Hz, 1 H), 5.91 (dd, J=9.73, 2.40 Hz, 1 H), 4.54 (s, 2 H), 3.92-4.05 (m, 1 H), 3.75-3.88 (m, 1 H), 3.26 (q, J=6.48 Hz, 2 H), 2.49-2.65 (m, 1 H), 2.20 (s, 3 H), 1.61-1.90 (m, 3 H), 1.44 (s, 9 H), 1.07 (t, J=7.07 Hz, 3 H).

### Ethyl-{4-methyl-5-[3-(4H-[1,2,4]triazol-3-yl)-1H-indazol-5-yl]-pyridin-3-ylmethyl}-amine (98)

To a solution of 98a, (0.26 g, 0.51 mmol) and triethylsilane (0.2 mL) in dichloromethane (9 mL) was added trifluoroacetic acid (1 mL) at RT. After stirring at RT for 67 hours, the mixture was diluted with toluene and concentrated to dryness. The resulting residue was triturated with TBME and collected by filtration to yield the title compound 98 (0.32 g, 82%) as the 3.5 TFA salt: 1H NMR (400 MHz, ACETONITRILE-D3) δ ppm 8.83 (s,1 H), 8.51 (s, 1 H), 8.24 (s, 1 H), 8.01 (s, 1 H), 7.65 (d, J=8.59 Hz, 1 H), 7.26 (dd, J=8.46, 1.39 Hz, 1 H), 4.43 (s, 2 H), 3.40 (q, J=7.33 Hz, 2 H), 2.24 (s, 3 H), 1.44 (t, J=7.33 Hz, 3 H).

Anal. Calcd for C₁₈H₁₉N₇.0.9 H₂O.3.5 TFA: C: 40.51; H: 3.40; N: 12.97. Found: C: 40.27; H: 3.01; N: 12.67.

### EXAMPLES 99-270

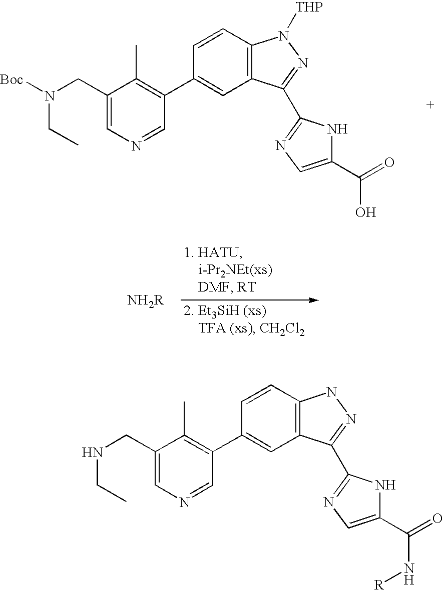

### Examples 99-270 were prepared in a library format by acylation of R-amines with 2-[5-{5-[ethyl-t-butoxy carbonylamino)-methyl]-4-methyl-pyridin-3-yl}-1-(tetrahydro-pyran-2-yl)-1H-indazole-3-yl]-3H-imidazole-4-carboxylic acid followed by deprotection

To a solution of the amines in DMF (1.0 equiv.), was added a solution of the carboxylic acid in a 10% v/v mixture of DIPEA in DMF (1.0 equiv.) and HATU in DMF (1.0 equiv.). The reactions were stirred at RT for 16 h. The solvents were removed, and an excess of CH₂Cl₂, TFA, and triethylsilane were added. The reaction mixtures were allowed to stir overnight at RT. The solvents were removed, and the residues were reconstituted in DMSO. The identity of the compounds were confirmed by low resolution mass spectrometry.

| Example | Structure | Name | MW | MW Found | % Inhib @ 10 nM |
|---|---|---|---|---|---|
| 99 | 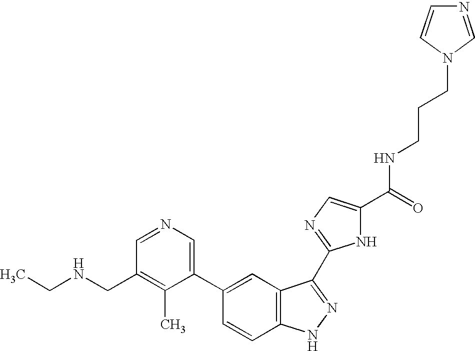 | 2-(5-{5-[(ethylamino)methy]-4- methylpyridin-3-yl}-1H-indazol-3-yl)-N- [3-(1H-imidazol-1-yL)propyl]-1H- imidazole-5-carboxamide | 483.58 | 484.58 | 39 |
| 100 | 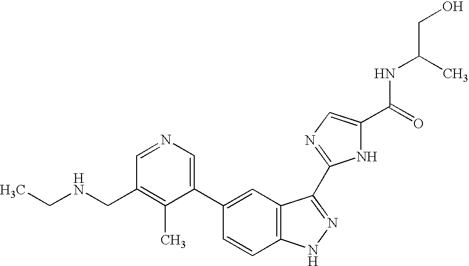 | 2-(5-{5-[(ethylamino)methyl]-4- ethylpyridin-3-yl}-1H-indazol-3-yl)-N- (2-hydroxy-1-methylethyl)-1H- imidazole-5-carboxamide | 433.51 | 434.51 | 65 |
| 101 | 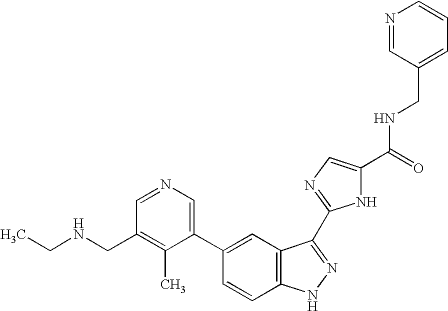 | 2-(5-{5-[(ethylamino)methyl]-4- ethylpyridin-3-yl}-1H-indazol-3-yl)-N- (pyridin-3-ylmethyl)-1H-imidazole-5- carboxamide | 466.55 | 467.55 | 54 |
| 102 | 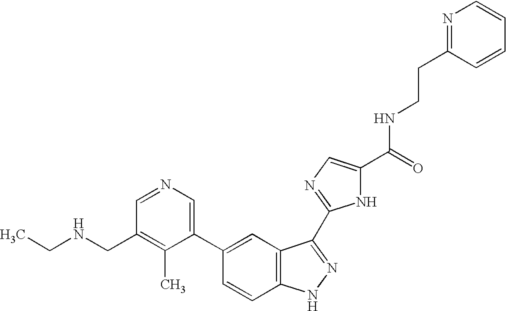 | 2-(5-{5-[(ethylamino)methyl]-4- ethylpyridin-3-yl}-1H-indazol-3-yl)-N- (2-pyridin-2-ylethyl)-1 H-imidazole-5- carboxamide | 480.57 | 481.57 | 81 |
| 103 | 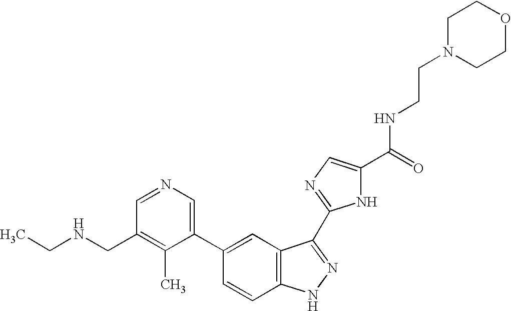 | 2-(5-{5-[(ethylamino)methyl]-4- methylpyridin-3-yl}-1H-indazol-3-yl)-N- (2-morpholin-4-ylethyl)-1H-imidazole- 5-carboxamide | 488.59 | 489.59 | 40 |
| 104 | 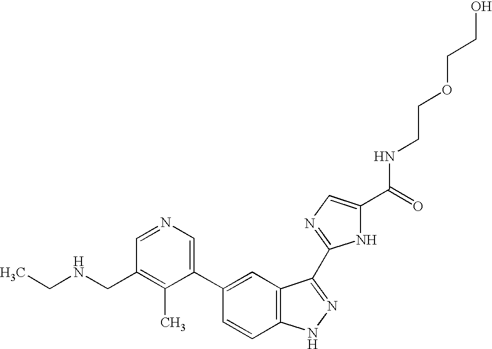 | 2-(5-{5-[(ethylamino)methyl]-4- ethylpyridin-3-yl}-1H-indazol-3-yl)-N- [2-(2-hydroxyethoxy)ethyl]-1H- imidazole-5-carboxamide | 463.54 | 464.54 | 54 |
| 105 | 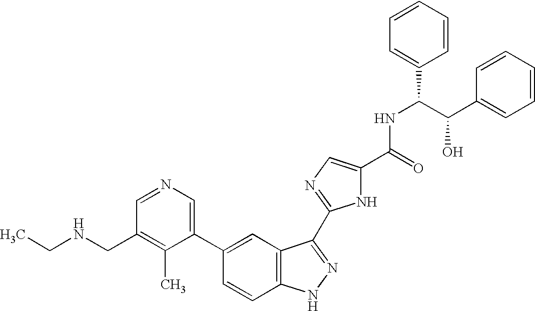 | 2-(5-{5-[(ethylamino)methyl]-4- methylpyridin-3-yl}-1H-indazol-3-yl)-N- [(1R,2S)-2-hydroxy-1,2-diphenylethyl]- 1H-imidazole-5-carboxamide | 571.68 | 572.68 | 74 |
| 106 | 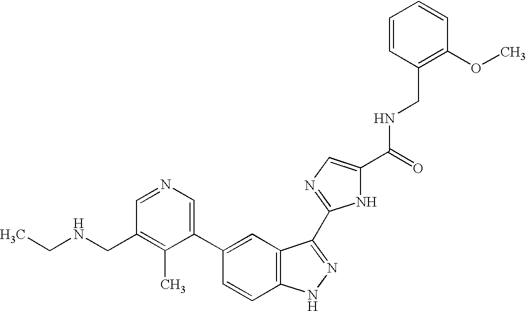 | 2-(5-{5-[(ethylamino)methyl]-4- ethylpyndin-3-yl}-1H-indazol-3-yl)-N- (2-methoxybenzyl)-1H-imidazole-5- carboxamide | 495.58 | 496.58 | 78 |
| 107 | 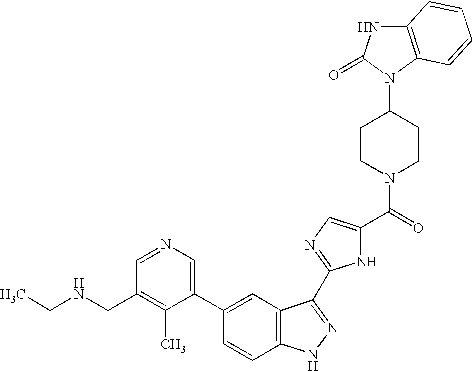 | 1-(1-{[2-(5-{5-[(ethylamino)methyl]-4- methylpyridin-3-yl}-1H-indazal-3-yl)- 1H-imidazol-5-yl]carbonyl}piperidin-4- 1,3-dihydro-2H-benzimidazol-2-one | 575.67 | 576.67 | 59 |
| 108 | 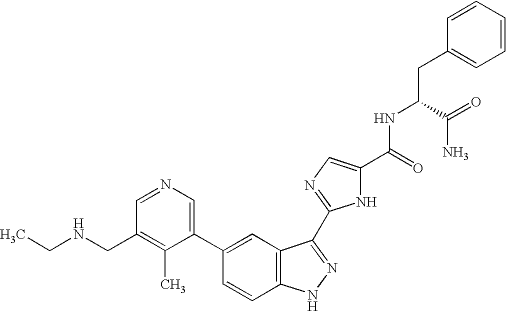 | N-{[2-(5-{5-[(ethylamino)methyl]-4- methylpyridin-3-yl}-1H-indazol-3-yl)- 1H-imidazol-5-yl]carbonyl}- D- phenylalaninamide | 522.61 | 523.61 | 57 |
| 109 | 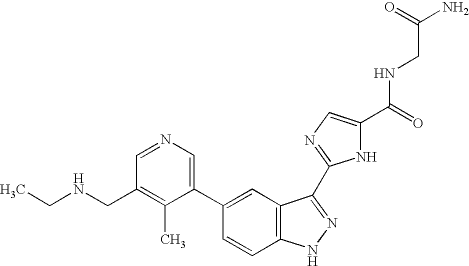 | N-(2-amino-2-oxoethyl)-2-(5-{5- [(ethylamino)methyl]-4-methylpyridin- 3-yl}-1H-indazol-3-yl)-1H-imidazole-5- carboxamide | 432.49 | 433.49 | 69 |
| 110 | 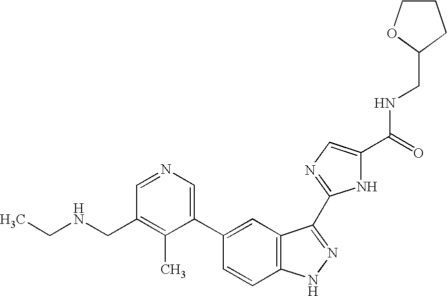 | 2-(5-{5-[(ethylamino)methyl]-4- methylpyridin-3-yl}-1H-indazol-3-yl)-N- (tetrahydrofuran-2-ylmethyl)-1H- imidazole-5-carboxamide | 459.55 | 460.55 | 66 |
| 111 | 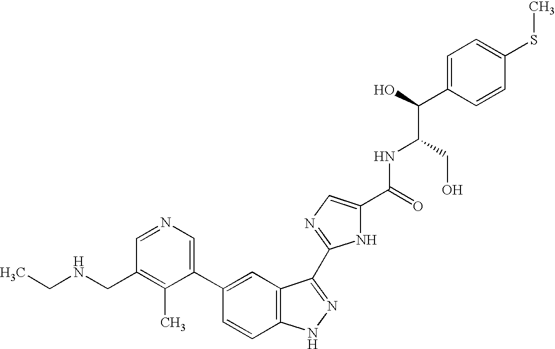 | 2-(5-{5-[(ethylamino)methyl]-4- methylpyridin-3-yl}-1H-indazol-3-yl)-N- {(1S,2S)-2-hydroxy-1- (hydroxymethyl)-2-[4- (methylthio)phenyl]ethyl}-1H- imidazole-5-carboxamide | 571.70 | 572.70 | 73 |
| 112 | 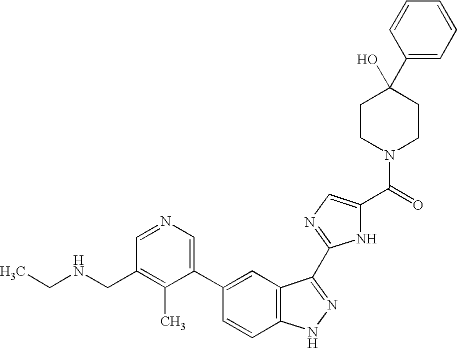 | 1-{[2-(5-{5-[(ethylamino)methyl]-4- methylpyridin-3-yl}-1H-indazol-3-yl)- 1H-imidazol-5-yl]carbonyl}-4- phenylpiperidin-4-ol | 535.65 | 536.65 | 80 |
| 113 | 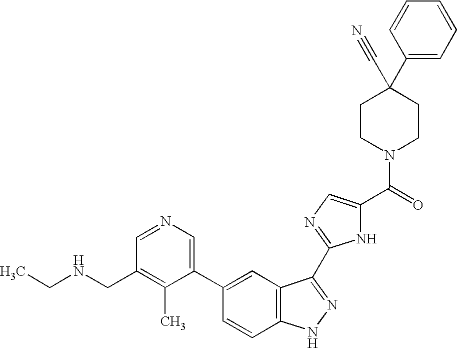 | 1-{[2-(5- {5-[(ethylamino)methyl]-4- methylpyridin-3-yl}-1H-indazol-3-yl)- 1H-imidazol-5-yl]carbonyl}-4- phenylpiperidine-4-carbonitrile | 544.66 | 545.66 | 61 |
| 114 | 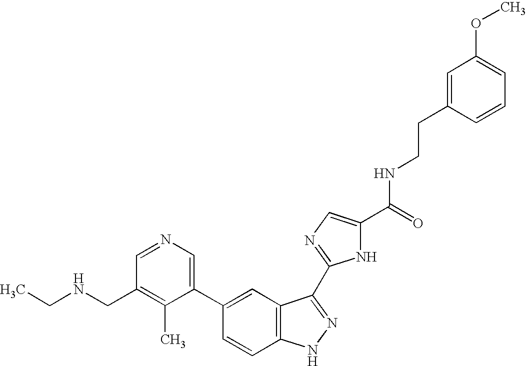 | 2-(5-{5-[(ethylamino)methyl]-4- methylpyridin-3-yl}-1H-indazol-3-yl)-N- [2-(3-methoxyphenyl)ethyl]-1H- imidazole-5-carboxamide | 509.61 | 510.61 | 64 |
| 115 | 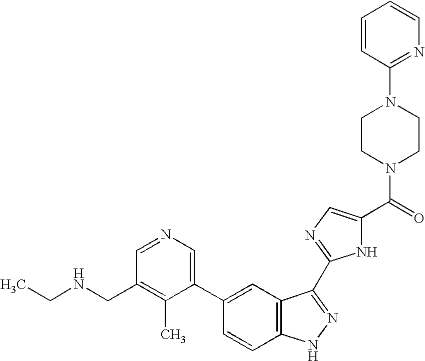 | ethyl{[4-methyl-5-(3-{5-[(4-pyridin-2- piperazin-1-yl)carbonyl]-1H-imidazol- yl}-1H-indazol-5-yl)pyridin-3- yl]methyl}amine | 521.63 | 522.63 | 72 |
| 116 | 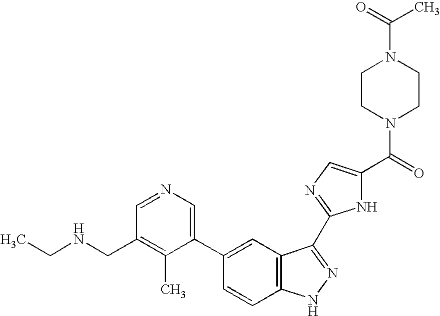 | 5-(3-{5-[(4-acetylpiperazin-1- carbonyl]-1H-imidazol-2-yl}-1H- indazol-5-yl)-4-methylpyridin-3- yl]methyl}ethylamine | 486.58 | 487.58 | 66 |
| 117 | 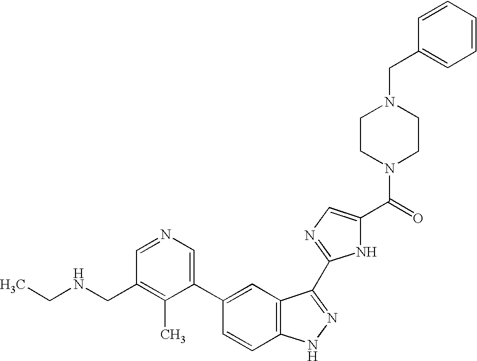 | {(5-(3-{5-[(4-benzylpiperazin-1- carbonyl]-1H-imidazol-2-yl}-1H- indazol-5-yl)-4-methylpyridin-3- yl]methyl}ethylamine | 534.66 | 535.66 | 72 |
| 118 | 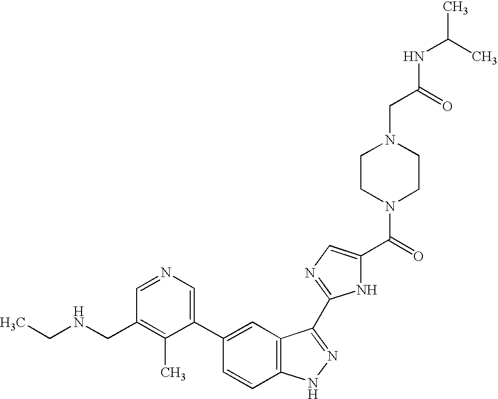 | 2-(4-{[2-(5-{5-[(ethylamino)methyl]-4- methylpyridin-3-yl}-1H-indazol-3-yl)- 1H-imidazol-5-yl]carbonyl}piperazin-1- yl)-N-isopropylacetamide | 543.67 | 544.67 | 44 |
| 119 | 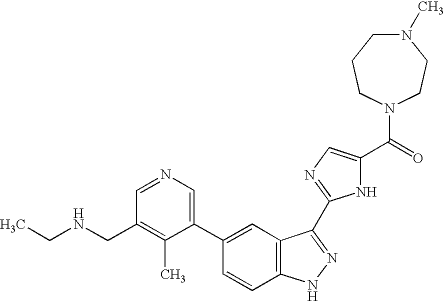 | ethyl{(4-methyl-5-(3-{5-[(4-methyl-1,4- diazepan-1-yl)carbonyl]-1H-imidazol- 2-yl}-1H-indazol-5-yl)pyridin-3- yl]methyl}amine | 472.59 | 473.59 | 38 |
| 120 | 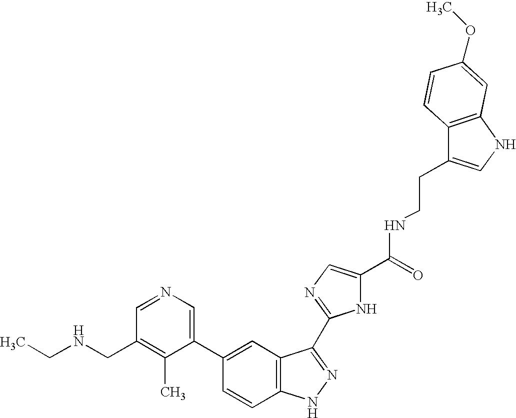 | 2-(5-{5-[(ethylamino)methyl]-4- methylpyridin-3-yl}-1H-indazol-3-yl)-N- 2-(6-methoxy-1H-indol-3-yl)ethyl]-1H- midazole-5-carboxamide | 548.65 | 549.65 | 45 |
| 121 | 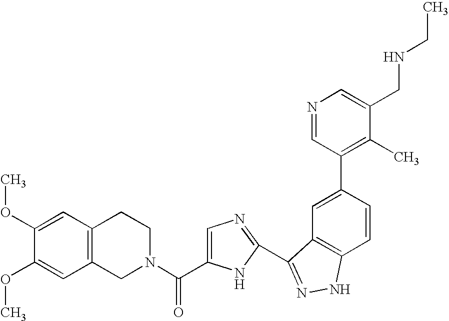 | {[5-(3-{5-[(6,7-dimethoxy-3,4- dihydroisoquinolin-2(1H)-yl)carbonyl}- 1H-imidazol-2-yl}-1H-indazol-5-yl)-4- methylpyridin-3-yl]methyl)ethylamine | 551.65 | 552.65 | 69 |
| 122 | 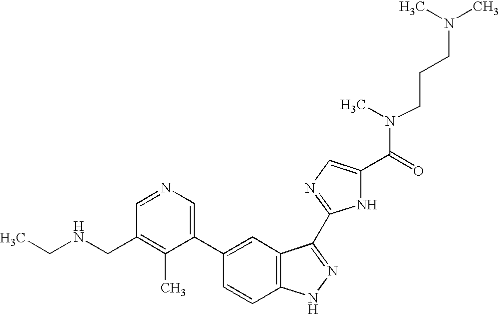 | N-[3-dimethylamino)propyl]-2-(5-{5- [(ethylamino)methyl]-4-methylpyridin- 3-yl}-1H-indazol-3-yl)-N-methy-1H- imidazole-5-carboxamide | 474.61 | 475.61 | 30 |
| 123 | 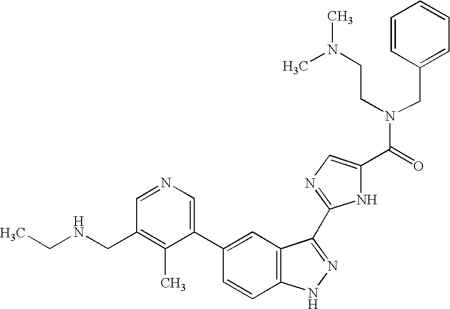 | N-benzyl-N-[2-(dimethylamino)ethyl]- 2-(5-{5-[(ethylamino)methyl]-4- methylpyridin-3-yl}-1H-indazol-3-yl)- 1H-imidazale-5-carboxamide | 536.68 | 537.68 | 54 |
| 124 | 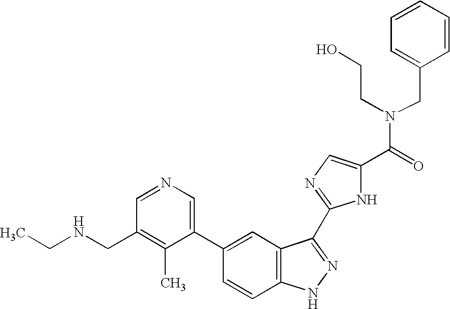 | N-benzyl-2-(5-{5-[(ethylamino)methyl]- 4-methylpyridin-3-yl}-1H-indazol-3-yl)- N-(2-hydroxyethyl)-1H-imidazole-5- carboxamide | 509.61 | 510.61 | 56 |
| 125 | 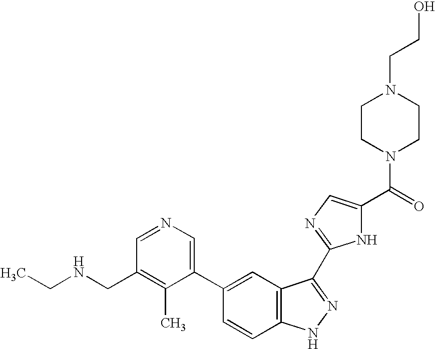 | 2-(4-{[2-(5-{5-[(ethylamino)methyl]-4- methylpyridin-3-yl}-1H-indazol-3-yl)- 1H-imidazol-5-yl]carbonyl}piperazin-1- yl)ethanol | 488.59 | 489.59 | 40 |
| 126 | 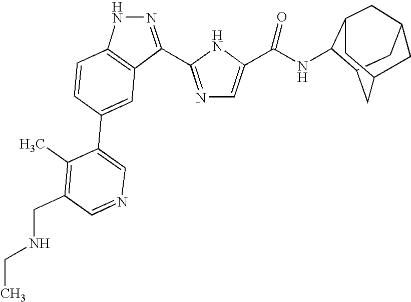 | N-2-adamantyl-2-(5-{5- [(ethylamino)methyl]-4-methylpyridin- 3-yl}-1H-indazol-3-yl)-1H-imidazole-5- carboxamide | 509.65 | 510.65 | 89 |
| 127 | 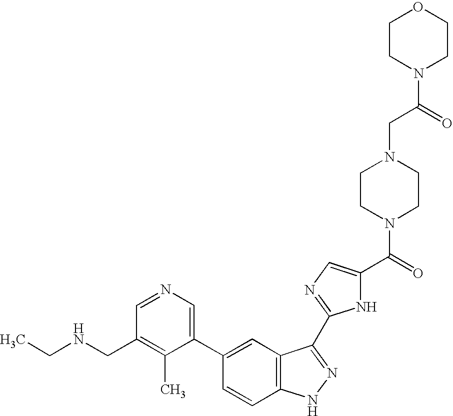 | ethyl({4-methyl-5-[3-(5-{[4-(2- morpholin-4-yl-2-oxoethyl)piperazin-1- yl]carbonyl}-1H-imidazol-2-yl)-1H- indazol-5-yl]pyridin-3-yl}methyl)amine | 571.68 | 572.68 | 26 |
| 128 | 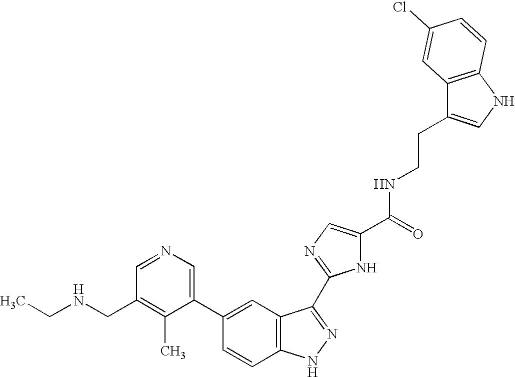 | N-[2-(5-chloro-1H-indol-3-YL)methyl]2- (5-{5-[(ethylamino)methyl]-4- methylpyridin-3-yl}-1H-indazol-3-yl)- 1H-imidazole-5-carboxamide | 553.07 | 554.07 | 65 |
| 129 | 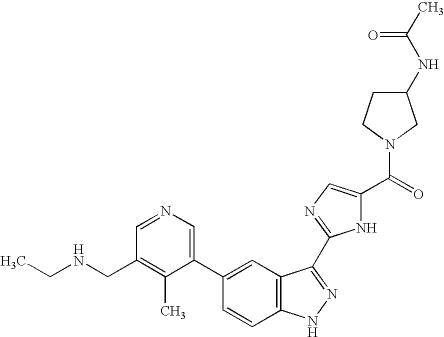 | N-(1-{[2-(5 {5- [(ethylamino)methyl]-4- ethylpyridin-3-yl}-1H-indazol-3-yl)- 1H-imidazol-5-yl]carbonyl)pyrrolidin-3- yl)acetamide | 486.58 | 487.58 | 55 |
| 130 | 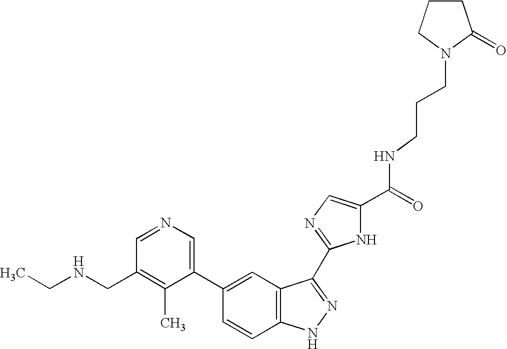 | 2-(5-{5-[(ethylamino)methyl]-4- methylpyridin-3-yl}-1H-indazol-3-yl)-N- 3-(2-oxapyrrolidin-1-yl)propyl]-1H- imidazole-5-carboxamide | 500.60 | 501.60 | 28 |
| 131 | 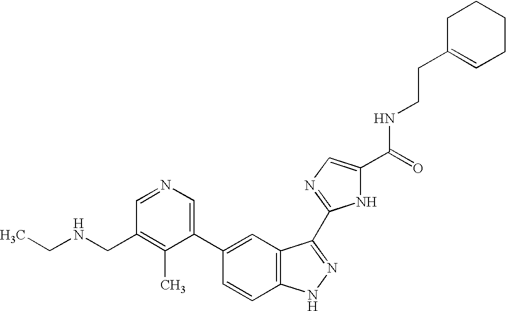 | N-(2-cyclohex-1-en-1-ylethyl)-2-(5-{5- (ethylamino)methyl]-4-methylpyridin- 3-yl}-1H-indazol-3-yl)-1H-imidazole-5- carboxamide | 483.62 | 484.62 | 57 |
| 132 | 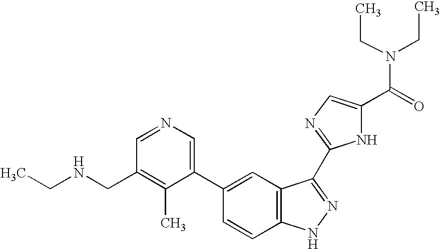 | N,N-diethyl-2-(5-{5- [(ethylamino)methyl]-4-methylpyridin- 3-yl}-1H-indazol-3-yl)-1H-imidazxole-5- carboxamide | 431.54 | 432.54 | 80 |
| 133 | 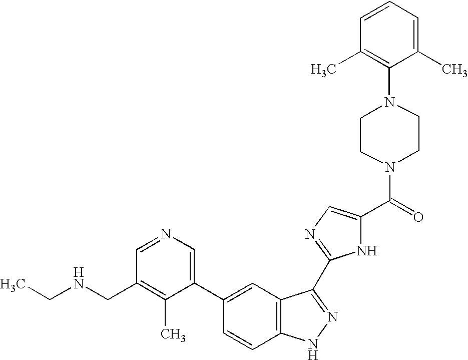 | ({5-[3-(5-{[4-(2,6- dimethylphenyl)piperazin-1- indazol-5-yl-4-methylpyridin-3- yl]methyl)ethylamine | 548.69 | 549.69 | 67 |
| 134 | 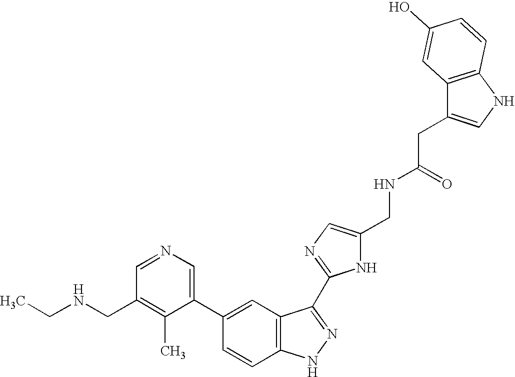 | 2-(5-{5-[(ethylamino)methyl]-4- methylpyridin-3-yl}-1H-indazol-3-yl)-N- [2-(5-hydroxy-1H-indol-3-yl)ethyl]-1- imidazole-5-carboxamide | 534.62 | 535.62 | 38 |
| 135 | 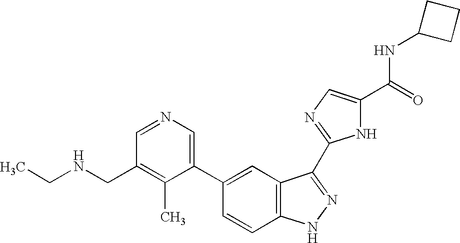 | N-cyclobutyl-2-(5-{5- [(ethylamino)methyl]-4-methylpyridin- 3-yl}-1H-indazol-3-yl)-1H-imidazole-5- carboxamide | 429.53 | 430.53 | 67 |
| 136 | 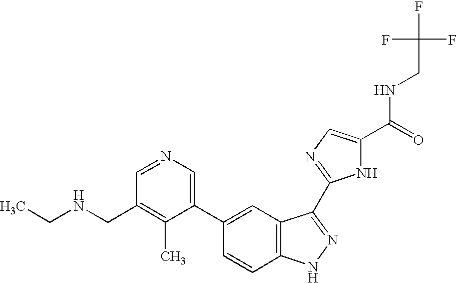 | 2-(5-{5-[(ethylamino)methyl]-4- methylpyridin-3-yl}-1H-indazol-3-yl)-N- (2,2,2-trifluoroethyl)-1H-imidazole-5- carboxamide | 457.46 | 458.46 | 72 |
| 137 | 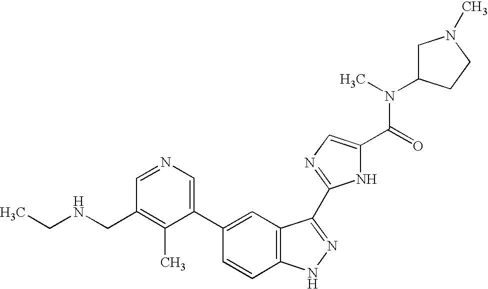 | 2-(5-{5-[(ethylamino)methyl]-4- methylpyridin-3-yl}-1H-indazol-3-yl)-N- methyl-N-(1-methylpyrrolidin-3-yl)-1H- imidazole-5-carboxamide | 472.59 | 473.59 | 37 |
| 138 | 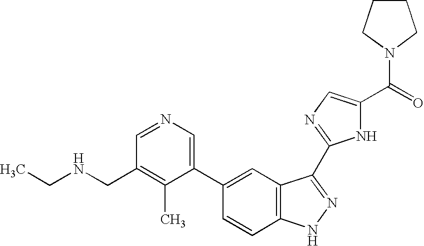 | ethyl[(4-methyl-5-{3-[5-(pyrrolidin-1- ylcarbonyl)-1H-imidazol-2-yl]-1H- indazol-5-yl}pyridin-3-yl)methyl]amine | 429.53 | 430.53 | 79 |
| 139 | 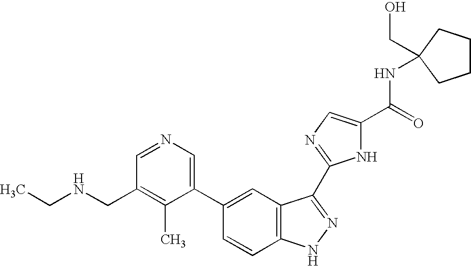 | 2-(5-{5-[(ethylamino)methyl]-4- methylpyridin-3-yl}-1H-indazol-3-yl)-N- [1-(hydroxymethyl)cyclopentyl]-1H- imidazole-5-carboxamide | 473.58 | 474.58 | 64 |
| 140 | 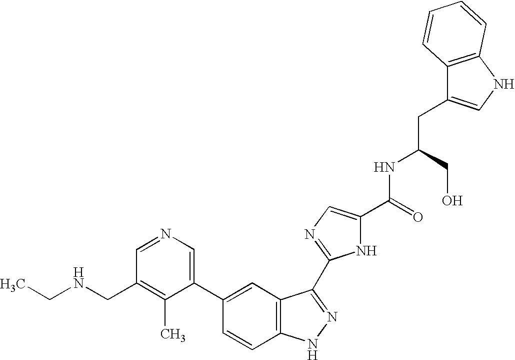 | 2-(5-{5-[(ethylamino)methyl]-4- methylpyridin-3-yl}-1H-indazol-3-yl)-N- [(1S)-2-hydroxy-1-(1H-indol-3- ylmethyl)ethyl]-1 H-imidazole-5- carboxamide | 548.65 | 549.65 | 31 |
| 141 | 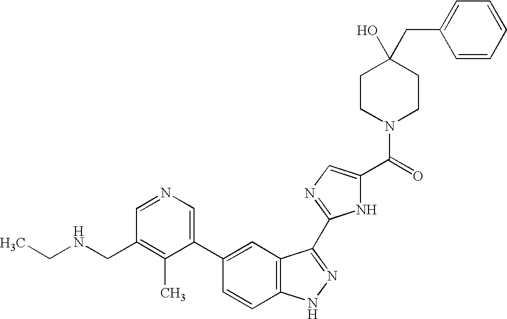 | 4-benzyl-1-{[2-(5-{5- [(ethylamino)methyl]-4-methylpyridin- 3-yl}-1H-indazol-3-yl)-1H-imidazol-5- yl]carbonyl}pipendin-4-ol | 549.68 | 550.68 | 71 |
| 142 | 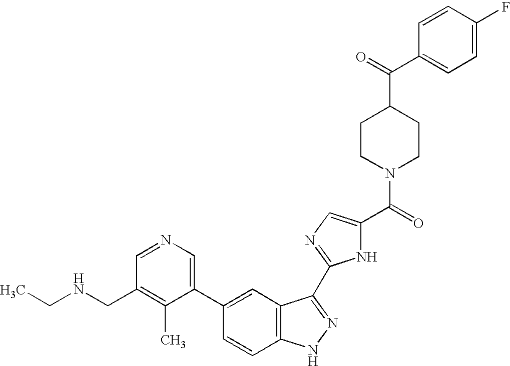 | (1-{[2-(5-{5-[(ethylamino)methyl]-4- methylpyridin-3-yl}-1H-indazol-3-yl)- 1H-imidazol-5-yl]carbonyl}piperidin-4- yl)(4-fluorophenyl)methanone | 565.65 | 566.65 | 65 |
| 143 | 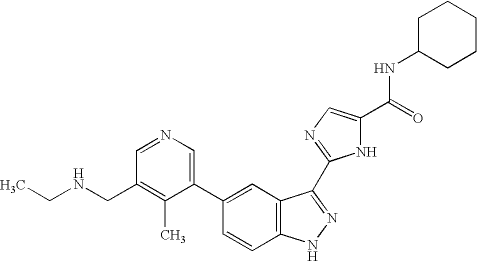 | N-cyclohexyl-2-(5-{5- [(ethylamino)methyl]-4-methylpyridin- 3-yl}-1H-indazol-3-yl)-1H-imidazole-5- carboxamide | 457.58 | 458.58 | 78 |
| 144 | 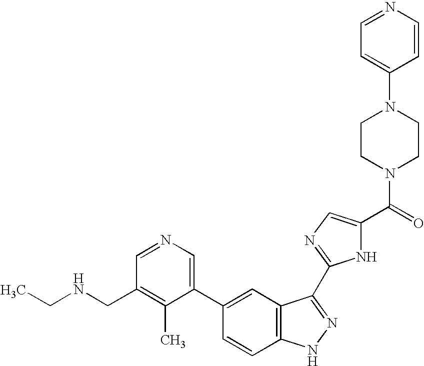 | ethyl{[4-methyl-5-(3-{5-[(4-pyridin-4- ylpiperazin-1-yl)carbonyl]-1H-imidazol- 2-yl}-1H-indazol-5-yl)pyridin-3- yl]methyl}amine | 521.63 | 522.63 | 30 |
| 145 | 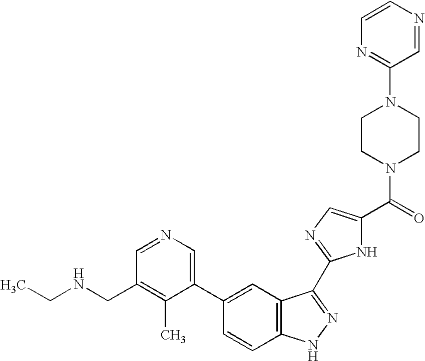 | ethyl{(4-methyl-5-(3-{5-[(4-pyrazin-2- ylpiperazin-1-yl)carbonyl]-1H-imidazol- 2-yl}-1H-indazol-5-yl)pyridin-3- ylmethyl}amine | 522.61 | 523.61 | 58 |
| 146 | 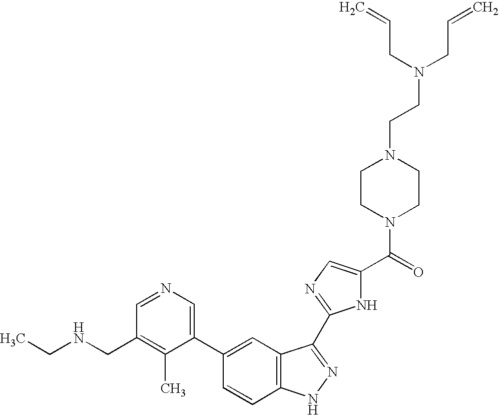 | diallyl[2-(4-{[2-(5-{5- [(ethylamino)methyl]-4.-methylpyridin- 3-yl}-1H-indazol-3-yl)-1H-imidazol-5- yl]carbonyl}piperazin-1-yl)ethyl]amine | 567.74 | 568.74 | 22 |
| 147 | 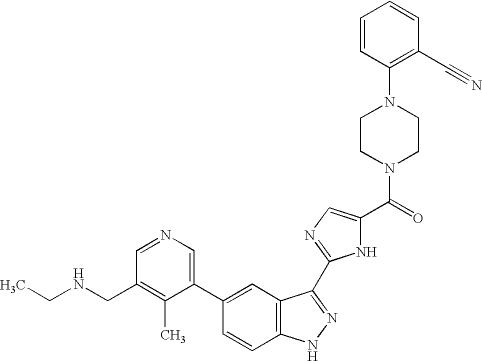 | 2-(4-{[2-(5-{5-[(ethylamino)methyl]-4- methylpyridin-3-yl}-1H-indazol-3-yl)- 1H-imidazol-5-yl]carbonyllpiperazin-1- yl)benzonitrile | 545.65 | 546.65 | 69 |
| 148 | 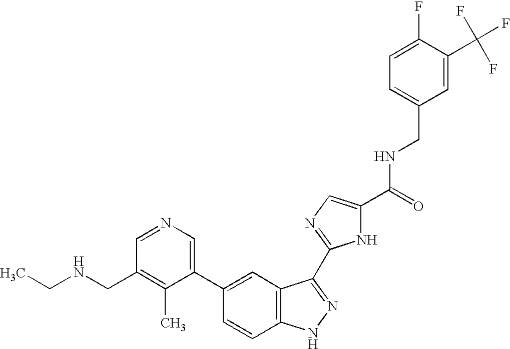 | 2-(5-{5-[(ethylamino)methyl]-4- methylpyridin-3-yl}-1H-indazol-3-yl)-N- 4-fluoro-3-(trifluoromethyl)benzyl]-1H- imidazole-5-carboxamide | 551.55 | 552.55 | 60 |
| 149 | 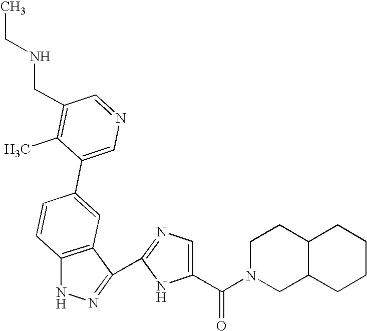 | ethyl[(4-methyl-5-{3-[5- (octahydroisoquinolin-2(1H)- ylcarbonyl)-1H-imidazol-2-yl]-1H- indazol-5-yl}pyridin-3-yl)methyl]amine | 497.64 | 498.64 | 69 |
| 150 | 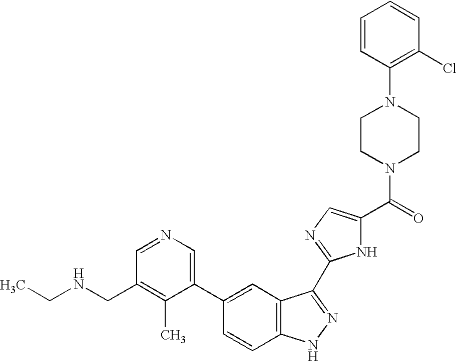 | ({5-[3-(5-{[4-(2-chlorophenyl)piperazin- 1-yl]carbonyl)-1H-imidazol-2-yl)-1H- indazol-5-yl]-4-methylpyridin-3- yl}methyl)ethylamine | 555.08 | 556.08 | 79 |
| 151 | 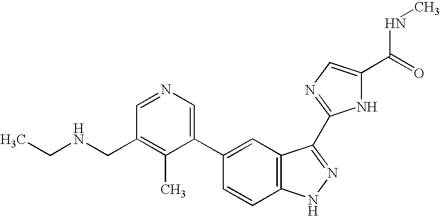 | 2-(5-{5-[(ethylamino)methyl]-4- methylpyridin-3-yl}-1H-indazol-3-yl)-N- methyl-1H-imidazole-5-carboxamide | 389.46 | 390.46 | 67 |
| 152 | 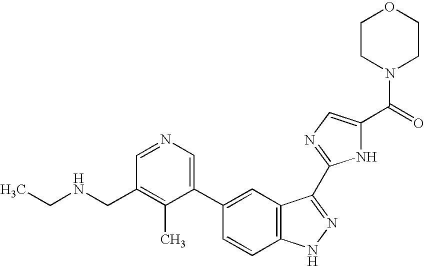 | ethyl[(4-methyl-5-{3-[5-(morpholin-4- ylcarbonyl)-1H-imidazol-2-yl]-1H- indazol-5-yl}pyridin-3-yl)methyl]amine | 445.52 | 446.52 | 67 |
| 153 | 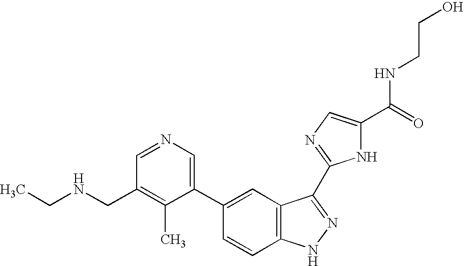 | 2-(5-{5-[(ethylamino)methyl]-4- methylpyridin-3-yl}-1H-indazol-3-yl)-N- (2-hydroxyethyl)-1H-imidazole-5- carboxamide | 419.49 | 420.49 | 70 |
| 154 | 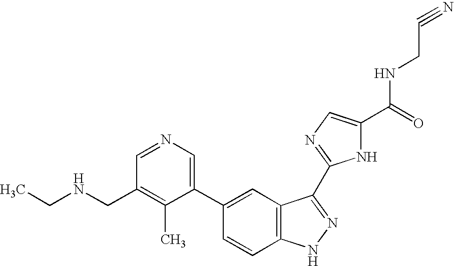 | N-(2-cyanoethyl)-2-(5-{5- [(ethylamino)methyl]-4-methylpyridin- 3-yl}-1H-indazxol-3-yl)-1H-imidazole-5- carboxamide | 428.50 | 429.50 | 71 |
| 155 | 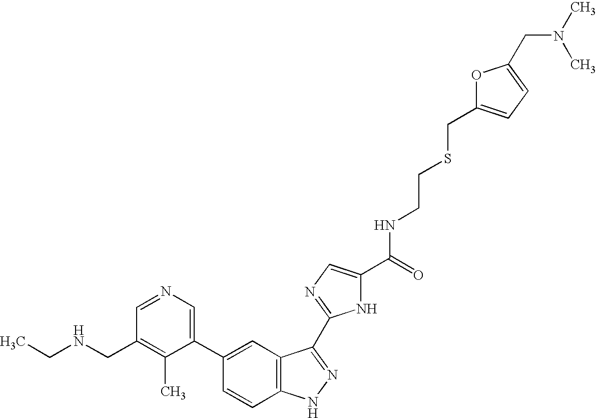 | N-{2-[({5-[(dimethylamino)methyl]-2- furyl}methyl)thio]ethyl}-2-(5-{5- [(ethylamino)methyl]-4-methylpyridin- 3-yl}-1H-indazol-3-yl)-1H-imidazoie-5- carboxamide | 572.73 | 573.73 | 37 |
| 156 | 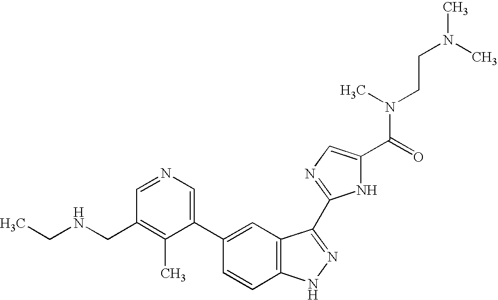 | N-[2-(dimethylamino)ethyl]-2-(5-{5- (ethylamino)methyl]-4-methylpyridin- 3-yl}-1H-indazol-3-yl)-N-methyl-1H- imidazole-5-carboxamide | 460.58 | 461.58 | 25 |
| 157 | 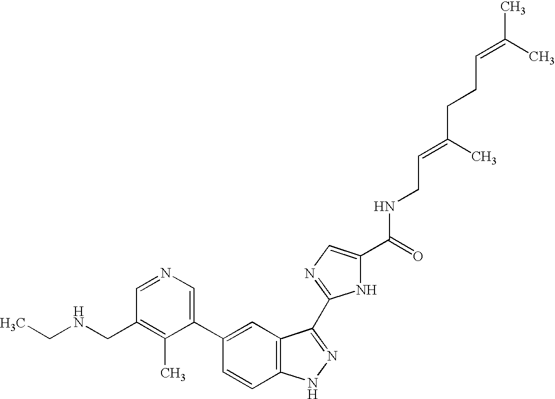 | N-[(2E)-3,7-dimethylocta-2,6-dien-1- yl]-2-(5-{5-[(ethylamino)methyl]-4- ethylpyridin-3-yl}-1H-indazol-3-yl)- 1H-imidazole-5-carboxamide | 511.67 | 512.67 | 28 |
| 158 | 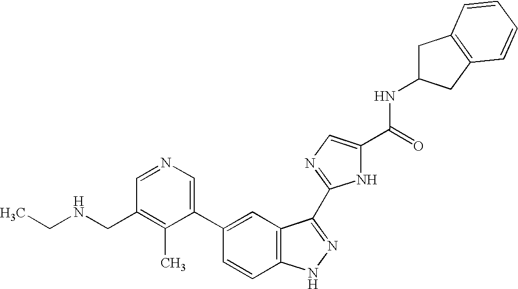 | N-(2,3-dihydro-1H-inden-2-yl)-2-(5-{5- [(ethylamino)methyl]-4-methylpyridin- 3-yl}-1H-indazol-3-yl)-1H-imidazole-5- carboxamide | 491.60 | 492.60 | 56 |
| 159 | 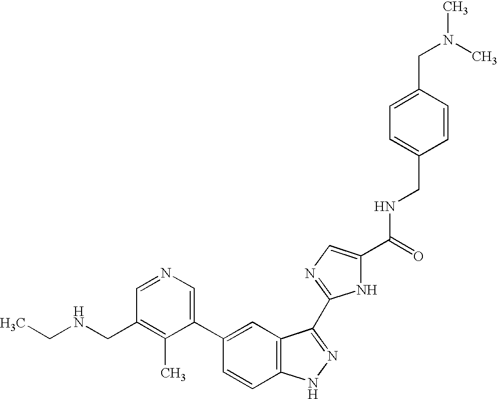 | N-{4[(dimethylamino)methyl]benzyl}- 2-(5-{5-[(ethylamino)methyl]-4- methylpyridin-3-yl}-1H-indazol-3-yl)- 1H-imidazole-5-carboxamide | 522.65 | 523.65 | 28 |
| 160 | 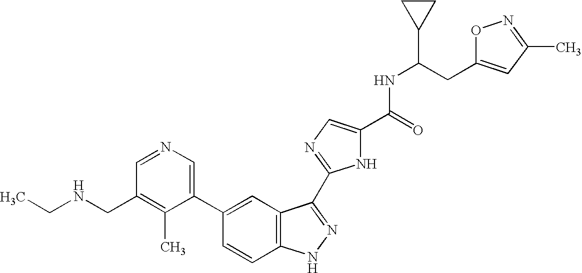 | N-[1-cyclopropyl-2-(3-methylisoxazol- 5-yl)ethyl]-2-(5-{5- [(ethylamino)methyl]-4-methylpyridin- 3-yl}-1H-indazol-3-yl)-1H-imidazole-5- carboxamide | 524.63 | 525.63 | 48 |
| 161 | 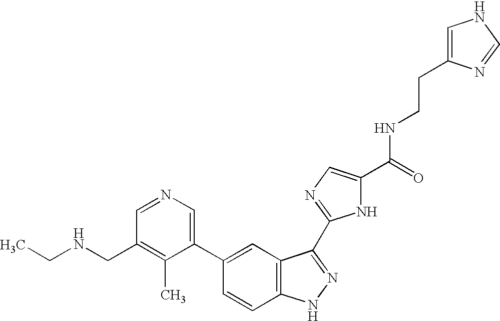 | 2-(5-{5-[(ethylamino)methyl]-4- methylpyridin-3yl-1H-indazol-3yl)-N- 2-(1H-imidazol-4-yl)ethyl]-1H- imidazole-5-carboxamide | 469.55 | 470.55 | 48 |
| 162 | 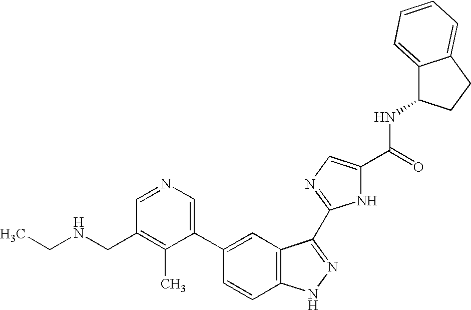 | N-](1S)-2,3-dihydro-1H-inden-1-yl]-2- (5-{5-[(ethylamino)methyl]-4- methylpyridin-3-yl}-1H-indazol-3-yl)- 1H-imidazole-5-carboxamide | 491.60 | 492.60 | 81 |
| 163 | 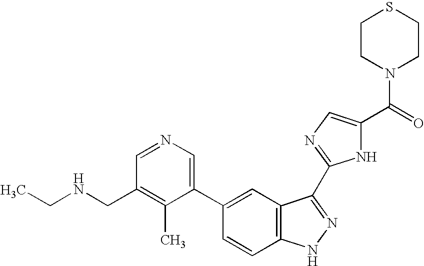 | ethyl[(4-methyl-5-{3-[5-(thiomorpholin- 4-ylcarbonyl)-1H-imidazol-2-yl]-1H- indazol-5-yl}pyridin-3-yl)methyl]amine | 461.59 | 462.59 | 60 |
| 164 | 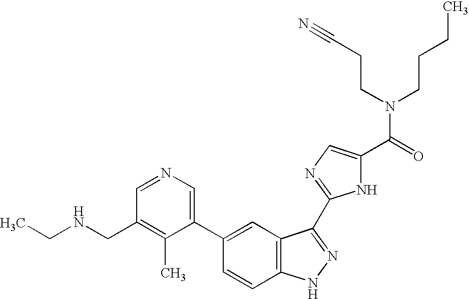 | N-butyl-N-(2-cyanoethyl)-2-(5-{5- [(ethylamino)methyl]4-methylpyridin- 3-yl}-1H-indazol-3-yl)-1H-imidaole-5- carboxamide | 484.60 | 485.60 | 69 |
| 165 | 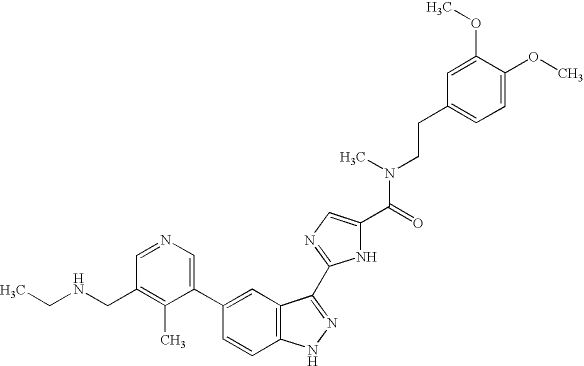 | N-[2-(3,4-dimethoxyphenyl)ethyl]-2-(5- {5-[(ethylamino)methyl]-4- methylpyridin-3-yl}-1H-indazol-3-yl)-N- methyl-1H-imidazole-5-carboxamide | 553.66 | 554.66 | 37 |
| 166 | 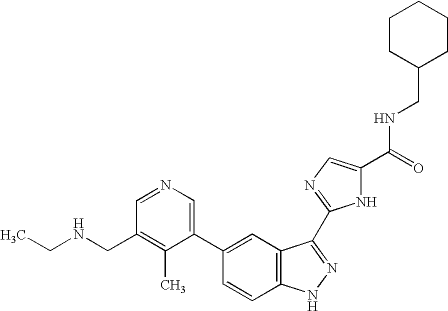 | N-(cyclohexylmethyl)-2-(5-{5- [(ethylamino)methyl]-4-methylpyridin- 3-yl}-1H-indazol-3-yl)-1H-imidazole-5- carboxamide | 471.61 | 472.61 | 73 |
| 167 | 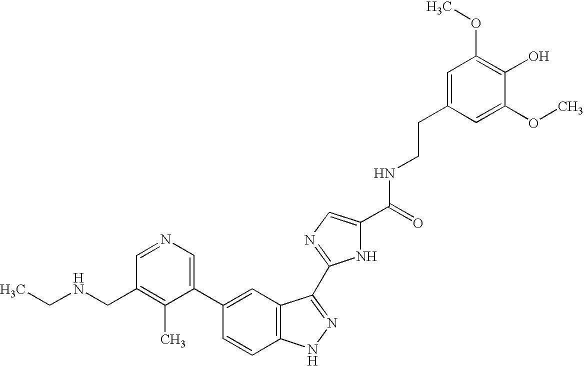 | 2-(5-{5-[(ethylamino)methyl]-4- methylpyridin-3-yl}-1H-indazol-3-yl)-N- 2-(4-hydroxy-3,5- dimethoxyphenyl)ethyl]-1H-imidazole- 5-carboxamide | 555.64 | 556.64 | 28 |
| 168 | 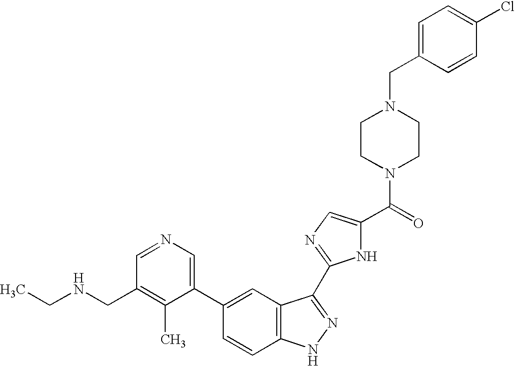 | ({5-[3-(5-{[4-(4-chlorobenzyl)piperazin- 1-yl]carbonyl}-1H-imidazol-2-yl)-1H_ indazol-5-yl]-4-methylpyridin-3- yl}-methyl)ethylamine | 569.11 | 570.11 | 71 |
| 169 | 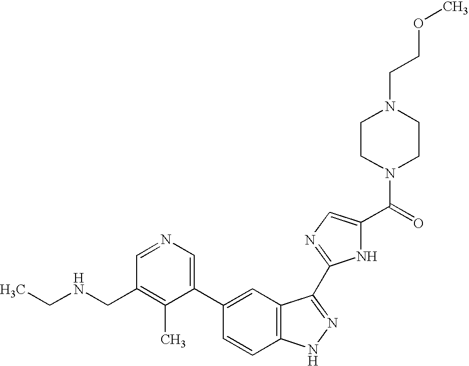 | ethyl({5-[3-(5-{[4-(2- methoxyethyl)piperazin-1-yl]carbonyl}- 1H-imidazol-2-yl)-1H-indazol-5-yl]-4- methylpyridin-3-yl}methyl)amine | 502.62 | 503.62 | 37 |
| 170 | 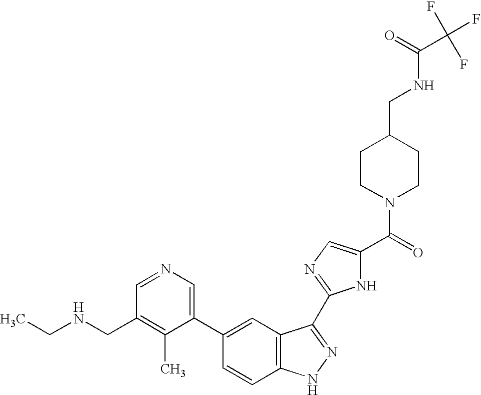 | N-[(1-{[2-(5-{5-[(ethylamino)methyl]-4- methylpyridin-3yl}-1H-indazol-3-yl)- 1H-imidazol-5-yl]carbonyl}piperidin-4- yl)methyl]-2,2,2-trifluoroacetamide | 568.60 | 569.60 | 29 |
| 171 | 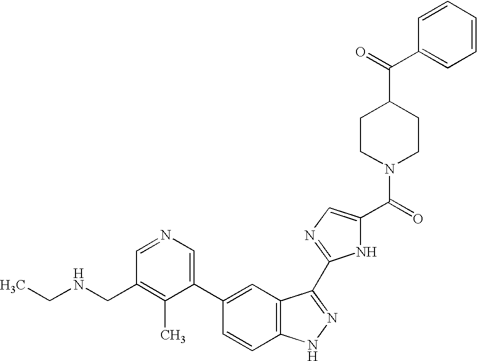 | (1-{[2-(5-{5[(ethylamino)methyl]-4- methylpyridin-3-yl}-1H-indazol-3-yl)- 1H-imidazol-5-yl]carbonyl}piperidin-4- yl)(phenyl)methanone | 547.66 | 548.66 | 65 |
| 172 | 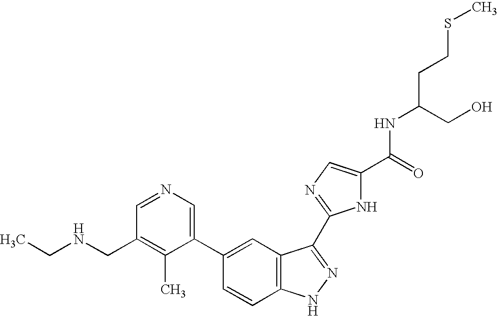 | 2-(5-{5-[(ethylamino)methyl]-4- methylpyridin-3-yl}-1H-indazol-3-yl)-N- 1-(hydroxymethyl)-3- (methylthio)propyl]-1H-imidazole-5- carboxamide | 493.63 | 494.63 | 36 |
| 173 | 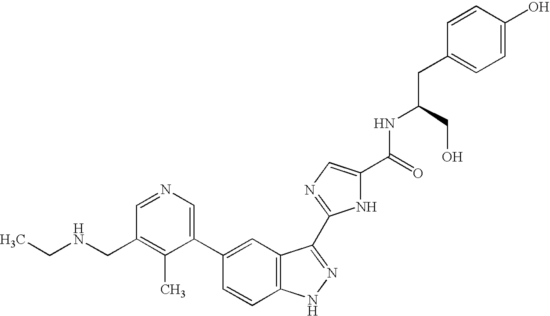 | 2-(5-{5-[(ethylamino)methyl]-4- methylpyridin-3-yl}-1H-indazol-3-yl)-N- (1S)-2-hydroxy-1-(4- hydroxybenzyl)ethyl]-1H-imidazole-5- carboxamide | 525.61 | 526.61 | 45 |
| 174 | 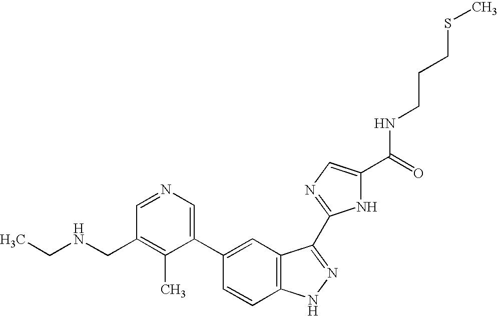 | 2-(5-{5-[(ethylamino)methyl]-4- methylpyridin-3-yl}-1H-indazol-3-yl)-N- [3-(methylthio)propyl]1H-imidazole-5- carboxamide | 463.61 | 464.61 | 41 |
| 175 | 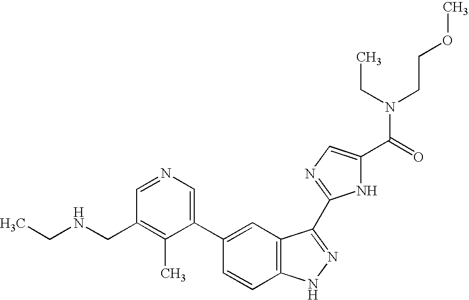 | N-ethyl-2-(5-{5-[(ethylamino)methyl]-4- methylpyridin-3-yl}-1H-indazol-3-yl)-N- (2-methoxyethyl)-1H-imidazole-5- carboxamide | 461.57 | 462.57 | 68 |
| 176 | 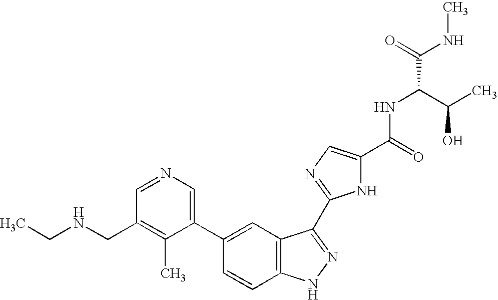 | 2-(5-{5-[(ethylamino)methyl]-4- methylpyridin-3-yl}-1H-indazol-3-yl)-N- {(1S,2R)-2-hydroxy-1- [(methylamino)carbonyl]propyl}-1H- imidazole-5-carboxamide | 490.57 | 491.57 | 59 |
| 177 | 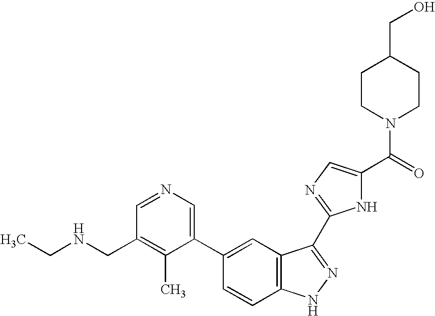 | (1-{[2-(5-{5-[(ethylamino)methyl]-4- methylpyridin-3-yl}-1H-indazol-3-yl)- 1H-imidazol-5-yl]carbonyl}piperidin-4- yl)methanol | 473.58 | 474.58 | 63 |
| 178 | 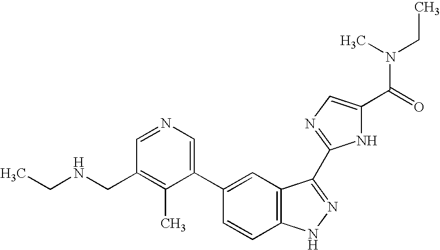 | N-ethyl-2-(5-{5-[(ethylamino)methyl]-4- methylpyridin-3-yl}-1H-indazol-3-yl)-N- methyl-1H-imidazole-5-carboxamrde | 417.51 | 418.51 | 73 |
| 179 | 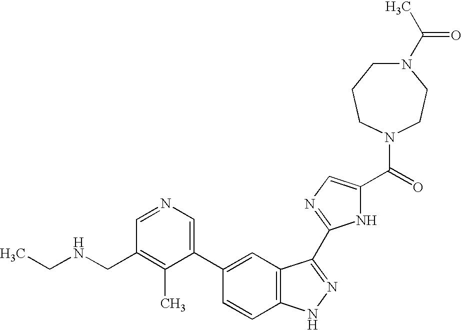 | {[5-(3-(5-[(4-acetyl-1,4-diazepan-1- yl)carbonyl]-1H-imidazol-2-yl]-1H- indazol-5-yl)-4-methylpyridin-3- yl]methyl}ethylamine | 500.60 | 501.60 | 53 |
| 180 | 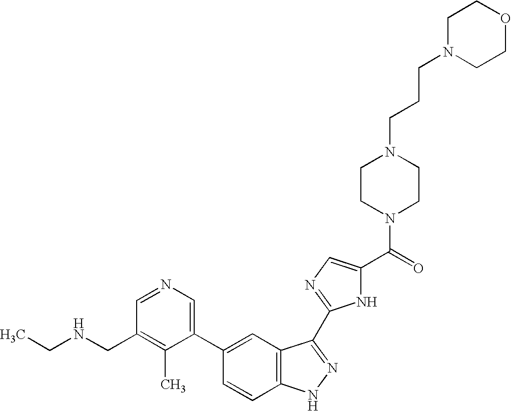 | ethyl({4-methyl-5-[3-(5-{[4-(3- morpholin-4-ylpropyl)piperazin-1- yl]carbonyl}-1H-imidazol-2-yl)-1H- indazol-5-yqpyridin-3-yl}methyl)amine | 571.73 | 572.73 | 22 |
| 181 | 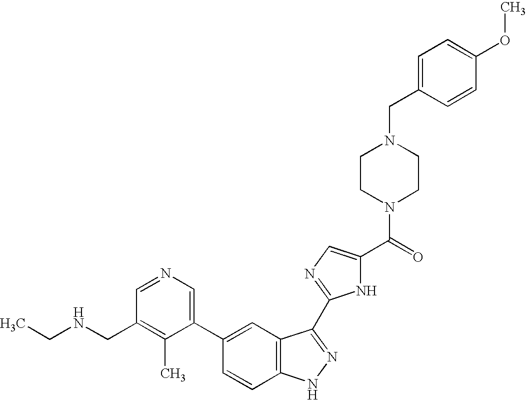 | ethyl({5-[3-(5-{[4-(4- methoxybenzyl)piperazin-1- yl]carbonyl}1H-imidazol-2-yl)-1H- indazol-5-yl]-4-methylpyridin-3- yl}methyl)amine | 564.69 | 565.69 | 57 |
| 182 | 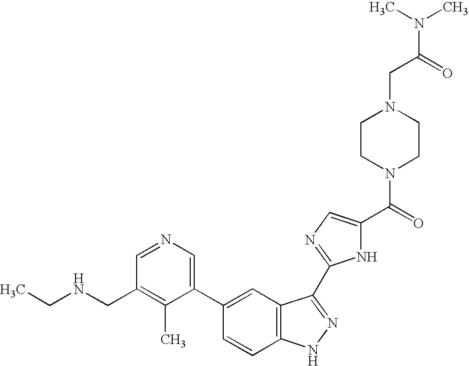 | 2-(4-{[2-(5-{5-[(ethylamino)methyl]-4- methylpyrridin-3-yl}-1H-indazol-3-yl- 1H-imidazol-5-yl]carbonyl}piperazin-1- yl)-N-N-dimethyacetamide | 529.65 | 530.65 | 40 |
| 183 | 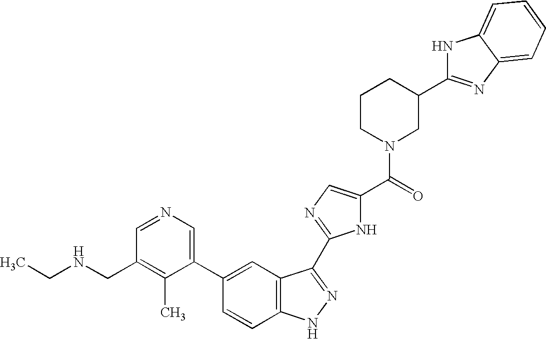 | ({5-[3-(5-{[3-(1H-benzimidazol-2- yl)piperidin-1-yl]carbonyl}-1H- imidazol-2-yl)-1H-indazol-5-yl]-4- methylpyridin-3-yl}methyl)ethylamine | 559.67 | 560.67 | 42 |
| 184 | 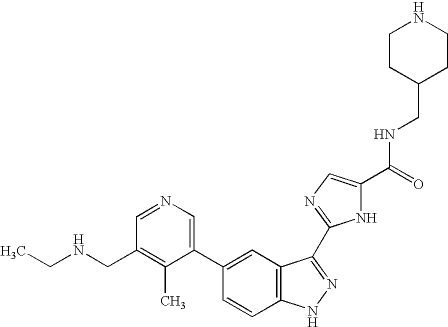 | 2-(5-{5-[(ethylamino)methyl]-4- methylpyridin-3-yl}-1H-indazol-3-yl)-N- (piperidin-4-ylmethylpyridin-1H- imidazole-5-carboxamide | 472.59 | 473.59 | 21 |
| 185 | 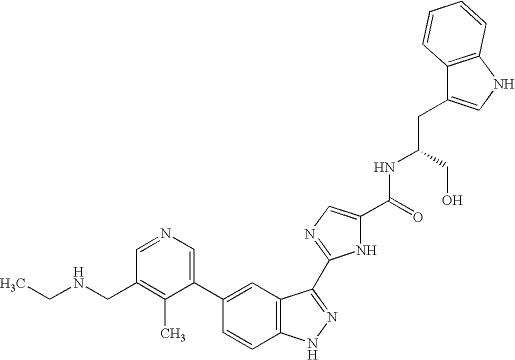 | 2-(5-{5-[(ethylamino)methyl]-4- methylpyridin-3-yl}-1H-indazol-3-yl)-N- [(1R)-2-hydroxy-1-(1H-indol-3- ylmethyl)ethyl]-1H-imidazole-5- carboxamide | 548.65 | 549.65 | 52 |
| 186 | 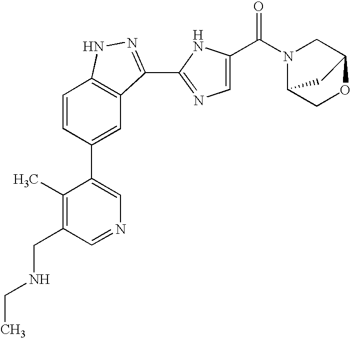 | ethyl{[4-methyl-5-(3-{5-[(1S,4S)-2-oxa- 5-azabicyclo[2.2.1]hept-5-ylcarbonyl]- 1H-imidazol-2-yl)-1H-indazol-5- yl)pyridin-3-yl]methyl}amine | 457.54 | 458.54 | 61 |
| 187 | 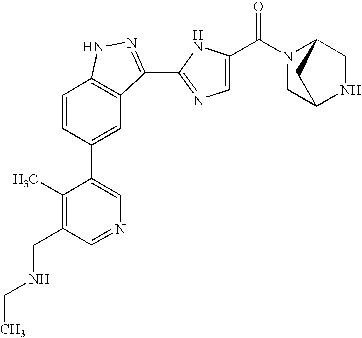 | {[5-(3-{5-[(1S,4S)-2,5- diazabicyclo[2.2.1]hept-2-ylcarbonyl]- 1H-imidazol-2-yl}-1H-indazol-5-yl)-4- ethylpyridin-3-yl]methyl)ethylamine | 456.55 | 457.55 | 44 |
| 188 | 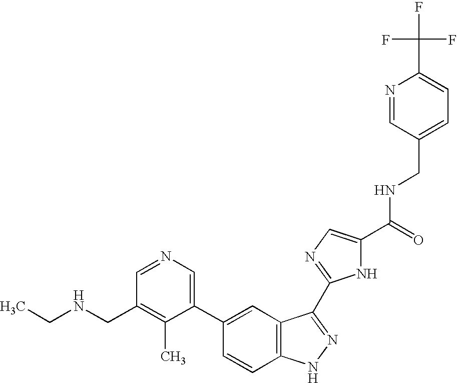 | 2-(5-{5-[(ethylamino)methyl]-4- methylpyridin-3-yl}-1H-indazol-3-yl)-N- {(6-(trifluoromethyl)pyridin-3- yl]methyl}-1H-imidazole-5- carboxamide | 534.54 | 535.54 | 41 |
| 189 | 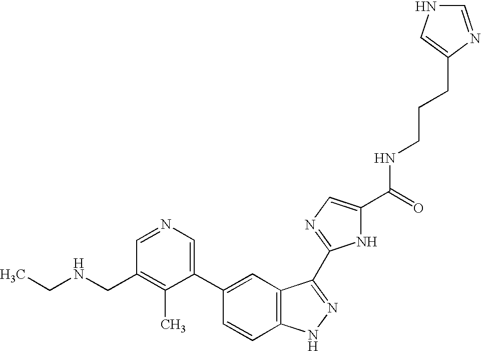 | 2-(5-{5-[(ethylamino)methyl]-4- methylpyridin-3-yl}-1H-indazol-3-yl)-N- [3-(1H-imidazol-4-yl)propyl]-1H- imidazole-5-carboxamide | 483.58 | 484.58 | 59 |
| 190 | 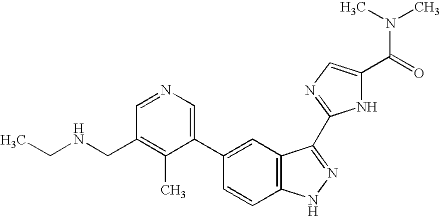 | 2-(5-{5-[(ethylamino)methyl}-4- methylpyridin-3-yl}-1H-indazol-3-yl)- N,N-dimethyl-1H-imidazole-5- carboxamide | 403.49 | 404.49 | 76 |
| 191 | 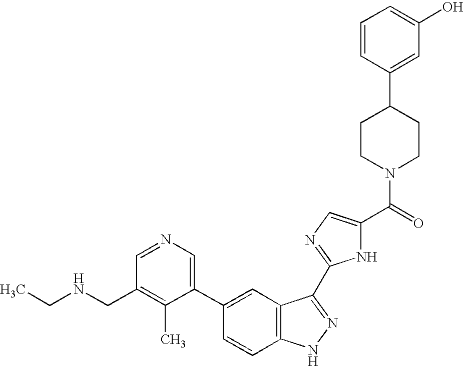 | 3-(1-{[2-(5-{5-[(ethylamino)methyl]-4- methylpyridin-3-yl}-1H-indazol-3-yl)- 1H-imidazol-5-yl]carbonyl}piperidin-4- yl)phenol 535.65 | 536.65 | 74 | |
| 192 | 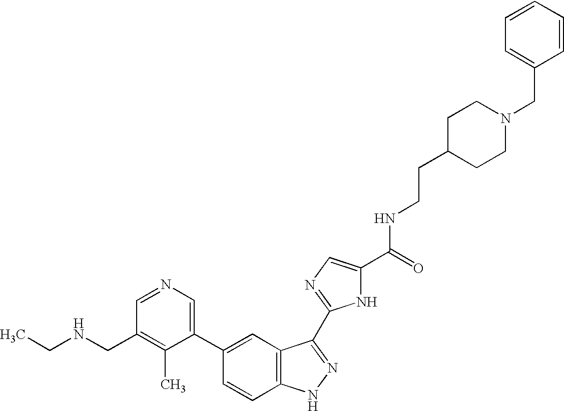 | N-[2-(1-benzylpiperidin-4-yl)ethyl]-2- (5-{5-[(ethylamino)methyl]-4- methylpyridin-3-yl}-1H-indazol-3-yl)- 1H-imidazole-5-carboxamide | 576.75 | 577.75 | 30 |
| 193 | 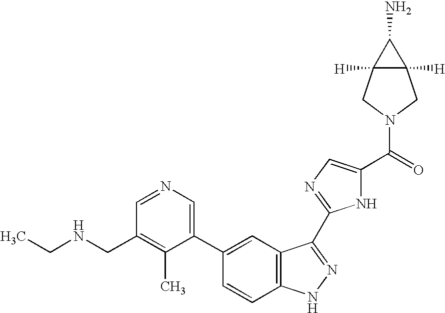 | (1R,5S)-3-[[2-(5-{5- (ethylamino)methyl]-4-methylpyridin- 3-yl}-1H-indazol-3-yl)-1H-imidazol-5- yl]carbonyl}-3-azabicyclo[3.1.0]hexan- 6-amine | 456.55 | 457.55 | 52 |
| 194 | 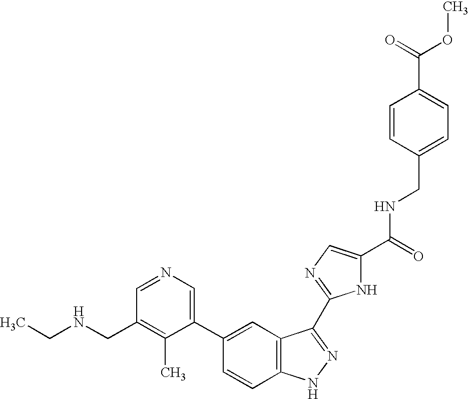 | methyl-4-[({[2-(5-{5- [(ethylamino)methyl]-4-methylpyridin- 3-yl}-1H-indazol-3-yl)-1H-imidazol-5- yl]carbonyl}amino)methyl]benzoate | 523.59 | 524.59 | 65 |
| 195 | 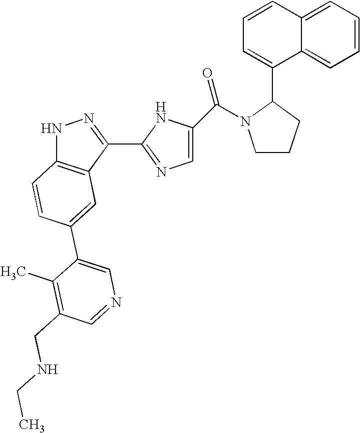 | ethyl({4-methyl-5-(3-(5-{[2-(1- naphthyl)pyrrolidin-1-yl]carbonyl}-1H- imidazol-2-yl)-1 H-indazol-5-yl]pyridin- 3-yl]methyl)amine | 555.68 | 556.68 | 32 |
| 196 | 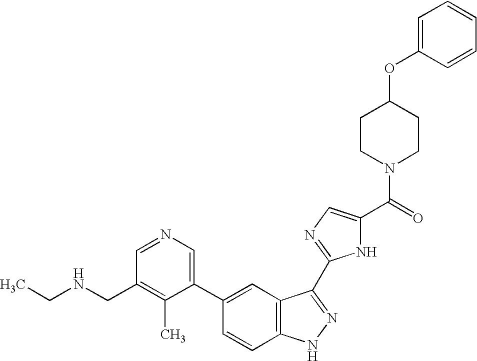 | ethyl{[4-methyl-5-(3-{5-[(4- phenoxypiperidin-1-yl)carbonyl]-1H- imidazol-2-yl]-1H-indazol-5-yl)pyridin- 3-yl]methyl}amine | 535.65 | 536.65 | 71 |
| 197 | 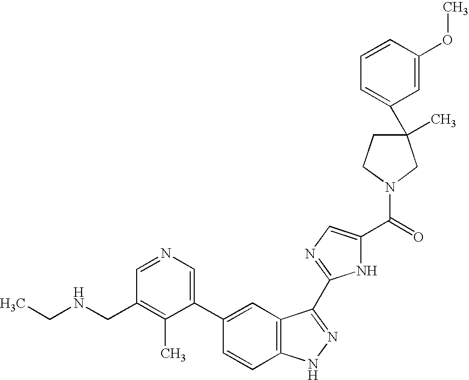 | ethyl({5-[3-(5-{[3-(3-methoxyphenyl)-3- methylpyrrolidin-1-yl]carbonyl}-1H- imidazol-2-yl)-1H-indazol-5-yl]-4- methylpyridin-3-yl}methyl)amine | 549.68 | 550.68 | 67 |
| 198 | 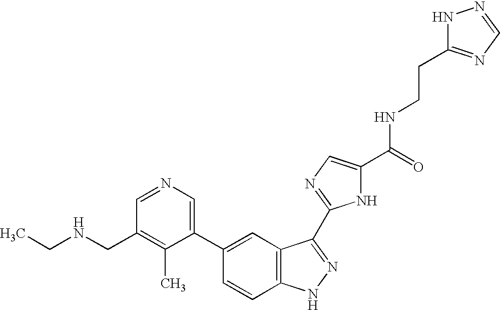 | 2-(5-{5-[(ethylamino)methyl]-4- methylpyridin-3-yl}-1H-indazol-3-yl)-N- [2-(1H-1,2,4-triazol-5-yl)ethyl]-1H- imidazole-5-carboxamide | 470.54 | 471.54 | 55 |
| 199 | 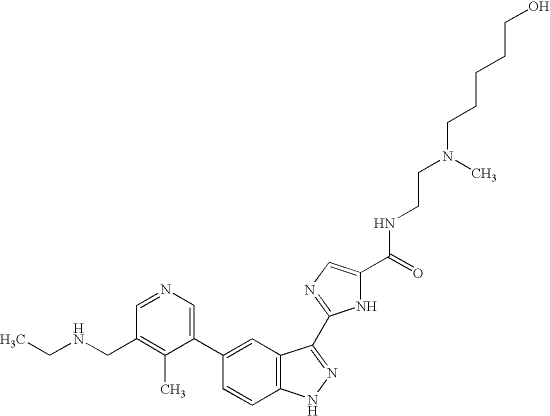 | 2-(5-{5-[(ethylamino)methyl]-4- methylpyridin-3-yl}-1H-indazol-3-yl)-N- {2-[(5- hydroxypentyl)(methyl)amino]ethyl}- 1H-imidazole-5-carboxamide | 518.66 | 519.66 | 25 |
| 200 | 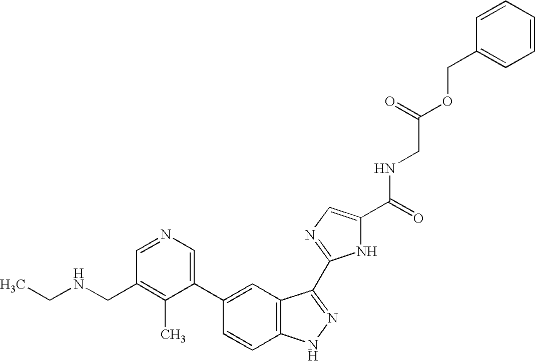 | benzyl N-{[2-(5-{5- [(ethylamino)methyl]-4-methylpyridin- 3-yl}-1H-indazol-3-yl)-1H-imidazol-5- yl]carbonyl}glycinate | 523.59 | 524.59 | 63 |
| 201 | 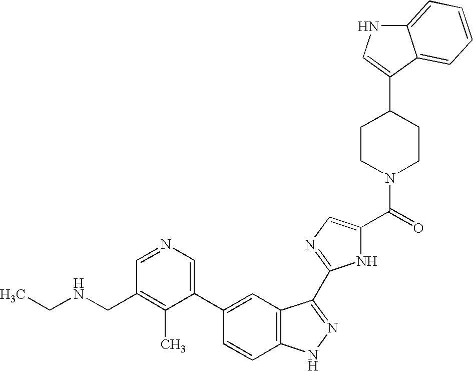 | ethyl({5-(3-(5-{[4-(1H-indol-3- yl)pipendin-1-yl]carbonyl}-1H- imidazol-2-yl)-1H-indazol-5-yl]-4- methylpyridin-3-yl}methyl)amine | 558.69 | 559.69 | 67 |
| 202 | 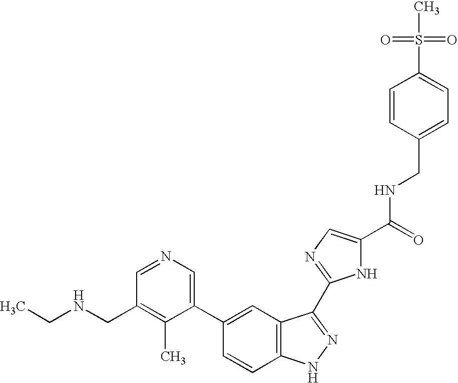 | 2-(5-{5-[(ethylamino)methyl]-4- methylpyridin-3-yl}-1H-indazol-3-yl)-N- [4-(methylsulfonyl)benzyl]-1H- imidazole-5-carboxamide | 543.65 | 544.65 | 49 |
| 203 | 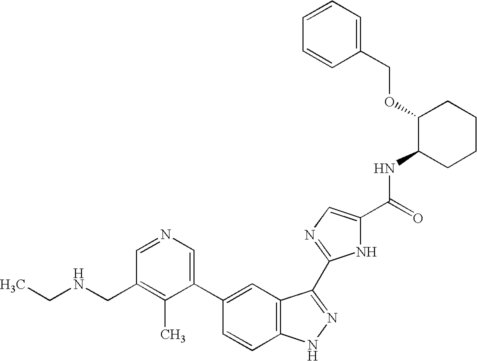 | N-[(1R,2R)-2-(benzyloxy)cyclohexyl]- 2-(5-{5-[(ethylamino)methyl]-4- methylpyridin-3-yl)-1H-indazol-3-yl)- 1H-imidazole-5-carboxamide | 563.70 | 564.70 | 31 |
| 204 | 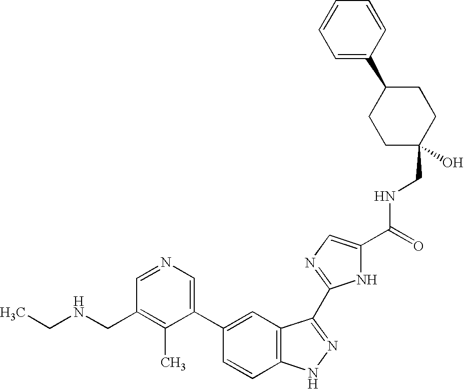 | 2-(5-{5-[(ethylamino)methyl]-4- methylpyridin-3-yl}-1H-indazol-3-yl)-N- [(trans-1-hydroxy-4- phenylcyclohexyl)methyl]-1H- imidazole-5-carboxamide | 563.70 | 564.70 77 | |
| 205 | 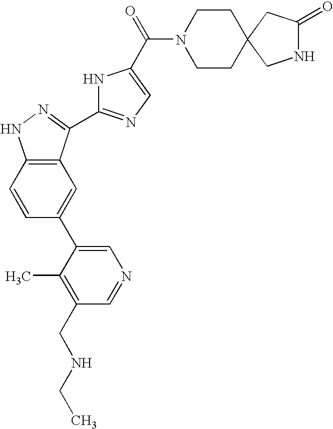 | 8-{[2-(5-{5-[(ethylamino)methyl]-4- methylpyridin-3-yl}-1H-indazol-3-yl)- 1H-imidazol-5-yl]carbanyl}-2,8- diazaspiro[4.5]decan-3-one | 512.61 | 513.61 | 40 |
| 206 | 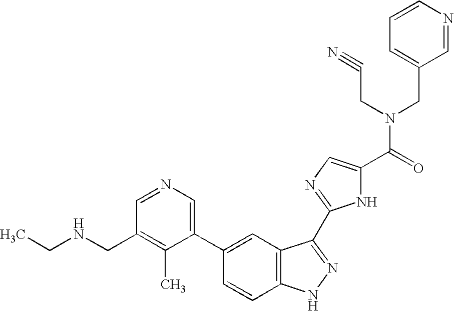 | N-(2-cyanoethyl)-2-(5-{5- N(ethylamino)methyl8 -4-methylpyridin- 3-yl}-1H-indazol-3-yl)-N-(pyridin-3- ylmethyl)-1H-imidazole-5- carboxamide | 519.61 | 520.61 | 71 |
| 207 | 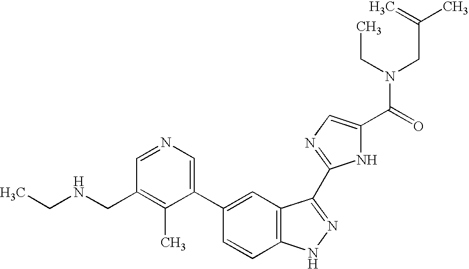 | N-ethyl-2-(5-{5-[(ethylamino)methyl]-4- ethylpyridin-3-yl}-1H-indazol-3-yl)-N- (2-methylprop-2-en-1-yl)-1H- imidazole-5-carboxamide | 457.58 | 458.58 | 69 |
| 208 | 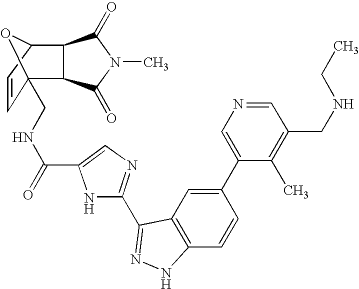 | 2-(5-{5-[(ethylamino)methyl]-4- methylpyridin-3-yl}-1H-indazol-3-yl)-N- {[(3aR,7aS)-2-methyl-13-dioxo- 1,2,3,3a,7,7a-hexahydro-4H-4,7- epoxyisoindol-4-yl]methyl}-1H- imidazole-5-carboxamide | 566.62 | 567.62 | 55 |
| 209 | 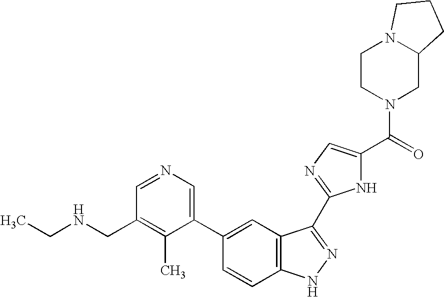 | ethyl[(5-{3-[5-(hexahydropyrrolo[1,2- a]pyrazin-2-(1H)-ylcarbonyl)-1H- imidazol-2-yl]-1H-indazol-5-yl}-4- ethylpyridin-3-yl)methyl]amine | 484.60 | 485.60 | 37 |
| 210 | 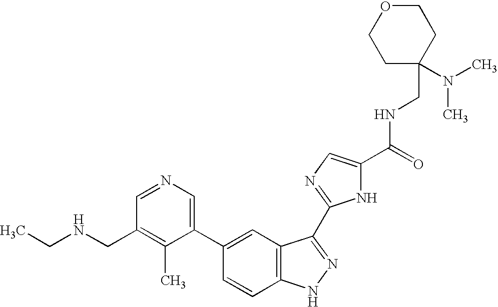 | N-{[4-(dimethylamino)tetrahydro-2H- yran-4-yl]methyl)-2-(5-{5- (ethylamino)methyl]-4-methylpyridin- yl}-1H-indazol-3-yl)-1H-imidazole-5- carboxamide | 516.65 | 517.65 | 35 |
| 211 | 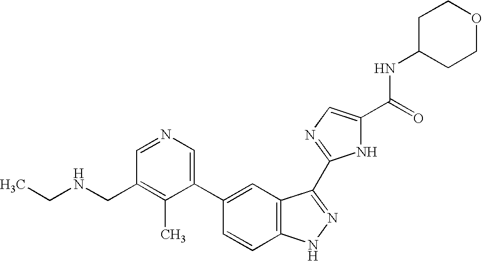 | 2-(5-{5-[(ethylamino)methyl]-4- methylpyridin-3-yl}-1H-indazol-3-yl)-N- (tetrahydro-2H-pyran-4-yl)-1H- imidazole-5-carboxamide | 459.55 | 460.55 | 57 |
| 212 | 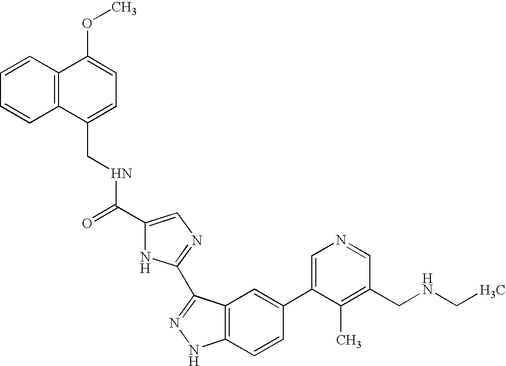 | 2-(5-{5-[(ethylamino)methyl]-4- methylpyridin-3-yl}-1H-indazol-3-yl)-N- (4-methoxy-1-naphthyl)methyl]-1H- imidazole-5-carboxamide | 545.64 | 546.64 | 71 |
| 213 | 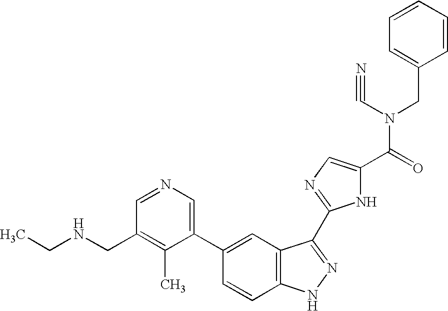 | N-benzyl-N-(cyanomethyl)-2-(5-{5- [(ethylamino)methyl]-4-methylpyridin- 3-yl}-1H-indazol-3-yl)-1H-imidazale-5- carboxamide | 504.60 | 505.60 | 68 |
| 214 | 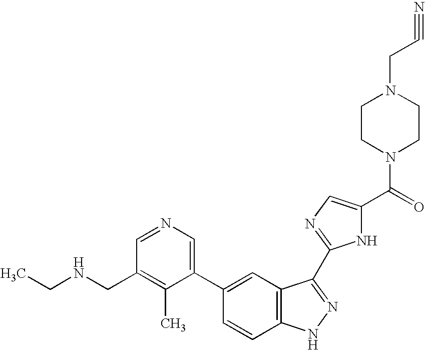 | 3-(4-{[2-(5-{5-[(ethylamino)methyl]-4- methylpyridin-3-yl}-1H-indazol-3-yl)- 1H-imidazol-5-yl]carbonyl}piperazin-1- yl)propanenitrile | 497.60 | 498.60 | 40 |
| 215 | 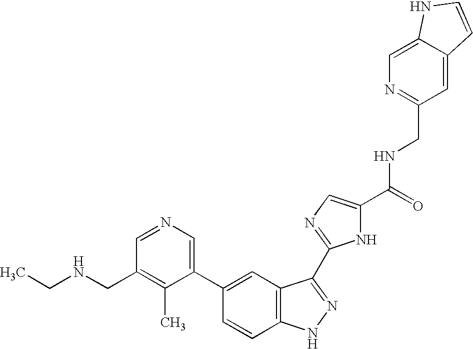 | 2-(5-{5-[(ethylamino)methyl]-4- methylpyridin-3-yl}-1H-indazol-3-yl)-N- (1H-pyrrolo]2,3-c]pyridin-5-ylmethyl)- 1H-imidazole-5-carboxamide | 505.58 | 506.58 | 72 |
| 216 | 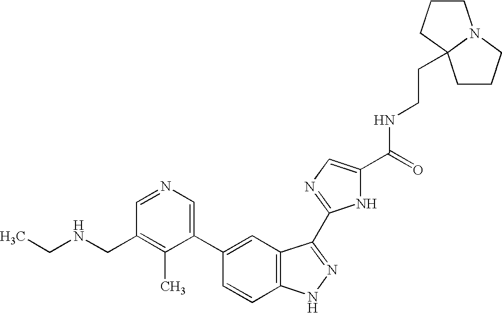 | 2-(5-{5-[(ethylamino)methyl]-4- methylpyridin-3-yl}-1H-indazol-3-yl)-N- [2-(tetrahydro-1H-pyrrolizin-7a(5H)- yl)ethyl]-1H-imidazole-5-carboxamide | 512.66 | 513.66 | 13 |
| 217 | 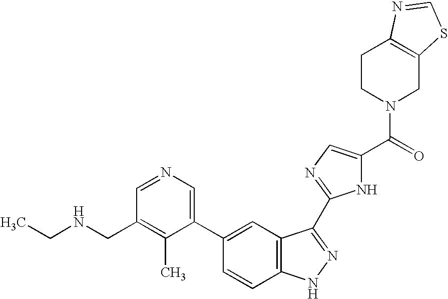 | [(5-{3-[5-(6,7-dihydro[1,3]thiazolo[5,4- c]pyridin-5(4H)-ylcarbonyl)-1H- imidazol-2-yl]-1H-indazol-5-yl}-4- methylpyridin-3-yl)methyl]ethylamine | 498.61 | 499.61 | 79 |
| 218 | 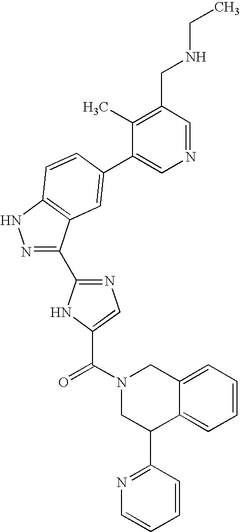 | ethyl{[4-methyl-5-(3-{5-[(4-pyridin-2-yl- 3,4-dihydroisoquinolin-2(1H)- yl)carbonyl]-1H-imidazxol-2-yl}-1H- indazol-5-yl)pyridin-3-yl]methyl}amine | 568.68 | 569.68 | 47 |
| 219 | 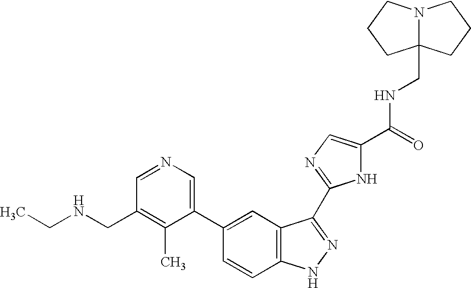 | 2-(5-{5-[(ethylamino)methyl]-4- methylpyridin-3-yl}-1H-indazol-3-yl)-N- (tetrahydro-1H-pyrrolizin-7a(5H)- ylmethyl)-1H-imidazole-5- carboxamide | 498.63 | 499.63 | 20 |
| 220 | 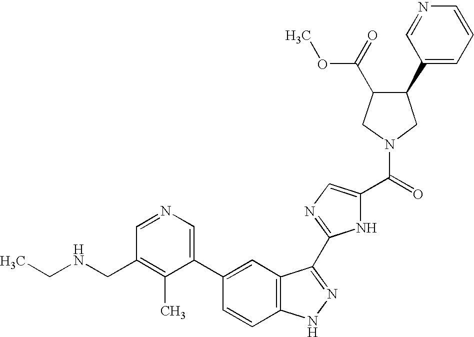 | methyl(3R,4S)-1-{[2-(5-{5- [(ethylamino)methyl]-4-methylpyridin- 3-yl}-1H-indazol-3-yl)-1H-imidazol-5- yl]carbonyl}-4-pyridin-3-ylpyrrolidine-3- carboxylate | 564.65 | 565.65 | 46 |
| 221 | 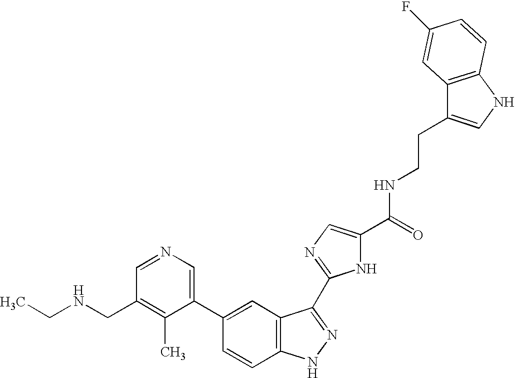 | 2-(5-{5-[(ethylamino)methyl]-4- methylpyridin-3-yl}-1H-indazol-3-yl)-N- [2-(5-fluoro-1H-indol-3-yl)ethyl]-1H- imidazole-5-carboxamide | 536.61 | 537.61 | 56 |
| 222 | 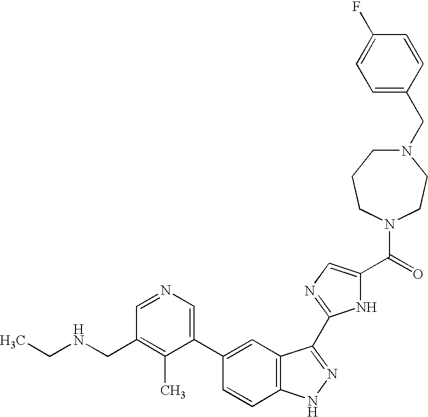 | ethyl({5-[3-(5- {[4-(4-fluorobenzyl)-1,4- diazxepan-1-yl]carbonyl}1H-imidazol- 2-yl)-1H-indazol-5-yl]-4-methylpyridin- 3-yl}methyl)amine | 566.68 | 567.68 | 30 |
| 223 | 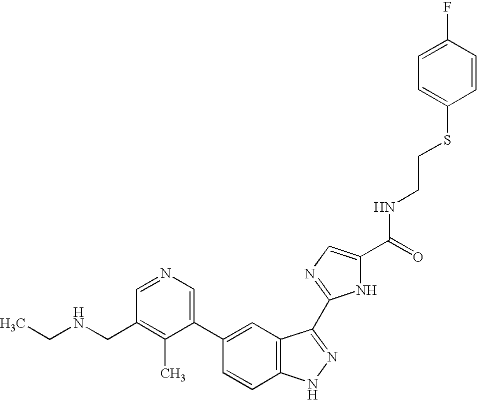 | 2-(5-{5-[(ethylamina)methyl]-4- ethylpyridin-3-yl}-1H-indazol-3-yl)-N- 2-[(4-fluorophenyl)thio]ethyl}-1H- imidazole-5-carboxamide | 529.64 | 530.64 | 71 |
| 224 | 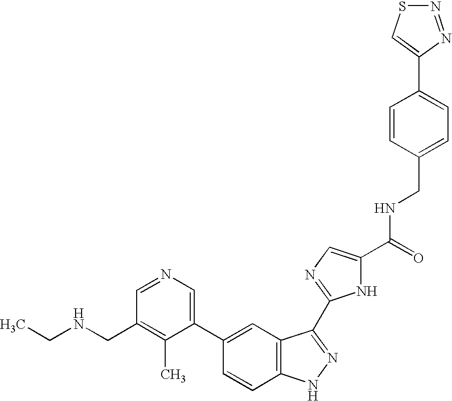 | 2-(5-{5-[(ethylamino)methyl]-4- ethylpyridin-3-yl}-1H-indazol-3-yl)-N- [4-(1,2,3-thiadiazol-4-yl)benzyl]-1H- imidazole-5-carboxamide | 549.66 | 550.66 | 72 |
| 225 | 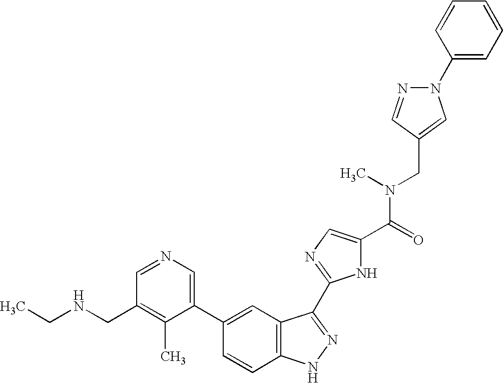 | 2-(5-{5-[(ethylamino)methyl]-4- methylpyridin-3-yl}-1H-indazol-3-yl)-N- methyl-N-[(1-phenyl-1H-pyrazol-4- yl)methyl]-1H-imidazole-5- carboxamide | 545.65 | 546.65 | 70 |
| 226 | 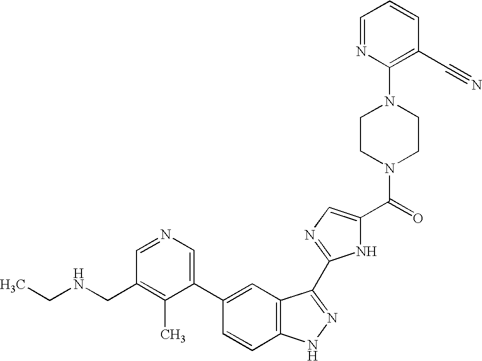 | 2-(4-{[2-(5-{5-[(ethylamino)methyl]-4- methylpyridin-3-yl56 -1H-indazol-3-yl)- 1H-imidazol-5-yl]carbonyl}piperazin-1- yl)nicotinonitrile | 546.64 | 547.64 | 62 |
| 227 | 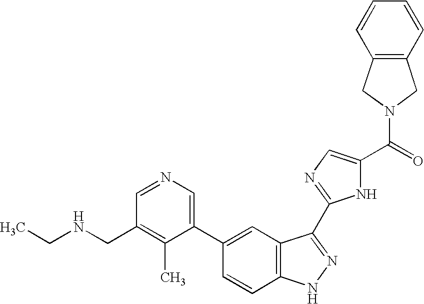 | [(5-{3-[5-(1,3-dihydro-2H-isoindol-2- ylcarbonyl)-1H-imidazol-2-yl]-1H- indazol-5-yl}-4-methylpyridin-3- yl)methyl]ethylamine | 477.57 | 478.57 | 84 |
| 228 | 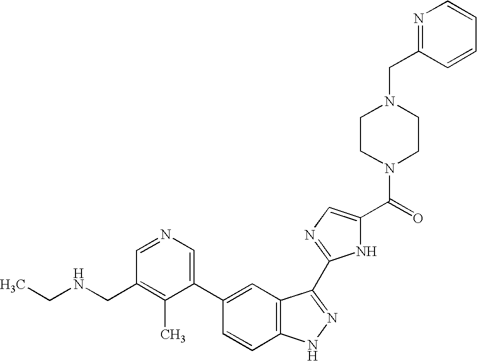 | ethyl({4-methyl-5-[3-(5-{[4-(pyridin-2- ylmethyl)piperazin-1-yl]carbonyl)-1H- imidazol-2-yl)-1H-indazol-5-yl]pyridin- 3-yl}methyl)amine | 535.65 | 536.65 | 50 |
| 229 | 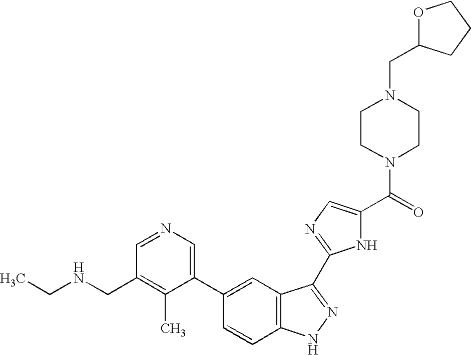 | ethyl({4-methyl-5-[3-(5-{[4- (tetrahydrofuran-2-ylmethyl)piperazin- 1-yl]carbonyl}-1H-imidazol-2-yl)-1H- indazol-5-yl]pyridin-3-yl}methyl)amine | 528.66 | 529.66 | 33 |
| 230 | 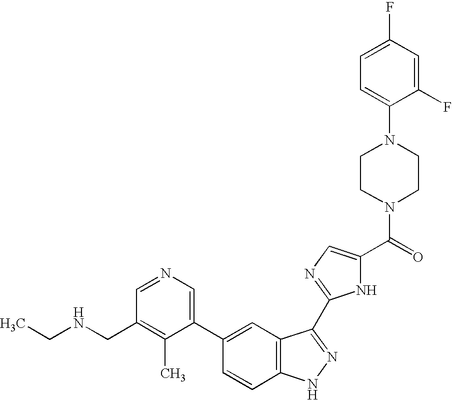 | ({5-[3-{[4-(2,4- difluorophenyl)piperazin-1- yl]carbonyl}-1H-imidazol-2-yl)-1H- indazol-5-yl]-4-methylpyridin-3- yl}methyl)ethylamine | 556.62 | 557.62 | 74 |
| 231 | 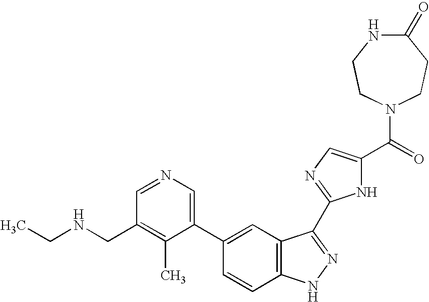 | 1-{[2-(5-{5-[(ethylamino)methyl]-4- methylpyridin-3-yl}-1H-indazol-3-yl)- 1H-imidazol-5-yl]carbonyl}-1,4- diazepan-5-one | 472.55 | 473.55 | 67 |
| 232 | 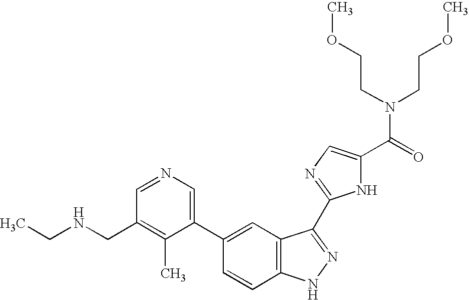 | 2-(5-{5-[(ethylamino)methyl]-4- methylpyridin-3-yl}-1H-indazol-3-yl)- N,N-bis(2-methoxyethyl)-1H- imidazole-5-carboxamide | 491.59 | 492.59 | 58 |
| 233 | 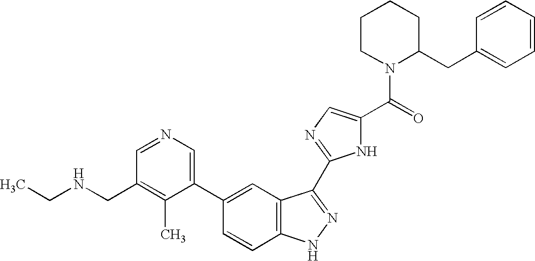 | {[5-(3-{5-[(2-benzylpipendin-1- yl)carbonyl]-1H-imidazal-2-yl)-1H- indazol-5-yl)-4-methylpyridin-3- yl]methyl}ethylamine | 533.68 | 534.68 | 32 |
| 234 | 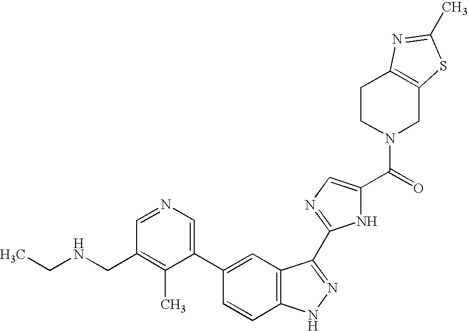 | ethyl{[4-methyl-5-(3-{5-[(2-methyl-6,7- dihydro[1,3]thiazolo[5,4-c]pyridin- 5-(4H)-yl)carbonyl]-1H-imidazol-2-yl)- 1H-indazol-5-yl)pyridin-3- yl]methyl}amine | 512.64 | 513.64 | 74 |
| 235 | 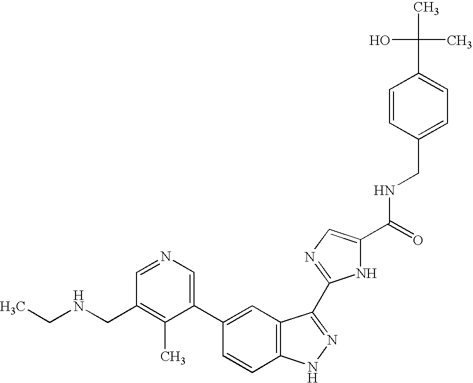 | 2-(5-{5-[(ethylamino)methyl]-4- methylpyridin-3-yl}-1H-indazol-3-yl)-N- [4-(1-hydroxy-1-methylethyl)benzyl]- 1H-imidazole-5-carboxamide | 523.64 | 524.64 | 63 |
| 236 | 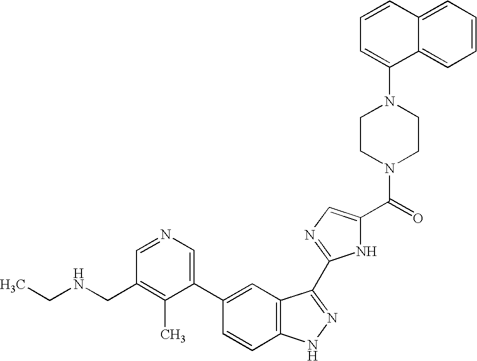 | ethyl({4-methyl-5-[3-(5-{[4-(1- naphthyl)piperazin-1-yl]carbonyl}-1H- imidazol-2-yl)-1H-indazol-5-yl]pyridin- 3-yl}methyl)amine | 570.70 | 571.70 | 74 |
| 237 | 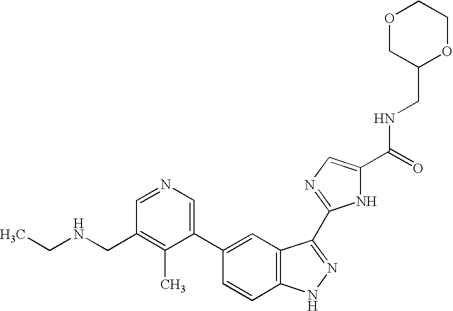 | N-(1,4-dioxan-2-ylmethyl)-2-(5-{5- [(ethyamino)methy]-4-methypyridin- 3-yl}-1H-indazol-3-yl)-1H-imidazole-5- carboxamide | 475.55 | 476.55 | 47 |
| 238 | 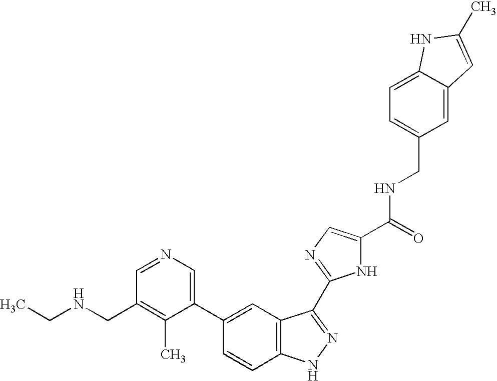 | 2-(5-{5-[(ethylamino)methy]-4- methylpyridin-3-yl}-1H-indazol-3-yl)-N- [(2-methyl-1H-indol-5-yl)methyl]-1H- imidazole-5-carboxamide | 518.62 | 519.62 | 75 |
| 239 | 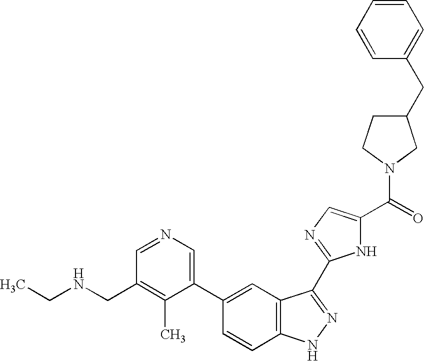 | {(5-[3-{5-[(3-benzylpyrrolidin-1- yl)carbonyl-1H-imidazol-2-yl}-1H- indazol-5-yl)-4-methylpyridin-3- yl]methyl}ethylamine | 519.65 | 520.65 | 75 |
| 240 | 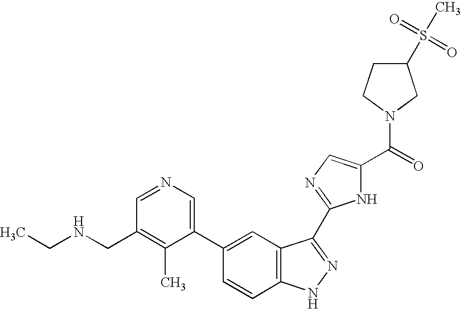 | ethyl({4-methyl-5-[3-(5-{[3- (methylsulfonyl)pyrrolidin-1- carbonyl}-imidazol-2-yl)-1H- indazol-5-yl]pyridin-3-yl}methyl)amine | 507.62 | 508.62 | 71 |
| 241 | 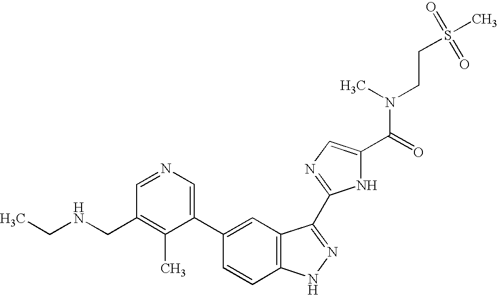 | 2-(5-{5-[(ethylamino)methyl]-4- ethylpyridin-3-yl}-1H-indazol-3-yl)-N- methyl-N-[2-(methylsulfonyl)ethyl]-1H- imidazole-5-carboxamide | 495.61 | 496.61 | 59 |
| 242 | 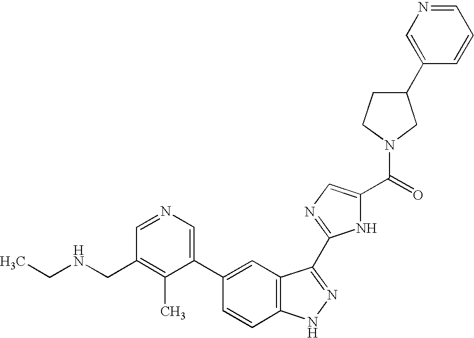 | ethyl{[4-methyl-5-(3-{5-[(3-pyridin-3- ylpyrrolidin-1-yl)carbonyl]-1H-imidazol- 2-yl}-1H-indazol-5-yl)pyridin-3- yl]methyl}amine | 506.61 | 507.61 | 66 |
| 243 | 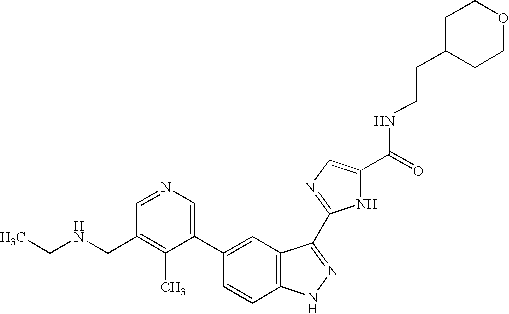 | 2-(5-{5-[(ethylamino)methyl]-4- methylpyridin-3-yl}-1H-indazol-3-yl)-N- [2-(tetrahydro-2H-pyran-4-yl)ethyl]-1H- imidazole-5-carboxamide | 487.60 | 488.60 | 34 |
| 244 | 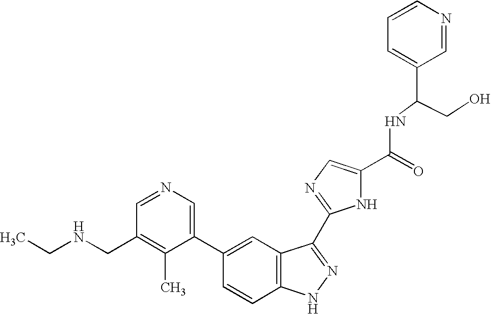 | 2-(5-{5-[(ethylamino)methyl]-4- ethylpyridin-3-yl}-1H-indazol-3-yl)-N- (2-hydroxy-1-pyridin-3-ylethyl)-1H- imidazole-5-carboxamide | 496.57 | 497.57 | 55 |
| 245 | 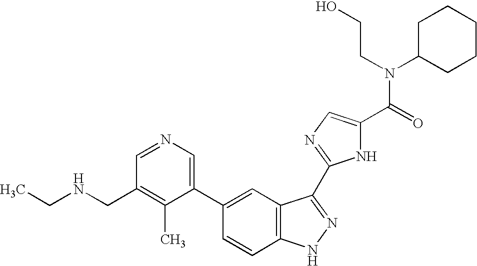 | N-cyclohexyl-2-(5-{5- [(ethylamino)methyl]-4-methylpyridin- 3-yl}-1H-indazol-3-yl)-N-(2- hydroxyethyl)-1H-imidazole-5- carboxamide | 501.63 | 502.63 | 30 |
| 246 | 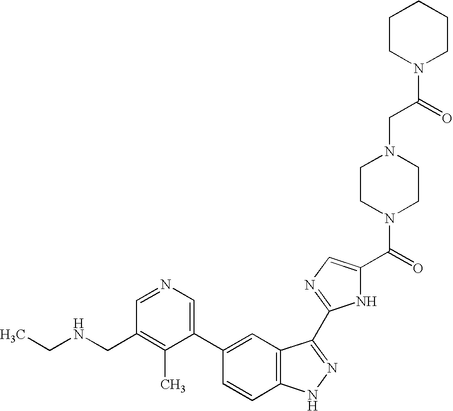 | ethyl({4-methyl-5-[3-(5-{[4-(2-oxo-2- piperidin-1-ylethyl)piperazin-1- yl]carbonyl}-1H-imidazol-2-yl)-1H- indazol-5-yl]pyridin-3-yl}methyl)amine | 569.71 | 570.71 | 26 |
| 247 | 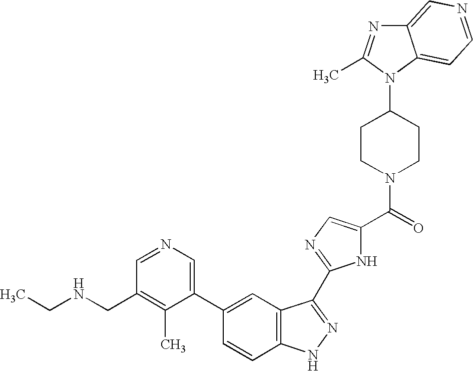 | ethyl({4-methyl-5-[3-(5-{[4-(2-methyl- 1H-imidazol[4,5-c]pyridin-1- yl)piperidin-1-yl]carbonyl}-1H- imidazol-2-yl)-1H-indazol-5-yl]pyridin- 3-yl}methyl)amine | 574.69 | 575.69 | 26 |
| 248 | 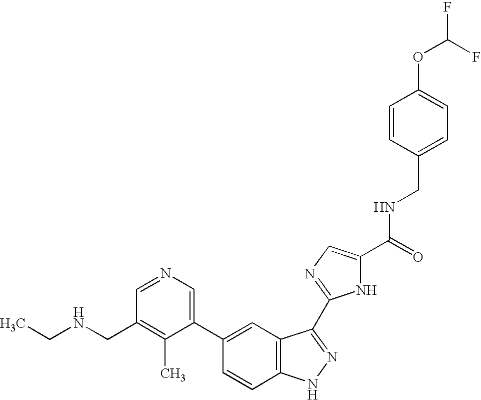 | N-[4-(difluoromethoxy)benzyl]-2-(5-{5- [(ethylamino)methyl]-4-methylpyridin- 3-yl}-1H-indazol-3-yl)-1H-imidazole-5- carboxamide | 531.56 | 532.56 | 67 |
| 249 | 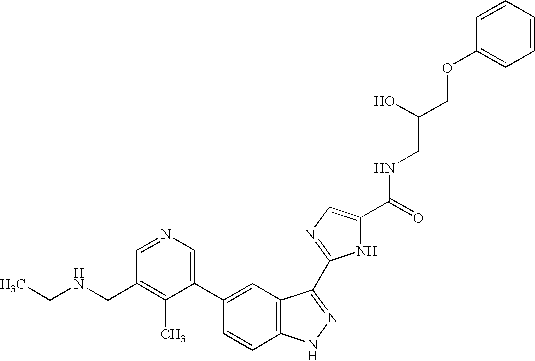 | 2-(5-{5-[(ethylamino)methyl]-4- methylpyridin-3-yl}-1H-indazol-3-yl)-N- (2-hydroxy-3-phenoxypropyl)-1H- imidazole-5-carboxamide | 525.61 | 526.61 | 69 |
| 250 | 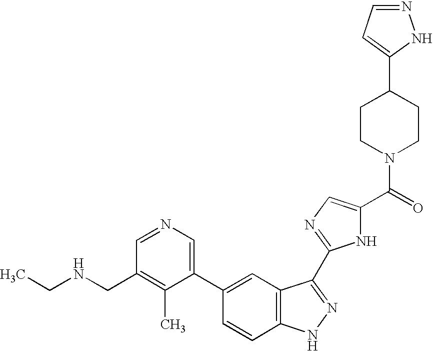 | ethyl({4-methyl-5-[3-(5-{[4-(1H- pyrazol-5-yl)piperidin-1-yl]carbonyl}- H-imidazol-2-yl)-1 H-indazol-5- yl]pyridin-3-yl}methyl)amine | 509.61 | 510.61 | 62 |
| 251 | 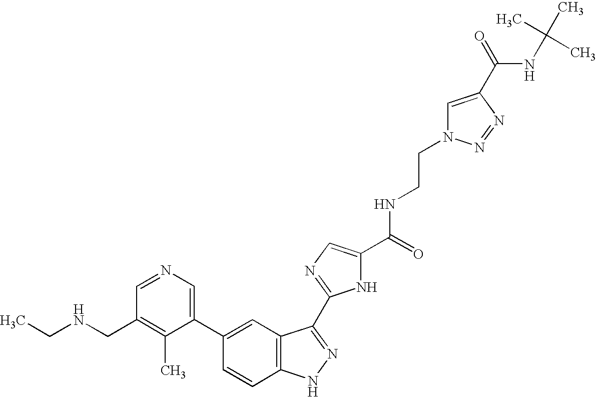 | N-(tert-butyl)-1-[2-({[2-(5-{5- (ethylamino)methyl]-4-methylpyridin- 3-yl}-1H-indazol-3-yl)-1H-imidazol-5- yl]carbonyl}amino)ethyl]-1H-1,2,3- triazole-4-carboxamide | 569.67 | 570.67 | 27 |
| 252 | 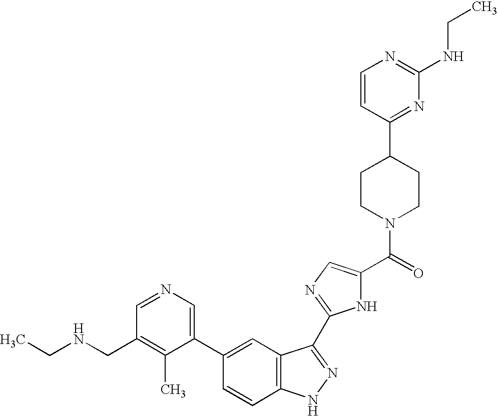 | N-ethyl-4-(1-{[2-(5-{5- [(ethylamino)methyl]-4-methylpyridin- 3-yl}-1H-indazol-3-yl)-1H-imidazol-5- yl]carbonyl}piperidin-4-yl)pyrimidin-2- amine | 564.69 | 565.69 | 41 |
| 253 | 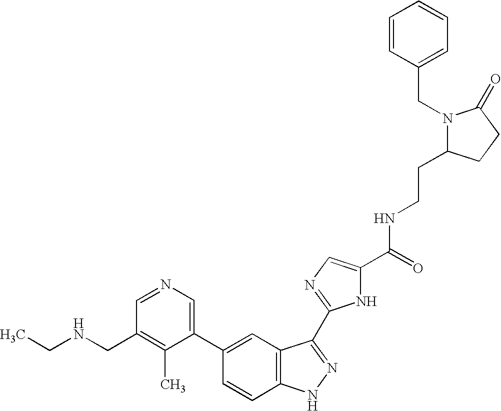 | N-[2-(1-benzyl-5-oxopyrrolidin-2- yl)ethyl]-2-(5-{5-[(ethylamina)methyl]- 4-methylpyridin-3-yl}-1H-indazol-3-yl)- 1H-imidazole-5-carboxamide | 576.70 | 577.70 | 18 |
| 254 | 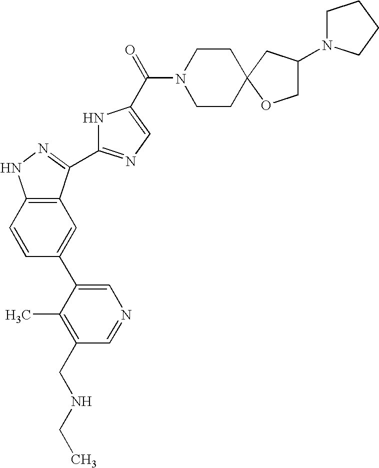 | ethyl{[4-methyl-5-(3-{5-[(3-pyrrolidin-1- yl-1-oxa-8-azaspiro[4.5]dec-8- yl)carbonyl]-1H-imidazol-2-yl}-1H- indazol-5-yl)pyridin-3-yl]methyl}amine | 568.72 | 569.72 | 11 |
| 255 | 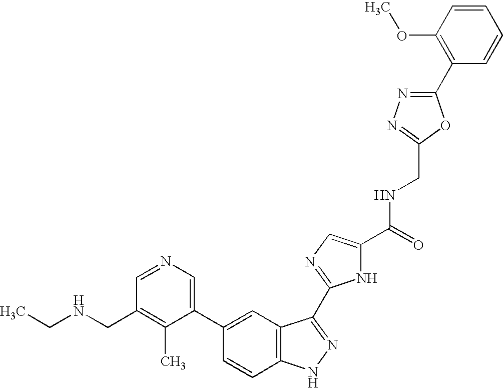 | 2-(5-{5-[(ethylamino)methyl]-4- methylpyridin-3-yl}-1H-indazol-3-yl)-N- {[5-(2-methoxyphenyl)-1,3,4- oxadiazol-2-yl]methyl}-1H-imidazole- 5-carboxamide | 563.62 | 564.62 | 40 |
| 256 | 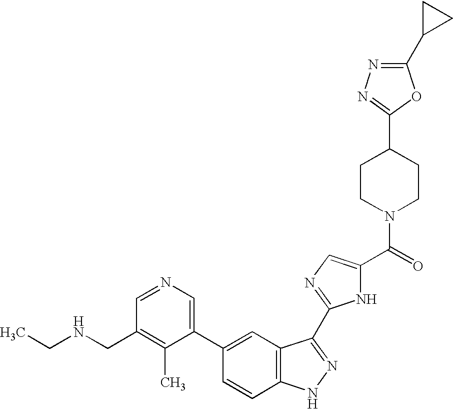 | ({5-[3-(5-[[4-(5-cyclopropyl-1,3,4- oxadiazol-2-yl)piperidin-1-yl]carbonyl}- 1H-imidazol-2-yl)-1H-indazol-5-yl]-4- methylpyridin-3-yl}methyl)ethylamine | 551.65 | 552.65 | 46 |
| 257 | 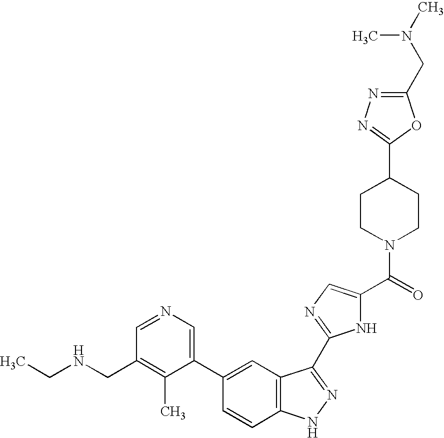 | {[5-(3-{5-[(4-{5- [(dimethylamino)methyl]-1,3,4- oxadiazol-2-yl}piperidin-1-yl)carbonyl]- 1H-imidazol-2-yl}-1H-indazol-5-yl)-4- ethylpyridin-3-yl]methyl}ethylamine | 568.68 | 569.68 | 37 |
| 258 | 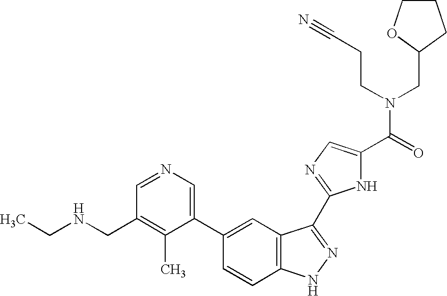 | N-(2-cyanoethyl)-2-(5-{5- (ethylamino)methyl]-4-methylpyridin- 3-ayl}-1H-indazxol-3-yl)-N- (tetrahydrofuran-2-ylmethyl)-1H- midazole-5-carboxamide | 512.61 | 513.61 | 49 |
| 259 | 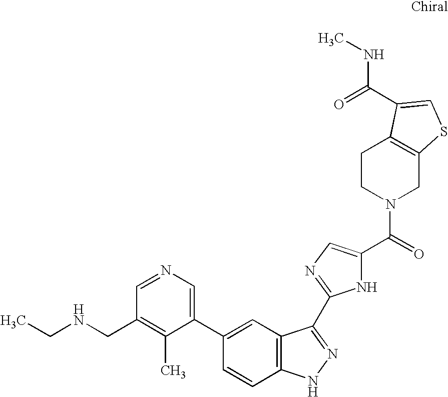 | 6-{[2-(5-{5-[(ethylamino)methyl]-4- methylpyridin-3-yl}-1H-indazol-3-yl)- 1H-imidazol-5-yl]carbonyl}-N-methyl- 4,5,6,7-tetrahydrothieno[2,3- c]pyridine-3-carboxamlde | 554.68 | 555.68 | 69 |
| 260 | 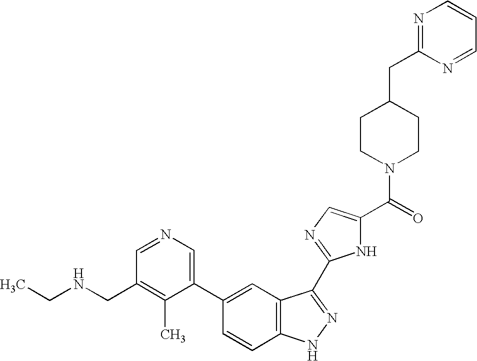 | ethyl({4-methyl-5-[3-(5-{[4-(pyrimidin- 2-ylmethyl)piperidin-1-yl]carbonyl}-1H- imidazol-2-yl)-1H-indazol-5-yl]pyridin- 3-yl}methyl)amine | 535.65 | 536.65 | 33 |
| 261 | 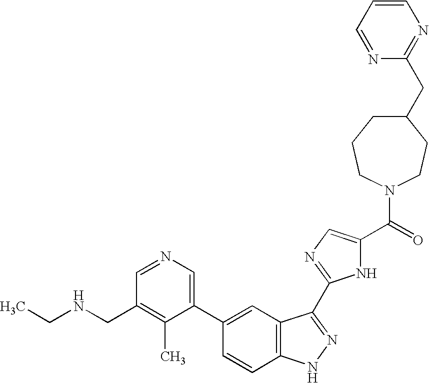 | ethyl({4-methyl-5-[3-(5-{[4-(pyrimidin- 2-ylmethyl)azepan-1-yl]carbonyl}-1H- imidazol-2-yl)-1H-indazol-5-yl]pyridin- 3-yl}methyl)amine | 549.68 | 550.68 | 30 |
| 262 | 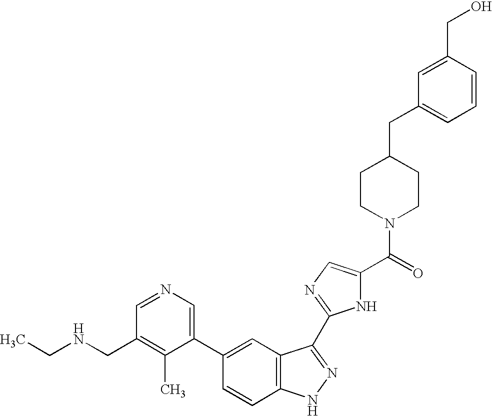 | (3-[(1-{[2-(5-{5-[(ethylamino)methyl]-4- methylpyridin-3-yl}-1H-indazol-3-yl)- 1H-imidazol-5-yl]carbonyl}piperidin-4- yl)methyl]phenyl}methanol | 563.70 | 564.70 | 59 |
| 263 | 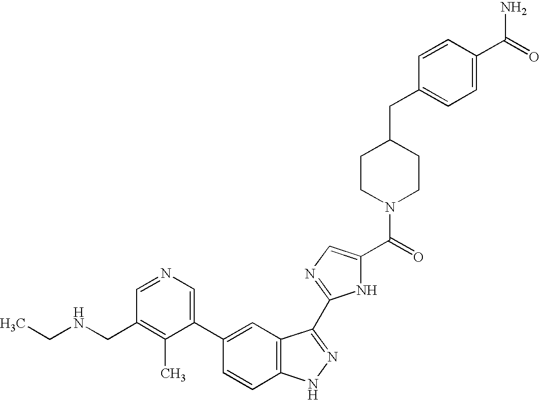 | 4-[(1-{[2-(5-{5-[(ethylamino)methyl]-4- methylpyridin-3-yl}-1H-indazol-3-y)- 1H-imidazol-5-yl]carbonyl}piperidin-4- yl)methyl]benzamide | 576.70 | 577.70 | 69 |
| 264 | 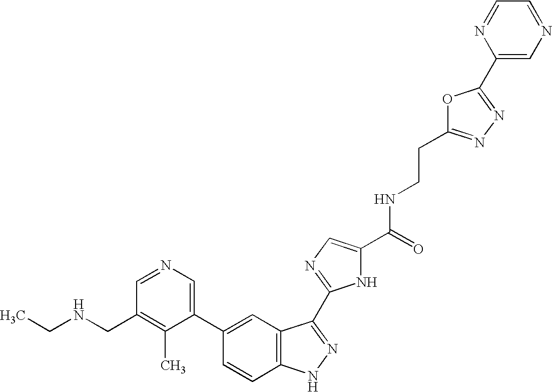 | 2-(5-{5-[(ethylamlno)methyl]-4- methylpyridin-3-yl}-1H-indazol-3-yl)-N- 2-(5-pyrazin-2-yl-1,3,4-oxadiazol-2- yl)ethyl]-1H-imidazole-5-carboxamide | 549.60 | 550.60 | 29 |
| 265 | 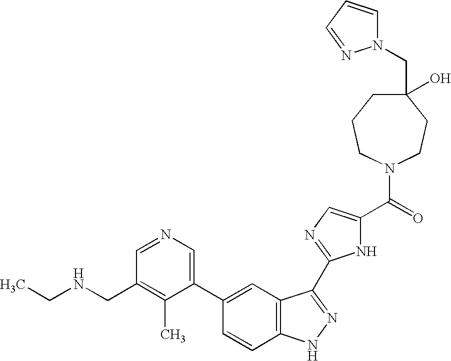 | 1-{[2-(5-{5-[(ethylamino)methyl]-4- methylpyridin-3-yl}-1H-indazol-3-yl)- 1H-imidazol-5-yl]carbonyl}-4-(1H- pyrazol-1-ylmethyl)azepan-4-ol | 553.67 | 554.67 | 29 |
| 266 | 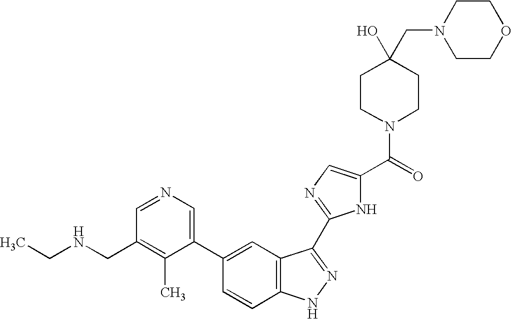 | 1-{[2-(5-{5-[(ethylamino)methyl]-4- methylpyridin-3-yl}-1H-indazol-3-yl)- 1H-imidazol-5-yl]carbonyl}-4- (morpholin-4-ylmethyl)piperidin-4-ol | 558.68 | 559.68 | 18 |
| 267 | 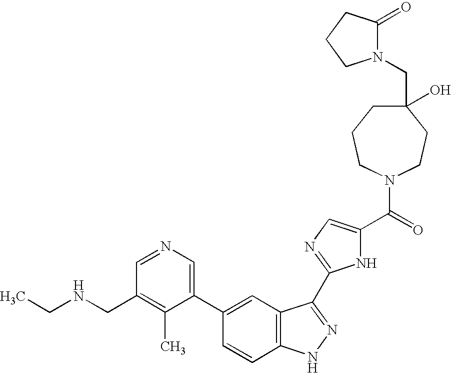 | 1-[(1-{[2-(5-{5-[(ethylamino)methyl]-4- methylpyridin-3-yl}-1H-indazol-3-yl)- 1H-imidazol-5-yl]carbonyl}-4- hydroxyazepan-4-yl)methyl]pyrrolidin- 2-one | 570.69 | 571.69 | 21 |
| 268 | 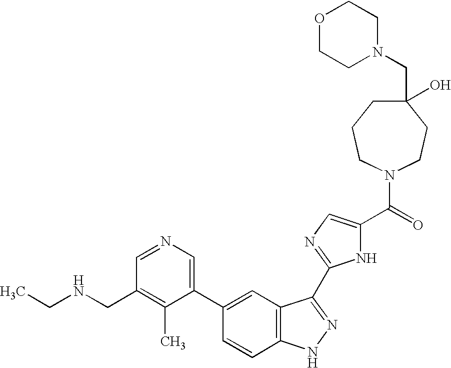 | 1-{[2-(5-{5-[(ethylamino)methyl]-4- methylpyridin-3yl-1H-indazol-3yl)- 1H-imidazol-5-yl]carbonyl}-4- (morpholin-4-ylmethyl)azepan-4-ol | 572.71 | 573.71 | 15 |
| 269 | 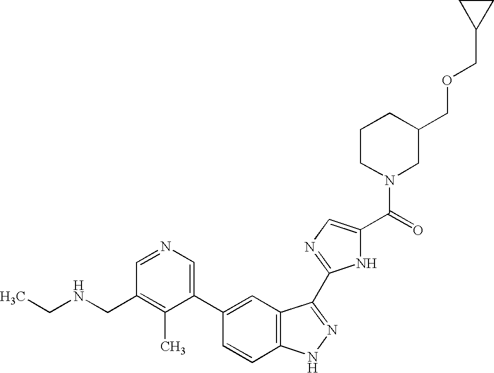 | [(5-(3-[5-({3- [(cyclopropylmethoxy)methyl]piperidin- 1-yl}carbonyl)-1H-imidazol-2-yl]-1H- indazol-5-yl}-4-methylpyridin-3- yl)methyl]ethylamine | 527.67 | 528.67 | 34 |
| 270 | 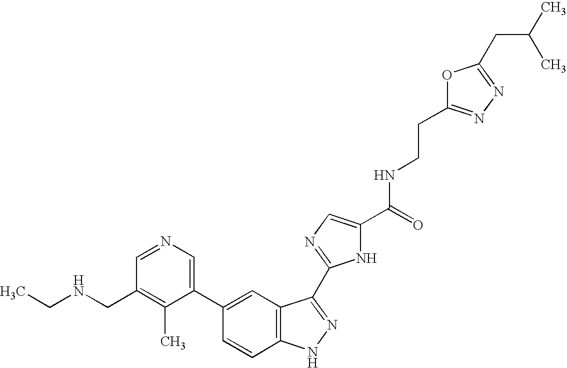 | 2-(5-{5-[(ethylamino)methyl]-4- methylpyridin-3-yl}-1H-indazol-3-yl)-N- [2-(5-isobutyl-1,3,4-oxadiazol-2- yl)ethyl]-1H-imidazole-5-carboxamide | 527.63 | 528.63 | 27 |

### Biochemical and Biological Evaluation

Cyclin-dependent kinase activity was measured by quantifying the enzyme-catalyzed, time-dependent incorporation of radioactive phosphate from [³²P]ATP or [³³P]ATP into a protein substrate. Unless noted otherwise, assays were performed in 96-well plates in a total volume of 50 μL, in the presence of 10 mM HEPES (N-[2-hydroxyethyl]piperazine-N′-[2-ethanesulfonic acid]) (pH 7.4), 10 mM MgCl₂, 25 μM adenosine triphosphate (ATP), 1 mg/mL ovalbumin, 5 μg/mL leupeptin, 1 mM dithiothreitol, 10 mM beta-glycerophosphate, 0.1 mM sodium vanadate, 1 mM sodium fluoride, 2.5 mM ethylene glycol-bis(β-aminoethyl ethKer)-N,N,N′N′-tetraacetic acid (EGTA), 2% (v/v) dimethylsulfoxide, and 0.03-0.4 μCi [^{32/33}P]ATP per reaction. Reactions were initiated with enzyme, incubated at 30° C., and terminated after 20 minutes by the addition of ethylenediaminetetraacetic acid (EDTA) to 250 mM. The phosphorylated substrate was then captured on a nitrocellulose or phosphocellulose membrane using a 96-well filtration manifold, and unincorporated radioactivity was removed by repeated washing with 0.85% phosphoric acid. Radioactivity was quantified by exposing the dried membranes to a phosphorimager.

Apparent Kᵢ values were measured by assaying enzyme activity in the presence of different inhibitor compound concentrations and subtracting the background radioactivity measured in the absence of enzyme. Inhibition data were fit to an equation for competitive inhibition using Kaleidagraph (Synergy Software), or were fit to an equation for competitive tight-binding inhibition using the software KineTic (BioKin, Ltd.).

### Inhibition of CDK4/Cyclin D Retinoblastoma Kinase Activity

A complex of human CDK4 and cyclin D3, or a complex of human CDK4 and genetically truncated (1-264) cyclin D3, was purified using traditional biochemical chromatographic techniques from insect cells that had been co-infected with the corresponding baculovirus expression vectors (see e.g., Meijer and Kim, “Chemical Inhibitors of Cyclin-Dependent Kinases,” Methods in Enzymol, vol. 283 (1997), pp. 113-128.). The enzyme complex (5 or 50 nM) was assayed with 0.3-0.5 μg of purified recombinant retinoblastoma protein fragment (Rb) as a substrate. The engineered Rb fragment (residues 386-928 of the native retinoblastoma protein; 62.3 kDa) contains the majority of the phosphorylation sites found in the native 106-kDa protein, as well as a tag of six histidine residues for ease of purification. Phosphorylated Rb substrate was captured by microfiltration on a nitrocellulose membrane and quantified using a phosphorimager as described above. For measurement of tight-binding inhibitors, the enzyme complex concentration was lowered to 5 nM, and the assay duration was extended to 60 minutes, during which the time-dependence of product formation was linear.

### Inhibition of CDK2/Cyclin A Retinoblastoma Kinase Activity

CDK2 was purified using published methodology (Rosenblatt et al., “Purification and Crystallization of Human Cyclin-dependent Kinase 2,” J. Mol. Biol., vol. 230,1993, pp. 1317-1319) from insect cells that had been infected with a baculovirus expression vector. Cyclin A was purified from E. coli cells expressing full-length recombinant cyclin A, and a truncated cyclin A construct was generated by limited proteolysis and purified as described previously (Jeffrey et al., “Mechanism of CDK activation revealed by the structure of a cyclin A-CDK2 complex,” Nature, vol. 376 (Jul. 27, 1995), pp. 313-320). A complex of CDK2 and proteolyzed cyclin A was prepared and purified by gel filtration. The substrate for this assay was the same Rb substrate fragment used for the CDK4 assays, and the methodology of the CDK2/cyclin A and the CDK4/cyclin D3 assays was essentially the same, except that CDK2 was present at 150 nM or 5 nM. Kᵢ values were measured as described above.

The stimulation of cell proliferation by growth factors such as VEGF and others is dependent upon their induction of autophosphorylation of each of their respective receptor's tyrosine kinases. Therefore, the ability of a protein kinase inhibitor to block cellular proliferation induced by these growth factors is directly correlated with its ability to block receptor autophosphorylation. To measure the protein kinase inhibition activity of the compounds, the following constructs were used.

### Coupled Spectrophotometric (FAK) Assay

Tyrosine kinase assays were monitored using a Beckman DU 650 Spectrophotometer. Production of ADP was coupled to oxidation of NADH using phosphoenolpyruvate (PEP) through the actions of pyruvate kinase (PK) and lactic dehydrogenase (LDH). The oxidation of NADH was monitored by following the decrease in absorbance at 340 nm (ε₃₄₀=6.22 cm⁻¹ mM⁻¹). Typical reaction solutions contained: 1 mM PEP, 250 μM NADH, 50 units of LDH/mL, 20 units of PK/mL, 5 mM DTT, in 200 mM Hepes, pH 7.5 and varying concentrations of poly(E₄Y₁), ATP and MgCl₂. Assays were initiated with 40 nM of cdFGFR1.

Results of assays performed on compounds, which include the specific examples described above are provided in Examples 99-274 above, and in Table 1 below. Unless indicated otherwise in a particular entry, the units and assays used are as indicated in the applicable column of the table.

### Inhibition of Cell Growth: Assessment of Cytotoxicity

Inhibition of cell growth was measured using the tetrazolium salt assay, which is based on the ability of viable cells to reduce 3-(4,5-dimethylthiazol-2-yl)-2,5-[2H]-diphenyltetrazolium bromide (MTT) to formazan (Mossman, Journal of Immunological Methods, vol. 65 (1983), pp. 55-58). The water-insoluble purple formazan product was then detected spectrophotometrically. The HCT 116 cell line was grown in 96-well plates. Cells were plated in the appropriate medium at a volume of 135 μl/well in McCoy's 5A Medium. Plates were incubated for four hours before addition of inhibitor compounds. Different concentrations of inhibitor compounds were added in 0.5% (v/v) dimethylsulfoxide (15 μL/well), and cells were incubated at 37° C. (5% CO₂) for four to six days (depending on cell type). At the end of the incubation, MTT was added to a final concentration of 0.2 mg/mL, and cells were incubated for 4 hours more at 37° C. After centrifugation of the plates and removal of medium, the absorbance of the formazan (solubilized in dimethylsulfoxide) was measured at 540 nm. The concentration of inhibitor compound causing 50% inhibition of growth was determined from the linear portion of a semi-log plot of inhibitor concentration versus percentage inhibition. All results were compared to control cells treated only with 0.5% (v/v) dimethylsulfoxide.

The examples above illustrate compounds according to Formula I and assays that may readily be performed to determine their activity levels against the various kinase complexes. It will be apparent that such assays or other suitable assays known in the art may be used to select an inhibitor having a desired level of activity against a selected target.

### Measurement of Kinetic Solubility

Kinetic solubility was determined with a 30 mm stock solution from the compound(s) of interest. One standard, in a 5% dmso/acetonitirile solution, and one sample, in 100 mm sodium phosphate pH 6.5 buffer, was prepared using a Zymark ALH 3000 liquid handler at a target concentration of 120 μg/mL. Samples and standards were agitated for 30 minutes and allowed to equilibrate at room temperature for 4-6 hours. Samples and standards were then centrifuged and an aliquot was removed for HPLC analysis. Samples and standards were analyzed by HPLC and a final concentration for the sample was determined based on a one-point calibration curve from the corresponding standard. Results were reported as μg's/mL. The dynamic range of the assay and reporting format was <10 μg/mL, while quantitative results varied between 10 and >120 μg'/mL.

TABLE 1

| Example No. | CDK2 Ki | solubility |
|---|---|---|
| 1 | ++ | ++ |
| 2 | + | + |
| 3 | + | + |
| 4 | + | + |
| 5 | ++ | + |
| 6 | + | NI |
| 7 | + | + |
| 8 | + | + |
| 9 | − | + |
| 10 | − | + |
| 11 | + | ++ |
| 12 | + | NI |
| 13 | − | + |
| 14 | − | ++ |
| 15 | − | ++ |
| 16 | − | NI |
| 17 | + | NI |
| 18 | − | NI |
| 19 | ++ | + |
| 20 | ++ | + |
| 21 | ++ | NI |
| 22 | ++ | ++ |
| 23 | ++ | ++ |
| 24 | ++ | ++ |
| 25 | ++ | + |
| 26 | ++ | − |
| 27 | ++ | − |
| 28 | ++ | + |
| 29 | ++ | + |
| 30 | ++ | + |
| 31 | ++ | + |
| 32 | ++ | ++ |
| 33 | ++ | NI |
| 34 | ++ | NI |
| 35 | ++ | + |
| 36 | ++ | NI |
| 37 | ++ | NI |
| 38 | ++ | NI |
| 39 | ++ | NI |
| 40 | ++ | NI |
| 41 | ++ | ++ |
| 42 | + | ++ |
| 43 | + | + |
| 44 | + | ++ |
| 45 | ++ | ++ |
| 46 | ++ | ++ |
| 47 | ++ | ++ |
| 48 | ++ | ++ |
| 49 | ++ | ++ |
| 50 | ++ | + |
| 51 | ++ | + |
| 52 | ++ | NI |
| 53 | ++ | + |
| 54 | ++ | ++ |
| 55 | ++ | ++ |
| 56 | ++ | NI |
| 57 | ++ | NI |
| 58 | ++ | NI |
| 59 | ++ | NI |
| 60 | ++ | NI |
| 61 | ++ | + |
| 62 | ++ | ++ |
| 63 | ++ | ++ |
| 64 | ++ | ++ |
| 65 | ++ | ++ |
| 66 | ++ | ++ |
| 67 | ++ | + |
| 68 | ++ | − |
| 69 | ++ | + |
| 70 | ++ | NI |
| 71 | ++ | + |
| 72 | ++ | − |
| 73 | + | ++ |
| 74 | ++ | ++ |
| 75 | ++ | ++ |
| 76 | + | ++ |
| 77 | − | ++ |
| 78 | ++ | NI |
| 79 | ++ | NI |
| 80 | ++ | NI |
| 81 | ++ | NI |
| 82 | ++ | NI |
| 83 | ++ | ++ |
| 84 | + | NI |
| 85 | ++ | ++ |
| 86 | + | ++ |
| 87 | + | ++ |
| 88 | − | NI |
| 89 | ++ | NI |
| 90 | ++ | ++ |
| 91 | − | NI |
| 92 | ++ | NI |
| 93 | ++ | NI |
| 94 | ++ | + |
| 95 | ++ | + |
| 96 | + | ++ |
| 97 | + | ++ |

CDK2 Ki ++ <50 nM + 50-200 nM − >200 nM
solubility (pH 6.5) ++ >120 ug/ml + 20-120 ug/ml − <20 ug/ml
NI = not indicated

The exemplary compounds described above may be formulated into pharmaceutical compositions according to the following general examples.

### Parenteral Composition

To prepare a parenteral pharmaceutical composition suitable for administration by injection, 100 mg of a water-soluble salt of a compound of Formula I is dissolved in DMSO and then mixed with 10 mL of 0.9% sterile saline. The mixture is incorporated into a dosage unit form suitable for administration by injection.

### Oral Composition

To prepare a pharmaceutical composition for oral delivery, 100 mg of a compound of Formula I is mixed with 750 mg of lactose. The mixture is incorporated into an oral dosage unit for, such as a hard gelatin capsule, which is suitable for oral administration.

While the invention has been illustrated by reference to specific and preferred embodiments, those skilled in the art will recognize that variations and modifications may be made through routine experimentation and practice of the invention. Thus, the invention is intended not to be limited by the foregoing description, but to be defined by the appended claims and their equivalents.

1. A compound or pharmaceutically acceptable salt or solvate of the Formula I: 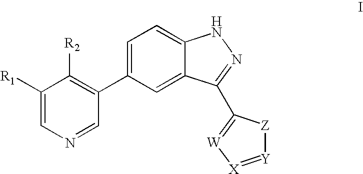
wherein W is —C— or —N—;
X and Y are independently —N—, —C—R³, —C—R⁴;
Z is —C—, —NH—, —O—, or —S—;
wherein R¹ and R² are selected from the group consisting of H, C₁-C₆alkyl, NR⁹R¹⁰, (C₁-C₆alkyl)NR⁹R¹⁰, OR⁹, (C₁-C₆alkyl)OR⁹, and R¹ and R² may together optionally cyclize to form a C₃-C₁₀cycloalkyl or a 4-10 membered heterocyclic;
wherein R³ and R⁴ are independently H, halo, cyano, nitro, trifluoromethoxy, trifluoromethyl, azido, hydroxy, or a group, optionally substituted with at least one R⁹, selected from C₁-C₆ alkoxy, C₁-C₁₀ alkyl, C₂-C₆ alkenyl, C₂-C₆ alkynyl, —C(O)R⁵, C(O)OR⁵, OC(O)R⁵, NR⁵C(O)R⁶, C(O)NR⁷R⁸, (CR⁵R⁶)ₜNR⁷R⁸, NR⁵OR⁶, —SO₂NR⁷R⁸, S(O)ⱼ(C₁-C₆ alkyl) wherein j is an integer from 0 to 2, (CR⁵R⁶)ₜ(C₆-C₁₀ aryl), (CR⁵R⁶)ₜ(C₃-C₁₀cycloalkyl), (CR⁵R⁶)ₜ(4-10 membered heterocyclic), (CR⁵R⁶)_{q}C(O)(CR⁷R⁸)ₜ(C₆-C₁₀ aryl), (CR⁵R⁶)_{q}C(O)(CR⁷R⁸)ₜ(C₃-C₁₀ cycloalkyl), (CR⁵R⁶)_{q}C(O)(CR⁷ R⁸)ₜ(4-10 heterocyclic), (CR⁵R⁶)ₜO(CR⁷R⁸)_{q}(C₆-C₁₀ aryl), (CR⁵R⁶)ₜO(CR⁷R⁸)_{q}(C₃-C₁₀ cycloalkyl), (CR⁵R⁶)ₜO(CR⁷R⁸)_{q}(4-10 membered heterocyclic), (CR⁵R⁶)_{q}SO₂(CR⁷R⁸)ₜ(C₆-C₁₀ aryl), (CR⁵R⁶)_{q}SO₂(CR⁷R⁸)ₜ(C₃-C₁₀ cycloalkyl) and (CR⁵R⁶)_{q}SO₂(CR⁷R⁸)ₜ(4-10 membered heterocyclic), wherein q and t are each independently an integer from 0 to 5, wherein when R³ and R⁴ are each attached to different carbons, may together optionally cyclize to form a fused 6-membered cycloalkyl ring; wherein R¹, R², R³, and R⁴ are not H at the same time;
wherein each R⁵, R⁶, R⁷ and R⁸ is independently H or a group, optionally substituted with at least one R⁹, selected from C₁-C₆ alkyl, C₁-C₈alkenyl, OR¹⁰, (C₁-C₆alkyl)-OR¹⁰, NR¹⁰R¹¹, (C₁-C₆alkyl)-NR¹⁰OR¹¹, C(O)NR¹⁰R¹¹, (C₁-C₆alkyl)C(O)NR¹⁰OR¹¹, (C₁-C₆alkyl)-SR¹⁰, aryl, 3-10 membered cycloalkyl, 4-10 membered heterocyclic, (C₁-C₆alkyl)-aryl, (C₁-C₆alkyl)-cycloalkyl, and (C₁-C₆alkyl)-4-10 membered heterocyclic, or wherein when R⁷ and R⁸ are both attached to the same N, may together cyclize to form a 4-10 membered heterocyclic;
wherein R⁹ may be halo, CF₃, CN, C₁-C₆alkyl, OR², (C₁-C₆alkyl)-OR¹², COR¹², COOR¹², CONR¹²R¹³, NR¹²R¹³, SR¹², SO₂R¹², NHC(O)CF₃, aryl, haloaryl, O-aryl, (C₁-C₆alkyl)-aryl, or (C₁-C₆alkyl)-4-10 membered heterocyclic;
wherein each R¹⁰, R¹¹, R¹² and R¹³ is independently H, C₁-C₆alkyl, C₁-C₆alkoxyl, C₁-C₆alkenyl, aryl, (C₁-C₆alkyl)-aryl, or when attached to the same N may optionally cyclize to form a 4-10 membered heterocyclic; and
wherein 1 or 2 ring carbon atoms of any of the foregoing cycloalkyl or heterocyclic moieties are optionally substituted with an oxo (═O) moiety.

2. The compound, or pharmaceutically acceptable salt or solvate according to claim 1, wherein W is N, Z is NH, and X and Y are CH, C—R³ or C—R⁴.

3. The compound, or pharmaceutically acceptable salt or solvate according to claim 2, wherein R¹ is ethylaminomethyl and R² is methyl.

4. The compound, or pharmaceutically acceptable salt or solvate according to claim 1, wherein W is N, Z is NH, X is CH, Y is C-R³ where R³is CONR⁷R⁸, R¹ is ethylaminomethyl and R² is ethyl.

5. The compound, or pharmaceutically acceptable salt or solvate according to claim 3, wherein X is C—R³ and Y is C—R⁴.

6. The compound or pharmaceutically acceptable salt or solvate according to claim 5, wherein R³ and R⁴ are C₁-C₆alkyl.

7. A pharmaceutical composition comprising the compound, or pharmaceutically acceptable salt or solvate, according to claim 1 and a pharmaceutically acceptable carrier.

8. A compound, pharmaceutically acceptable salt or solvate selected from the group consisting of 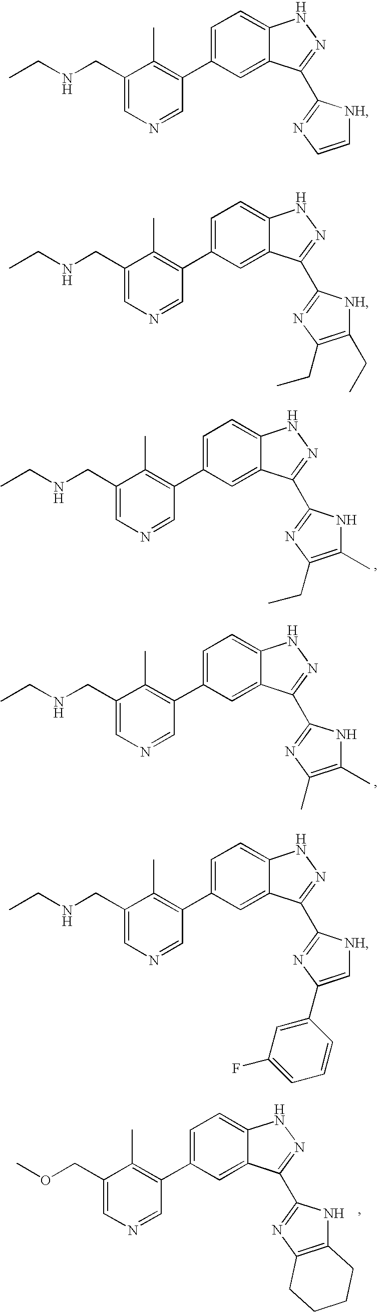 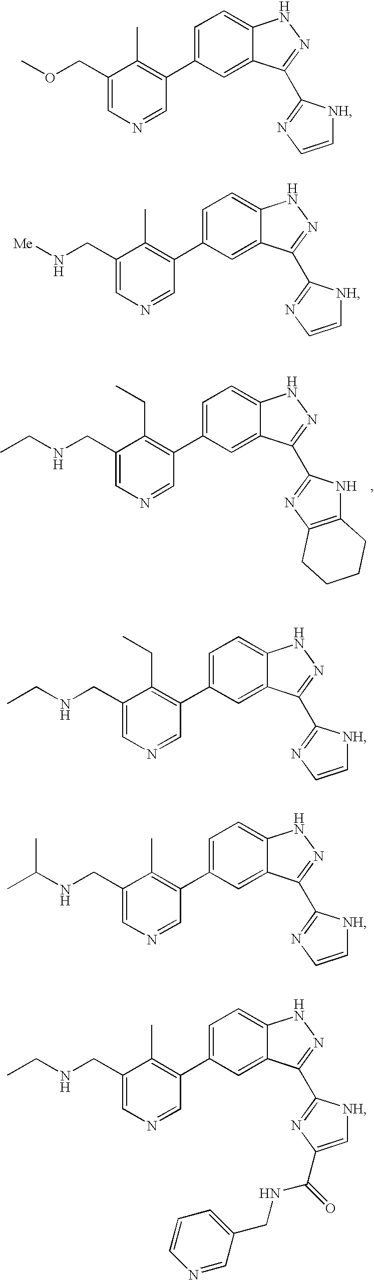 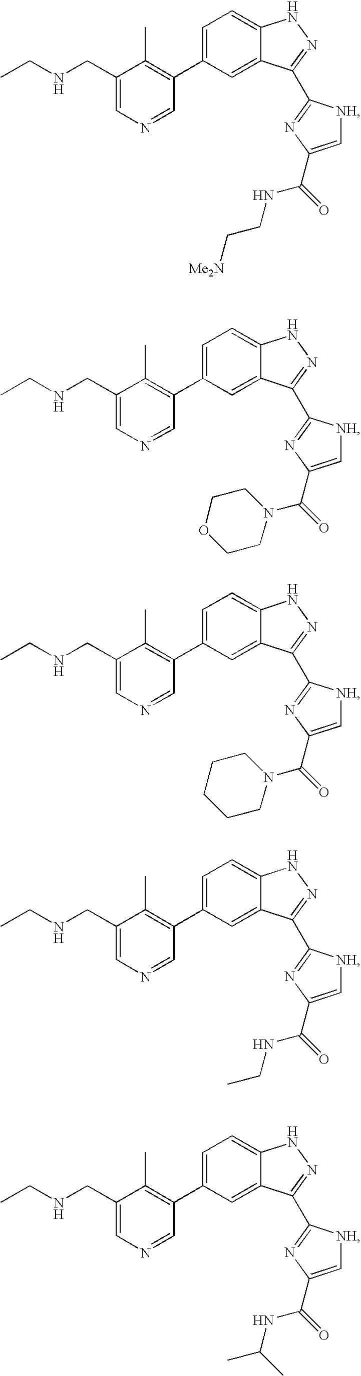 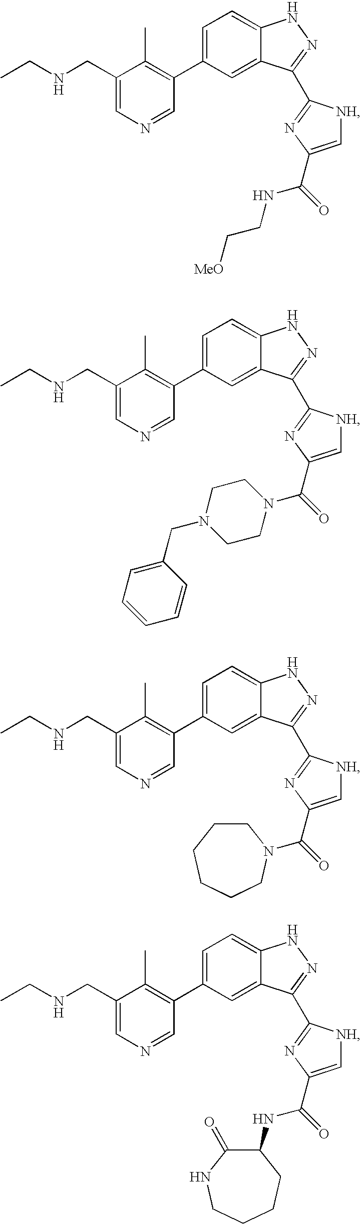 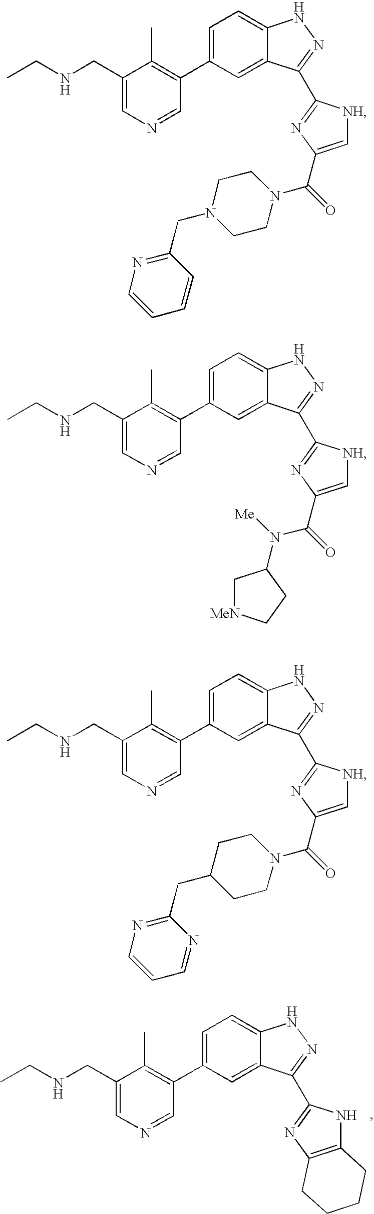 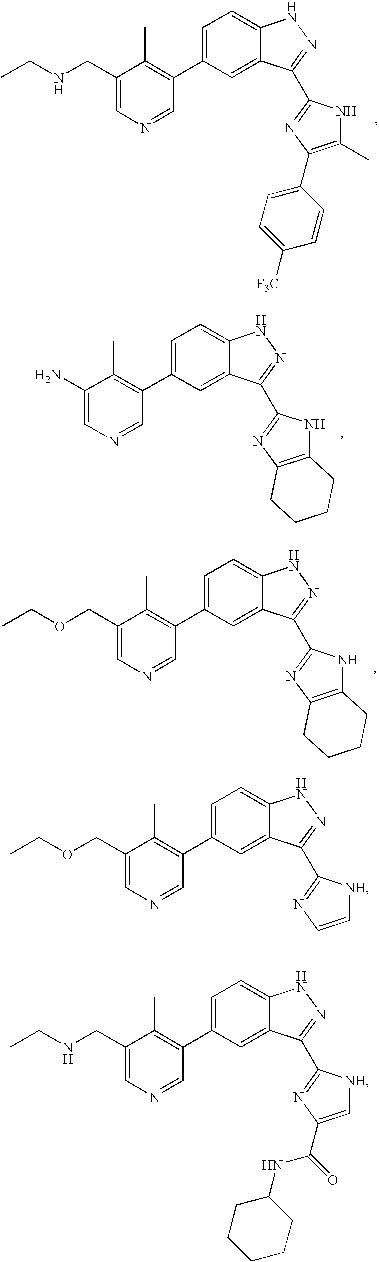 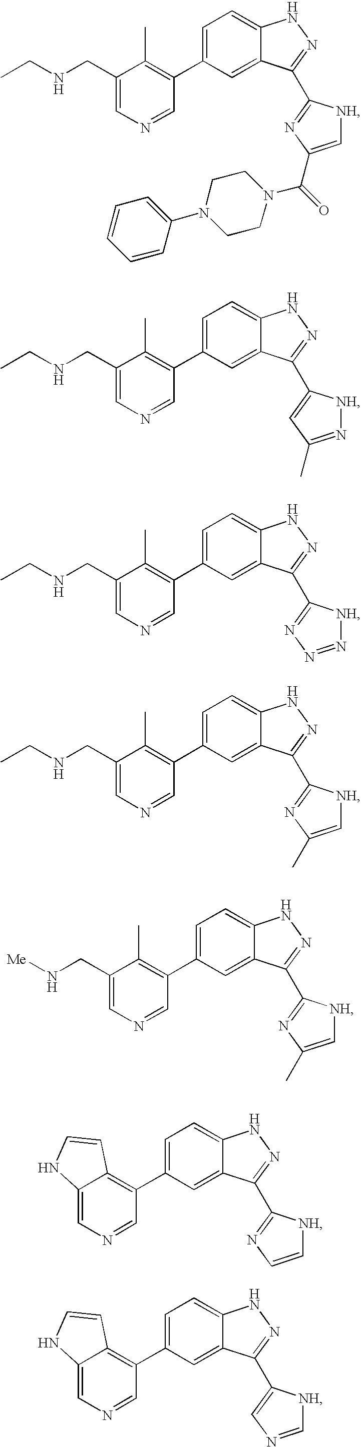 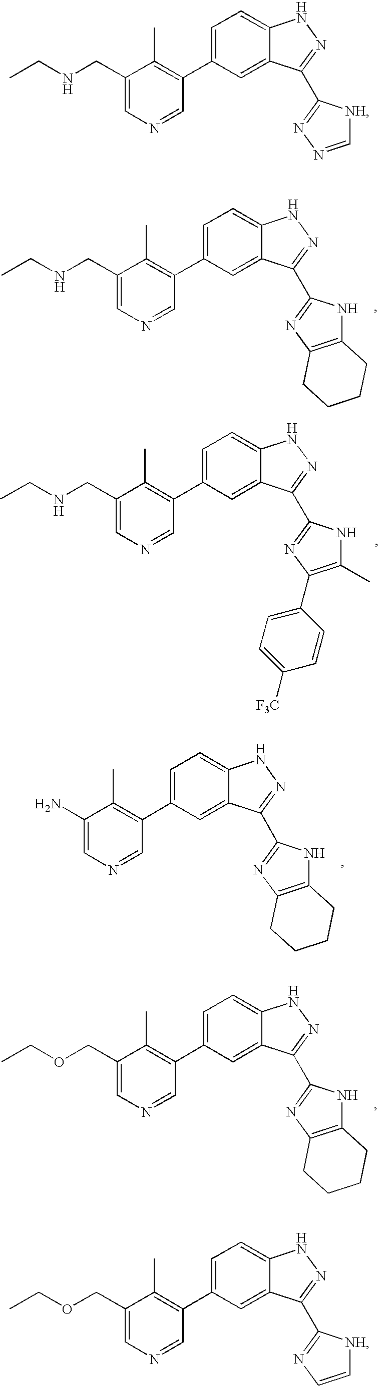 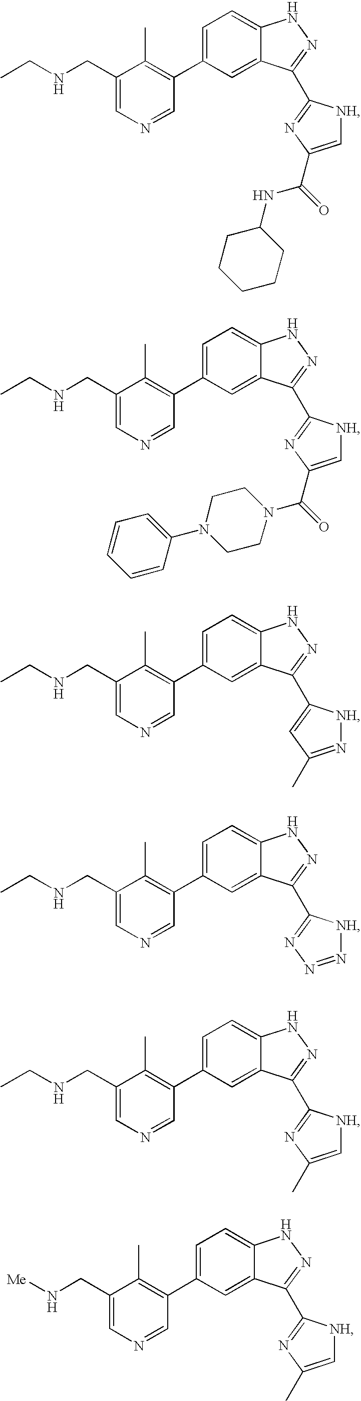 [chemical structure]
